(19) United States
(12) Patent Application Publication
(10) Pub. No.: US 2014/0155415 A1
(43) Pub. Date: Jun. 5, 2014

(54) BENZODIOXEPINE AND BENZODIOXINE COMPOUNDS THAT INTERACT WITH GLUCOKINASE REGULATORY PROTEIN FOR THE TREATMENT OF DIABETES

(71) Applicants: Michael David Bartberger, Sherman Oaks, CA (US); Michael D. Croghan, San Diego, CA (US); Christopher H. Fotsch, Thousand Oaks, CA (US); Mark H. Norman, THousand Oaks, CA (US); Lewis Dale Pennington, Ventura, CA (US); Andreas Reichelt, Moorpark, CA (US); David J. St. Jean, JR., Camarillo, CA (US); Christopher M. Tegley, Thousand Oaks, CA (US)

(72) Inventors: Michael David Bartberger, Sherman Oaks, CA (US); Michael D. Croghan, San Diego, CA (US); Christopher H. Fotsch, Thousand Oaks, CA (US); Mark H. Norman, THousand Oaks, CA (US); Lewis Dale Pennington, Ventura, CA (US); Andreas Reichelt, Moorpark, CA (US); David J. St. Jean, JR., Camarillo, CA (US); Christopher M. Tegley, Thousand Oaks, CA (US)

(21) Appl. No.: 14/110,129

(22) PCT Filed: Apr. 4, 2012
(86) PCT No.: PCT/US12/32192

§ 371 Date: Dec. 11, 2013

Related U.S. Application Data
(60) Provisional application No. 61/472,059, filed on Apr. 5, 2011.

(51) Int. Cl. A61K 31/506; A61K 31/357; C07D 405/14; A61K 31/443; C07D 409/14; A61K 31/155; A61K 31/427; C07D 417/14; C07D 409/12; A61K 31/4436; C07D 495/04; A61K 31/4365; C07D 407/12; A61K 31/381

(52) U.S. Cl. CPC A61K 31/506; C07D 407/12; A61K 31/357; C07D 405/14; A61K 31/443; C07D 409/14; A61K 31/381; A61K 31/427; C07D 417/14; C07D 409/12; A61K 31/4436; C07D 495/04; A61K 31/4365; A61K 31/155
USPC 514/256; 549/350; 514/450; 546/284.1; 514/337; 544/333; 549/60; 514/444; 548/205; 514/365; 549/58; 514/443; 546/281.1; 546/114; 514/301

(57) ABSTRACT

The present invention relates to compounds of formula I or II,

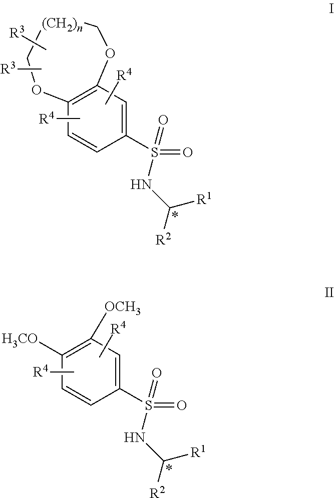

or pharmaceutically acceptable salts thereof, that interact with glucokinase regulatory protein. In addition, the present invention relates to methods of treating type 2 diabetes, and other diseases and/or conditions where glucokinase regulatory protein is involved using the compounds, or pharmaceutically acceptable salts thereof, and pharmaceutical compositions that contain the compounds, or pharmaceutically acceptable salts thereof.

### FIELD OF THE INVENTION

The present invention relates to compounds of formula I, II, III or IV, or pharmaceutically acceptable salts thereof, as defined herein, that interact with glucokinase regulatory protein. In addition, the present invention relates to methods of treating type 2 diabetes, and other diseases and/or conditions where glucokinase regulatory protein is involved using the compounds, or the pharmaceutically acceptable salts thereof, and pharmaceutical compositions that contain the compounds, or pharmaceutically acceptable salts thereof.

### BACKGROUND OF THE INVENTION

Glucokinase (GK) is a member of a family of four hexokinases that are critical in the cellular metabolism of glucose. Specifically GK, also known as hexokinase IV or hexokinase D, facilitates glucose induced insulin secretion from pancreatic β-cells as well as glucose conversion into glycogen in the liver. GK has a unique catalytic activity that enables the enzyme to be active within the physiological range of glucose (from 5 mM glucose to 10 mM glucose).

Genetically modified mouse models support the role of GK playing an important role in glucose homeostasis. Mice lacking both copies of the GK gene die soon after birth from severe hyperglycemia, whereas mice lacking only one copy of the GK gene present with only mild diabetes. Mice that are made to overexpress the GK gene in their livers are hypoglycemic.

Numerous human mutations in the GK gene have been identified, with the vast majority of them resulting in proteins with impaired or absent enzymatic activity. These loss-of-function mutations are thought to contribute to the hyperglycemia seen with maturity-onset diabetes of the young type II (MODY-2). A small fraction of these mutations result in a GK with increased catalytic function. These individuals present with moderate to severe hypoglycemia.

GK activity in the liver is transiently regulated by glucokinase regulatory protein (GKRP). GK catalytic activity is inhibited when GK is bound to GKRP. This interaction is antagonized by increasing concentrations of both glucose and fructose-1-phosphate (F1P). The complex of the two proteins is localized primarily to the nuclear compartment of a cell. Post prandially as both glucose and fructose levels rise, GK released from GKRP translocates to the cytoplasm. Cytoplasmic GK is now free of the inhibitory effects of GKRP and able to kinetically respond to glucose. Evidence from the Zucker diabetic fatty rat (ZDF) indicates that their glucose intolerance may be a result of this mechanism failing to function properly.

A compound that acts directly on GKRP to disrupt its interaction with GK and hence elevate levels of cytoplasmic GK is a viable approach to modulate GK activity. Such an approach would avoid the unwanted hypoglycemic effects of over stimulation of GK catalytic activity, which has been seen in the development of GK activators. A compound having such an effect would be useful in the treatment of diabetes and other diseases and/or conditions in which GKRP and/or GK plays a role. The present invention provides compounds that bind GKRP and disrupts its interaction with GK.

### SUMMARY OF THE INVENTION

In aspect 1, the present invention provides compounds of formula I or II, or pharmaceutically acceptable salts thereof, wherein:

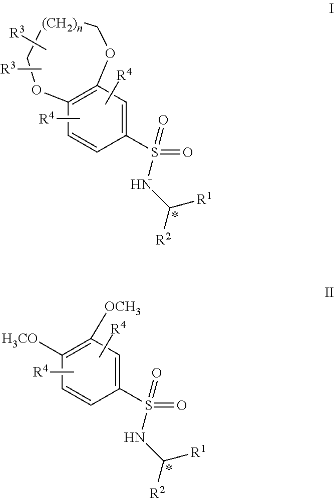

the symbol * represents a chiral center that has the R configuration or is a mixture of the R and S configurations;

n is 0 or 1;

R¹ is a five or six membered monocyclic aryl group or monocyclic heteroaryl group, wherein the heteroatoms are independently selected from N, O or S, which groups can be optionally substituted at the (1) positions with respect to its point of attachment to the rest of the compound, when possible, with a substituent selected from —C₁₋₈alkyl, —C₁₋₈alkoxy, halo, —SC₁₋₈alkyl, —C₂₋₈alkenyl, —C₂₋₈alkynyl, —C₁₋₈hydroxyalkyl, haloalkyl, perfluoroalkyl or —OCF₃; or at the (2) positions, when possible, with a substituent selected from fluorine,

—OH or —NH₂; or at the (3) position, when possible, with fluorine;

R² is a nine or ten membered bicyclic aryl group or bicyclic heteroaryl group, wherein the heteroatoms are independently selected from N, O or S, which groups can be optionally substituted with from 1 to 7 substituents independently selected from halo, C₁₋₆alkyl, C₁₋₆alkoxy, —OH or a five or six membered aryl or heteroaryl group, which aryl or heteroaryl group that can be optionally substituted with from 1 to 3 substituents independently selected from C₁₋₆ alkyl, C₁₋₆alkoxy, —OH, —NH₂, —C(CF₃)(OH)(CF₃) or —C(CF₃)(OH)(CH₃);

each R³ is independently hydrogen, —OH or halo when n is 1, or hydrogen, —OH, halo or —CH₃ when n is 0; and

each R⁴ is independently hydrogen or halo;

provided that the compound is not N-(1-benzofuran-2-yl(phenyl)methyl)-3,4-dihydro-2H-1,5-benzodioxepine-7-sulfonamide.

In aspect 2, the present invention provides compounds in accordance with aspect 1, or pharmaceutically acceptable salts thereof, wherein:

the compounds have the formula III or IV;

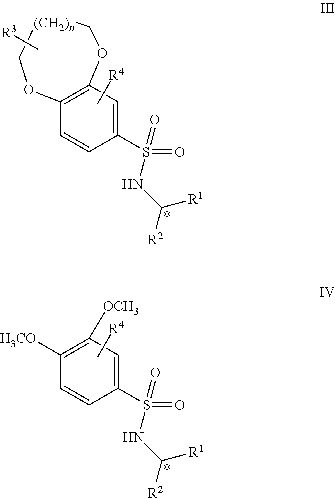

R² is a nine or ten membered bicyclic aryl group or bicyclic heteroaryl group, wherein the heteroatoms are independently selected from N, O or S, which groups can be optionally substituted with from 1 to 7 substituents independently selected from C₁₋₆alkyl, C₁₋₆alkoxy, —OH or a five or six membered aryl or heteroaryl group, which aryl or heteroaryl group that can be optionally substituted with from 1 to 3 substituents independently selected from C₁₋₆ alkyl, C₁₋₆alkoxy, —OH, —NH₂, or —C(CF₃)(OH)(CH₃);

R³ is hydrogen or halo when n is 1, or hydrogen, halo, or —CH₃ when n is 0; and

R⁴ is hydrogen or halo.

In aspect 3, the present invention provides compounds of aspects 1 or 2, or pharmaceutically acceptable salts thereof, wherein R¹ is phenyl, substituted phenyl, pyridyl, substituted pyridyl, thienyl, substituted thienyl, pyrimidinyl or is substituted pyrimidinyl.

In aspect 4, the present invention provides compounds of aspects 1 or 2, or pharmaceutically acceptable salts thereof, wherein R² is benzofuranyl, substituted benzofuranyl, benzothiophenyl, substituted benzothiophenyl, benzothiazolyl, substituted benzothiazolyl, thieno[2,3-c]pyridinyl, substituted thieno[2,3-c]pyridinyl, benzoxazolyl, substituted benzoxazolyl, thieno[3,2-c]pyridinyl, substituted, thieno[3,2-c]pyridinyl, quinolinyl, substituted quinolinyl, naphthalenyl, substituted naphthalenyl, thieno[3,2-b]pyridinyl, substituted thieno[3,2-b]pyridinyl, thieno[2,3-b]pyridinyl, substituted thieno[2,3-b]pyridinyl, [1,3]thiazolo[5,4-b]pyridinyl, or substituted [1,3]thiazolo[5,4-b]pyridinyl.

In aspect 5, the present invention provides compounds of aspects 1 or 2, or pharmaceutically acceptable salts thereof, wherein R³ is hydrogen or halogen.

In aspect 6, the present invention provides compounds of aspects 1 or 2, or pharmaceutically acceptable salts thereof, wherein R⁴ is hydrogen.

In aspect 7, the present invention provides compounds of aspects 1 or 2, or pharmaceutically acceptable salts thereof, that have the R configuration at the stereocenter designated with a *.

In aspect 8, the present invention provides compounds of aspects 1 or 2, or pharmaceutically acceptable salts thereof, that is a mixture of the R and S configurations at the stereocenter designated with a *.

In aspect 9, the present invention provides compounds of aspects 1 or 2, or to pharmaceutically acceptable salts thereof, that is a racemic mixture of the R and S configurations at the stereocenter designated with a *.

In aspect 10, the present invention provides compounds of aspects 1 or 2, or pharmaceutically acceptable salts thereof, wherein:

R¹ is phenyl, substituted phenyl, pyridyl, substituted pyridyl, thienyl, substituted thienyl, pyrimidinyl or substituted pyrimidinyl; and

R² is benzofuranyl, substituted benzofuranyl, benzothiophenyl, substituted benzothiophenyl, benzothiazolyl, substituted benzothiazolyl, thieno[2,3-c]pyridinyl, substituted thieno[2,3-c]pyridinyl, benzoxazolyl, substituted benzoxazolyl, thieno[3,2-c]pyridinyl, substituted, thieno[3,2-c]pyridinyl, quinolinyl, substituted quinolinyl, naphthalenyl, substituted naphthalenyl, thieno[3,2-b]pyridinyl, substituted thieno[3,2-b]pyridinyl, thieno[2,3-b]pyridinyl, substituted thieno[2,3-b]pyridinyl, [1,3]thiazolo[5,4-b]pyridinyl, or substituted [1,3]thiazolo[5,4-b]pyridinyl.

In aspect 11, the present invention provides compounds of aspects 1 or 2, or pharmaceutically acceptable salts thereof, wherein:

R¹ is phenyl, substituted phenyl, pyridyl, substituted pyridyl, thienyl, substituted thienyl, pyrimidinyl or substituted pyrimidinyl;

R² is benzofuranyl, substituted benzofuranyl, benzothiophenyl, substituted benzothiophenyl, benzothiazolyl, substituted benzothiazolyl, thieno[2,3-c]pyridinyl, substituted thieno[2,3-c]pyridinyl, benzoxazolyl, substituted benzoxazolyl, thieno[3,2-c]pyridinyl, substituted, thieno[3,2-c]pyridinyl, quinolinyl, substituted quinolinyl, naphthalenyl, substituted naphthalenyl, thieno[3,2-b]pyridinyl, substituted thieno[3,2-b]pyridinyl, thieno[2,3-b]pyridinyl, substituted thieno[2,3-b]pyridinyl, [1,3]thiazolo[5,4-b]pyridinyl, or substituted [1,3]thiazolo[5,4-b]pyridinyl.

R³ is hydrogen or halogen;

R⁴ is hydrogen; and the compound has the R configuration at the stereocenter designated with a *.

In aspect 12, the present invention provides compounds, or pharmaceutically acceptable salts, selected from:
N-(1-benzofuran-2-yl(2-methylphenyl)methyl)-3,4-dihydro-2H-1,5-benzodioxepine-7-sulfonamide;
N-(1-benzofuran-2-yl(4-fluoro-2-methylphenyl)methyl)-3,4-dihydro-2H-1,5-benzodioxepine-7-sulfonamide;
N-(1-benzofuran-2-yl(5-fluoro-2-methylphenyl)methyl)-3,4-dihydro-2H-1,5-benzodioxepine-7-sulfonamide;
N-(1-benzofuran-2-yl(2-fluoro-6-methylphenyl)methyl)-3,4-dihydro-2H-1,5-benzodioxepine-7-sulfonamide;
N-(1-benzofuran-2-yl(2-ethylphenyl)methyl)-3,4-dihydro-2H-1,5-benzodioxepine-7-sulfonamide;
N-(1-benzofuran-2-yl(2-ethenylphenyl)methyl)-3,4-dihydro-2H-1,5-benzodioxepine-7-sulfonamide;
N-(1-benzofuran-2-yl(2-ethynylphenyl)methyl)-3,4-dihydro-2H-1,5-benzodioxepine-7-sulfonamide;
N-(1-benzofuran-2-yl(2-(1-methylethyl)phenyl)methyl)-3,4-dihydro-2H-1,5-benzodioxepine-7-sulfonamide;
N-(1-benzofuran-2-yl (2,6-dimethylphenyl)methyl)-3,4-dihydro-2H-1,5-benzodioxepine-7-sulfonamide;
N-(1-benzofuran-2-yl(2-propylphenyl)methyl)-3,4-dihydro-2H-1,5-benzodioxepine-7-sulfonamide;
N-(1-benzofuran-2-yl(2-(trifluoromethyl)phenyl)methyl)-3,4-dihydro-2H-1,5-benzodioxepine-7-sulfonamide;
N-(1-benzofuran-2-yl(3-hydroxyphenyl)methyl)-3,4-dihydro-2H-1,5-benzodioxepine-7-sulfonamide;
N-((3-aminophenyl)(1-benzofuran-2-yl)methyl)-3,4-dihydro-2H-1,5-benzodioxepine-7-sulfonamide;
N-(1-benzofuran-2-yl(2-(hydroxymethyl)phenyl)methyl)-3,4-dihydro-2H-1,5-benzodioxepine-7-sulfonamide;
N-(1-benzofuran-2-yl(2-(2-hydroxyethyl)phenyl)methyl)-3,4-dihydro-2H-1,5-benzodioxepine-7-sulfonamide;
N-(1-benzofuran-2-yl(2-methoxyphenyl)methyl)-3,4-dihydro-2H-1,5-benzodioxepine-7-sulfonamide;
N-(1-benzofuran-2-yl(2-(trifluoromethoxy)phenyl)methyl)-3,4-dihydro-2H-1,5-benzodioxepine-7-sulfonamide;
N-(1-benzofuran-2-yl(2-(methylsulfanyl)phenyl)methyl)-3,4-dihydro-2H-1,5-benzodioxepine-7-sulfonamide;
N-(1-benzofuran-2-yl(2-chlorophenyl)methyl)-3,4-dihydro-2H-1,5-benzodioxepine-7-sulfonamide;
N-(1-benzofuran-2-yl(2-iodophenyl)methyl)-3,4-dihydro-2H-1,5-benzodioxepine-7-sulfonamide;
N-(1-benzofuran-2-yl(2-bromophenyl)methyl)-3,4-dihydro-2H-1,5-benzodioxepine-7-sulfonamide;
N-(1-benzofuran-2-yl(2-fluorophenyl)methyl)-3,4-dihydro-2H-1,5-benzodioxepine-7-sulfonamide;
N-(1-benzofuran-2-yl(3-fluorophenyl)methyl)-3,4-dihydro-2H-1,5-benzodioxepine-7-sulfonamide;
N-(1-benzofuran-2-yl(2-pyridinyl)methyl)-3,4-dihydro-2H-1,5-benzodioxepine-7-sulfonamide;
N-(1-benzofuran-2-yl(3-methyl-2-pyridinyl)methyl)-3,4-dihydro-2H-1,5-benzodioxepine-7-sulfonamide;
N-(1-benzofuran-2-yl(2-chloro-3-pyridinyl)methyl)-3,4-dihydro-2H-1,5-benzodioxepine-7-sulfonamide;
N-(1-benzofuran-2-yl(3-chloro-4-pyridinyl)methyl)-3,4-dihydro-2H-1,5-benzodioxepine-7-sulfonamide;
N-(1-benzofuran-2-yl(2-pyrimidinyl)methyl)-3,4-dihydro-2H-1,5-benzodioxepine-7-sulfonamide;
N-(1-benzofuran-2-yl(3-methoxy-2-pyridinyl)methyl)-3,4-dihydro-2H-1,5-benzodioxepine-7-sulfonamide;
N-(1-benzofuran-2-yl(2-thiophenyl)methyl)-3,4-dihydro-2H-1,5-benzodioxepine-7-sulfonamide;
N-(1-benzofuran-2-yl(3-thiophenyl)methyl)-3,4-dihydro-2H-1,5-benzodioxepine-7-sulfonamide;
N-(1-benzofuran-2-yl(3-methyl-2-thiophenyl)methyl)-3,4-dihydro-2H-1,5-benzodioxepine-7-sulfonamide;
N-(1-benzofuran-2-yl(1,3-thiazol-2-yl)methyl)-3,4-dihydro-2H-1,5-benzodioxepine-7-sulfonamide;
N-(1-benzofuran-2-yl(phenyl)methyl)-3-fluoro-3,4-dihydro-2H-1,5-benzodioxepine-7-sulfonamide;
N-(1-benzothiophen-2-yl(2-methylphenyl)methyl)-3,4-dihydro-2H-1,5-benzodioxepine-7-sulfonamide;
N-(1-benzothiophen-2-yl(2-ethylphenyl)methyl)-3,4-dihydro-2H-1,5-benzodioxepine-7-sulfonamide;
N-(1-benzothiophen-2-yl(2-ethenylphenyl)methyl)-3,4-dihydro-2H-1,5-benzodioxepine-7-sulfonamide;
N-(1-benzothiophen-2-yl(2-ethynylphenyl)methyl)-3,4-dihydro-2H-1,5-benzodioxepine-7-sulfonamide;
N-(1-benzothiophen-2-yl(2-methoxyphenyl)methyl)-3,4-dihydro-2H-1,5-benzodioxepine-7-sulfonamide;
N-(1-benzothiophen-2-yl(2-(methylsulfanyl)phenyl)methyl)-3,4-dihydro-2H-1,5-benzodioxepine-7-sulfonamide;
N-(1-benzothiophen-2-yl(2-chlorophenyl)methyl)-3,4-dihydro-2H-1,5-benzodioxepine-7-sulfonamide;
N-(1-benzothiophen-2-yl(3-methoxy-2-pyridinyl)methyl)-3,4-dihydro-2H-1,5-benzodioxepine-7-sulfonamide;
N-((3-methyl-1-benzofuran-2-yl)(phenyl)methyl)-3,4-dihydro-2H-1,5-benzodioxepine-7-sulfonamide;
N-((3-methyl-1-benzofuran-2-yl)(2-methylphenyl)methyl)-3,4-dihydro-2H-1,5-benzodioxepine-7-sulfonamide;
N-(phenyl(thieno[2,3-b]pyridin-2-yl)methyl)-3,4-dihydro-2H-1,5-benzodioxepine-7-sulfonamide;
N-((2-methoxyphenyl)(thieno[2,3-b]pyridin-2-yl)methyl)-3,4-dihydro-2H-1,5-benzodioxepine-7-sulfonamide;
N-((2-(methylsulfanyl)phenyl)(thieno[2,3-b]pyridin-2-yl)methyl)-3,4-dihydro-2H-1,5-benzodioxepine-7-sulfonamide;
N-((3-methoxy-2-pyridinyl)(thieno[2,3-b]pyridin-2-yl)methyl)-3,4-dihydro-2H-1,5-benzodioxepine-7-sulfonamide;
N-((3-methoxy-4-pyridinyl)(thieno[2,3-b]pyridin-2-yl)methyl)-3,4-dihydro-2H-1,5-benzodioxepine-7-sulfonamide;
N-(1,3-benzothiazol-2-yl(2-methoxyphenyl)methyl)-3,4-dihydro-2H-1,5-benzodioxepine-7-sulfonamide;
N-(1,3-benzothiazol-2-yl(2-(methylsulfanyl)phenyl)methyl)-3,4-dihydro-2H-1,5-benzodioxepine-7-sulfonamide;
N-(1,3-benzothiazol-2-yl(3-methoxy-2-pyridinyl)methyl)-3,4-dihydro-2H-1,5-benzodioxepine-7-sulfonamide;
N-(1,3-benzoxazol-2-yl(phenyl)methyl)-3,4-dihydro-2H-1,5-benzodioxepine-7-sulfonamide;
N-(2-naphthalenyl(phenyl)methyl)-3,4-dihydro-2H-1,5-benzodioxepine-7-sulfonamide;
N-(phenyl(2-quinolinyl)methyl)-3,4-dihydro-2H-1,5-benzodioxepine-7-sulfonamide;
N-(1-benzothiophen-2-yl(phenyl)methyl)-3,4-dihydro-2H-1,5-benzodioxepine-7-sulfonamide;
N-((5-methoxy-1-benzothiophen-2-yl)(phenyl)methyl)-3,4-dihydro-2H-1,5-benzodioxepine-7-sulfonamide;
N-(1,3-benzothiazol-2-yl(phenyl)methyl)-3,4-dihydro-2H-1,5-benzodioxepine-7-sulfonamide;
N-(phenyl(thieno[2,3-c]pyridin-2-yl)methyl)-3,4-dihydro-2H-1,5-benzodioxepine-7-sulfonamide;
N-(phenyl(thieno[3,2-b]pyridin-2-yl)methyl)-3,4-dihydro-2H-1,5-benzodioxepine-7-sulfonamide;
N-(phenyl(thieno[3,2-c]pyridin-2-yl)methyl)-3,4-dihydro-2H-1,5-benzodioxepine-7-sulfonamide;
N-((5-hydroxy-1-benzothiophen-2-yl)(phenyl)methyl)-3,4-dihydro-2H-1,5-benzodioxepine-7-sulfonamide;
N-((2-(methylsulfanyl)phenyl)(thieno[2,3-c]pyridin-2-yl)methyl)-3,4-dihydro-2H-1,5-benzodioxepine-7-sulfonamide;
N-((2-(methylsulfanyl)phenyl)([1,3]thiazolo[5,4-b]pyridin-2-yl)methyl)-3,4-dihydro-2H-1,5-benzodioxepine-7-sulfonamide;
N-((2-methoxyphenyl)(thieno[2,3-c]pyridin-2-yl)methyl)-3,4-dihydro-2H-1,5-benzodioxepine-7-sulfonamide;
N-((2-methoxyphenyl)([1,3]thiazolo[5,4-b]pyridin-2-yl)methyl)-3,4-dihydro-2H-1,5-benzodioxepine-7-sulfonamide;
N-((2-chlorophenyl)([1,3]thiazolo[5,4-b]pyridin-2-yl)methyl)-3,4-dihydro-2H-1,5-benzodioxepine-7-sulfonamide;
N-((3-methoxy-2-pyridinyl)([1,3]thiazolo[5,4-b]pyridin-2-yl)methyl)-3,4-dihydro-2H-1,5-benzodioxepine-7-sulfonamide;
N-(phenyl(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N-(phenyl(7-(2-pyridinyl)-1-benzothiophen-2-yl)methyl)-3,4-dihydro-2H-1,5-benzodioxepine-7-sulfonamide;
N-((7-chloro-1-benzothiophen-2-yl)(phenyl)methyl)-3,4-dihydro-2H-1,5-benzodioxepine-7-sulfonamide;
N-(phenyl(7-phenyl-1-benzothiophen-2-yl)methyl)-3,4-dihydro-2H-1,5-benzodioxepine-7-sulfonamide;
N-(1-benzothiophen-2-yl(2-(methylsulfanyl)phenyl)methyl)-3,4-dihydro-2H-1,5-benzodioxepine-7-sulfonamide; or
N-(1-benzothiophen-2-yl(2-(methylsulfanyl)phenyl)methyl)-2,3-dihydro-1,4-benzodioxine-6-sulfonamide.

In aspect 13, the present invention provides compounds, or pharmaceutically acceptable salts, selected from:
N-(1-benzothiophen-2-yl(2-chlorophenyl)methyl)-3,3-difluoro-3,4-dihydro-2H-1,5-benzodioxepine-7-sulfonamide;
N-(1-benzothiophen-2-yl(2-chlorophenyl)methyl)-3-hydroxy-3,4-dihydro-2H-1,5-benzodioxepine-7-sulfonamide;
N-(1-benzothiophen-2-yl(2-chlorophenyl)methyl)-3-fluoro-3,4-dihydro-2H-1,5-benzodioxepine-7-sulfonamide;
N-((6-amino-2-pyridinyl)(1-benzofuran-2-yl)methyl)-3,4-dihydro-2H-1,5-benzodioxepine-7-sulfonamide;
N-((7-chlorothieno[2,3-c]pyridin-2-yl)(phenyl)methyl)-3,4-dihydro-2H-1,5-benzodioxepine-7-sulfonamide;
N-((2-chlorophenyl)(7-(4-(2,2,2-trifluoro-1-hydroxy-1-(trifluoromethyl)ethyl)-2-pyridinyl)-1-benzothiophen-2-yl)methyl)-3,4-dihydro-2H-1,5-benzodioxepine-7-sulfonamide;
N-((2-chlorophenyl)(7-(4-(2,2,2-trifluoro-1-hydroxy-1-(trifluoromethyl)ethyl)-2-pyridinyl)-1-benzothiophen-2-yl)methyl)-3,4-dimethoxybenzenesulfonamide;
N-((2-chlorophenyl)(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N-((2-chlorophenyl)(7-(4-(2,2,2-trifluoro-1-hydroxy-1-methylethyl)-2-pyridinyl)-1-benzothiophen-2-yl)methyl)-3,4-dimethoxybenzenesulfonamide;
N-((2-chlorophenyl)(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N-((2-chlorophenyl)(7-(4-(2,2,2-trifluoro-1-hydroxy-1-methylethyl)-2-pyridinyl)-1-benzothiophen-2-yl)methyl)-3,4-dimethoxybenzenesulfonamide; or
N-((2-amino-4-pyridinyl)(1-benzofuran-2-yl)methyl)-3,4-dihydro-2H-1,5-benzodioxepine-7-sulfonamide.

In aspect 14, the present invention provides compounds, or pharmaceutically acceptable salts, selected from:
N—((R)-1-benzofuran-2-yl(phenyl)methyl)-3,4-dihydro-2H-1,5-benzodioxepine-7-sulfonamide;
N—((S)-1-benzofuran-2-yl(phenyl)methyl)-3,4-dihydro-2H-1,5-benzodioxepine-7-sulfonamide;
N—((R)-1-benzofuran-2-yl(2-methylphenyl)methyl)-3,4-dihydro-2H-1,5-benzodioxepine-7-sulfonamide;
N—((R)-1-benzofuran-2-yl(4-fluoro-2-methylphenyl)methyl)-3,4-dihydro-2H-1,5-benzodioxepine-7-sulfonamide;
N—((R)-1-benzofuran-2-yl(5-fluoro-2-methylphenyl)methyl)-3,4-dihydro-2H-1,5-benzodioxepine-7-sulfonamide;
N—((R)-1-benzofuran-2-yl(2-fluoro-6-methylphenyl)methyl)-3,4-dihydro-2H-1,5-benzodioxepine-7-sulfonamide;
N—((R)-1-benzofuran-2-yl(2-ethylphenyl)methyl)-3,4-dihydro-2H-1,5-benzodioxepine-7-sulfonamide;
N—((R)-1-benzofuran-2-yl(2-ethenylphenyl)methyl)-3,4-dihydro-2H-1,5-benzodioxepine-7-sulfonamide;
N—((R)-1-benzofuran-2-yl(2-ethynylphenyl)methyl)-3,4-dihydro-2H-1,5-benzodioxepine-7-sulfonamide;
N—((R)-1-benzofuran-2-yl(2-(1-methylethyl)phenyl)methyl)-3,4-dihydro-2H-1,5-benzodioxepine-7-sulfonamide;
N—((R)-1-benzofuran-2-yl (2,6-dimethylphenyl)methyl)-3,4-dihydro-2H-1,5-benzodioxepine-7-sulfonamide;
N—((R)-1-benzofuran-2-yl(2-propylphenyl)methyl)-3,4-dihydro-2H-1,5-benzodioxepine-7-sulfonamide;
N—((R)-1-benzofuran-2-yl(2-(trifluoromethyl)phenyl)methyl)-3,4-dihydro-2H-1,5-benzodioxepine-7-sulfonamide;
N—((R)-1-benzofuran-2-yl(3-hydroxyphenyl)methyl)-3,4-dihydro-2H-1,5-benzodioxepine-7-sulfonamide;
N—((R)— (3-aminophenyl)(1-benzofuran-2-yl)methyl)-3,4-dihydro-2H-1,5-benzodioxepine-7-sulfonamide;
N—((R)-1-benzofuran-2-yl(2-(hydroxymethyl)phenyl)methyl)-3,4-dihydro-2H-1,5-benzodioxepine-7-sulfonamide;
N—((R)-1-benzofuran-2-yl(2-(2-hydroxyethyl)phenyl)methyl)-3,4-dihydro-2H-1,5-benzodioxepine-7-sulfonamide;
N—((R)-1-benzofuran-2-yl(2-methoxyphenyl)methyl)-3,4-dihydro-2H-1,5-benzodioxepine-7-sulfonamide;
N—((R)-1-benzofuran-2-yl(2-(trifluoromethoxy)phenyl)methyl)-3,4-dihydro-2H-1,5-benzodioxepine-7-sulfonamide;
N—((R)-1-benzofuran-2-yl(2-(methylsulfanyl)phenyl)methyl)-3,4-dihydro-2H-1,5-benzodioxepine-7-sulfonamide;
N—((R)-1-benzofuran-2-yl(2-chlorophenyl)methyl)-3,4-dihydro-2H-1,5-benzodioxepine-7-sulfonamide;
N—((R)-1-benzofuran-2-yl(2-iodophenyl)methyl)-3,4-dihydro-2H-1,5-benzodioxepine-7-sulfonamide;
N—((R)-1-benzofuran-2-yl(2-bromophenyl)methyl)-3,4-dihydro-2H-1,5-benzodioxepine-7-sulfonamide;
N—((R)-1-benzofuran-2-yl(2-fluorophenyl)methyl)-3,4-dihydro-2H-1,5-benzodioxepine-7-sulfonamide;
N—((R)-1-benzofuran-2-yl(3-fluorophenyl)methyl)-3,4-dihydro-2H-1,5-benzodioxepine-7-sulfonamide;
N—((R)-1-benzofuran-2-yl(2-pyridinyl)methyl)-3,4-dihydro-2H-1,5-benzodioxepine-7-sulfonamide;
N—((R)-1-benzofuran-2-yl(3-methyl-2-pyridinyl)methyl)-3,4-dihydro-2H-1,5-benzodioxepine-7-sulfonamide;
N—((R)-1-benzofuran-2-yl(2-chloro-3-pyridinyl)methyl)-3,4-dihydro-2H-1,5-benzodioxepine-7-sulfonamide;
N—((R)-1-benzofuran-2-yl(3-chloro-4-pyridinyl)methyl)-3,4-dihydro-2H-1,5-benzodioxepine-7-sulfonamide;
N—((R)-1-benzofuran-2-yl(2-pyrimidinyl)methyl)-3,4-dihydro-2H-1,5-benzodioxepine-7-sulfonamide;
N—((R)-1-benzofuran-2-yl(3-methoxy-2-pyridinyl)methyl)-3,4-dihydro-2H-1,5-benzodioxepine-7-sulfonamide;
N—((R)-1-benzofuran-2-yl(2-thiophenyl)methyl)-3,4-dihydro-2H-1,5-benzodioxepine-7-sulfonamide;
N—((R)-1-benzofuran-2-yl(3-thiophenyl)methyl)-3,4-dihydro-2H-1,5-benzodioxepine-7-sulfonamide;
N—((R)-1-benzofuran-2-yl(3-methyl-2-thiophenyl)methyl)-3,4-dihydro-2H-1,5-benzodioxepine-7-sulfonamide;
N—((R)-1-benzofuran-2-yl(1,3-thiazol-2-yl)methyl)-3,4-dihydro-2H-1,5-benzodioxepine-7-sulfonamide;
N—((R)-1-benzofuran-2-yl(phenyl)methyl)-3-fluoro-3,4-dihydro-2H-1,5-benzodioxepine-7-sulfonamide;
N—((R)-1-benzothiophen-2-yl(2-methylphenyl)methyl)-3,4-dihydro-2H-1,5-benzodioxepine-7-sulfonamide;
N—((R)-1-benzothiophen-2-yl(2-ethylphenyl)methyl)-3,4-dihydro-2H-1,5-benzodioxepine-7-sulfonamide;
N—((R)-1-benzothiophen-2-yl(2-ethenylphenyl)methyl)-3,4-dihydro-2H-1,5-benzodioxepine-7-sulfonamide;
N—((R)-1-benzothiophen-2-yl(2-ethynylphenyl)methyl)-3,4-dihydro-2H-1,5-benzodioxepine-7-sulfonamide;
N—((R)-1-benzothiophen-2-yl(2-methoxyphenyl)methyl)-3,4-dihydro-2H-1,5-benzodioxepine-7-sulfonamide;
N—((R)-1-benzothiophen-2-yl(2-(methylsulfanyl)phenyl)methyl)-3,4-dihydro-2H-1,5-benzodioxepine-7-sulfonamide;
N—((R)-1-benzothiophen-2-yl(2-chlorophenyl)methyl)-3,4-dihydro-2H-1,5-benzodioxepine-7-sulfonamide;
N—((R)-1-benzothiophen-2-yl(3-methoxy-2-pyridinyl)methyl)-3,4-dihydro-2H-1,5-benzodioxepine-7-sulfonamide;
N—((R)-(3-methyl-1-benzofuran-2-yl)(phenyl)methyl)-3,4-dihydro-2H-1,5-benzodioxepine-7-sulfonamide;
N—((R)-(3-methyl-1-benzofuran-2-yl)(2-methylphenyl)methyl)-3,4-dihydro-2H-1,5-benzodioxepine-7-sulfonamide;
N—((R)-phenyl(thieno[2,3-b]pyridin-2-yl)methyl)-3,4-dihydro-2H-1,5-benzodioxepine-7-sulfonamide;
N—((R)-(2-methoxyphenyl)(thieno[2,3-b]pyridin-2-yl)methyl)-3,4-dihydro-2H-1,5-benzodioxepine-7-sulfonamide;
N—((R)-(2-(methylsulfanyl)phenyl)(thieno[2,3-b]pyridin-2-yl)methyl)-3,4-dihydro-2H-1,5-benzodioxepine-7-sulfonamide;
N—((R)-(3-methoxy-2-pyridinyl)(thieno[2,3-b]pyridin-2-yl)methyl)-3,4-dihydro-2H-1,5-benzodioxepine-7-sulfonamide;
N—((R)-(3-methoxy-4-pyridinyl)(thieno[2,3-b]pyridin-2-yl)methyl)-3,4-dihydro-2H-1,5-benzodioxepine-7-sulfonamide;
N—((R)-1,3-benzothiazol-2-yl(2-methoxyphenyl)methyl)-3,4-dihydro-2H-1,5-benzodioxepine-7-sulfonamide;
N—((R)-1,3-benzothiazol-2-yl(2-(methylsulfanyl)phenyl)methyl)-3,4-dihydro-2H-1,5-benzodioxepine-7-sulfonamide;
N—((R)-1,3-benzothiazol-2-yl(3-methoxy-2-pyridinyl)methyl)-3,4-dihydro-2H-1,5-benzodioxepine-7-sulfonamide;
N—((R)-1,3-benzoxazol-2-yl(phenyl)methyl)-3,4-dihydro-2H-1,5-benzodioxepine-7-sulfonamide;
N—((R)-2-naphthalenyl(phenyl)methyl)-3,4-dihydro-2H-1,5-benzodioxepine-7-sulfonamide;
N—((R)-phenyl(2-quinolinyl)methyl)-3,4-dihydro-2H-1,5-benzodioxepine-7-sulfonamide;
N—((R)-1-benzothiophen-2-yl(phenyl)methyl)-3,4-dihydro-2H-1,5-benzodioxepine-7-sulfonamide;
N—((R)-(5-methoxy-1-benzothiophen-2-yl)(phenyl)methyl)-3,4-dihydro-2H-1,5-benzodioxepine-7-sulfonamide;
N—((R)-1,3-benzothiazol-2-yl(phenyl)methyl)-3,4-dihydro-2H-1,5-benzodioxepine-7-sulfonamide;
N—((R)-phenyl(thieno[2,3-c]pyridin-2-yl)methyl)-3,4-dihydro-2H-1,5-benzodioxepine-7-sulfonamide;
N—((R)-phenyl(thieno[3,2-b]pyridin-2-yl)methyl)-3,4-dihydro-2H-1,5-benzodioxepine-7-sulfonamide;
N—((R)-phenyl(thieno[3,2-c]pyridin-2-yl)methyl)-3,4-dihydro-2H-1,5-benzodioxepine-7-sulfonamide;
N—((R)-(5-hydroxy-1-benzothiophen-2-yl)(phenyl)methyl)-3,4-dihydro-2H-1,5-benzodioxepine-7-sulfonamide;
N—((R)-2-(methylsulfanyl)phenyl)(thieno[2,3-c]pyridin-2-yl)methyl)-3,4-dihydro-2H-1,5-benzodioxepine-7-sulfonamide;
N—((R)-2-(methylsulfanyl)phenyl)([1,3]thiazolo[5,4-b]pyridin-2-yl)methyl)-3,4-dihydro-2H-1,5-benzodioxepine-7-sulfonamide;
N—((R)-2-methoxyphenyl)(thieno[2,3-c]pyridin-2-yl)methyl)-3,4-dihydro-2H-1,5-benzodioxepine-7-sulfonamide;
N—((R)-2-methoxyphenyl)([1,3]thiazolo[5,4-b]pyridin-2-yl)methyl)-3,4-dihydro-2H-1,5-benzodioxepine-7-sulfonamide;
N—((R)-2-chlorophenyl)([1,3]thiazolo[5,4-b]pyridin-2-yl)methyl)-3,4-dihydro-2H-1,5-benzodioxepine-7-sulfonamide;
N—((R)-3-methoxy-2-pyridinyl)([1,3]thiazolo[5,4-b]pyridin-2-yl)methyl)-3,4-dihydro-2H-1,5-benzodioxepine-7-sulfonamide;
N—((R)-phenyl(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N—((R)-phenyl(7-(2-pyridinyl)-1-benzothiophen-2-yl)methyl)-3,4-dihydro-2H-1,5-benzodioxepine-7-sulfonamide;
N—((R)-7-chloro-1-benzothiophen-2-yl)(phenyl)methyl)-3,4-dihydro-2H-1,5-benzodioxepine-7-sulfonamide;
N—((R)-phenyl(7-phenyl-1-benzothiophen-2-yl)methyl)-3,4-dihydro-2H-1,5-benzodioxepine-7-sulfonamide;
N—((R)-1-benzothiophen-2-yl(phenyl)methyl)-3,4-dimethoxybenzenesulfonamide; or
N—((R)-1-benzothiophen-2-yl(phenyl)methyl)-2,3-dihydro-1,4-benzodioxine-6-sulfonamide.

In aspect 15, the present invention provides compounds, or pharmaceutically acceptable salts, selected from:
N—((R)-1-benzothiophen-2-yl(2-chlorophenyl)methyl)-3,3-difluoro-3,4-dihydro-2H-1,5-benzodioxepine-7-sulfonamide;
N—((R)-1-benzothiophen-2-yl(2-chlorophenyl)methyl)-3-hydroxy-3,4-dihydro-2H-1,5-benzodioxepine-7-sulfonamide;
N—((R)-1-benzothiophen-2-yl(2-chlorophenyl)methyl)-3-fluoro-3,4-dihydro-2H-1,5-benzodioxepine-7-sulfonamide;
N—((R)-(2-chlorophenyl)(7-(4-(2,2,2-trifluoro-1-hydroxy-1-(trifluoromethyl)ethyl)-2-pyridinyl)-1-benzothiophen-2-yl)methyl)-3,4-dihydro-2H-1,5-benzodioxepine-7-sulfonamide;
N—((R)-(2-chlorophenyl)(7-(4-(2,2,2-trifluoro-1-hydroxy-1-(trifluoromethyl)ethyl)-2-pyridinyl)-1-benzothiophen-2-yl)methyl)-3,4-dimethoxybenzenesulfonamide;
N—((R)-(2-chlorophenyl)(7-(4-((1R)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N—((R)-(2-chlorophenyl)(7-(4-((1R)-2,2,2-trifluoro-1-hydroxy-1-methylethyl)-2-pyridinyl)-1-benzothiophen-2-yl)methyl)-3,4-dimethoxybenzenesulfonamide;
N—((R)-(2-chlorophenyl)(7-(4-((1S)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N—((R)-(2-Chlorophenyl)(7-(4-((1S)-2,2,2-trifluoro-1-hydroxy-1-methylethyl)-2-pyridinyl)-1-benzothiophen-2-yl)methyl)-3,4-dimethoxybenzenesulfonamide; or
N-((2-amino-4-pyridinyl)(1-benzofuran-2-yl)methyl)-3,4-dihydro-2H-1,5-benzodioxepine-7-sulfonamide.

In aspect 16, the present invention provides methods of treating type 2 diabetes, hyperglycemia, impaired glucose tolerance, insulin resistance, retinopathy, nephropathy, neuropathy, cataracts, glaucoma, Syndrome X, or polycystic ovarian syndrome, the methods comprising administering to a patient in need thereof a therapeutically effective amount of a compound in accordance with any one of aspects 1 to 15, or a pharmaceutically acceptable salt thereof.

In aspect 17, the present invention provides the methods of aspect 16 wherein the method is the treatment of type 2 diabetes.

In aspect 18, the present invention provides the methods of aspect 16 wherein the methods further comprise administering metformin.

In aspect 19, the present invention provides the methods of aspect 16 wherein the methods further comprise administering a DPP-IV inhibitor, or a pharmaceutically acceptable salt thereof.

In aspect 20, the present invention provides the methods of aspect 16 wherein the methods further comprise administering a sulfonylurea, or a pharmaceutically acceptable salt thereof.

In aspect 21, the present invention provides the methods of aspect 16 wherein the methods further comprise administering a PPARγ agonist, or a pharmaceutically acceptable salt thereof.

In aspect 22, the present invention provides pharmaceutical compositions comprising a compound in accordance with any one of aspects 1 to 15, or a is pharmaceutically acceptable salt thereof, and a pharmaceutically acceptable excipient.

### DETAILED DESCRIPTION

The present invention provides compounds of formula I, II, III or IV as defined above, or pharmaceutically acceptable salts thereof. The present invention also provides pharmaceutical compositions comprising a compound of formula I, II, III or IV, or pharmaceutically acceptable salts thereof, and methods of treating diseases and/or conditions, such as diabetes, using a compound of formula I, II, III or IV, or pharmaceutically acceptable salts thereof.

The term “alkyl” means a straight or branched chain hydrocarbon. Representative examples of alkyl groups include methyl, ethyl, propyl, isopropyl, butyl, isobutyl, tert-butyl, sec-butyl, pentyl and hexyl. Typical alkyl groups are alkyl groups having from 1 to 8 carbon atoms, which groups are commonly represented as C₁₋₈alkyl.

The term “alkoxy” means an alkyl group bonded to an oxygen atom. Representative examples of alkoxy groups include methoxy, ethoxy, tert-butoxy, propoxy and isobutoxy. Common alkoxy groups are C₁₋₈alkoxy.

The term “halogen” or “halo” means chlorine, fluorine, bromine or iodine.

The term “alkenyl” means a branched or straight chain hydrocarbon having one or more carbon-carbon double bonds. Representative examples alkenyl groups include ethenyl, propenyl, allyl, butenyl and 4-methylbutenyl. Common alkenyl groups are C₂₋₈alkenyl.

The term “alkynyl” means a branched or straight chain hydrocarbon having one or more carbon-carbon triple bonds. Representative examples of alkynyl groups include ethynyl, propynyl (propargyl) and butynyl. Common alkynyl groups are C₂₋₈ alkynyl.

The term “cycloalkyl” means a cyclic, nonaromatic hydrocarbon. Examples of cycloalkyl groups include cyclopropyl, cyclobutyl, cyclopentyl, cyclohexyl and cycloheptyl. A cycloalkyl group can contain one or more double bond. Examples of cycloalkyl groups that contain double bonds include cyclopentenyl, cyclohexenyl, cyclohexadienyl and cyclobutadienyl. Common cycloalkyl groups are C₃₋₈cycloalkyl groups.

The term “perfluoroalkyl” means an alkyl group in which all of the hydrogen atoms have been replaced with fluorine atoms. Common perfluoroalkyl groups are C₁₋₈perfluoroalkyl. An example of a common perfluoroalkyl group is —CF₃.

The term “acyl” means a group derived from an organic acid by removal of the hydroxy group (—OH). For example, the acyl group CH₃C(═O)— is formed by the removal of the hydroxy group from CH₃C(═O)OH.

The term “aryl” means a cyclic, aromatic hydrocarbon. Examples of aryl groups include phenyl and naphthyl. Common aryl groups are six to thirteen membered rings.

The term “heteroatom” as used herein means an oxygen, nitrogen or sulfur atom.

The term “heteroaryl” means a cyclic, aromatic hydrocarbon in which one or more carbon atoms of an aryl group have been replaced with a heteroatom. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, indolyl, triazolyl, pyridazinyl, indazolyl, purinyl, quinolizinyl, isoquinolyl, quinolyl, naphthyridinyl, quinoxalinyl, isothiazolyl and benzo[b]thienyl. Common heteroaryl groups are five to thirteen membered rings that contain from 1 to 4 heteroatoms. Heteroaryl groups that are five and six membered rings that contain 1 to 3 heteroatoms are particularly common

The term “heterocycloalkyl” means a cycloalkyl group in which one or more of the carbon atoms has been replaced with a heteroatom. If the heterocycloalkyl group contains more than one heteroatom, the heteroatoms may be the same or different. Examples of heterocycloalkyl groups include tetrahydrofuryl, morpholinyl, piperazinyl, piperidinyl and pyrrolidinyl. It is also possible for the heterocycloalkyl group to have one or more double bonds, but is not aromatic. Examples of heterocycloalkyl groups containing double bonds include dihydrofuran. Common heterocycloalkyl groups are three to ten membered rings containing from 1 to 4 heteroatoms. Heterocycloalkyl groups that are five and six membered rings that contain 1 to 3 heteroatoms are particularly common

It is also noted that the cyclic ring groups, i.e., aryl, heteroaryl, cycloalkyl, and heterocycloalkyl, can comprise more than one ring. For example, the naphthyl group is a fused bicyclic ring system. It is also intended that the present invention include ring groups that have bridging atoms, or ring groups that have a spiro orientation.

Representative examples of five to six membered aromatic rings, optionally having one or two heteroatoms, are phenyl, furyl, thienyl, pyrrolyl, oxazolyl, thiazolyl, imidazolyl, pyrazolyl, isoxazolyl, isothiazolyl, pyridinyl, pyridiazinyl, pyrimidinyl, and pyrazinyl.

Representative examples of partially saturated, fully saturated or fully unsaturated five to eight membered rings, optionally having one to three heteroatoms, are cyclopentyl, cyclohexyl, cycloheptyl, cyclooctyl and phenyl. Further exemplary five membered rings are furyl, thienyl, pyrrolyl, 2-pyrrolinyl, 3-pyrrolinyl, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadizaolyl, 1,2,4-oxadiazolyl, 1,2,5-oxadiazolyl, 1,3,4oxadiazolyl, 1,2,3-triazolyl, 1,2,4-trizaolyl, 1,3,4-thiadiazolyl, 3H-1,2,3-dioxazolyl, 1,2,4-dioxazolyl, 1,3,2-dioxazolyl, 1,3,4-dioxazolyl, 5H-1,2,5-oxathiazolyl, and 1,3-oxathiolyl.

Further exemplary six membered rings are 2H-pyranyl, 4H-pyranyl, pyridinyl, piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-triazinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-(3 oxathiazinyl, and 1,4,2-oxadiazinyl.

Further exemplary seven membered rings are azepinyl, oxepinyl, thiepinyl and 1,2,4-triazepinyl.

Further exemplary eight membered rings are cyclooctyl, cyclooctenyl and cyclooctadienyl.

Exemplary bicyclic rings consisting of two fused partially saturated, fully saturated or fully unsaturated five and/or six membered rings, optionally having one to four heteroatoms, are indolizinyl, indolyl, isoindolyl, indolinyl, cyclopenta(b)pyridinyl, pyrano(3,4-b)pyrrolyl, benzofuryl, isobenzofuryl, benzo[b]thienyl, benzo[c]thienyl, 1H-indazolyl, indoxazinyl, benzoxazolyl, anthranilyl, benzimidazolyl, benzthiazolyl, purinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, pyrido(3,4-b)pyridinyl, pyrido(3,2-b)pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

A cyclic ring group may be bonded to another group in more than one way. If no particular bonding arrangement is specified, then all possible arrangements are intended. For example, the term “pyridyl” includes 2-, 3-, or 4-pyridyl, and the term “thienyl” includes 2-, or 3-thienyl.

The term “substituted” means that a hydrogen atom on a molecule or group is replaced with a group or atom. Typical substituents include: halogen, C₁₋₈alkyl, hydroxyl, C₁₋₈alkoxy, —NR^{x}R^{x}, nitro, cyano, halo or perhaloC₁₋₈alkyl, C₂₋₈alkenyl, C₂₋₈alkynyl, —SR^{x}, —S(═O)₂R^{x}, —C(═O)OR^{x}, —C(═O)R^{x}, wherein each R^{x} is independently hydrogen or C₁-C₈ alkyl. It is noted that when the substituent is —NR^{x}R^{x}, the R^{x} groups may be joined together with the nitrogen atom to form a ring.

The term “oxo”, when used as a substituent, means the ═O group, which is is typically attached to a carbon atom.

A group or atom that replaces a hydrogen atom is also called a substituent.

Any particular molecule or group can have one or more substituent depending on the number of hydrogen atoms that can be replaced.

The symbol “—” represents a covalent bond and can also be used in a radical group to indicate the point of attachment to another group. In chemical structures, the symbol is commonly used to represent a methyl group in a molecule.

The location of substituents on a cyclic group can be identified using a numbering system to show the substituents' point of attachment on the cyclic group with respect to the cyclic group's point of attachment to the rest of the molecule. For example, below five and six membered rings are drawn to show the identifying numbers of the different possible points of substitution. It is noted that the cyclic group can contain heteroatoms.

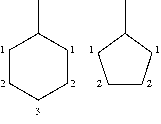

The compounds of the present invention include compounds, or pharmaceutically acceptable salts thereof, that have the R configuration at the designated stereocenter. The compounds of the present invention, or the pharmaceutically acceptable salts thereof, are also intended to include mixtures of the R and S isomer at the designated stereocenter, including racemic mixtures. A preferred mixture is one that comprises substantially pure R isomer. Another preferred mixture is a racemic mixture. Still another preferred mixture is one that comprises more R isomer than S isomer.

It is believed that the compounds of the present invention that show the greatest ability to disrupt the interaction of GKRP with GK have the R configuration at the stereocenter designated with a * symbol in formula I, II, III or IV.

The term “therapeutically effective amount” means an amount of a compound that ameliorates, attenuates or eliminates one or more symptom of a particular disease or condition, or prevents or delays the onset of one of more symptom of a particular disease or condition.

The term “patient” means animals, such as dogs, cats, cows, horses, sheep and humans. Particular patients are mammals. The term patient includes males and females.

The term “pharmaceutically acceptable” means that the referenced substance, such as a compound of the present invention or a formulation containing a compound of the present invention, or a particular excipient, are suitable for administration to a patient.

The terms “treating”, “treat” or “treatment” and the like include preventative (e.g., prophylactic) and palliative treatment.

The term “patient in need thereof” means a patient who has or is at risk of having a GKRP/GK mediated disease or condition, such as type 2 diabetes.

The term “excipient” means any pharmaceutically acceptable additive, carrier, diluent, adjuvant, or other ingredient, other than the active pharmaceutical ingredient (API), which is typically included for formulation and/or administration to a patient.

The compounds of the present invention are administered to a patient in a therapeutically effective amount. The compounds can be administered alone or as part of a pharmaceutically acceptable composition or formulation. In addition, the compounds or compositions can be administered all at once, as for example, by a bolus injection, multiple times, such as by a series of tablets, or delivered substantially uniformly over a period of time, as for example, using transdermal delivery. It is also noted that the dose of the compound can be varied over time.

In addition, the compounds of the present invention can be administered alone, in combination with other compounds of the present invention, or with other pharmaceutically active compounds. The other pharmaceutically active compounds can be intended to treat the same disease or condition as the compounds of the present invention or a different disease or condition. If the patient is to receive or is receiving multiple pharmaceutically active compounds, the compounds can be administered simultaneously, or sequentially. For example, in the case of tablets, the active compounds may be found in one tablet or in separate tablets, which can be administered at once or sequentially in any order. In addition, it should be recognized that the compositions may be different forms. For example, one or more compound may be delivered via a tablet, while another is administered via injection or orally as a syrup. All combinations, delivery methods and administration sequences are contemplated.

The compounds of the present invention may used in the manufacture of a medicament for the treatment of a disease and/or condition mediated by GKRP/GK, such as type 2 diabetes.

The compounds of the present invention may be used in combination with other pharmaceutically active compounds. It is noted that the term “pharmaceutically active compounds” can include biologics, such as proteins, antibodies and peptibodies. Examples of other pharmaceutically active compounds include, but are not limited to: (a) dipeptidyl peptidase IV (DPP-IV) inhibitors such as Vildagliptin (Novartis), Sitagliptin (Merck&Co.), Saxagliptin (BMS) Allogliptin (Takeda); (b) insulin sensitizers including (i) PPARγ agonists such as the glitazones (e.g., troglitazone, pioglitazone, edaglitazone, rosiglitazone, and the like) and other PPAR ligands, including PPARα/γ dual agonists such as muraglitazar (BMS) and tesaglitazar (AstraZeneca), and PPARα agonists such as fenofibric acid derivatives (gemfibrozil, clofibrate, fenofibrate and bezafibrate), (ii) biguanides such as metformin and phenformin, and (iii) protein tyrosine phosphatase-1B (PTP-1B) inhibitors; (c) insulin or insulin mimetics; (d) incretin and incretin mimetics such as (i) Exenatide available from Amylin Pharmaceuticals, (i) amylin and amylin mimetics such as pramlintide acetate, available as Symlin®, (iii) GLP-1, GLP-1 mimetics, and GLP-1 receptor agonists, (iv) GIP, GIP mimetics and GIP receptor agonists; (e) sulfonylureas and other insulin secretagogues, such as tolbutamide, glyburide, gliclazide, glipizide, glimepiride, meglitinides, and repaglinide; (f) α-glucosidase inhibitors (such as acarbose and miglitol); (g) glucagon receptor antagonists; (h) PACAP, PACAP mimetics, and PACAP receptor agonists; (i) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants such as cholestyramine, colestipol and dialkylaminoalkyl derivatives of a cross-linked dextran, (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists such as muraglitazar (BMS) and tesaglitazar (AstraZeneca), (vi) inhibitors of cholesterol absorption, such as beta-sitosterol and ezetimibe, (vii) acyl CoA:cholesterol acyltransferase inhibitors such as avasimibe, and (viii) anti-oxidants such as probucol; (j) PPARδ agonists such as GW-501516 from GSK; (k) anti-obesity compounds such as fenfluramine, dexfenfluramine, phentemine, sibutramine, orlistat, neuropeptide Y1 or Y5 antagonists, MTP inhibitors, squalene synthase inhibitor, lipoxygenase inhibitor, ACAT inhibitor, Neuropeptide Cannabinoid CB-1 receptor antagonists, CB-1 receptor inverse agonists and antagonists, fatty acid oxidation inhibitors, appetite suppressants (1) adrenergic receptor agonists, melanocortin receptor agonists, in particular—melanocortin-4 receptor agonists, ghrelin antagonists, and melanin-concentrating hormone (MCH) receptor antagonists; (m) ileal bile acid transporter inhibitors; (n) agents intended for use in inflammatory conditions such as aspirin, non steroidal anti-inflammatory drugs, glucocorticoids, azalfidine, and selective cyclooxygenase-2 inhibitors; (o) antihypertensive agents such as ACE inhibitors (enalapril, lisinopril, captopril, quinapril, fosinoprol, ramipril, spirapril, tandolapril), angiotensin-II (AT-1) receptor blockers (losartan, candesartan, irbesartan, valsartan, telmisartan, eprosartan), beta blockers and calcium channel blockers; and (p) glucokinase activators (GKAs); (q) agents which can be used for the prevention, delay of progression or treatment of neurodegenerative disorders, cognitive disorders or a drug for improving memory such as anti-inflammatory drugs, antioxidants, neuroprotective agents, glutamate receptor antagonists, acetylcholine esterase inhibitors, butyrylcholinesterase inhibitors, MAO inhibitors, dopamine agonists or antagonists, inhibitors of gamma and beta secretases, inhibitors of amyloid aggregation, amyloid beta peptide, antibodies to amyloid beta peptide, inhibitors of acetylcholinesterase, glucokinase activators, agents directed at modulating GABA, NMDA, cannabinoid, AMPA, kainate, phosphodiesterase (PDE), PKA, PKC, CREB or nootropic systems; (r) leukocyte growth promotors intended for the treatment and prevention of reduced bone marrow production, infectious diseases, hormone dependent disorders, inflammatory diseases, HIV, allergies, leukocytopenia, and rheumatism; (s) SGLT2 inhibitor; (t) glycogen phosphorylase inhibitor; (u) aP2 inhibitors; (v) aminopeptidase N inhibitor (w) vasopeptidase inhibitors like neprilysin inhibitors and/or ACE inhibitors or dual NEP/ACE inhibitor; (x) growth hormone secretagogue for enhancing growth hormone levels and for treating growth retardation/dwarfism or metabolic disorders or where the disorder is an injury, or a wound in need of healing, or a mammalian patient recovering from surgery; (y) 5-HT 3 or 5-HT 4 receptor modulators (tegaserod, cisapride, nor-cisapride, renzapride, zacopride, mosapride, prucalopride, buspirone, norcisapride, cilansetron, ramosetron, azasetron, ondansetron, etc.); (Za) aldose reductase inhibitors; (Zb) sorbitol dehydrogenase inhibitors; (Zc) AGE inhibitors; (Zd) erythropoietin agonist such as EPO, EPO mimetics, and EPO receptor agonists. The compounds of the present invention may also be used in combination with GPR40 agonists.

Examples of glucokinase activators that can be used in combination with the compounds of the present invention include those set forth in WO 2009/042435, published Apr. 2, 2009. Examples of specific compounds, or pharmaceutically acceptable salts thereof, disclosed in the published application that may be used in combination with the compounds of the present invention, or pharmaceutically acceptable salts thereof, include compounds selected from:
(S)-1-(5-(5-bromo-3-(2-methylpyridin-3-yloxy)pyridin-2-ylamino)-1,2,4-thiadiazol-3-yl)ethane-1,2-diol;
(S)-1-(5-(5-trifluoromethyl-3-(2-methylpyridin-3-yloxy)pyridin-2-ylamino)-1,2,4-thiadiazol-3-yl)ethane-1,2-diol;
(S)-1-(5-(5-phenylthio-3-(2-methylpyridin-3-yloxy)pyridin-2-ylamino)-1,2,4-thiadiazol-3-yl)ethane-1,2-diol;
(S)-1-(5-(5-phenylthio-3-(pyridin-3-yloxy)pyridin-2-ylamino)-1,2,4-thiadiazol-3-yl)piperidin-1-yl)ethane-1,2-diol;
(S)-1-(5-(3-(2-methylpyridin-3-yloxy)-5-(pyridin-2-ylthio)pyridin-2-ylamino)-1,2,4-thiadiazol-3-yl)ethane-1,2-diol;
(S)-1-(5-(5(2hydroxyethylthio)-3-(2-methylpyridin-3-yloxy)pyridin-2-ylamino)-1,2,4-thiadiazol-3-yl)ethane-1,2-diol;
(S)-1-(5-(4-fluorophenoxy)-5-pyridin-2-ylthio)pyridin-2-ylamino)-1,2,4-thiadiazol-3-yl)ethane-1,2-diol;
(R)-1-(2-(5-bromo-3-(4-fluorophenoxy)pyridin-2-ylamino)thiazol-4-yl)ethane-1,2-diol;
(S)-1-(2-(5-bromo-3-(4-fluorophenoxy)pyridin-2-ylamino)thiazol-4-yl)ethane-1,2-diol;
(R)-1-(2-(3-(4-fluorophenoxy)-5-(pyridin-2-ylthio)pyridin-2-ylamino)thiazol-4-yl)-ethane-1,2-diol;
(1S)-1-(5-(5-bromo-3-(5,6,7,8-tetrahydroquinolin-5-yloxy)pyridin-2-ylamino)-1,2,4-thiadiazol-3-yl)ethane-1,2-diol;
(S)-1-(5-(5-bromo-3-(1-(2-hydroxyethyl)-1H-pyrazol-4-yloxy)pyridin-2-ylamino)-1,2,4-thiadiazol-3-yl)ethane-1,2-diol;
(R)-1-(2-(5-bromo-3-(2-methylpyridin-3-yloxy)pyridin-2-ylamino)thiazol-4-yl)-ethane-1,2-diol;
(S)-1-(5-(5-(2-hydroxyethylthio)-3-(pyridin-3-yloxy)pyridin-2-ylamino)-1,2,4-thiadiazol-3-yl)ethane-1,2-diol;
(S)-1-(5-(5-bromo-3-(1-methyl-1H-pyrazol-4-yloxy)pyridin-2-ylamino)-1,2,4-thiadiazol-3-yl)ethane-1,2-diol;
(S)-1-(5-(3-(1-methyl-1H-pyrazol-4-yloxy)-5-(2-methylpyridin-3-ylthio)pyridin-2-ylamino)-1,2,4-thiadiazol-3-yl)ethane-1,2-diol;
(S)-1-(5-(5-(2-methylpyridin-3-ylthio)-3-(1,3,5-trimethyl-1H-pyrazol-4-yloxy)-pyridin-2-ylamino)-1,2,4-thiadiazol-3-yl)ethane-1,2-diol;

| | |
|---|---|
| 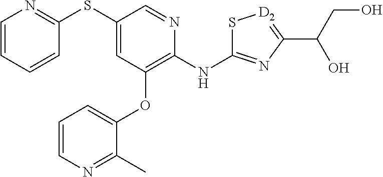 | D² = CH D² = N |
| 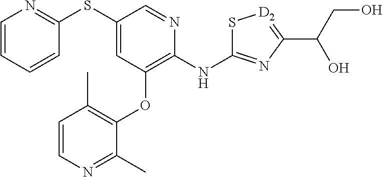 | D² = CH D² = N |
| 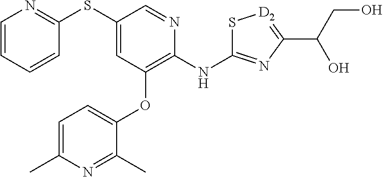 | D² = CH D² = N |
| 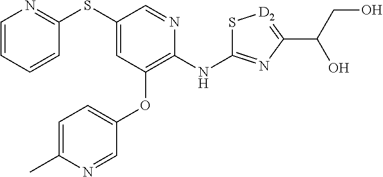 | D² = CH D² = N |
| 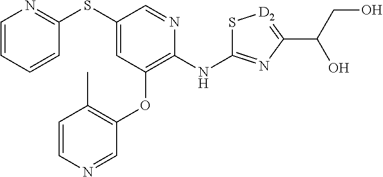 | D² = CH D² = N |
| 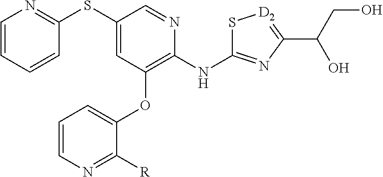 | D² = CH R = Et, iPr, CH₂OH, CH₂CH₂OH, or CF₃ D² = N R = Et, iPr, CH₂OH, CH₂CH₂OH, or CF₃ |
| 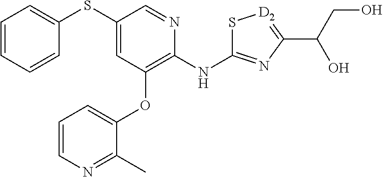 | D² = CH D² = N |
| 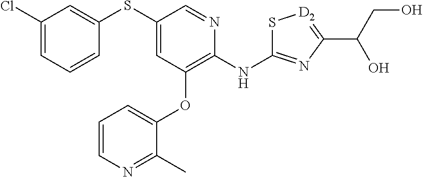 | D² = CH D² = N |
| 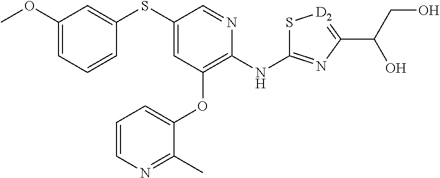 | D² = CH D² = N |
| 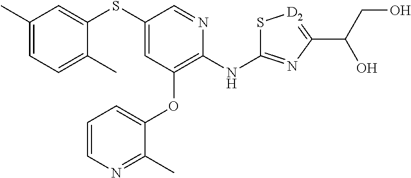 | D² = CH D² = N |
| 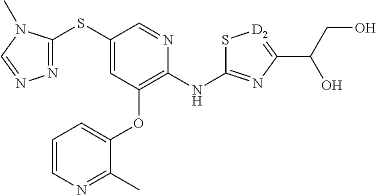 | D² = CH D² = N |
| 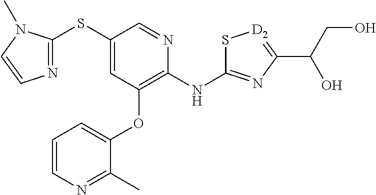 | D² = CH D² = N |
| 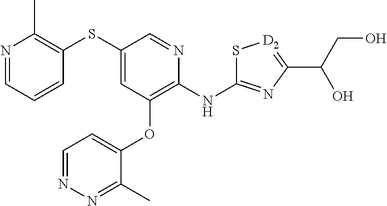 | D² = CH D² = N |
| 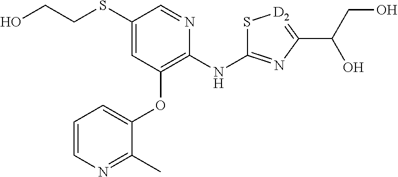 | D² = CH D² = N |
| 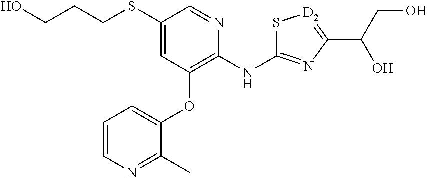 | D² = CH D² = N |
| 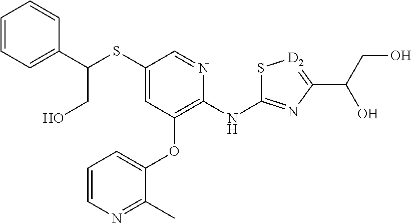 | D² = CH D² = N |
| 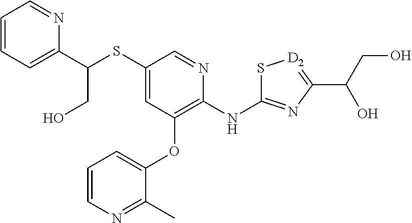 | D² = CH D² = N |
| 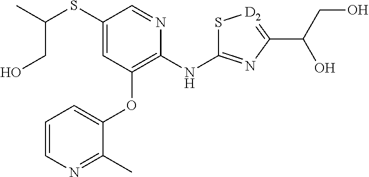 | D² = CH D² = N |
| 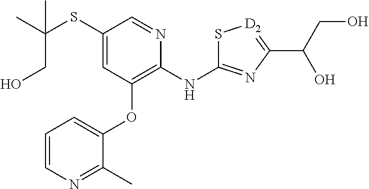 | D² = CH D² = N |
| 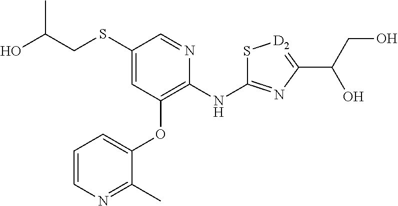 | D² = CH D² = N |
| 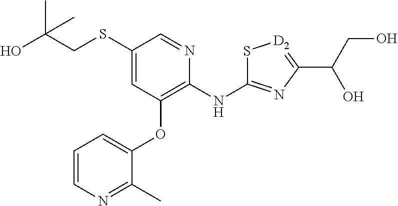 | D² = CH D² = N |
| 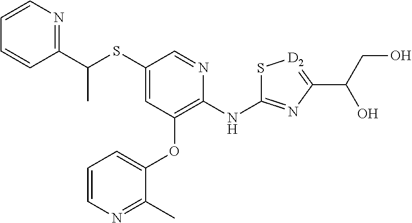 | D² = CH D² = N |
| 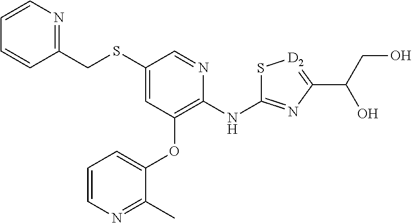 | D² = CH D² = N |
| 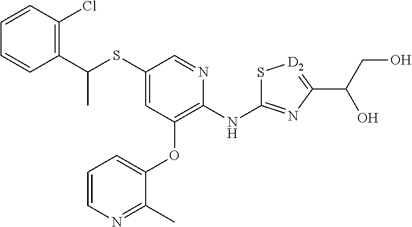 | D² = CH D² = N |
| 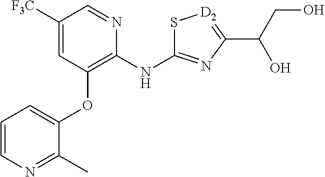 | D² = CH D² = N |
| 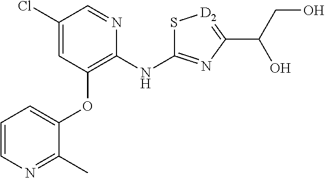 | D² = CH D² = N |
| 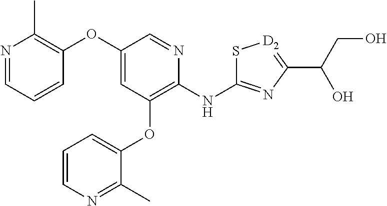 | D² = CH D² = N |
| 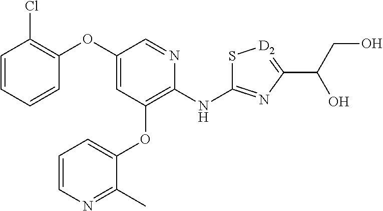 | D² = CH D² = N |
| 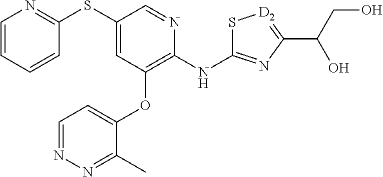 | D² = CH D² = N |
| 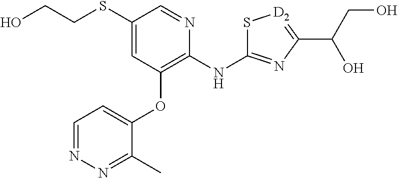 | D² = CH D² = N |
| 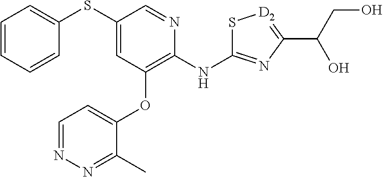 | D² = CH D² = N |
| 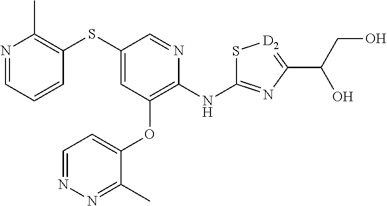 | D² = CH D² = N |
| 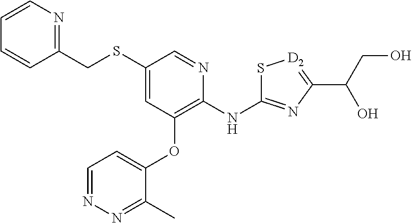 | D² = CH D² = N |
| 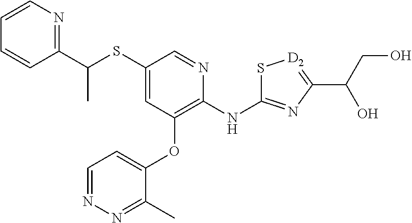 | D² = CH D² = N |
| 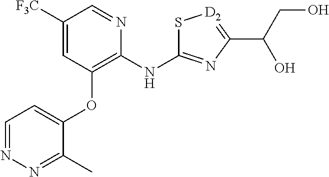 | D² = CH D² = N |
| 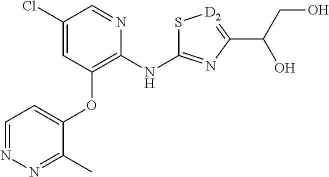 | D² = CH D² = N |
| 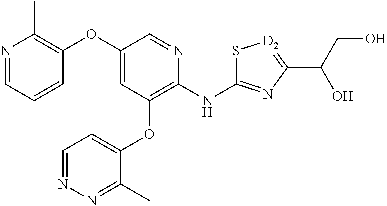 | D² = CH D² = N |
| 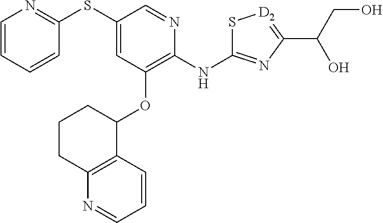 | D² = CH D² = N |
| 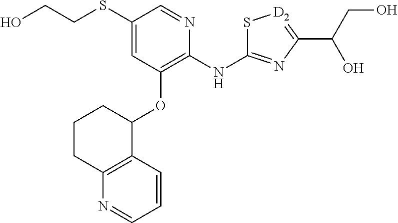 | D² = CH D² = N |
| 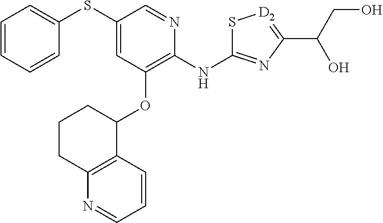 | D² = CH D² = N |
| 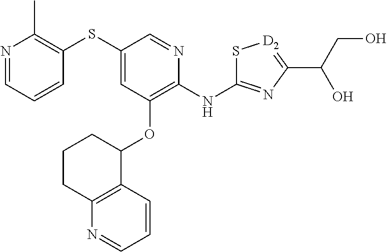 | D² = CH D² = N |
| 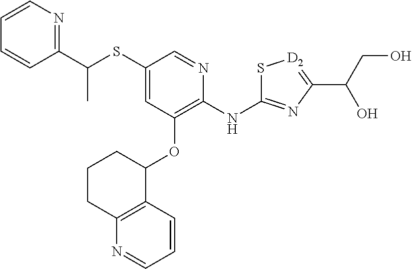 | D² = CH D² = N |
| 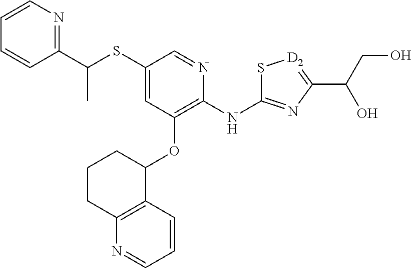 | D² = CH D² = N |
| 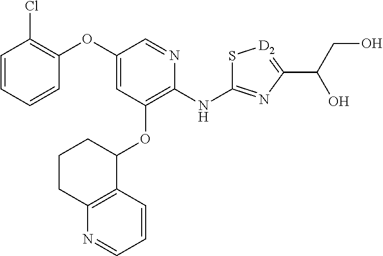 | D² = CH D² = N |
| 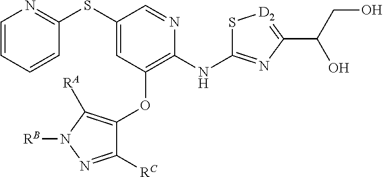 | D² = CH R^{A}, R^{B}, R^{C} are independently H or Me D² = N R^{A}, R^{B}, R^{C} are independently H or Me |
| 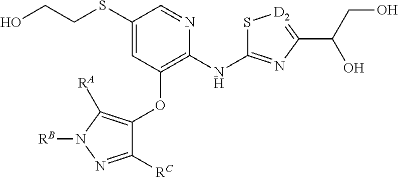 | D² = CH R^{A}, R^{B}, R^{C} are independently H or Me D² = N R^{A}, R^{B}, R^{C} are independently H or Me |
| 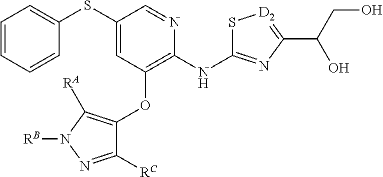 | D² = CH R^{A}, R^{B}, R^{C} are independently H or Me D² = N R^{A}, R^{B}, R^{C} are independently H or Me |
| 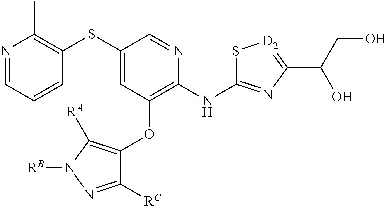 | D² = CH R^{A}, R^{B}, R^{C} are independently H or Me D² = N R^{A}, R^{B}, R^{C} are independently H or Me |
| 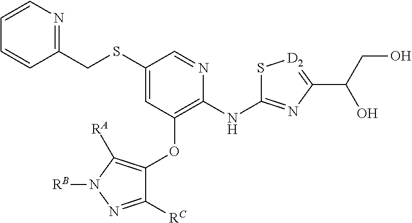 | D² = CH R^{A}, R^{B}, R^{C} are independently H or Me D² = N R^{A}, R^{B}, R^{C} are independently H or Me |
| 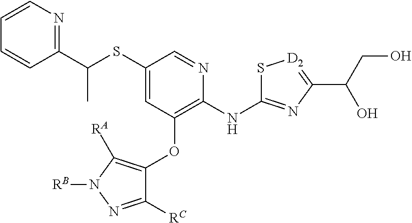 | D² = CH R^{A}, R^{B}, R^{C} are independently H or Me D² = N R^{A}, R^{B}, R^{C} are independently H or Me |
| 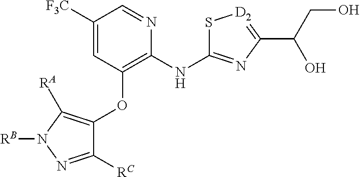 | D² = CH R^{A}, R^{B}, R^{C} are independently H or Me D² = N R^{A}, R^{B}, R^{C} are independently H or Me |
| 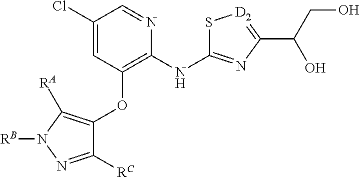 | D² = CH R^{A}, R^{B}, R^{C} are independently H or Me D² = N R^{A}, R^{B}, R^{C} are independently H or Me |
| 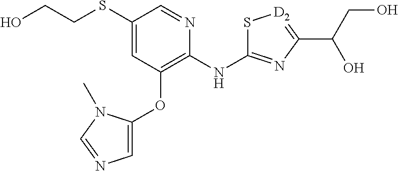 | D² = CH D² = N |
| 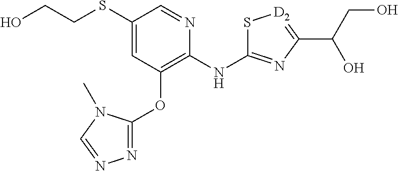 | D² = CH D² = N |
| 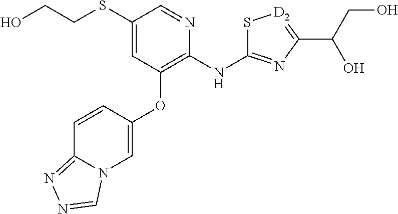 | D² = CH D² = N |
| 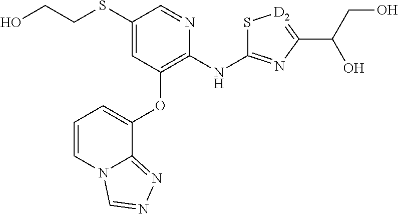 | D² = CH D² = N |
| 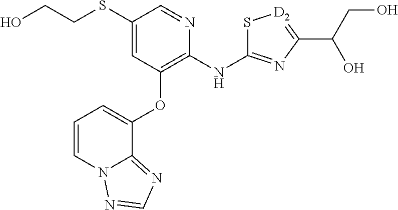 | D² = CH D² = N |
| 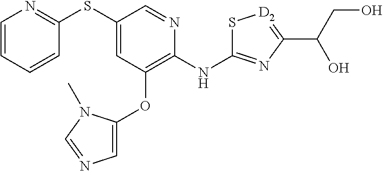 | D² = CH D² = N |
| 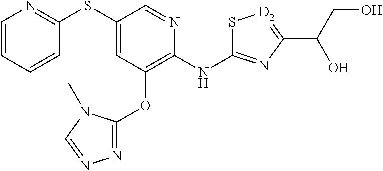 | D² = CH D² = N |
| 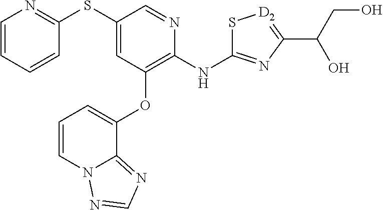 | D² = CH D² = N |
| 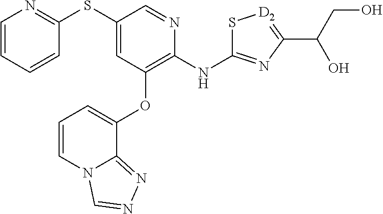 | D² = CH D² = N |
| 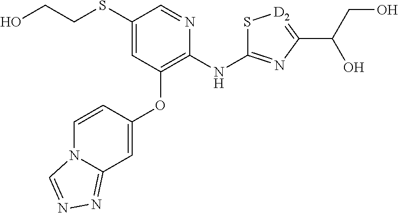 | D² = CH D² = N |
| 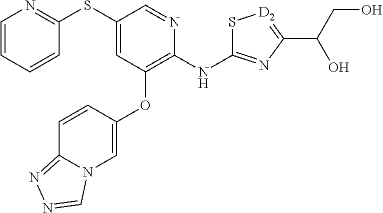 | D² = CH D² = N |
| 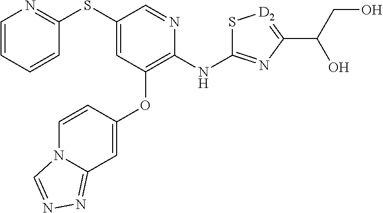 | D² = CH D² = N |
| 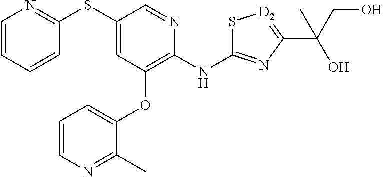 | D² = CH D² = N |
| 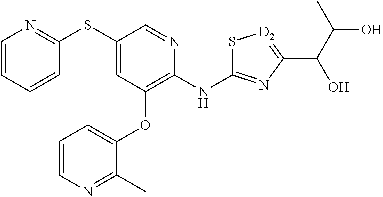 | D² = CH D² = N |
| 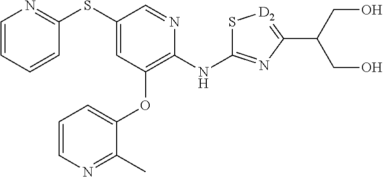 | D² = CH D² = N |
| 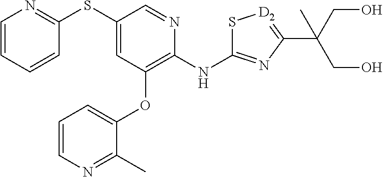 | D² = CH D² = N |
| 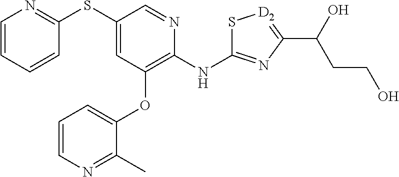 | D² = CH D² = N |
| 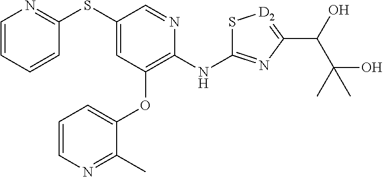 | D² = CH D² = N |
| 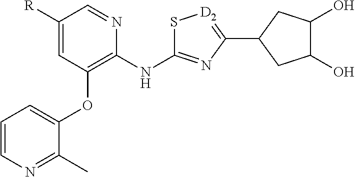 | D² = CH R = S-pyrid-2-yl, CF₃, S-2- methylpyrid-3-yl D² = N R = S-pyrid-2-yl, CF₃, S-2- methylpyrid-3-yl |
| 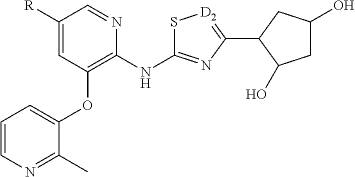 | D² = CH R = S-pyrid-2-yl, CF₃, S-2- methylpyrid-3-yl D² = N R = S-pyrid-2-yl, CF₃, S-2- methylpyrid-3-yl |
| 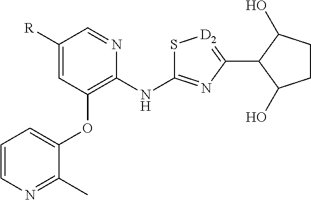 | D² = CH R = S-pyrid-2-yl, CF₃, S-2- methylpyrid-3-yl D² = N R = S-pyrid-2-yl, CF₃, S-2- methylpyrid-3-yl |
| 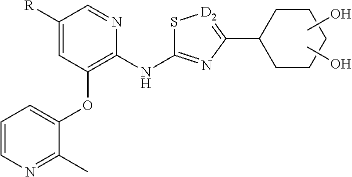 | D² = CH R = S-pyrid-2-yl, CF₃, S-2- methylpyrid-3-yl D² = N R = S-pyrid-2-yl, CF₃, S-2- methylpyrid-3-yl |
| 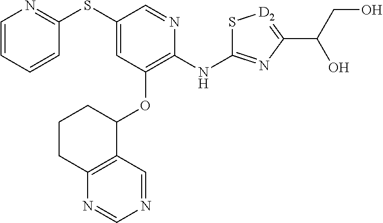 | D² = CH D² = N |
| 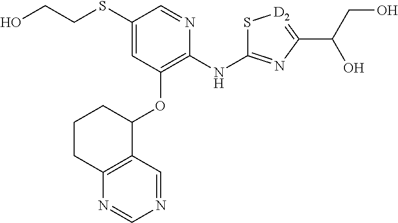 | D² = CH D² = N |
| 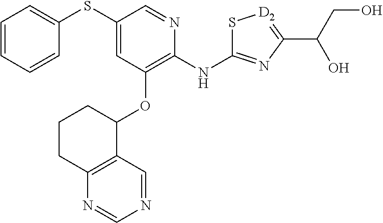 | D² = CH D² = N |
| 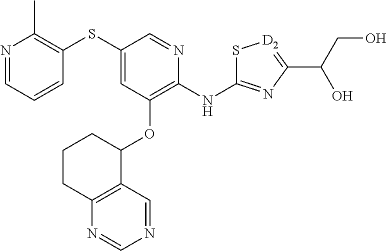 | D² = CH D² = N |
| 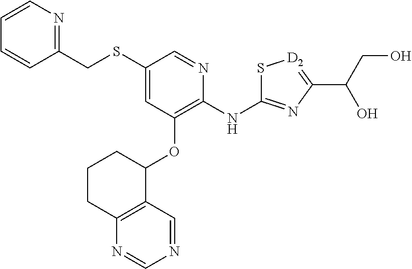 | D² = CH D² = N |
| 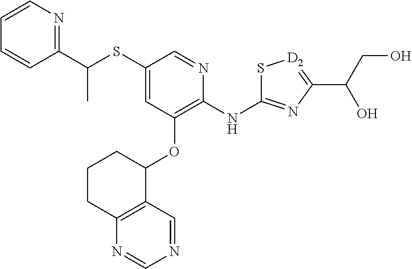 | D² = CH D² = N |
| 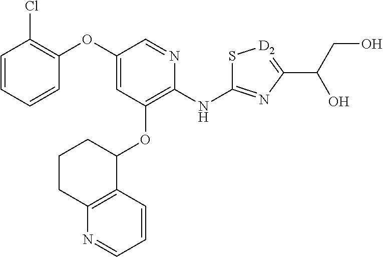 | D² = CH D² = N |
| 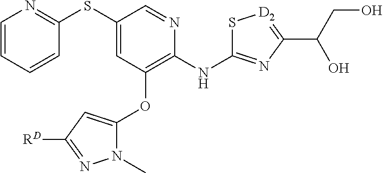 | D² = CH R^{D} = H, Me, or CF₃ D² = N R^{D} = H, Me, or CF₃ |
| 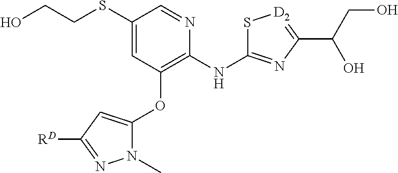 | D² = CH R^{D} = is H, CF₃ or (1-6C alkyl) D² = N R^{D} = is H, CF₃ or (1-6C alkyl) |
| 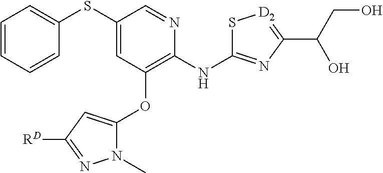 | D² = CH R^{D} = is H, CF₃ or (1-6C alkyl) D² = N R^{D} = is H, CF₃ or (1-6C alkyl) |
| 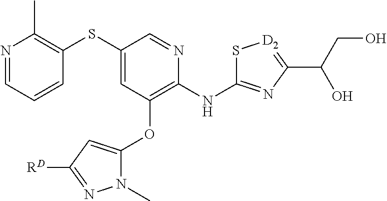 | D² = CH R^{D} = is H, CF₃ or (1-6C alkyl) D² = N R^{D} = is H, CF₃ or (1-6C alkyl) |
| 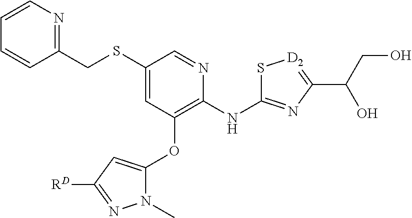 | D² = CH R^{D} = is H, CF₃ or (1-6C alkyl) D² = N R^{D} = is H, CF₃ or (1-6C alkyl) |
| 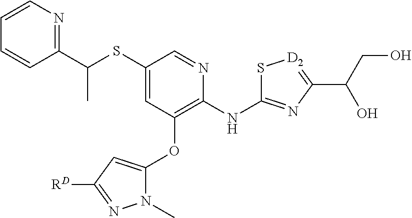 | D² = CH R^{D} = is H, CF₃ or (1-6C alkyl) D² = N R^{D} = is H, CF₃ or (1-6C alkyl) |
| 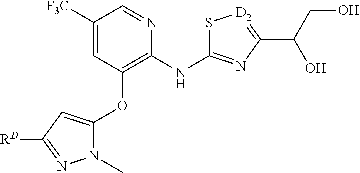 | D² = CH R^{D} = is H, CF₃ or (1-6C alkyl) D² = N R^{D} = is H, CF₃ or (1-6C alkyl) |
| 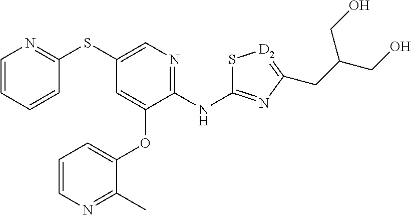 | D² = CH D² = N |
| 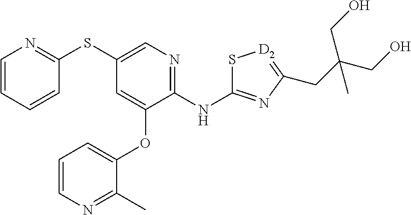 | D² = CH D² = N |
| 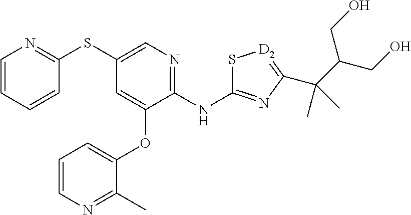 | D² = CH D² = N |
| 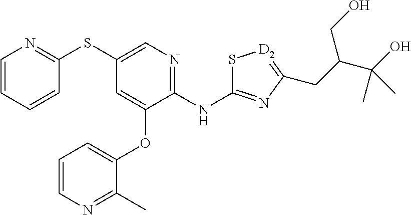 | D² = CH D² = N |
| 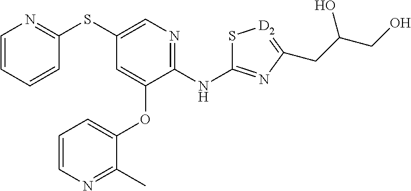 | D² = CH D² = N |
| 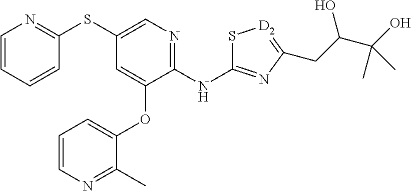 | D² = CH D² = N |
| 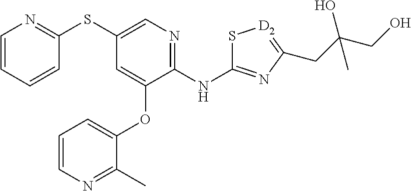 | D² = CH D² = N |
| 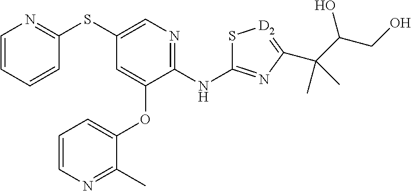 | D² = CH D² = N |
| 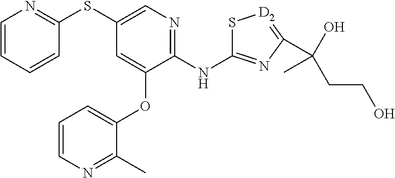 | D² = CH D² = N |
| 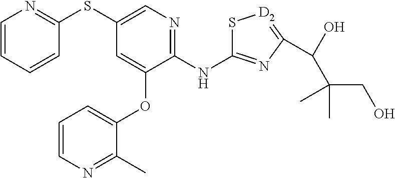 | D² = CH D² = N |
| 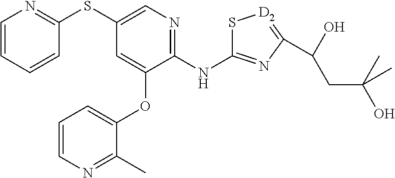 | D² = CH D² = N |
| 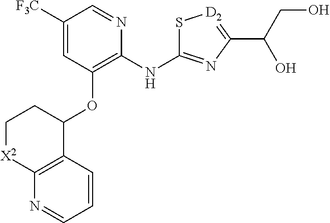 | D² = CH X = CH₂ D² = CH₂ X = O D² = N X = CH₂ D² = N X = O |
| 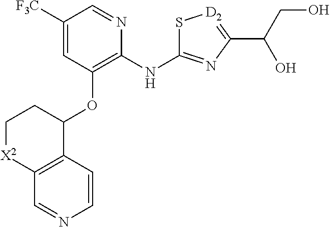 | D² = CH X = CH₂ D² = CH₂ X = O D² = N X = CH₂ D² = N X = O |
| 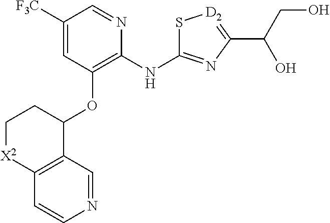 | D² = CH X = CH₂ D² = CH₂ X = O D² = N X = CH₂ D² = N X = O |
| 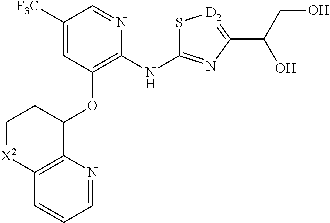 | D² = CH X = CH₂ D² = CH₂ X = O D² = N X = CH₂ D² = N X = O |
| 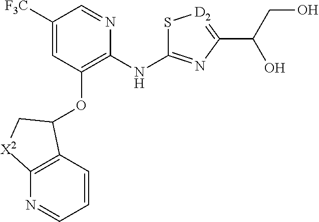 | D² = CH X = CH₂ D² = CH₂ X = O D² = N X = CH₂ D² = N X = O |
| 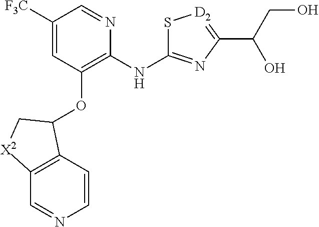 | D² = CH X = CH₂ D² = CH₂ X = O D² = N X = CH₂ D² = N X = O |
| 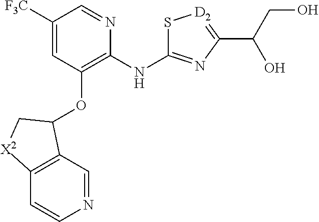 | D² = CH X = CH₂ D² = CH₂ X = O D² = N X = CH₂ D² = N X = O |
| 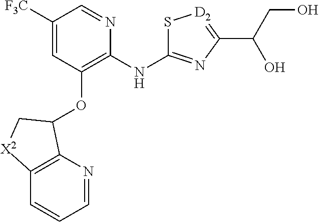 | D² = CH X = CH₂ D² = CH₂ X = O D² = N X = CH₂ D² = N X = O |
| 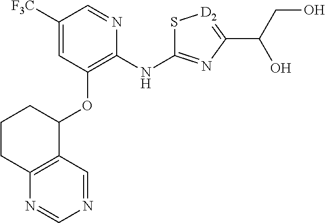 | D² = CH D² = N |
| 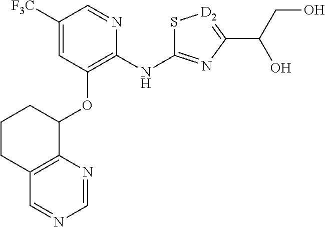 | D² = CH D² = N |
| 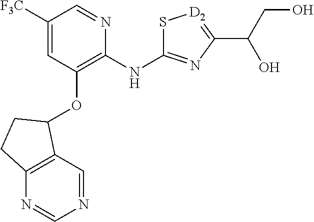 | D² = CH D² = N |
| 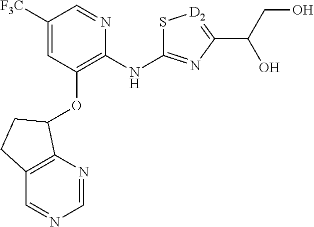 | D² = CH D² = N |
| 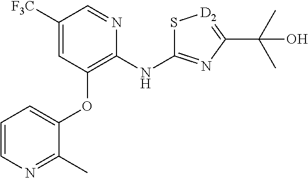 | D² = CH D² = N |
| 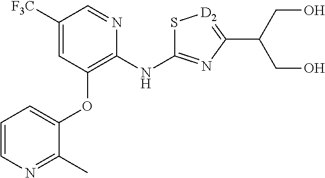 | D² = CH D² = N |
| 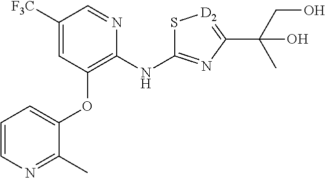 | D² = CH D² = N |
| 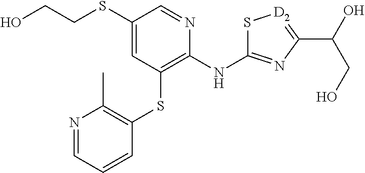 | D² = CH D² = N |
| 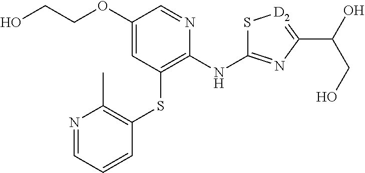 | D² = CH D² = N |

(S)-1-(5-(3-(2-methylpyridin-3-yloxy)-5-(pyridin-2-ylthio)pyridin-2-ylamino)-1,2,4-thiadiazol-3-yl)ethane-1,2-diol;
(S)-1-(5-(3-(2,6-dimethylpyridin-3-yloxy)-5-(pyridin-2-ylthio)pyridin-2-ylamino)-1,2,4-thiadiazol-3-yl)ethane-1,2-diol;
(S)-1-(5-(5-(cyclopropylmethylthio)-3-(2-methylpyridin-3-yloxy)pyridin-2-yl-amino)-1,2,4-thiadiazol-3-yl)ethane-1,2-diol;
(S)-1-(5-(3-(2-ethylpyridin-3-yloxy)-5-(pyridin-2-ylthio)pyridin-2-ylamino)-1,2,4-thiadiazol-3-yl)ethane-1,2-diol;
(S)-1-(5-(5-(3-methoxypropylthio)-3-(2-methylpyridin-3-yloxy)pyridin-2-ylamino)-1,2,4-thiadiazol-3-yl)ethane-1,2-diol;
(S)-1-(5-(3-(1-Ethyl-1H-pyrazol-5-yloxy)-5-(pyridin-2-ylthio)pyridin-2-ylamino)-1,2,4-thiadiazol-3-yl)ethane-1,2-diol;
(S)-1-(5-(3-(1-ethyl-1H-pyrazol-5-yloxy)-5-(pyridin-2-ylthio)pyridin-2-ylamino)-1,2,4-thiadiazol-3-yl)-2-methylpropane-1,2-diol;
(S)-1-(5-(5-(3-methylpyridin-2-ylthio)-3-(2-methylpyridin-3-yloxy)pyridin-2-yl-amino)-1,2,4-thiadiazol-3-yl)ethane-1,2-diol;
(S)-1-(5-(3-(2,4-dimethylpyridin-3-yloxy)-5-(pyridin-2-ylthio)pyridin-2-ylamino)-1,2,4-thiadiazol-3-yl)ethane-1,2-diol;
(S)-2-methyl-1-(5-(3-(2-methylpyridin-3-yloxy)-5-(pyridin-2-ylthio)pyridin-2-ylamino)-1,2,4-thiadiazol-3-yl)propane-1,2-diol;
(S)-1-(5-(5-(2-methoxyethylthio)-3-(2-methylpyridin-3-yloxy)pyridin-2-ylamino)-1,2,4-thiadiazol-3-yl)ethane-1,2-diol;
(1S,2S)-1-(5-(3-(2-ethylpyridin-3-yloxy)-5-(pyridin-2-ylthio)pyridin-2-ylamino)-1,2,4-thiadiazol-3-yl)-3-methoxypropane-1,2-diol;
(S)-2-methyl-1-(5-(5-(pyridin-2-ylthio)-3-(1,3,5-trimethyl-1H-pyrazol-4-yloxy)-pyridin-2-ylamino)-1,2,4-thiadiazol-3-yl)propane-1,2-diol;
(S)-1-(5-(5-(pyridin-2-ylthio)-3-(1,3,5-trimethyl-1H-pyrazol-4-yloxy)pyridin-2-yl-amino)-1,2,4-thiadiazol-3-yl)ethane-1,2-diol;
(S)-1-(5-(5-(2-methoxyethylthio)-3-(1,3,5-trimethyl-1H-pyrazol-4-yloxy)pyridin-2-yl-amino)-1,2,4-thiadiazol-3-yl)ethane-1,2-diol;
(R)-1-(5-(3-(2-methylpyridin-3-yloxy)-5-(pyridin-2-ylthio)pyridin-2-ylamino)-1,2,4-thiadiazol-3-yl)ethane-1,2-diol;
(S)-2-(5-(3-(2-methylpyridin-3-yloxy)-5-(pyridin-2-ylthio)pyridin-2-ylamino)-1,2,4-thiadiazol-3-yl)propane-1,2-diol; or
(R)-2-(5-(3-(2-methylpyridin-3-yloxy)-5-(pyridin-2-ylthio)pyridin-2-ylamino)-1,2,4-thiadiazol-3-yl)propane-1,2-diol, or the pharmaceutically acceptable salts thereof.

Other compounds that may be used in combination with the compounds of the present invention include the IL1-R1 compounds set forth in U.S. Pat. No. 7,438,910. A particular disease that can be treated with the combination is type 2 diabetes.

The compounds of the present invention can also be used in combination with FGF-21 compounds, and particularly for the treatment of type 2 diabetes. Examples of FGF-21 compounds are disclosed in U.S. Pat. No. 7,671,180; U.S. Pat. No. 7,667,008; U.S. Pat. No. 7,459,540; U.S. Pat. No. 7,696,172; PCT application publication no. WO 2010/042747; and PCT application publication no. WO 2009/149171.

The compounds of the present invention can be also be used in combination with anakinra, particularly for the treatment of type 2 diabetes.

In one particular aspect, the compounds of the present invention may be used in combination with metformin.

The compounds of the present invention are used in the treatment diseases or symptoms mediated by GKRP and/or GK (GKRP/GK). Examples of diseases or symptoms mediated by GKRP/GK include, but are not limited to, Type II (type 2) diabetes and related disorders, such as hyperglycemia, low or impaired glucose tolerance, insulin resistance, obesity, lipid disorders such as dyslipidemia, hyperlipidemia, hypertriglyceridemia, hypercholesterolemia, low HDL levels, high LDL levels, atherosclerosis, and vascular restenosis, irritable bowel syndrome, inflammatory bowel disease, including Crohn's disease and ulcerative colitis, other inflammatory conditions, pancreatitis, abdominal obesity, neurodegenerative disease, retinopathy, nephropathy, neuropathy, cataracts, glaucoma, glomerulosclerosis, foot ulcerations and ulcerative colitis, altered gastrointestinal motility, Syndrome X, ovarian hyperandrogenism, polycystic ovarian syndrome, premenstrual syndrome, other disorders where insulin resistance is a component. In Syndrome X, also known as Metabolic Syndrome, obesity is thought to promote insulin resistance, diabetes, dyslipidemia, hypertension, and increased cardiovascular risk, growth hormone deficiency, neutropenia, neuronal disorders, tumor invasion and metastasis, benign prostatic hypertrophy, gingivitis, osteoporosis, frailty of aging, intestinal injury, benign prostatic hypertrophy (BPH), and sperm motility/male contraception.

The compounds of the present invention are also useful for the prevention, delay of progression or the treatment of an early cardiac or early cardiovascular diseases or damages, renal diseases or damages, heart Failure, or heart Failure associated diseases like (i) cardiovascular diseases or damages e.g. cardiac hypertrophy, cardiac remodeling after myocardial infarction, pulmonary congestion and cardiac fibrosis in dilated or in hypertrophic cardiomyopathy, cardiomyopathy such as dilated cardiomyopathy or hypertrophic cardiomyopathy, mesanglial hypertrophy, or diabetic cardiomyopathy, left or right ventricular hypertrophy, arrhythmia, cardiac dysrhythmia, syncopy, angina pectoris, cardiac bypass reocclusion, intermittent claudication, diastolic and/or systolic dysfunction, diabetic myopathy, stroke prevention in congestive heart failure, hypertrophic medial thickening in arteries and/or large vessels, mesenteric vasculature hypertrophy or atherosclerosis, preferably atherosclerosis in mammalian patients with hypertension of diabetes; (ii) renal diseases or damages like renal hyperfiltration such as after portal renal ablation, proteinuria in chronic renal disease, renal arteriopathy as a consequence of hypertension, nephrosclerosis, hypertensive nephrosclerosis or mesanglial hypertrophy; (iii) Heart Failure to be treated is secondary to idiopathic dilated cardiomyopathy and/or coronary ischemic disease.

The compounds of the present invention can also be used for the prevention, the delay of the onset, the delay of progression or the treatment of neurodegenerative disorders, cognitive disorders and for improving memory (both short term and long term) and learning ability wherein the (i) neurodegenerative disorder is dementia, senile dementia, schizophrenia, mild cognitive impairment, Alzheimer related dementia, Huntington's chores, tardive dyskinesia, hyperkinesias, mania, Morbus Parkinson, Steel-Richard syndrome, Down's syndrome, myasthenia gravis, nerve and brain trauma, vascular amyloidosis, cerebral haemorrhage I with amyloidosis, brain inflammation, Friedrich ataxia, acute confusion disorders, acute confusion disorders with apoptotic necrocytosis, amyotrophic lateral sclerosis, glaucoma, and Alzheimer's disease; (ii) cognitive disorders like cognitive deficits associated with schizophrenia, age-induced memory impairment, cognitive deficits associated with psychosis, cognitive impairment associated with diabetes, cognitive deficits associated with post-stroke, memory defects associated hypoxia, cognitive and attention deficits associated with senile dementia, attention deficits disorders, memory problems associated with mild cognitive impairment, impaired cognitive function associated with vascular dementia, cognitive problems associated with brain tumors, Pick's disease, cognitive deficits due to autism, cognitive deficits post electroconvulsive therapy, cognitive deficits associated with traumatic brain injury, amnesic disorders, deliriums, vitamin deficiency, dementias, impaired cognitive function associated with Parkinson's disease, attention-deficit disorders; (iii) prevention of memory impairment as a result of Alzheimer disease, Creutzfeld-Jakob disease, Pick disease, Huntington disease, AIDS, brain injury, brain aneurysm, epilepsy, stroke, toxicant exposure, mental retardation in children, Huntington's disease; (iv) to improve learning speed and potential in educational and rehabilitation contexts.

The compounds of the present invention can also be used for stimulating an immune response in a subject having or at risk of having cancer wherein the cancer is selected from the group consisting of basal cell carcinomas including cancers of the binary tract, bladder, urinary system, bone, brain, breast, cervical, endometrial, ovarian, uterine, choriocarcinoma, central nervous system, colon and rectal cancers, connective tissue cancer, cancer of the digestive system, esophageal, gastric, stomach, larynx, liver, pancreatic, colorectal, renal cancers; cancers of the urinary system; cancers of eye, head and neck, oral cavity, skin, prostate; cancers of biliary tract, testicular, thyroid; intra-epithelial neoplasm, leukemia, acute myeloid leukemia, acute lymphoid leukemia, chronic myeloid leukemia, chronic lymphoid leukemia; and other cancers of the respiratory system, lung, small cell lung, non-small cell lung; lymphoma, Hodgkin's lymphoma, Non-Hodgkin's lymphoma; melanoma, myeloma, neuroblastoma, retinoblastoma, fibrosarcoma (bone or connective tissue sarcoma), rhabdomyosarcoma; and other cancers including neoplastic conditions, adipose cell tumors, adipose cell carcinomas, such as liposarcoma.

The compounds of the present invention can also be used for the treatment or prophylaxis of chronic inflammatory diseases such as autoimmune disorders like rheumatoid arthritis, systemic lupus erythematosus, multiple sclerosis, psoriasis, allergies or asthma.

The compounds of the present invention can also be used in the treatment of pain, neuropathic pain, rheumatoid pain, osteoarthritis pain, anesthesia adjunct in mammalian patients undergoing surgery, chronic pain in advanced cancer, treatment of refractory diarrhea, biliary pain caused by gallstones.

The compounds of the present invention can also be used for the treatment of mammalian patients undergoing islet/pancreas transplantation, for the prevention or the delay of transplant rejection, or allograft rejection in transplantation, for improving pancreatic function by increasing the number and size of pancreatic beta-cells in the treatment of Type 1 diabetes patients, and for improving pancreatic function by increasing the number and size of pancreatic beta-cells in general.

Furthermore, the compounds of the present invention can be used for the treatment of mammalian patients with acne, skin disorders (e.g. pigmentation disorders or psoriasis), scleroderma, mycoses; anxiety, anxiety neurosis, major depression disorder, drug abuse, alcohol addiction, insomnia, chronic fatigue, sleep apnea; anorexia nervosa; epilepsy; migraine; encephalomyelitis; osteoarthritis, osteoporosis, calcitonin-induced osteoporosis; male and female sexual dysfunction, infertility; Type 1 diabetes; immunosuppression, HIV infection; hematopoiesis, anemia; and for weight reduction.

Additionally, the compounds of the present invention are useful for the prevention, delay of progression or treatment of (i) bacterial infections from Escherichia coli, Staphylococcus, Streptoococcus, Pseudomonas, Clostridium difficile infection, Legionella, Pneumococcus, Haemophilus, Klebsiella, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, Helicobacter pylori, and anthrax infection; (ii) mycobacterial infection from tuberculosis and leprosy; (iii) viral infection from HIV, Herpes simplex virus 1, Herpes simplex virus 2, Cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox and SARS; (iv) fungal infection from candidiasis, ringworm, histoplasmosis, blastomycosis, paracoccidioidomycosis, cryptococcosis, aspergillosis, chromomycosis, mycetoma infections, pseudallescheriasis, Tinea versicolor infection; (v) parasite infection from amebiasis, Trypanosoma cruzi, Fascioliasis, Leishmaniasis, Plasmodium, Onchocerciasis, Paragonimiasis, Trypanosoma brucei, Pneumocystis, Trichomonas vaginalis, Taenia, Hymenolepsis, Echinococcus, Schistosomiasis, neurocysticerosis, Necator americanus, and Trichuris trichuria.

Since one aspect of the present invention contemplates the treatment of the disease/conditions with a combination of pharmaceutically active compounds that may be administered separately, the invention further relates to combining separate pharmaceutical compositions in kit form. The kit comprises two separate pharmaceutical compositions: a compound of the present invention, and a second pharmaceutical compound. The kit comprises a container for containing the separate compositions such as a divided bottle or a divided foil packet. Additional examples of containers include syringes, boxes and bags. Typically, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing physician or veterinarian.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a compound of the present invention can consist of one tablet or capsule, while a daily dose of the second compound can consist of several tablets or capsules and vice versa. The memory aid should reflect this and aid in correct administration of the active agents.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of the present invention and other pharmaceutically active compounds, if desired, can be administered to a patient either orally, rectally, parenterally, (for example, intravenously, intramuscularly, or subcutaneously) intracisternally, intravaginally, intraperitoneally, intravesically, locally (for example, powders, ointments or drops), or as a buccal or nasal spray. All methods that are used by those skilled in the art to administer a pharmaceutically active agent are contemplated.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents.

Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. They may also contain opacifying agents, and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferable suppositories, which can be prepared by mixing the compounds of the present invention with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

Dosage forms for topical administration of a compound of the present invention include ointments, powders, sprays and inhalants. The active compound or fit compounds are admixed under sterile condition with a physiologically acceptable carrier, and any preservatives, buffers, or propellants that may be required. Opthalmic formulations, eye ointments, powders, and solutions are also contemplated as being within the scope of this invention.

The compounds of the present invention can be administered to a patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that can be used depends on a number of factors, including the requirements of the patient, the severity of the condition or disease being treated, and the pharmacological activity of the compound being administered. The determination of dosage ranges and optimal dosages for a particular patient is within the ordinary skill in the art.

The compounds of the present invention can be administered as pharmaceutically acceptable salts, esters, amides or prodrugs. The term “salts” refers to inorganic and organic salts of compounds of the present invention. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitiate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977).

Examples of pharmaceutically acceptable esters of the compounds of the present invention include C₁-C₈ alkyl esters. Acceptable esters also include C₅-C₇ cycloalkyl esters, as well as arylalkyl esters such as benzyl. C₁-C₄ alkyl esters are commonly used. Esters of compounds of the present invention may be prepared according to methods that are well known in the art.

Examples of pharmaceutically acceptable amides of the compounds of the present invention include amides derived from ammonia, primary C₁-C₈ alkyl amines, and secondary C₁-C₈ dialkyl amines. In the case of secondary amines, the amine may also be in the form of a 5 or 6 membered heterocycloalkyl group containing at least one nitrogen atom. Amides derived from ammonia, C₁-C₃ primary alkyl amines and C₁-C₂ dialkyl secondary amines are commonly used. Amides of the compounds of the present invention may be prepared according to methods well known to those skilled in the art.

The term “prodrug” means compounds that are transformed in vivo to yield a compound of the present invention. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

To illustrate, if the compound of the invention contains a carboxylic acid functional group, a prodrug can comprise an ester formed by the replacement of the hydrogen atom of the acid group with a group such as (C₁-C₈ alkyl, (C₂-C1₂)alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)aminomethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂₋₃)alkyl.

Similarly, if a compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate).

The compounds of the present invention may contain asymmetric or chiral centers, and therefore, exist in different stereoisomeric forms. It is contemplated that all stereoisomeric forms of the compounds as well as mixtures thereof, including racemic mixtures, form part of the present invention. In addition, the present invention contemplates all geometric and positional isomers. For example, if the compound contains a double bond, both the cis and trans forms (designated as S and E, respectively), as well as mixtures, are contemplated.

Mixture of stereoisomers, such as diastereomeric mixtures, can be separated into their individual stereochemical components on the basis of their physical chemical differences by known methods such as chromatography and/or fractional crystallization. Enantiomers can also be separated by converting the enantiomeric mixture into a diasteromeric mixture by reaction with an appropriate optically active compound (e.g., an alcohol), separating the diastereomers and converting (e.g., hydrolyzing) the individual diastereomers to the corresponding pure enantiomers. Also, some compounds may be atropisomers (e.g., substituted biaryls).

The compounds of the present invention may exist in unsolvated as well as solvated forms with pharmaceutically acceptable solvents such as water (hydrate), ethanol, and the like. The present invention contemplates and encompasses both the solvated and unsolvated forms.

It is also possible that compounds of the present invention may exist in different tautomeric forms. All tautomers of compounds of the present invention are contemplated. For example, all of the tautomeric forms of the tetrazole moiety are included in this invention. Also, for example, all keto-enol or imine-enamine forms of the compounds are included in this invention.

Those skilled in the art will recognize that the compound names and structures contained herein may be based on a particular tautomer of a compound. While the name or structure for only a particular tautomer may be used, it is intended that all tautomers are encompassed by the present invention, unless stated otherwise.

It is also intended that the present invention encompass compounds that are synthesized in vitro using laboratory techniques, such as those well known to synthetic chemists; or synthesized using in vivo techniques, such as through metabolism, fermentation, digestion, and the like. It is also contemplated that the compounds of the present invention may be synthesized using a combination of in vitro and in vivo techniques.

The present invention also includes isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁶O, ¹⁷O, ³¹P, ³²P, ³⁵S, ¹⁸F, and ³⁶Cl.

Compounds of the present invention that contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detection. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of this invention can generally be prepared by substituting a readily available isotopically labeled reagent for a non-isotopically labeled reagent.

The compounds of the present invention may exist in various solid states including crystalline states and as an amorphous state. The different crystalline states, also called polymorphs, and the amorphous states of the present compounds are contemplated as part of this invention.

In synthesizing compounds of the present invention, it may be desirable to use certain leaving groups. The term “leaving groups” (“LG”) generally refer to groups that are displaceable by a nucleophile. Such leaving groups are known in the art. Examples of leaving groups include, but are not limited to, halides (e.g., I, Br, F, Cl), sulfonates (e.g., mesylate, tosylate), sulfides (e.g., SCH₃), N-hydroxsuccinimide, N-hydroxybenzotriazole, and the like. Examples of nucleophiles include, but are not limited to, amines, thiols, alcohols, Grignard reagents, anionic species (e.g., alkoxides, amides, carbanions) and the like.

All patents and other publications recited herein are hereby incorporated by reference.

The examples presented below illustrate specific embodiments of the present invention. These examples are meant to be representative and are not intended to limit the scope of the claims in any manner.

### EXAMPLES

The following abbreviations may be used herein:
dba 1,5-diphenylpenta-1,4-dien-3-one
LAH lithium aluminum hydride
TMS trimethylsilyl
TLC Thin layer chromatography
MHz megahertz
br. broad
s singlet
d doublet
t triplet
dt doublet of triplet
dd doublet of doublet
quin quintuplet
q quartet
˜ about
+ve or pos. ion positive ion
Δ heat
Ac acetyl
Ac₂O acetic anhydride
ACN acetonitrile
Aq aqueous
BOC tert-butyloxycarbonyl
Bu butyl
Bz or Bn benzyl
Calcd or Calc'd calculated
Conc. concentrated
DCM dichloromethane
DEA diethylamine
DIEA diisopropylethylamine
DMAP 4-dimethylaminopyridine
DME dimethoxyl ethyl ether
DMF N,N-dimethylformamide
DMSO dimethyl sulfoxide
DTT dithiothreitol
ESI or ES electrospray ionization
Et ethyl
Et₂O diethyl ether
Et₃N triethylamine
EtOAc ethyl acetate
EtOH ethyl alcohol
g grams
h hour
Hex hexanes
HOAc acetic acid
HPLC high pressure liquid chromatography
IPA or iPrOH isopropyl alcohol
iPr₂NEt N-ethyl diisopropylamine
LCMS, LC-MS or LC/MS liquid chromatography mass spectroscopy
LDA lithium diisopropylamide
m/z mass divided by charge
Me methyl
MeCN acetonitrile
Met iodomethane
MeOH methyl alcohol
mg milligrams
min minutes
mL milliliters
MS mass spectra
MsCl mesylchloride
NMR nuclear magnetic resonance
RT or rt room temperature
Sat. or sat'd or satd saturated
SFC supercritical fluid chromatography
TFA trifluoroacetic acid
Tris tris(hydroxymethyl)aminomethane

The following synthetic schemes show generally how to make compounds of the present invention.

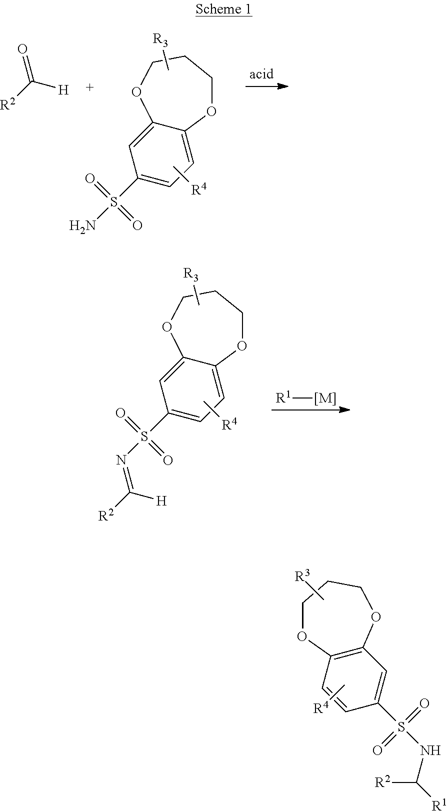

The N-sulfonylimine intermediates can be synthesized by the condensation of a substituted aldehyde with a sulfonamide in an anhydrous solvent (such as toluene or benzene) in the presence of either an inorganic or organic acid (such as Montmorillonite K10 (Sigma-Aldrich, St. Louis, Mo.) or p-toluenesulfonic acid). The sulfonamide final product can be synthesized by nucleophilic addition of an aryl anion species (such as [M]=Mg or Li salts) or metal-catalyzed cross-coupling of an aryl metal species (such as [M]=B or Sn salts).

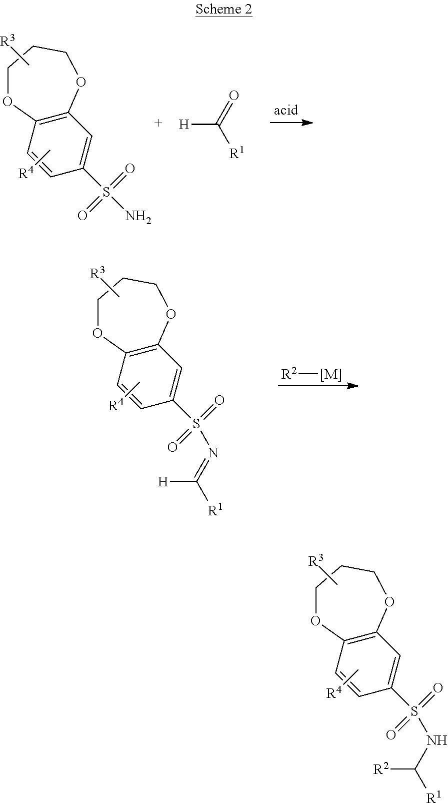

The N-sulfonylimine intermediates can be synthesized by the condensation of a substituted aldehyde with a sulfonamide in an anhydrous solvent (such as toluene or benzene) in the presence of either an inorganic or organic acid (such as Montmorillonite K10 (Sigma-Aldrich, St. Louis, Mo.) or p-toluenesulfonic acid). The sulfonamide final product can be synthesized by nucleophilic addition of an aryl anion species (such as [M]=Mg or Li salts) or metal-catalyzed cross-coupling of an aryl metal species (such as [M]=B or Sn salts).

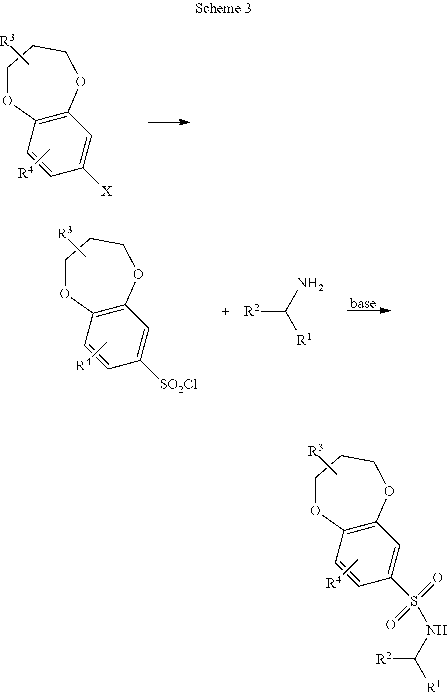

Sulfonyl chloride intermediates can be synthesized by metallation of an aryl or aryl halide species (such as X═Br or I) followed by coupling of the aryl metal species with SO₂ and subsequent oxidative chlorination of the intermediate sulfinic acid salt. The sulfonamide final product can be synthesized by nucleophilic addition of a benzylic amine species to the sulfonyl chloride in the presence of a base (such as Et₃N or i-Pr₂NEt).

### Intermediate 1

### N—((S,1E)-(2-Chlorophenyl)methylidene)-2-methyl-2-propanesulfinamide

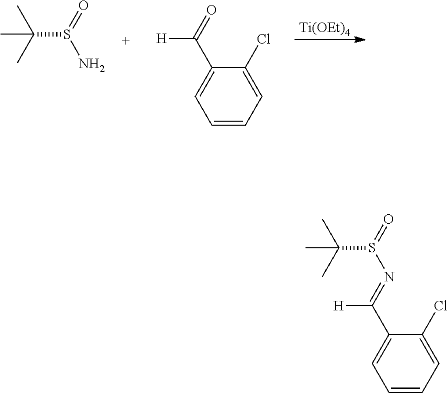

A 1 L round-bottomed flask was charged with (S)-2-methyl-2-propanesulfinamide (26 g, 210 mmol, AK Scientific, Mountain View, Calif.), dichloromethane (430 mL), 2-chlorobenzaldehyde (30 g, 210 mmol, Sigma-Aldrich, St. Louis, Mo.), and titanium(IV) tetraethanolate (220 mL, 1100 mmol, Sigma-Aldrich, St. Louis, Mo.), and then the reaction mixture was stirred for 22 h. After that time, the reaction mixture was added to water (1 L), dichloromethane (1.5 L) was added, the mixture was agitated vigorously, filtered through a pad of diatomaceous earth (100 g), the two layers comprising the filtrate were separated, the organic material was dried (sodium sulfate), filtered, and the filtrate was concentrated under a vacuum. The residue was subjected to flash chromatography on silica gel (500 g of silica gel, 9:1 hexane-ethyl acetate) to afford N—((S,1E)-(2-chlorophenyl)methylidene)-2-methyl-2-propanesulfinamide (46 g) as a clear yellow oil.

### Intermediate 2

### (R)-1-(1-Benzothiophen-2-yl)-1-(2-chlorophenyl)methanamine hydrochloride

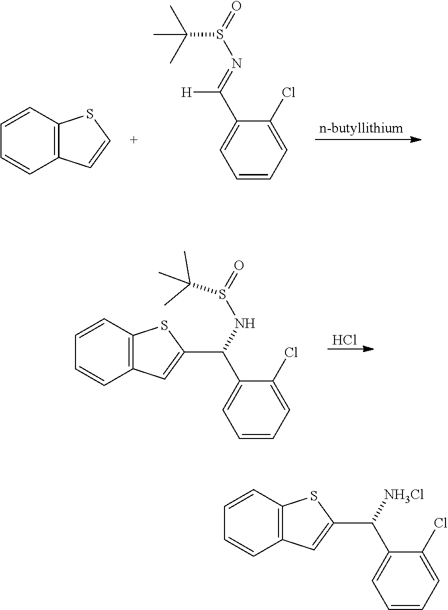

Step 1.

A 150 mL round-bottomed flask was charged with 1-benzothiophene (0.79 g, 5.9 mmol, Sigma-Aldrich, St. Louis, Mo.) and tetrahydrofuran (26 mL). The solution was cooled to −78° C., n-butyllithium (2.6 mL of a 2.5 M solution with toluene, 6.4 mmol, Sigma-Aldrich, St. Louis, Mo.) was added, and then the reaction mixture was stirred for 15 min. After that time, a solution of N—((S,E)-(2-chlorophenyl)methylidene)-2-methyl-2-propanesulfinamide (Intermediate 1) (1.3 g, 5.3 mmol) and tetrahydrofuran (26 mL) was added, and then the reaction mixture was stirred for 90 min. After that time, saturated aqueous ammonium chloride was (53 mL) added. The reaction vessel was removed from the cooling bath, the mixture was allowed to warm to room temperature, partitioned between ethyl acetate (53 mL) and more saturated aqueous ammonium chloride (53 mL), the layers were separated, the organic material was washed sequentially with saturated aqueous ammonium chloride (110 mL) and brine (110 mL), dried (sodium sulfate), filtered, the filtrate was concentrated, the residue was dissolved with dichloromethane (20 mL), silica gel (10 g) was added, and the volatiles were removed under a vacuum. The residue was subjected to flash chromatography on silica gel (120 g of silica gel, gradient elution of 9:1 to 4:1 to 3:1 to 2:1 hexane-ethyl acetate). The isolated material (1.4 g) was dissolved with dichloromethane (20 mL), silica gel (7.0 g) was added, and the volatiles were removed under a vacuum. The residue was subjected to flash chromatography on silica gel (120 g of silica gel, gradient elution of 3:1 to 2:1 hexane-ethyl acetate) to give (S)—N—((R)-1-benzothiophen-2-yl(2-chlorophenyl)methyl)-2-methyl-2-propanesulfinamide (1.3 g) as a colorless solid.

Step 2.

A 15 mL round-bottomed flask was charged with (S)—N—((R)-1-benzothiophen-2-yl(2-chlorophenyl)methyl)-2-methyl-2-propanesulfinamide (from Step 1) (1.3 g, 3.4 mmol), methanol (3.4 mL), and hydrogen chloride (1.7 mL of a 4.0 M solution with 1,4-dioxane, 6.9 mmol, Sigma-Aldrich, St. Louis, Mo.), and then the reaction mixture was stirred for 80 min. After that time, the reaction mixture was concentrated under a vacuum, diethyl ether (50 mL) was added to the residue, the resulting slurry was sonicated for 5 min, filtered, and the filter cake was collected to afford (R)-1-(1-benzothiophen-2-yl)-1-(2-chlorophenyl)methanamine hydrochloride (0.90 g) as an off-white solid.

### Intermediate 3

### 2-(2-(2-((R)-Amino(2-chlorophenyl)methyl)-1-benzothiophen-7-yl)-4-pyridinyl)-1,1,1,3,3,3-hexafluoro-2-propanol hydrochloride

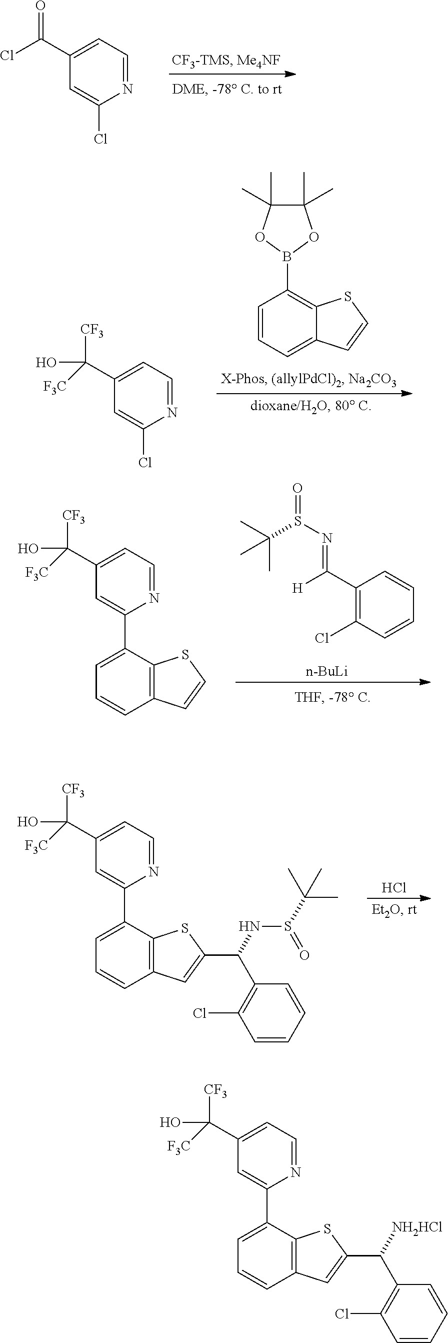

Step 1.

Trimethyl(trifluoromethyl)silane (2.52 mL, 17.05 mmol, Alfa Aesar, Ward Hill, Mass.) was added to a mixture of 2-chloro-4-pyridinecarbonyl chloride (1.00 g, 5.68 mmol, Sigma-Aldrich, St. Louis, Mo.) and tetramethylammonium fluoride (1.59 g, 17.05 mmol, Sigma-Aldrich, St. Louis, Mo.) in DME (15 mL) at −78° C. and the mixture was stirred for 17 h while it warmed up to room temperature. The mixture was cooled to −78° C., tetramethylammonium fluoride (0.529 g, 5.68 mmol) followed by trimethyl(trifluoromethyl)silane (0.840 mL, 5.68 mmol) was added and the mixture was stirred for 4 h while it warmed up to room temperature. The mixture was cooled to 0° C., quenched by the addition of saturated aqueous NaHCO₃ (20 mL), and extracted with DCM (3×30 mL). The combined organic layers were dried (MgSO₄), filtered, and concentrated under reduced pressure. The resulting yellow oil was purified by flash chromatography (100 g of silica gel, 10% to 30% EtOAc/hexanes) to deliver 2-(2-chloro-4-pyridinyl)-1,1,1,3,3,3-hexafluoro-2-propanol (1.29 g) as an off-white solid.

Step 2.

A mixture of 2-(2-chloro-4-pyridinyl)-1,1,1,3,3,3-hexafluoro-2-propanol (1.27 g, 4.55 mmol), 2-(1-benzothiophen-7-yl)-4,4,5,5-tetramethyl-1,3,2-dioxaborolane (2.01 g, 7.73 mmol, See Wang, B.; Akay, S.; Yang, W.; Wang, J. Chem. Biol. Drug Des. 2007, 70, 279-289), dicyclohexyl(2′,4′,6′-tris(1-methylethyl)-2-biphenylyl)phosphane (108 mg, 0.227 mmol, Sigma-Aldrich, St. Louis, Mo.), chloro(2-propen-1-yl)palladium dimer (41.6 mg, 0.114 mmol, Strem, Newburyport, Mass.), and sodium carbonate monohydrate (204 mg, 13.65 mmol, Mallinckrodt Baker, Phillipsburg, N.J.) in dioxane/water (4:1, 10 mL) was stirred at 80° C. for 5 h. Dicyclohexyl(2′,4′,6′-tris(1-methylethyl)-2-biphenylyl)phosphane (108 mg, 0.227 mmol) and chloro(2-propen-1-yl)palladium dimer (41.6 mg, 0.114 mmol) were added and the mixture was stirred at 80° C. for 2 h. The mixture was cooled to room temperature, diluted with MeOH/DCM, absorbed onto silica gel, and purified by flash chromatography (100 g of silica gel, 10% to 30% EtOAc/hexanes) to deliver 2-(2-(1-benzothiophen-7-yl)-4-pyridinyl)-1,1,1,3,3,3-hexafluoro-2-propanol (1.50 g) as an off-white solid.

Step 3.

Butyllithium (3.32 mL of a 2.5M solution in toluene, 8.29 mmol, Sigma-Aldrich, St. Louis, Mo.) was added to a solution of 2-(2-(1-benzothiophen-7-yl)-4-pyridinyl)-1,1,1,3,3,3-hexafluoro-2-propanol (1.49 g, 3.95 mmol) in THF (20 mL) at −78° C. and the mixture was stirred at −78° C. for 0.5 h. A solution of (S)—N-((1E)-(2-chlorophenyl)methylidene)-2-methyl-2-propanesulfinamide (Intermediate 1) (1.25 mg, 5.13 mmol) in THF (4 mL) at −78° C. was added dropwise via cannula and the resulting mixture was stirred at −78° C. for 2.5 h. Saturated aqueous NH₄Cl (10 mL) was added and the mixture was warmed to room temperature. The mixture was partitioned between half-saturated aqueous NH₄Cl (30 mL) and EtOAc (40 mL), the layers were separated, and the aqueous layer was extracted with EtOAc (2×40 mL). The combined organic layers were dried (MgSO₄), filtered, and concentrated under reduced pressure. The resulting yellow oil was purified by flash chromatography (100 g of silica gel, 30% to 70% EtOAc/hexanes) and again by by flash chromatography (80 g of silica gel, 30% to 70% EtOAc/hexanes) to deliver (S)—N—((R)-(2-chlorophenyl)(7-(4-(2,2,2-trifluoro-1-hydroxy-1-(trifluoromethyl)ethyl)-2-pyridinyl)-1-benzothiophen-2-yl)methyl)-2-methyl-2-propanesulfinamide (1.26 g) as a white solid.

Step 4.

Hydrogen chloride (7.92 mL of a 1 M solution in Et₂O, 7.92 mmol, Sigma-Aldrich, St. Louis, Mo.) was added to a solution of (S)—N—((R)-(2-chlorophenyl)(7-(4-(2,2,2-trifluoro-1-hydroxy-1-(trifluoromethyl)ethyl)-2-pyridinyl)-1-benzothiophen-2-yl)methyl)-2-methyl-2-propanesulfinamide (1.23 g, 1.98 mmol) in Et₂O (12 mL) and the resulting suspension was stirred at room temperature for 5 h. The solid was removed by filtration, washed with Et₂O (10 mL) and dried under reduced pressure to deliver 2-(2-(2-((R)-amino(2-chlorophenyl)methyl)-1-benzothiophen-7-yl)-4-pyridinyl)-1,1,1,3,3,3-hexafluoro-2-propanol hydrochloride (Intermediate 1) (1.05 g) as a light yellow solid.

### Intermediates 4 and 5

### 2-(2-Chloro-4-pyridinyl)-1,1,1-trifluoro-2-propanol

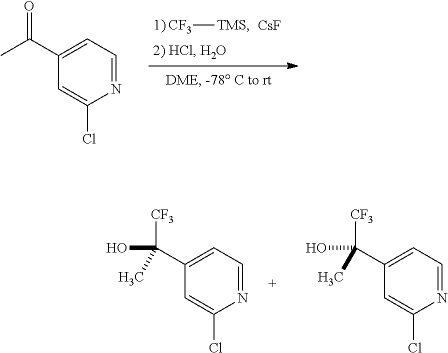

A mixture of 1-(2-chloro-4-pyridinyl)ethanone (14.8 g, 95.0 mmol, Maybridge, Tintagel, United Kingdom) and trimethyl(trifluoromethyl)silane (18.3 mL, 124 mmol, Alfa Aesar, Ward Hill, Mass.) in DME (100 mL) was cooled until the inside temperature of the reaction mixture reached −78° C. Cesium fluoride (0.867 g, 5.71 mmol, Strem, Newburyport, Mass.) was added and the reaction was warmed to room temperature. 10% Aqueous HCl (200 mL) was added and the mixture was stirred at room temperature for 20 h. Water (300 mL) was added and the resulting mixture was extracted with Et₂O (4×150 mL). The combined organic layers were washed with brine (200 mL), dried (Na₂SO₄), and concentrated under reduced pressure. The residue was purified by flash chromatography (100 g of silica gel, 25% EtOAc/hexane) to deliver 2-(2-chloro-4-pyridinyl)-1,1,1-trifluoro-2-propanol (19.0 g) as a light yellow solid (racemic mixture).

The racemic mixture was resolved using preparative SFC (Chiralcel® OJ-H column (Chiral Technologies, Inc, Westchester, Pa.) (two 250 mm×30 mm, 5 μm columns run in series) eluting with 90% liquid CO₂ in 10% mixture of hexane/isopropanol (75:25 v/v) containing 20 mM ammonia in methanol at a flow rate of 120 mL/min) to give two products in greater than 98% enantiomeric excess.

The absolute configurations at the chiral carbinol center of these two enantiomeric products were determined via vibrational circular dichroism (VCD) studies (See Stephens, P. J.; Devlin, F. J.; Pan, J.-J. Chirality 2008, 20, 643-663).

1. Computational: The conformational ensemble of the enantiomer with the (S)-configuration was initially generated via stochastic search with the MMFF94 molecular mechanics force field as implemented in the MOE (2008.10) package (Chemical Computing Group, Montreal, CA). This was followed by geometry optimization, vibrational frequency, and VCD rotational strength determination of the structurally unique conformers using the B3PW91 density functional and the 6-311G(2d,2p) basis set as implemented in the Gaussian 03 quantum chemical program system (Gaussian, Inc., Wallingford, Conn.). The resultant VCD line spectra were convolved utilizing a Lorentzian lineshape function (4 cm⁻¹ half-width), and summed over the conformational ensemble based on the relative free energies (and thus population) of each conformer.

2. Experimental: VCD spectra of each enantiomer were measured on a Biotools/BoMem ChiralIR instrument (Biotools, Inc., Jupiter, Fla.) at concentrations of ca. 30 mg/mL in CDCl₃ in a 100 μM BaF2 cell over 4 hr acquisition time at 4 cm⁻¹ resolution.

Comparison of the predicted and experimental VCD spectra (from 1. and 2. above, respectively) afforded the absolute configuration assignment.

First eluting peak (peak #1): (2R)-2-(2-chloro-4-pyridinyl)-1,1,1-trifluoro-2-propanol (Intermediate 4).

Second eluting peak (peak #2): (25)-2-(2-chloro-4-pyridinyl)-1,1,1-trifluoro-2-propanol (Intermediate 5).

### Intermediate 6

### (2R)-2-(2-(2-((R)-Amino(2-chlorophenyl)methyl)-1-benzothiophen-7-yl)-4-pyridinyl)-1,1,1-trifluoro-2-propanol

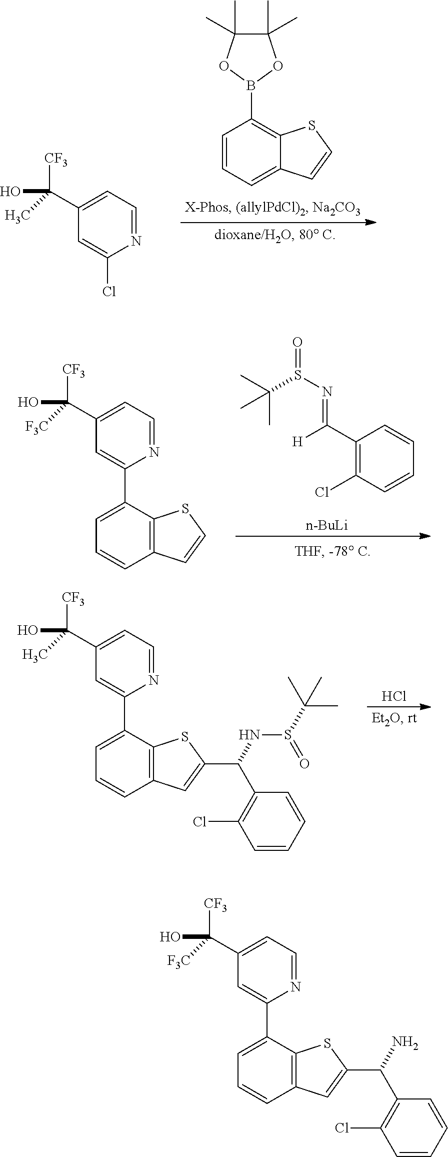

Step 1.

A mixture of (2R)-2-(2-chloro-4-pyridinyl)-1,1,1-trifluoro-2-propanol (Intermediate 4) (3.00 g, 13.3 mmol), 2-(1-benzothiophen-7-yl)-4,4,5,5-tetramethyl-1,3,2-dioxaborolane (6.23 g, 23.9 mmol, See Wang, B.; Akay, S.; Yang, W.; Wang, J. Chem. Biol. Drug Des. 2007, 70, 279-289), dicyclohexyl(2′,4′,6′-tris(1-methylethyl)-2-biphenylyl)phosphane (0.697 g, 1.46 mmol, Sigma-Aldrich, St. Louis, Mo.), chloro(2-propen-1-yl)palladium dimer (0.122 g, 0.332 mmol, Strem, Newburyport, Mass.), and sodium carbonate (4.23 g, 39.9 mmol, Mallinckrodt Baker, Phillipsburg, N.J.) in dioxane/water (4:1, 40 mL) was stirred at 80° C. for 2 h. The mixture was cooled to room temperature, water (300 mL) was added, and the resulting mixture was extracted with EtOAc (2×350 mL). The combined organic layers were washed with brine (200 mL), dried (Na₂SO₄), filtered, and concentrated under reduced pressure. The residue was purified by flash chromatography (100 g of silica gel, 25% EtOAc/hexane) and again by flash chromatography (150 g of silica gel, 10% EtOAc/hexane) to afford (2R)-2-(2-(1-benzothiophen-7-yl)-4-pyridinyl)-1,1,1-trifluoro-2-propanol (2.8 g) as a light yellow solid.

Step 2.

Butyllithium (8.44 mL of a 1.6 M solution in hexanes, 13.5 mmol, Sigma-Aldrich, St. Louis, Mo.) was added to a solution of (2R)-2-(2-(1-benzothiophen-7-yl)-4-pyridinyl)-1,1,1-trifluoro-2-propanol (1.82 g, 5.63 mmol) in THF (27 mL) at −78° C. and the mixture was stirred at −78° C. for 10 min. A solution of (S)—N-((1E)-(2-chlorophenyl)methylidene)-2-methyl-2-propanesulfinamide (Intermediate 1) (2.06 g, 8.44 mmol) in THF (8 mL) was added dropwise via cannula and the resulting mixture was stirred at −78° C. for 2 h. Saturated aqueous NH₄Cl (4 mL) was added and the mixture was warmed to room temperature. The mixture was partitioned between half-saturated aqueous NH₄Cl (50 mL; can be made by taking 25 mL of a saturated solution and adding 25 mL of water) and EtOAc (50 mL), the layers were separated, and the aqueous layer was extracted with EtOAc (2×50 mL). The combined organic layers were dried (MgSO₄), filtered, and concentrated under reduced pressure. The resulting yellow oil was absorbed onto silica gel and purified by flash chromatography (100 g of silica gel, 10% to 60% EtOAc/hexanes) and again by flash chromatography (120 g of silica gel, 30% to 60% EtOAc/hexanes) to deliver (S)—N—((R)-(2-chlorophenyl)(7-(4-((1R)-2,2,2-trifluoro-1-hydroxy-1-methylethyl)-2-pyridinyl)-1-benzothiophen-2-yl)methyl)-2-methyl-2-propanesulfinamide (1.87 g) as a white foam.

Step 3.

Hydrogen chloride (8.16 mL of a 2 M solution in Et₂O, 16.31 mmol, Sigma-Aldrich, St. Louis, Mo.) was added to a solution of (S)—N—((R)-(2-chlorophenyl)(7-(4-((1R)-2,2,2-trifluoro-1-hydroxy-1-methylethyl)-2-pyridinyl)-1-benzothiophen-2-yl)methyl)-2-methyl-2-propanesulfinamide (1.85 g, 3.26 mmol) in Et₂O (24 mL) and the resulting suspension was stirred at room temperature for 18 h. The solid was collected by filtration and washed with Et₂O (20 mL). The solid was dissolved in 2 M NH₄ in MeOH/DCM, absorbed onto silica gel, and purified by flash chromatography (100 g of silica gel, 30% to 70% EtOAc/hexanes) to deliver (2R)-2-(2-(2-((R)-amino(2-chlorophenyl)methyl)-1-benzothiophen-7-yl)-4-pyridinyl)-1,1,1-trifluoro-2-propanol (Intermediate 6) (1.34 g) as a white solid.

### Intermediate 7

### (2S)-2-(2-(2-((R)-amino(2-chlorophenyl)methyl)-1-benzothiophen-7-yl)-4-pyridinyl)-1,1,1-trifluoro-2-propanol

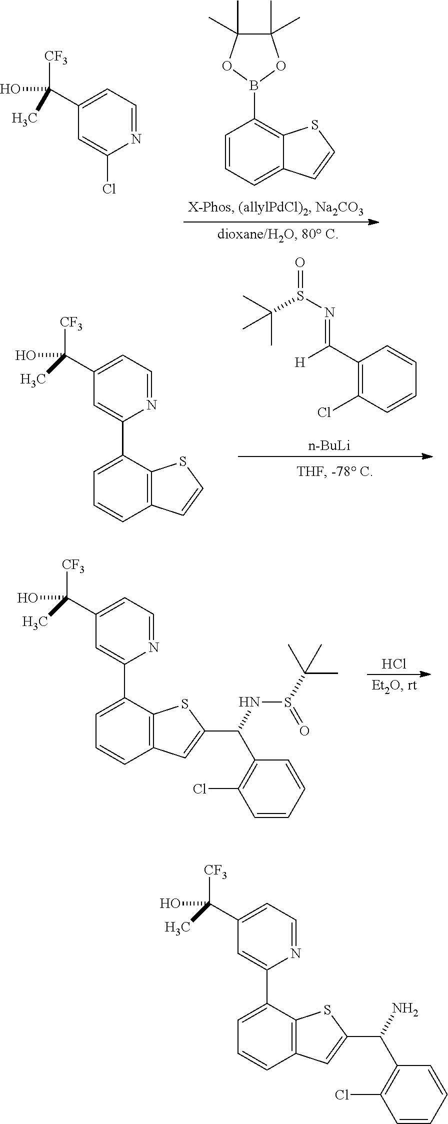

Step 1.

A mixture of (25)-2-(2-chloro-4-pyridinyl)-1,1,1-trifluoro-2-propanol (Intermediate 5) (1.07 g, 4.74 mmol), 2-(1-benzothiophen-7-yl)-4,4,5,5-tetramethyl-1,3,2-dioxaborolane (2.10 g, 8.06 mmol, See Wang, B.; Akay, S.; Yang, W.; Wang, J. Chem. Biol. Drug Des. 2007, 70, 279-289), dicyclohexyl(2′,4′,6′-tris(1-methylethyl)-2-biphenylyl)phosphane (226 mg, 0.474 mmol, Sigma-Aldrich, St. Louis, Mo.), chloro(2-propen-1-yl)palladium dimer (87 mg, 0.237 mmol, Strem, Newburyport, Mass.), and sodium carbonate monohydrate (1.76 g, 14.2 mmol, Mallinckrodt Baker, Phillipsburg, N.J.) in dioxane/water (4:1, 10 mL) was stirred at 80° C. for 7 h and at room temperature for 14 h. The mixture was concentrated under reduced pressure and the resulting residue was taken up in MeOH/DCM, absorbed onto silica gel, and purified by flash chromatography (100 g of silica gel, 10% to 30% EtOAc/hexanes) and again by flash chromatography (100 g of silica gel, 10% to 30% EtOAc/hexanes) to deliver (2S)-2-(2-(1-benzothiophen-7-yl)-4-pyridinyl)-1,1,1-trifluoro-2-propanol (769 mg) as a colorless oil.

Step 2.

Butyllithium (3.21 mL of a 1.6 M solution in hexane, 5.14 mmol, Sigma-Aldrich, St. Louis, Mo.) was added to a solution of (2S)-2-(2-(1-benzothiophen-7-yl)-4-pyridinyl)-1,1,1-trifluoro-2-propanol (755 mg, 2.34 mmol) in THF (2 mL) at −78° C. and the mixture was stirred at −78° C. for 30 min. A solution of (S)—N-((1E)-(2-chlorophenyl)methylidene)-2-methyl-2-propanesulfinamide (Intermediate 1) (740 mg, 3.04 mmol) in THF (1 mL) at −78° C. was added dropwise via cannula and the resulting mixture was stirred at −78° C. for 3 h. Saturated aqueous NH₄Cl (2 mL) was added and the mixture was warmed to room temperature. It was partitioned between half-saturated aqueous NH₄Cl (20 mL) and EtOAc (20 mL), the layers were separated, and the aqueous layer was extracted with EtOAc (2×20 mL). The combined organic layers were dried (MgSO₄), filtered, and concentrated under reduced pressure. The resulting yellow oil was purified by flash chromatography (80 g of silica gel, 10% to 60% EtOAc/hexanes) and again by flash chromatography (25 g of silica gel, 30% to 60% EtOAc/hexanes) to deliver (S)—N—((R)-(2-chlorophenyl)(7-(4-((1S)-2,2,2-trifluoro-1-hydroxy-1-methylethyl)-2-pyridinyl)-1-benzothiophen-2-yl)methyl)-2-methyl-2-propanesulfinamide (674 mg) as a light yellow foam.

Step 3.

Hydrogen chloride (2.90 mL of a 2 M solution in Et₂O, 5.80 mmol, Alfa Aesar, Ward Hill, Mass.) was added to a solution of (S)—N—((R)-(2-chlorophenyl)(7-(4-((1S)-2,2,2-trifluoro-1-hydroxy-1-methylethyl)-2-pyridinyl)-1-benzothiophen-2-yl)methyl)-2-methyl-2-propanesulfinamide (658 mg, 1.16 mmol) in Et₂O (8 mL) and the resulting suspension was stirred at room temperature for 6 h. The solid was removed by filtration, washed with Et₂O (10 mL), and dried under reduced pressure. The solid was dissolved in 2 M NH₄ in MeOH/DCM, absorbed onto silica gel, and purified by flash chromatography (100 g of silica gel, 30% to 70% EtOAc/hexanes) to deliver (2S)-2-(2-(2-((R)-amino(2-chlorophenyl)methyl)-1-benzothiophen-7-yl)-4-pyridinyl)-1,1,1-trifluoro-2-propanol (Intermediate 7) (505 mg) as a white foam.

### Example 1

### N-(1-benzofuran-2-yl(phenyl)methyl)-3,4-dihydro-2H-1,5-benzodioxepine-7-sulfonamide

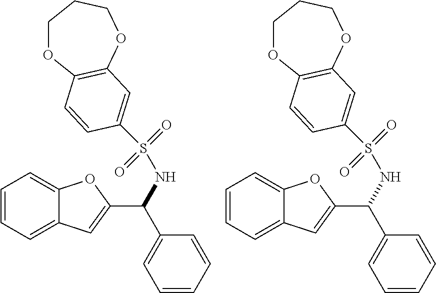

A racemic mixture of N-(1-benzofuran-2-yl(phenyl)methyl)-3,4-dihydro-2H-1,5-benzodioxepine-7-sulfonamide (Enamine, Kiev, Ukraine) was resolved using preparative SFC (Chiralcel® OJ-H column (Daicel, Inc., Fort Lee, N.J.) (150 mm×30 mm, 5 μm) eluting with 85% liquid CO₂ in 15% isopropanol (with 0.10% diethylamine) at a flow rate of 60 mL/min) to give two products in greater than 98% enantiomeric excess.

### N—((R)-1-benzofuran-2-yl(phenyl)methyl)-3,4-dihydro-2H-1,5-benzodioxepine-7-sulfonamide

First Eluting Peak (Peak #1)

¹H NMR (400 MHz, chloroform-d) δ 7.45 (d, J=7.4 Hz, 1H), 7.35-7.09 (m, 9H), 6.78 (d, J=8.2 Hz, 1H), 6.44 (s, 1H), 5.76 (d, J=8.2 Hz, 1H), 5.24 (d, J=8.0 Hz, 1H), 4.17-3.93 (m, 4H), 2.21-2.04 (m, 2H). m/z (ESI, +ve ion) 458.0 (M+Na)⁺. GK-GKRP EC₅₀ (LCMS/MS)=0.17 μM.

### N—((S)-1-benzofuran-2-yl(phenyl)methyl)-3,4-dihydro-2H-1,5-benzodioxepine-7-sulfonamide

Second Eluting Peak (Peak #2)

¹H NMR (400 MHz, chloroform-d) δ 7.45 (d, J=7.4 Hz, 1H), 7.35-7.09 (m, 9H), 6.78 (d, J=8.2 Hz, 1H), 6.44 (s, 1H), 5.76 (d, J=8.2 Hz, 1H), 5.24 (d, J=8.0 Hz, 1H), 4.17-3.93 (m, 4H), 2.21-2.04 (m, 2H). m/z (ESI, +ve ion) 458.0 (M+Na)⁺. GK-GKRP EC₅₀ (LCMS/MS)=4.6 μM.

### Intermediate A

### N-(1-benzofuran-2-ylmethylidene)-3,4-dihydro-2H-1,5-benzodioxepine-7-sulfonamide

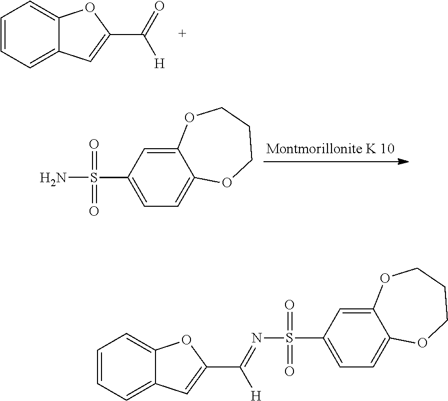 [SMILES: O=Cc1cc2ccccc2o1 ; NS(=O)(=O)c1ccc2c(c1)OCCCO2 ; O=S(=O)(/N=C/c1cc2ccccc2o1)c1ccc2c(c1)OCCCO2]

A 250 mL round-bottomed flask was charged with 1-benzofuran-2-carbaldehyde (4.0 g, 27 mmol, Sigma-Aldrich, St. Louis, Mo.), 3,4-dihydro-2H-1,5-benzodioxepine-7-sulfonamide (6.3 g, 27 mmol, Enamine, Kiev, Ukraine), Montmorillonite K 10 (4.0 g, Sigma-Aldrich, St. Louis, Mo.), and toluene (140 mL), fitted with a Dean-Stark trap and a reflux condenser, and then the reaction mixture was heated at reflux for 2 h. After that time, the reaction mixture was filtered, the filtrate was allowed to cool to room temperature, filtered, and the filter cake was collected to give N-(1-benzofuran-2-ylmethylidene)-3,4-dihydro-2H-1,5-benzodioxepine-7-sulfonamide (7.6 g) as a colorless solid. It is noted that the intermediates used in the synthesis of the Examples herein were generally used as obtained without further purification.

### Example 2

### N-(1-benzofuran-2-yl(2-methylphenyl)methyl)-3,4-dihydro-2H-1,5-benzodioxepine-7-sulfonamide

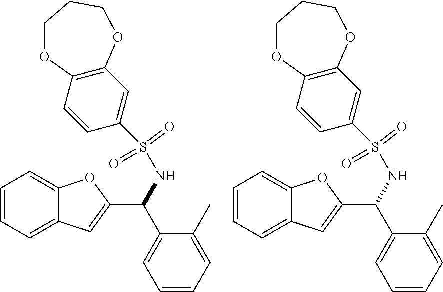

A 10 mL round-bottomed flask was charged with N-(1-benzofuran-2-ylmethylidene)-3,4-dihydro-2H-1,5-benzodioxepine-7-sulfonamide (intermediate A) (0.10 g, 0.28 mmol), tetrahydrofuran (2.8 mL), and bromo(2-methylphenyl)magnesium (0.15 mL of a 2.0 M solution with diethyl ether, 0.31 mmol, Sigma-Aldrich, St. Louis, Mo.), and then the reaction mixture was stirred for 15 min. After that time, methanol (1.0 mL) and silica gel (0.50 g) were added sequentially and the volatiles were removed under vacuum. The residue was subjected to flash chromatography on silica gel (6.5 g of silica gel, 4:1 hexane-ethyl acetate) to give N-(1-benzofuran-2-yl(2-methylphenyl)methyl)-3,4-dihydro-2H-1,5-benzodioxepine-7-sulfonamide (0.12 g) as a colorless solid (racemic mixture).

¹H NMR (400 MHz, chloroform-d) δ 7.42 (d, J=7.4 Hz, 1H), 7.32 (d, J=8.0 Hz, 1H), 7.30-7.19 (m, 3H), 7.19-7.11 (m, 4H), 7.11-7.04 (m, 1H), 6.78 (d, J=8.2 Hz, 1H), 6.36 (s, 1H), 5.97 (d, J=7.6 Hz, 1H), 5.13 (d, J=7.6 Hz, 1H), 4.21-4.02 (m, 3H), 4.02-3.92 (m, 1H), 2.41 (s, 3H), 2.21-2.00 (m, 2H). m/z (ESI, +ve ion) 472.0 (M+Na)⁺. GK-GKRP EC₅₀ (LCMS/MS)=0.11 μM.

This racemic mixture was resolved using preparative SFC (Chiralpak® AS-H column (Daicel, Inc., Fort Lee, N.J.) (150 mm×20 mm, 5 μm) eluting with 55% liquid CO₂ in 45% methanol at a flow rate of 75 mL/min) to give two products in greater than 99% enantiomeric excess.

### N—((S)-1-benzofuran-2-yl(2-methylphenyl)methyl)-3,4-dihydro-2H-1,5-benzodioxepine-7-sulfonamide

First Eluting Peak (Peak #1)

¹H NMR (400 MHz, DMSO-d₆) δ 8.85 (d, J=9.2 Hz, 1H), 7.50 (d, J=7.0 Hz, 1H), 7.41 (d, J=8.2 Hz, 1H), 7.32-7.03 (m, 8H), 6.87 (d, J=8.4 Hz, 1H), 6.44 (s, 1H), 5.83 (d, J=9.2 Hz, 1H), 4.13-3.84 (m, 4H), 2.30 (s, 3H), 2.15-1.94 (m, 2H). m/z (ESI, +ve ion) 471.9 (M+Na)⁺. GK-GKRP EC₅₀ (LCMS/MS)>50 μM.

### N—((R)-1-benzofuran-2-yl(2-methylphenyl)methyl)-3,4-dihydro-2H-1,5-benzodioxepine-7-sulfonamide

Second Eluting Peak (Peak #2)

¹H NMR (400 MHz, DMSO-d₆) δ 8.85 (d, J=9.2 Hz, 1H), 7.50 (d, J=7.4 Hz, 1H), 7.41 (d, J=8.0 Hz, 1H), 7.32-7.02 (m, 8H), 6.87 (d, J=8.4 Hz, 1H), 6.44 (s, 1H), 5.83 (d, J=9.2 Hz, 1H), 4.13-3.85 (m, 4H), 2.30 (s, 3H), 2.04 (t, J=5.4 Hz, 2H). m/z (ESI, +ve ion) 471.9 (M+Na)⁺. GK-GKRP EC₅₀ (LCMS/MS)=0.043 μM.

### Example 3

### N-(1-benzofuran-2-yl(4-fluoro-2-methylphenyl)methyl)-3,4-dihydro-2H-1,5-benzodioxepine-7-sulfonamide

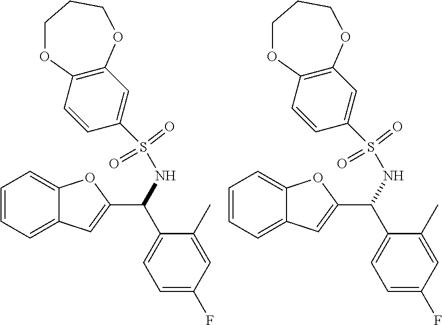

A 10 mL round-bottomed flask was charged with 1-bromo-4-fluoro-2-methylbenzene (0.088 mL, 0.70 mmol, Sigma-Aldrich, St. Louis, Mo.) and tetrahydrofuran (2.0 mL). The solution was cooled to −78° C., n-butyllithium (0.28 mL of a 2.5 M solution with hexane, 0.70 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.10 g, 0.28 mmol) and tetrahydrofuran (3.0 mL) was added. After stirring for 1 h, water (0.10 mL) was added, and the reaction mixture was warmed to room temperature and concentrated. The residue was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(4-fluoro-2-methylphenyl)methyl)-3,4-dihydro-2H-1,5-benzodioxepine-7-sulfonamide (0.11 g) as a yellow oil (racemic mixture).

¹H NMR (300 MHz, methanol-d₄) δ 7.53 (dd, J=3.0, 6.0 Hz, 1H), 7.45 (m, 1H), 7.36-7.14 (m, 5H), 7.09 (dd, J=3.0, 6.0 Hz, 1H), 6.95-6.78 (m, 3H), 6.38 (m, 1H), 5.92 (s, 1H), 4.15-3.90 (m, 4H), 2.41 (s, 3H), 2.15-2.07 (m, 2H). m/z (ESI, +ve ion) 490.0 (M+Na)⁺. GK-GKRP EC₅₀ (LCMS/MS)=0.44 μM. GK-GKRP IC₅₀ (Binding)=1.8 μM.

### Example 4

### N-(1-benzofuran-2-yl(5-fluoro-2-methylphenyl)methyl)-3,4-dihydro-2H-1,5-benzodioxepine-7-sulfonamide

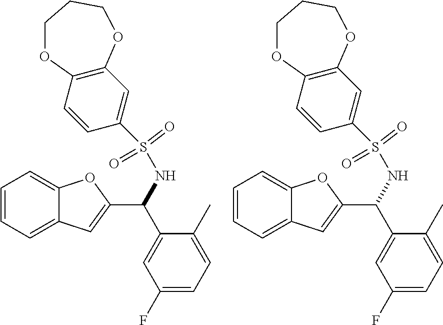

A 10 mL round-bottomed flask was charged with 2-bromo-4-fluoro-1-methylbenzene (0.087 mL, 0.70 mmol, Sigma-Aldrich, St. Louis, Mo.) and tetrahydrofuran (2.0 mL). The solution was cooled to −78° C., n-butyllithium (0.28 mL of a 2.5 M solution with hexane, 0.70 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.10 g, 0.28 mmol) and tetrahydrofuran (3.0 mL) was added. After stirring for 1 h, water (0.10 mL) was added, and the reaction mixture was warmed to room temperature and concentrated. The residue was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(5-fluoro-2-methylphenyl)methyl)-3,4-dihydro-2H-1,5-benzodioxepine-7-sulfonamide (0.032 g) as a yellow film (racemic mixture).

¹H NMR (300 MHz, methanol-d₄) δ 8.91 (d, J=9.0 Hz, 1H), 7.52 (m, 1H), 7.45 (m, 1H), 7.29-7.07 (m, 6H), 6.99 (td, J=3.0, 6.0 Hz 1H), 6.87 (d, J=9.0 Hz, 1H), 6.48 (s, 1H), 5.82 (d, J=9.0 Hz, 1H), 4.09-3.94 (m, 4H), 2.25 (s, 3H), 2.11-2.03 (m, 2H). m/z (ESI, +ve ion) 490.0 (M+Na)⁺. GK-GKRP EC₅₀ (LCMS/MS)=0.46 μM. GK-GKRP IC₅₀ (Binding)=1.4 μM.

### Example 5

### N-(1-benzofuran-2-yl(2-fluoro-6-methylphenyl)methyl)-3,4-dihydro-2H-1,5-benzodioxepine-7-sulfonamide

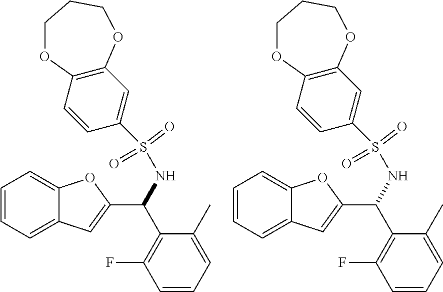

A 10 mL round-bottomed flask was charged with 2-bromo-1-fluoro-3-methylbenzene (0.13 mL, 0.70 mmol, Sigma-Aldrich, St. Louis, Mo.) and tetrahydrofuran (2.0 mL). The solution was cooled to −78° C., n-butyllithium (0.28 mL of a 2.5 M solution with hexane, 0.70 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.10 g, 0.28 mmol) and tetrahydrofuran (3.0 mL) was added. After stirring for 1 h, water (0.10 mL) was added, and the reaction mixture was warmed to room temperature and concentrated. The residue was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(2-fluoro-6-methylphenyl)methyl)-3,4-dihydro-2H-1,5-benzodioxepine-7-sulfonamide (0.013 g) as a yellow film (racemic mixture).

¹H NMR (300 MHz, methanol-d₄) δ 7.48 (m, 1H), 7.36-7.30 (m, 2H), 7.25-7.14 (m, 5H), 6.97 (d, J=6.0 Hz 1H), 6.86 (d, J=9.0 Hz, 1H), 6.80 (m, 1H), 6.51 (s, 1H), 6.11 (s, 1H), 4.20-4.15 (m, 2H), 4.13-4.08 (m, 2H), 2.41 (s, 3H), 2.20-2.12 (m, 2H). m/z (ESI, +ve ion) 490.0 (M+Na)⁺. GK-GKRP EC₅₀ (LCMS/MS)=0.79 μM. GK-GKRP IC₅₀ (Binding)=1.0 μM.

### Example 6

### N-(1-benzofuran-2-yl(2-ethylphenyl)methyl)-3,4-dihydro-2H-1,5-benzodioxepine-7-sulfonamide

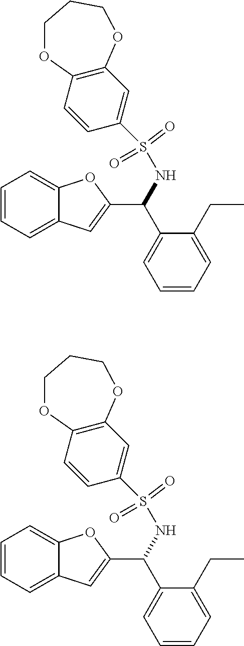 [SMILES: CCc1ccccc1[C@H](NS(=O)(=O)c1ccc2c(c1)OCCCO2)c1cc2ccccc2o1 ; CCc1ccccc1[C@@H](NS(=O)(=O)c1ccc2c(c1)OCCCO2)c1cc2ccccc2o1]

A 10 mL round-bottomed flask was charged with N-(1-benzofuran-2-ylmethylidene)-3,4-dihydro-2H-1,5-benzodioxepine-7-sulfonamide (intermediate A) (0.10 g, 0.28 mmol), tetrahydrofuran (2.8 mL), and bromo(2-ethylphenyl)magnesium (a total of 1.6 mL of a 0.50 M solution with tetrahydrofuran, added in three portions, 0.78 mmol, Rieke Metals, Lincoln, Nebr.), and then the reaction mixture was stirred for 50 min. After that time, methanol (1.0 mL) and silica gel (0.50 g) were added sequentially and the volatiles were removed under vacuum. The residue was subjected to flash chromatography on silica gel (6.5 g of silica gel, 4:1 hexane-ethyl acetate) to give N-(1-benzofuran-2-yl(2-ethylphenyl)methyl)-3,4-dihydro-2H-1,5-benzodioxepine-7-sulfonamide (0.11 g) as a colorless solid (racemic mixture).

¹H NMR (400 MHz, DMSO-d₆) δ 8.87 (d, J=9.4 Hz, 1H), 7.50 (d, J=7.2 Hz, 1H), 7.41 (d, J=8.2 Hz, 1H), 7.31 (d, J=7.6 Hz, 1H), 7.26-7.03 (m, 7H), 6.88 (d, J=8.4 Hz, 1H), 6.41 (s, 1H), 5.87 (d, J=9.4 Hz, 1H), 4.15-4.00 (m, 2H), 4.00-3.84 (m, 2H), 2.65 (q, J=7.4 Hz, 2H), 2.05 (t, J=5.7 Hz, 2H), 1.15 (t, J=7.5 Hz, 3H). m/z (ESI, +ve ion) 485.9 (M+Na)⁺. GK-GKRP EC₅₀ (LCMS/MS)=0.067 μM. GK-GKRP IC₅₀ (Binding)=0.22 μM.

This racemic mixture was resolved using preparative SFC (Chiralpak® AD-H column (Daicel, Inc., Fort Lee, N.J.) (150 mm×20 mm, 5 μm) eluting with 55% liquid CO₂ in 45% methanol at a flow rate of 75 mL/min) to give two products in greater than 99% enantiomeric excess.

### N—((S)-1-benzofuran-2-yl(2-ethylphenyl)methyl)-3,4-dihydro-2H-1,5-benzodioxepine-7-sulfonamide

First Eluting Peak (Peak #1)

¹H NMR (400 MHz, DMSO-d₆) δ 8.87 (br. s., 1H), 7.50 (d, J=7.2 Hz, 1H), 7.41 (d, J=8.2 Hz, 1H), 7.31 (d, J=7.4 Hz, 1H), 7.26-7.03 (m, 7H), 6.88 (d, J=8.4 Hz, 1H), 6.41 (s, 1H), 5.86 (br. s., 1H), 4.14-3.86 (m, 4H), 2.65 (q, J=7.3 Hz, 2H), 2.05 (br. s., 2H), 1.15 (t, J=7.5 Hz, 3H). m/z (ESI, +ve ion) 485.9 (M+Na)⁺. GK-GKRP EC₅₀ (LCMS/MS)>50 μM. GK-GKRP IC₅₀ (Binding)=4.8 μM.

### N—((R)-1-benzofuran-2-yl(2-ethylphenyl)methyl)-3,4-dihydro-2H-1,5-benzodioxepine-7-sulfonamide

Second Eluting Peak (Peak #2)

¹H NMR (400 MHz, DMSO-d₆) δ 8.87 (d, J=8.2 Hz, 1H), 7.50 (d, J=7.4 Hz, 1H), 7.41 (d, J=7.8 Hz, 1H), 7.31 (d, J=7.4 Hz, 1H), 7.28-7.04 (m, 7H), 6.88 (d, J=8.4 Hz, 1H), 6.42 (s, 1H), 5.87 (d, J=8.4 Hz, 1H), 4.13-3.85 (m, 4H), 2.65 (d, J=7.4 Hz, 2H), 2.05 (br. s., 2H), 1.15 (t, J=7.4 Hz, 3H). m/z (ESI, +ve ion) 485.9 (M+Na)⁺. GK-GKRP EC₅₀ (LCMS/MS)=0.040 μM. GK-GKRP IC₅₀ (Binding)=0.11 μM.

### Example 7

### N-(1-benzofuran-2-yl(2-ethenylphenyl)methyl)-3,4-dihydro-2H-1,5-benzodioxepine-7-sulfonamide

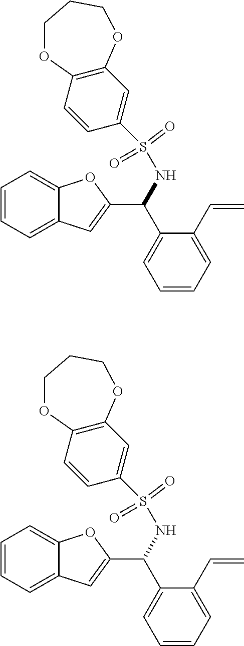 [SMILES: C=Cc1ccccc1[C@H](NS(=O)(=O)c1ccc2c(c1)OCCCO2)c1cc2ccccc2o1 ; C=Cc1ccccc1[C@@H](NS(=O)(=O)c1ccc2c(c1)OCCCO2)c1cc2ccccc2o1]

A 15 mL round-bottomed flask was charged with 1-bromo-2-ethenylbenzene (0.092 g, 0.50 mmol, Sigma-Aldrich, St. Louis, Mo.) and tetrahydrofuran (5.0 mL). The solution was cooled to −78° C., n-butyllithium (0.34 mL of a 1.6 M solution with hexane, 0.55 mmol, Sigma-Aldrich, St. Louis, Mo.) was added, and then the reaction mixture was stirred for 1 h. After that time, a solution of N-(1-benzofuran-2-ylmethylidene)-3,4-dihydro-2H-1,5-benzodioxepine-7-sulfonamide (intermediate A) (0.15 g, 0.42 mmol) in tetrahydrofuran (3.0 mL) was added. After stirring for 5 min, the mixture was warmed to room temperature, saturated aqueous sodium bicarbonate (5.0 mL) was added, the resulting mixture was partitioned between ethyl acetate (25 mL) and water (20 mL). The layers were separated, and the aqueous material was washed with ethyl acetate (2×25 mL). The combined organic extracts were washed with brine (25 mL), dried (magnesium sulfate), filtered. Silica gel (1.0 g) was added to the filtrate, and the volatiles were removed under a vacuum. The residue was subjected to flash chromatography on silica gel (Isco CombiFlash® system, 80 g RediSep® normal phase column, gradient elution of 10% to 35% ethyl acetate-hexane, Teledyne Isco, Lincoln, Nebr.) to give N-(1-benzofuran-2-yl(2-ethenylphenyl)methyl)-3,4-dihydro-2H-1,5-benzodioxepine-7-sulfonamide (0.057 g) as a colorless oil (racemic mixture).

¹H NMR (400 MHz, DMSO-d₆) δ 8.92 (d, J=9.2 Hz, 1H), 7.49 (dd, J=11.6, 7.3 Hz, 2H), 7.41 (d, J=8.2 Hz, 1H), 7.34-7.14 (m, 6H), 7.12-6.99 (m, 2H), 6.86 (d, J=8.4 Hz, 1H), 6.44 (s, 1H), 5.96 (d, J=9.2 Hz, 1H), 5.67 (dd, J=17.2, 1.2 Hz, 1H), 5.42-5.30 (m, 1H), 4.12-3.86 (m, 4H), 2.11-1.99 (m, 2H). m/z (ESI, +ve ion) 484.0 (M+Na)⁺. GK-GKRP EC₅₀ (LCMS/MS)=0.045 μM. GK-GKRP IC₅₀ (Binding)=0.098 μM.

### Example 8

### N-(1-benzofuran-2-yl(2-ethynylphenyl)methyl)-3,4-dihydro-2H-1,5-benzodioxepine-7-sulfonamide

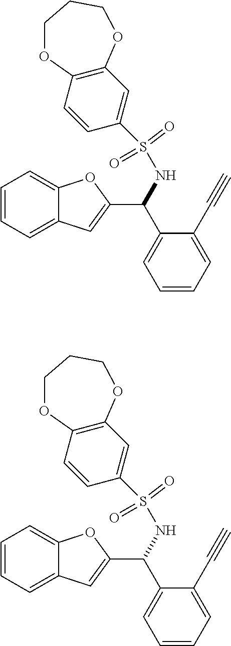 [SMILES: C#Cc1ccccc1[C@H](NS(=O)(=O)c1ccc2c(c1)OCCCO2)c1cc2ccccc2o1 ; C#Cc1ccccc1[C@@H](NS(=O)(=O)c1ccc2c(c1)OCCCO2)c1cc2ccccc2o1]

Step 1.

A 15 mL round-bottomed flask was charged with ((2-bromophenyl)ethynyl)(trimethyl)silane (0.12 g, 0.50 mmol, Sigma-Aldrich, St. Louis, Mo.) and tetrahydrofuran (2.1 mL). The solution was cooled to −78° C., n-butyllithium (0.31 mL of a 1.6 M solution with hexane, 0.50 mmol, Sigma-Aldrich, St. Louis, Mo.) was added, and then the reaction mixture was stirred for 2 h. After that time, a solution of N-(1-benzofuran-2-ylmethylidene)-3,4-dihydro-2H-1,5-benzodioxepine-7-sulfonamide (intermediate A) (0.15 g, 0.42 mmol) in tetrahydrofuran (2.0 mL) was added. After stirring for 10 min, saturated aqueous sodium bicarbonate (5.0 mL) was added, the mixture was warmed to room temperature and partitioned between ethyl acetate (50 mL) and water (50 mL). The layers were separated, the aqueous material was washed with ethyl acetate (50 mL), and the combined organic extract was washed with brine (50 mL), dried (sodium sulfate), filtered, and the filtrate was concentrated. The isolated material (0.22 g) was used in the next step of the synthesis without purification.

Step 2.

A 25 mL round-bottomed flask was charged with the isolated material (0.22 g) from Step 1, tetrahydrofuran (5.0 mL), and tetrabutylammonium fluoride (0.84 mL of a 1.0 M solution with tetrahydrofuran, 0.84 mmol, Sigma-Aldrich, St. Louis, Mo.), and then the reaction mixture was stirred for 1 h. After that time, the reaction mixture was partitioned between saturated aqueous ammonium chloride (50 mL) and ethyl acetate (50 mL), the layers were separated, the aqueous material was washed with ethyl acetate (50 mL), and the combined organic extract was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 20 min) to give N-(1-benzofuran-2-yl(2-ethynylphenyl)methyl)-3,4-dihydro-2H-1,5-benzodioxepine-7-sulfonamide (0.022 g) as a yellow solid (racemic mixture).

¹H NMR (400 MHz, DMSO-d₆) δ 8.99 (d, J=8.8 Hz, 1H), 7.52 (d, J=7.4 Hz, 2H), 7.43 (dd, J=13.8, 7.9 Hz, 2H), 7.37-7.30 (m, 1H), 7.29-7.14 (m, 4H), 7.12 (d, J=2.4 Hz, 1H), 6.87 (d, J=8.4 Hz, 1H), 6.34 (s, 1H), 6.17 (d, J=8.6 Hz, 1H), 4.48 (s, 1H), 4.21-3.84 (m, 4H), 2.12-1.99 (m, 2H). m/z (ESI, +ve ion) 482.0 (M+Na)⁺. GK-GKRP EC₅₀ (LCMS/MS)=0.072 μM. GK-GKRP IC₅₀ (Binding)=0.047 μM.

### Example 9

### N-(1-benzofuran-2-yl(2-(1-methylethyl)phenyl)methyl)-3,4-dihydro-2H-1,5-benzodioxepine-7-sulfonamide

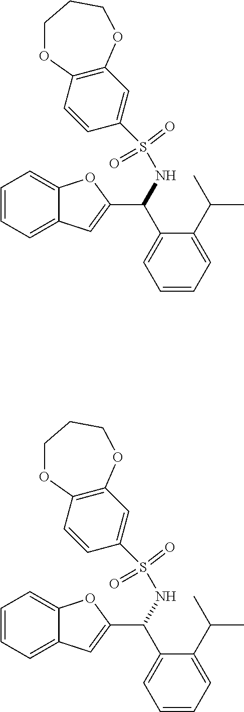 [SMILES: CC(C)c1ccccc1[C@H](NS(=O)(=O)c1ccc2c(c1)OCCCO2)c1cc2ccccc2o1 ; CC(C)c1ccccc1[C@@H](NS(=O)(=O)c1ccc2c(c1)OCCCO2)c1cc2ccccc2o1]

A 10 mL round-bottomed flask was charged with N-(1-benzofuran-2-ylmethylidene)-3,4-dihydro-2H-1,5-benzodioxepine-7-sulfonamide (intermediate A) (0.10 g, 0.28 mmol), tetrahydrofuran (2.8 mL), and bromo(2-(1-methylethyl)phenyl)magnesium (0.62 mL of a 0.50 M solution with tetrahydrofuran, 0.31 mmol, Novel Chemical Solutions, Crete, Nebr.), and then the reaction mixture was stirred for 15 min. After that time, methanol (1.0 mL) and silica gel (0.65 g) were added sequentially and the volatiles were removed under a vacuum. The residue was subjected to flash chromatography on silica gel (6.5 g of silica gel, 4:1 hexane-ethyl acetate) to give N-(1-benzofuran-2-yl(2-(1-methylethyl)phenyl)methyl)-3,4-dihydro-2H-1,5-benzodioxepine-7-sulfonamide (0.089 g) as a colorless solid (racemic mixture).

¹H NMR (400 MHz, DMSO-d₆) δ 8.91 (d, J=8.6 Hz, 1H), 7.50 (d, J=7.4 Hz, 1H), 7.42 (d, J=8.2 Hz, 1H), 7.34-7.14 (m, 6H), 7.14-7.00 (m, 2H), 6.89 (d, J=8.4 Hz, 1H), 6.41 (s, 1H), 5.96 (d, J=8.4 Hz, 1H), 4.15-3.85 (m, 4H), 3.25-3.10 (m, 1H), 2.05 (m, 2H), 1.20 (d, J=6.7 Hz, 3H), 1.10 (d, J=6.8 Hz, 3H). m/z (ESI, +ve ion) 499.9 (M+Na)⁺. GK-GKRP EC₅₀ (LCMS/MS)=0.28 μM. GK-GKRP IC₅₀ (Binding)=1.1 μM.

### Example 10

### N-(1-benzofuran-2-yl(2,6-dimethylphenyl)methyl)-3,4-dihydro-2H-1,5-benzodioxepine-7-sulfonamide

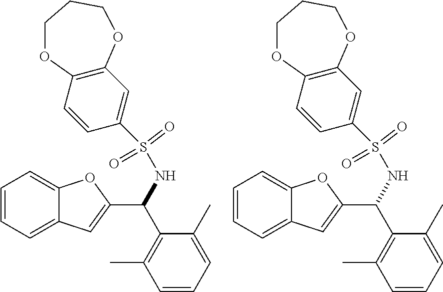

A 10 mL round-bottomed flask was charged with N-(1-benzofuran-2-ylmethylidene)-3,4-dihydro-2H-1,5-benzodioxepine-7-sulfonamide (0.10 g, 0.28 mmol), tetrahydrofuran (2.8 mL), and bromo(2,6-dimethylphenyl)magnesium (0.31 mL of a 1.0 M solution with tetrahydrofuran, 0.31 mmol, Sigma-Aldrich, St. Louis, Mo.), and then the reaction mixture was stirred for 1 h. After that time, methanol (1.0 mL) and silica gel (0.50 g) were added sequentially and the volatiles were removed under a vacuum. The residue was subjected to flash chromatography on silica gel (6.5 g of silica gel, 4:1 hexane-ethyl acetate) to give N-(1-benzofuran-2-yl(2,6-dimethylphenyl)methyl)-3,4-dihydro-2H-1,5-benzodioxepine-7-sulfonamide (0.12 g) as a colorless solid (racemic mixture).

¹H NMR (400 MHz, DMSO-d₆) δ 8.63 (d, J=7.0 Hz, 1H), 7.53 (d, J=6.8 Hz, 1H), 7.39 (d, J=7.8 Hz, 1H), 7.30 (dd, J=2.2, 8.4 Hz, 1H), 7.27-7.13 (m, 3H), 7.13-7.01 (m, 1H), 7.01-6.86 (m, 3H), 6.53 (s, 1H), 6.05 (d, J=6.7 Hz, 1H), 4.17 (t, J=5.6 Hz, 2H), 4.14-4.02 (m, 2H), 2.20 (s, 6H), 2.12 (dd, J=3.9, 5.7 Hz, 2H). m/z (ESI, +ve ion) 485.9 (M+Na)⁺. GK-GKRP EC₅₀ (LCMS/MS)=3.8 μM. GK-GKRP IC₅₀ (Binding)=4.1 μM.

### Example 11

### N-(1-benzofuran-2-yl(2-propylphenyl)methyl)-3,4-dihydro-2H-1,5-benzodioxepine-7-sulfonamide

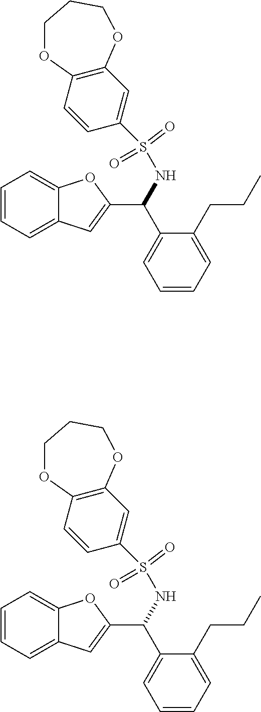 [SMILES: CCCc1ccccc1[C@H](NS(=O)(=O)c1ccc2c(c1)OCCCO2)c1cc2ccccc2o1 ; CCCc1ccccc1[C@@H](NS(=O)(=O)c1ccc2c(c1)OCCCO2)c1cc2ccccc2o1]

Step 1.

A 50 mL round-bottomed flask was charged with 1-bromo-2-(1-propen-1-yl)benzene (0.55 g, 2.8 mmol, Hibino, S.; Sugino, E.; Adachi, Y.; Nomi, K.; Sato, K.; Fukumoto, K. Heterocycles 1989, 28, 275-282), magnesium turnings (0.17 g, 7.0 mmol, Strem, Newburyport, Mass.), tetrahydrofuran (14 mL), and 1,2-dibromoethane (0.13 g, 0.70 mmol, Sigma-Aldrich, St. Louis, Mo.), and then the reaction mixture was heated at reflux for 90 min. After that time, the reaction mixture was cooled to room temperature and an aliquot (a total of 1.7 mL added in three portions) was transferred by syringe to a mixture of N-(1-benzofuran-2-ylmethylidene)-3,4-dihydro-2H-1,5-benzodioxepine-7-sulfonamide (intermediate A) (0.10 g, 0.28 mmol) and tetrahydrofuran (1.4 mL), and then the reaction mixture was stirred for 30 min. After that time, ethanol (1.0 mL) was added and the volatiles were removed under a vacuum. The isolated material (0.13 g) was used in the next step of the synthesis without purification.

Step 2.

A 10 mL round-bottomed flask was charged with the isolated material (0.13 g) from Step 1, ethanol (2.7 mL), and palladium 10 wt % (dry basis) on activated carbon (wet) Degussa type E101 NE/W (0.16 g, Sigma-Aldrich, St. Louis, Mo.), under a hydrogen atmosphere (1 atm; 101 kpascal), then the reaction mixture was stirred for 1 h. After that time, the reaction mixture was filtered, silica gel (0.65 g) was added to the filtrate, and the volatiles were removed under a vacuum. The residue was subjected to flash chromatography on silica gel (6.5 g of silica gel, 9:1 hexane-ethyl acetate), to give N-(1-benzofuran-2-yl(2-propylphenyl)methyl)-3,4-dihydro-2H-1,5-benzodioxepine-7-sulfonamide (0.082 g) as a colorless solid (racemic mixture).

¹H NMR (400 MHz, DMSO-d₆) δ 8.86 (d, J=7.0 Hz, 1H), 7.50 (d, J=7.4 Hz, 1H), 7.41 (d, J=8.0 Hz, 1H), 7.31 (d, J=7.4 Hz, 1H), 7.27-7.03 (m, 7H), 6.88 (d, J=8.4 Hz, 1H), 6.40 (s, 1H), 5.85 (d, J=6.7 Hz, 1H), 4.11-3.86 (m, 4H), 2.70-2.54 (m, 2H), 2.05 (m, 2H), 1.53 (m, 2H), 0.91 (t, J=7.3 Hz, 3H). m/z (ESI, +ve ion) 499.9 (M+Na)⁺. GK-GKRP EC₅₀ (LCMS/MS)=1.7 μM. GK-GKRP IC₅₀ (Binding)=1.8 μM.

### Example 12

### N-(1-benzofuran-2-yl(2-(trifluoromethyl)phenyl)methyl)-3,4-dihydro-2H-1,5-benzodioxepine-7-sulfonamide

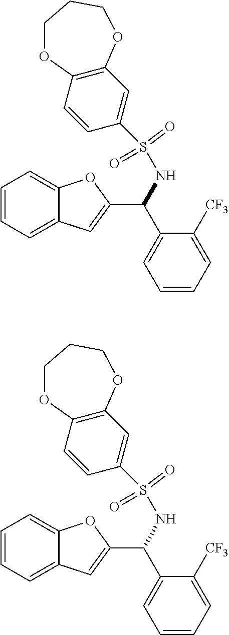 [SMILES: O=S(=O)(N[C@H](c1cc2ccccc2o1)c1ccccc1C(F)(F)F)c1ccc2c(c1)OCCCO2 ; O=S(=O)(N[C@@H](c1cc2ccccc2o1)c1ccccc1C(F)(F)F)c1ccc2c(c1)OCCCO2]

A microwave vial was charged with N-(1-benzofuran-2-ylmethylidene)-3,4-dihydro-2H-1,5-benzodioxepine-7-sulfonamide (intermediate A) (0.050 g, 0.14 mmol), magnesium turnings (0.016 g, 0.66 mmol, Strem, Newburyport, Mass.), 1-bromo-2-(trifluoromethyl)benzene (0.14 mL, 0.63 mmol, Fluka, Buchs, Switzerland), and tetrahydrofuran (2.0 mL), and then the reaction mixture was heated in a microwave reactor at 80° C. for 10 min. After that time, water (1.0 mL) was added, and the mixture was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(2-(trifluoromethyl)phenyl)methyl)-3,4-dihydro-2H-1,5-benzodioxepine-7-sulfonamide (0.027 g) as a pale yellow oil (racemic mixture).

¹H NMR (300 MHz, methanol-d₄) δ 7.84-7.75 (m, 2H), 7.68 (d, J=9.0 Hz, 1H), 7.60-7.43 (m, 4H), 7.38-7.35 (m, 1H), 7.31-7.14 (m, 3H), 6.85 (d, J=9.0 Hz, 1H), 6.25 (s, 1H), 6.12 (s, 1H), 4.15-4.12 (m, 2H), 4.04 (t, J=6.0 Hz, 2H), 2.14 (quin., J=6.0 Hz, 2H). m/z (ESI, +ve ion) 526.0 (M+Na)⁺. GK-GKRP EC₅₀ (LCMS/MS)=0.32 μM. GK-GKRP IC₅₀ (Binding)=1.0 μM.

### Example 13

### N-(1-benzofuran-2-yl(3-hydroxyphenyl)methyl)-3,4-dihydro-2H-1,5-benzodioxepine-7-sulfonamide

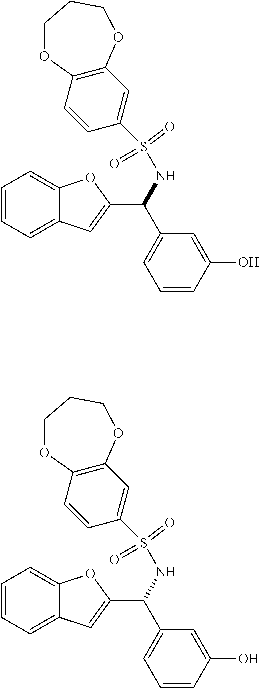 [SMILES: O=S(=O)(N[C@@H](c1cccc(O)c1)c1cc2ccccc2o1)c1ccc2c(c1)OCCCO2 ; O=S(=O)(N[C@H](c1cccc(O)c1)c1cc2ccccc2o1)c1ccc2c(c1)OCCCO2]

Step 1.

A 10 mL round-bottomed flask was charged with N-(1-benzofuran-2-ylmethylidene)-3,4-dihydro-2H-1,5-benzodioxepine-7-sulfonamide (intermediate a) (0.10 g, 0.28 mmol), tetrahydrofuran (2.8 mL), and (3-(benzyloxy)phenyl)(bromo)magnesium (a total of 1.2 mL of a 0.50 M solution with 2-methyltetrahydrofuran, added in two equal portions, 0.62 mmol, Novel Chemical Solutions, Crete, Nebr.), and then the reaction mixture was stirred for 19 h. After that time, methanol (1.0 mL) was added and the volatiles were removed under a vacuum. The isolated material (0.15 g) was used in the next step of the synthesis without purification.

Step 2.

A 10 mL round-bottomed flask was charged with the isolated material (0.15 g) from Step 1, ethanol (2.8 mL), and palladium 10 wt % (dry basis) on activated carbon (wet) Degussa type E101 NE/W (0.16 g, Sigma-Aldrich, St. Louis, Mo.), under a hydrogen atmosphere (1 atm), then the reaction mixture was stirred for 2 h. After that time, the reaction mixture was filtered, the filtrate was concentrated, the residue was dissolved with dichloromethane (5.0 mL), silica gel (0.50 g) was added, and the volatiles were removed under a vacuum. The residue was subjected to flash chromatography on silica gel (6.5 g of silica gel, 2:1 hexane-ethyl acetate), and then the isolated material was re-subjected to flash chromatography on silica gel (5.0 g of silica gel, 2:1 hexane-ethyl acetate) to give N-(1-benzofuran-2-yl(3-hydroxyphenyl)methyl)-3,4-dihydro-2H-1,5-benzodioxepine-7-sulfonamide (0.020 g) as a colorless solid (racemic mixture).

¹H NMR (400 MHz, chloroform-d) δ 7.43 (d, J=7.4 Hz, 1H), 7.35-7.24 (m, 3H), 7.24-7.07 (m, 3H), 6.87-6.65 (m, 4H), 6.43 (s, 1H), 5.69 (d, J=8.4 Hz, 1H), 5.55 (d, J=8.4 Hz, 1H), 5.33 (br. s., 1H), 4.10 (t, J=5.8 Hz, 2H), 4.04-3.93 (m, 2H), 2.10 (quin, J=5.8 Hz, 2H). m/z (ESI, +ve ion) 473.9 (M+Na)⁺. GK-GKRP EC₅₀ (LCMS/MS)=1.8 μM. GK-GKRP IC₅₀ (Binding)=2.5 μM.

### Example 14

### N-((3-aminophenyl)(1-benzofuran-2-yl)methyl)-3,4-dihydro-2H-1,5-benzodioxepine-7-sulfonamide

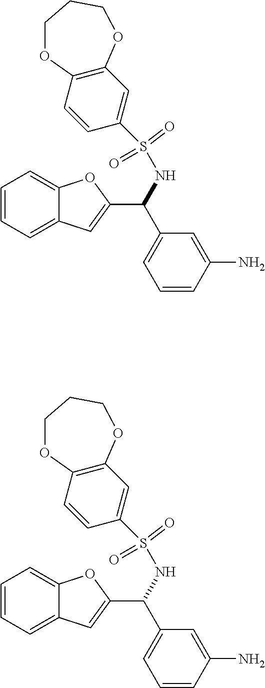 [SMILES: Nc1cccc([C@H](NS(=O)(=O)c2ccc3c(c2)OCCCO3)c2cc3ccccc3o2)c1 ; Nc1cccc([C@@H](NS(=O)(=O)c2ccc3c(c2)OCCCO3)c2cc3ccccc3o2)c1]

Step 1.

A 10 mL round-bottomed flask was charged with N-(1-benzofuran-2-ylmethylidene)-3,4-dihydro-2H-1,5-benzodioxepine-7-sulfonamide (intermediate A) (0.050 g, 0.14 mmol), 3-nitrobenzeneboronic acid (0.023 g, 0.14 mmol, Sigma-Aldrich, St. Louis, Mo.), 1,4-dioxane (2.0 mL), and bis(acetonitrile)(1,5-cyclooctadiene)rhodium(I) tetrafluoroborate (0.0053 g, 0.014 mmol, Sigma-Aldrich, St. Louis, Mo.), and then the reaction mixture was heated at 90° C. for 17 h. After that time, the reaction mixture was filtered and the filtrate was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(3-nitrophenyl)methyl)-3,4-dihydro-2H-1,5-benzodioxepine-7-sulfonamide (0.026 g) as an orange oil.

Step 2.

A 10 mL round-bottomed flask was charged with N-(1-benzofuran-2-yl(3-nitrophenyl)methyl)-3,4-dihydro-2H-1,5-benzodioxepine-7-sulfonamide (0.026 g, 0.054 mmol), ethanol (2.0 mL), water (0.20 mL), iron powder (0.030 g, 0.54 mmol, Sigma-Aldrich, St. Louis, Mo.), and ammonium chloride (0.00072 g, 0.013 mmol, Sigma-Aldrich, St. Louis, Mo.), and then the reaction mixture was heated at reflux for 2 h. After that time, the reaction mixture was cooled to room temperature and filtered, and the filtrate was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min), the isolated fraction was filtered through a Stratospheres SPE PL-HCO₃ MP-Resin column (500 mg/6.0 mL tube, 0.90 mmol nominal) (Agilent Technologies, Santa Clara, Calif.), and the filtrate was concentrated to give N-((3-aminophenyl)(1-benzofuran-2-yl)methyl)-3,4-dihydro-2H-1,5-benzodioxepine-7-sulfonamide (0.013 g) as a colorless solid (racemic mixture).

¹H NMR (300 MHz, methanol-d₄) δ 7.45 (m, 1H), 7.34-7.29 (m, 2H), 7.24-7.13 (m, 3H), 7.01 (t, J=6.0 Hz, 1H), 6.81 (d, J=9.0 Hz, 1H), 6.65-6.58 (m, 3H), 6.44 (s, 1H), 5.59 (s, 1H), 4.05 (t, J=3.0 Hz, 2H), 3.94 (t, J=3.0 Hz, 2H), 2.08 (quin, J=6.0 Hz, 2H). m/z (ESI, +ve ion) 451.0 (M+H)⁺. GK-GKRP EC₅₀ (LCMS/MS)=1.6 μM. GK-GKRP IC₅₀ (Binding)=1.2 μM.

### Example 15

### N-(1-benzofuran-2-yl(2-(hydroxymethyl)phenyl)methyl)-3,4-dihydro-2H-1,5-benzodioxepine-7-sulfonamide

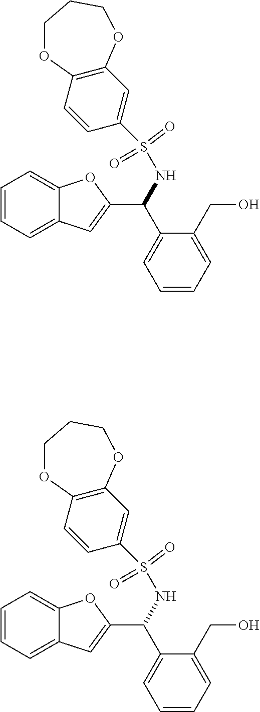 [SMILES: O=S(=O)(N[C@H](c1cc2ccccc2o1)c1ccccc1CO)c1ccc2c(c1)OCCCO2 ; O=S(=O)(N[C@@H](c1cc2ccccc2o1)c1ccccc1CO)c1ccc2c(c1)OCCCO2]

Step 1.

A 10 mL round-bottomed flask was charged with ((2-bromobenzyl)oxy)(tris(1-methylethyl))silane (0.58 g, 1.7 mmol, García Ruano, J. L.; Carreño, M. C.; Toledo, M. A.; Aguirre, J. M.; Aranda, M. T.; Fischer, J. Angew. Chem. Int. Ed. 2000, 39, 2736-2737), magnesium turnings (0.082 g, 3.4 mmol, Strem, Newburyport, Mass.), diethyl ether (2.8 mL), and 1,2-dibromoethane (0.063 g, 0.34 mmol, Sigma-Aldrich, St. Louis, Mo.), and then the reaction mixture was heated at reflux for 90 min. After that time, the reaction mixture was cooled to room temperature and transferred by syringe to a mixture of N-(1-benzofuran-2-ylmethylidene)-3,4-dihydro-2H-1,5-benzodioxepine-7-sulfonamide (intermediate A) (0.10 g, 0.28 mmol) and diethyl ether (2.8 mL), and then the reaction mixture was stirred for 15 min. After that time, saturated aqueous sodium bicarbonate (5.0 mL) was added, the mixture was partitioned between ethyl acetate (50 mL) and saturated aqueous sodium bicarbonate (50 mL), the layers were separated, the organic material was washed sequentially with saturated aqueous sodium bicarbonate (50 mL) and brine (50 mL), dried (sodium sulfate), filtered, and the filtrate was concentrated. The isolated material (0.87 g) was used in the next step of the synthesis without purification.

Step 2.

A 50 mL round-bottomed flask was charged with the isolated material (0.87 g) from Step 1, tetrahydrofuran (14 mL), and tetrabutylammonium fluoride (1.7 mL of a 1.0 M solution with tetrahydrofuran, 1.7 mmol, Sigma-Aldrich, St. Louis, Mo.), and then the reaction mixture was stirred for 2 h. After that time, the reaction mixture was partitioned between ethyl acetate (50 mL) and saturated aqueous sodium bicarbonate (50 mL), the layers were separated, the organic material was washed sequentially with saturated aqueous sodium bicarbonate (2×50 mL) and brine (2×50 mL), dried (sodium sulfate), filtered, and the filtrate was concentrated. The residue was subjected to flash chromatography on silica gel (33 g of silica gel, 2:1 hexane-ethyl acetate), and then the isolated material was re-subjected to flash chromatography on silica gel (24 g of silica gel, 99:1 dichloromethane-methanol) to give N-(1-benzofuran-2-yl(2-(hydroxymethyl)phenyl)methyl)-3,4-dihydro-2H-1,5-benzodioxepine-7-sulfonamide (0.29 g) as a colorless solid (racemic mixture).

¹H NMR (400 MHz, DMSO-d₆) δ 8.81 (d, J=8.4 Hz, 1H), 7.49 (d, J=7.6 Hz, 1H), 7.40 (d, J=8.2 Hz, 1H), 7.32 (d, J=7.6 Hz, 1H), 7.35 (d, J=7.6 Hz, 1H), 7.29-7.14 (m, 5H), 7.13 (d, J=2.0 Hz, 1H), 6.85 (d, J=8.4 Hz, 1H), 6.39 (s, 1H), 6.01 (d, J=7.8 Hz, 1H), 5.25 (br. s., 1H), 4.67-4.45 (m, 2H), 4.12-3.86 (m, 4H), 2.04 (m, 2H). m/z (ESI, +ve ion) 487.9 (M+Na)⁺. GK-GKRP EC₅₀ (LCMS/MS)=1.6 μM. GK-GKRP IC₅₀ (Binding)=4.6 μM.

### Example 16

### N-(1-benzofuran-2-yl(2-(2-hydroxyethyl)phenyl)methyl)-3,4-dihydro-2H-1,5-benzodioxepine-7-sulfonamide

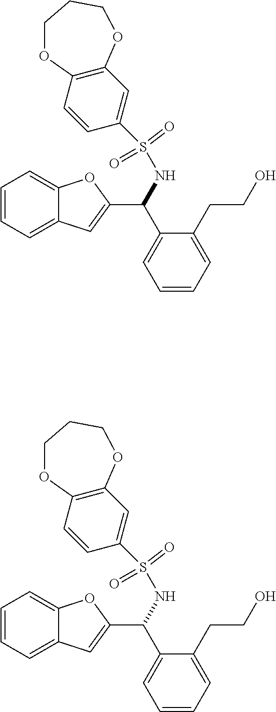 [SMILES: O=S(=O)(N[C@H](c1cc2ccccc2o1)c1ccccc1CCO)c1ccc2c(c1)OCCCO2 ; O=S(=O)(N[C@@H](c1cc2ccccc2o1)c1ccccc1CCO)c1ccc2c(c1)OCCCO2]

Step 1.

A 10 mL round-bottomed flask was charged with (2-(2-bromophenyl)ethoxy)(tert-butyl)dimethylsilane (0.53 g, 1.7 mmol, Combi-Blocks, San Diego, Calif.), magnesium turnings (0.082 g, 3.4 mmol, Strem, Newburyport, Mass.), diethyl ether (2.8 mL), and 1,2-dibromoethane (0.063 g, 0.34 mmol, Sigma-Aldrich, St. Louis, Mo.), and then the reaction mixture was heated at reflux for 90 min. After that time, the reaction mixture was cooled to room temperature and transferred by syringe to a mixture of N-(1-benzofuran-2-ylmethylidene)-3,4-dihydro-2H-1,5-benzodioxepine-7-sulfonamide (intermediate A) (0.10 g, 0.28 mmol) and diethyl ether (2.8 mL), and then the reaction mixture was stirred for 4 h. After that time, saturated aqueous sodium bicarbonate (5.0 mL) was added, the mixture was partitioned between ethyl acetate (50 mL) and saturated aqueous sodium bicarbonate (50 mL), the layers were separated, the organic material was washed sequentially with saturated aqueous sodium bicarbonate (50 mL) and brine (50 mL), dried (sodium sulfate), filtered, and the filtrate was concentrated. The isolated material (0.83 g) was used in the next step of the synthesis without purification.

Step 2.

A 50 mL round-bottomed flask was charged with the isolated material (0.83 g) from Step 1, tetrahydrofuran (14 mL), and tetrabutylammonium fluoride (1.7 mL of a 1.0 M solution with tetrahydrofuran, 1.7 mmol, Sigma-Aldrich, St. Louis, Mo.), and then the reaction mixture was stirred for 15 h. After that time, the reaction mixture was partitioned between ethyl acetate (50 mL) and saturated aqueous sodium bicarbonate (50 mL), the layers were separated, the organic material was washed sequentially with saturated aqueous sodium bicarbonate (2×50 mL) and brine (2×50 mL), dried (sodium sulfate), filtered, and the filtrate was concentrated. The residue was subjected to flash chromatography on silica gel (70 g of silica gel, 2:1 hexane-ethyl acetate) to give N-(1-benzofuran-2-yl(2-(2-hydroxyethyl)phenyl)methyl)-3,4-dihydro-2H-1,5-benzodioxepine-7-sulfonamide (0.43 g) as a colorless solid (racemic mixture).

¹H NMR (400 MHz, chloroform-d) δ 7.43 (d, J=7.4 Hz, 1H), 7.37-7.07 (m, 9H), 6.81 (d, J=8.0 Hz, 1H), 6.44 (s, 1H), 6.01 (d, J=6.8 Hz, 1H), 5.87 (d, J=7.0 Hz, 1H), 4.25-4.04 (m, 3H), 4.04-3.83 (m, 3H), 3.13-2.97 (m, 1H), 2.93-2.85 (m, 1H), 2.22-2.05 (m, 2H), 1.66 (br. s., 1H). m/z (ESI, +ve ion) 501.9 (M+Na)⁺. GK-GKRP EC₅₀ (LCMS/MS)=3.2 μM.

### Example 17

### N-(1-benzofuran-2-yl(2-methoxyphenyl)methyl)-3,4-dihydro-2H-1,5-benzodioxepine-7-sulfonamide

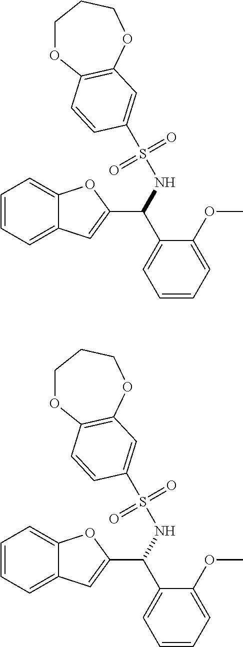 [SMILES: COc1ccccc1[C@H](NS(=O)(=O)c1ccc2c(c1)OCCCO2)c1cc2ccccc2o1 ; COc1ccccc1[C@@H](NS(=O)(=O)c1ccc2c(c1)OCCCO2)c1cc2ccccc2o1]

A 10 mL round-bottomed flask was charged with N-(1-benzofuran-2-ylmethylidene)-3,4-dihydro-2H-1,5-benzodioxepine-7-sulfonamide (intermediate A) (0.050 g, 0.14 mmol), tetrahydrofuran (2.0 mL), and bromo(2-methoxyphenyl)magnesium (0.42 mL of a 0.50 M solution with tetrahydrofuran, 0.21 mmol, Rieke Metals, Lincoln, Nebr.), and then the reaction mixture was stirred for 30 min. After that time, saturated aqueous ammonium chloride (2.0 mL) and dichloromethane (2.0 mL) were added sequentially, the mixture was filtered through a Radleys phase separation column (Brinkmann Instruments, Inc., Westbury, N.Y.), and the filtrate was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(2-methoxyphenyl)methyl)-3,4-dihydro-2H-1,5-benzodioxepine-7-sulfonamide (0.046 g) as a colorless solid (racemic mixture).

¹H NMR (300 MHz, methanol-d₄) δ 7.48-7.43 (m, 2H), 7.35-7.13 (m, 7H), 6.91-6.87 (m, 2H), 6.84 (d, J=9.0 Hz, 1H), 6.36 (s, 1H), 6.10 (s, 1H), 4.13 (t, J=6.0 Hz, 2H), 4.04 (t, J=6.0 Hz, 2H), 3.78 (s, 3H), 2.14 (quin, J=6.0 Hz, 2H). m/z (ESI, +ve ion) 488.0 (M+Na)⁺. GK-GKRP EC₅₀ (LCMS/MS)=0.095 μM. GK-GKRP IC₅₀ (Binding)=0.17 μM.

This racemic mixture was resolved using preparative SFC (Chiralpak® AD-H column (Daicel, Inc., Fort Lee, N.J.) (250 mm×21 mm, 5 μm) eluting with 60% liquid CO₂ in 40% methanol (with 20 mM NH₃) at a flow rate of 70 mL/min) to give two products in greater than 99% enantiomeric excess.

### N—((S)-1-benzofuran-2-yl(2-methoxyphenyl)methyl)-3,4-dihydro-2H-1,5-benzodioxepine-7-sulfonamide

First Eluting Peak (Peak #1)

¹H NMR (400 MHz, chloroform-d) δ 7.46-7.39 (m, 1H), 7.32 (d, J=8.2 Hz, 1H), 7.30-7.10 (m, 6H), 6.86 (t, J=7.4 Hz, 1H), 6.78 (dd, J=3.1, 8.6 Hz, 2H), 6.42 (s, 1H), 5.90 (d, J=9.2 Hz, 1H), 5.81 (d, J=9.2 Hz, 1H), 4.25-4.12 (m, 2H), 4.08 (dt, J=2.7, 5.9 Hz, 2H), 3.74 (s, 3H), 2.15 (quin, J=5.9 Hz, 2H). m/z (ESI, +ve ion) 487.9 (M+Na)⁺. GK-GKRP EC₅₀ (LCMS/MS)=6.1 μM. GK-GKRP IC₅₀ (Binding)=1.2 μM.

### N—((R)-1-benzofuran-2-yl(2-methoxyphenyl)methyl)-3,4-dihydro-2H-1,5-benzodioxepine-7-sulfonamide

Second Eluting Peak (Peak #2)

¹H NMR (400 MHz, chloroform-d) δ 7.43 (d, J=7.6 Hz, 1H), 7.32 (d, J=8.0 Hz, 1H), 7.30-7.11 (m, 6H), 6.86 (t, J=7.5 Hz, 1H), 6.78 (dd, J=3.3, 8.6 Hz, 2H), 6.42 (s, 1H), 5.90 (d, J=9.2 Hz, 1H), 5.81 (d, J=9.2 Hz, 1H), 4.16 (m, 2H), 4.08 (dt, J=2.7, 5.9 Hz, 2H), 3.74 (s, 3H), 2.15 (quin, J=5.9 Hz, 2H). m/z (ESI, +ve ion) 487.9 (M+Na)⁺. GK-GKRP EC₅₀ (LCMS/MS)=0.052 μM. GK-GKRP IC₅₀ (Binding)=0.052 μM.

### Example 18

### N-(1-benzofuran-2-yl(2-(trifluoromethoxy)phenyl)methyl)-3,4-dihydro-2H-1,5-benzodioxepine-7-sulfonamide

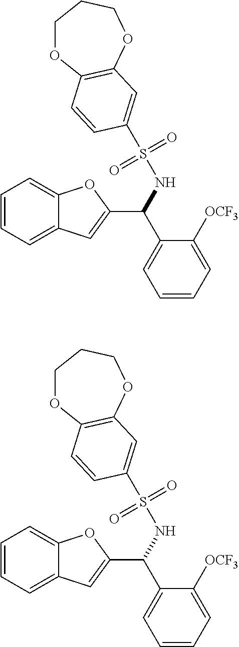 [SMILES: O=S(=O)(N[C@H](c1cc2ccccc2o1)c1ccccc1OC(F)(F)F)c1ccc2c(c1)OCCCO2 ; O=S(=O)(N[C@@H](c1cc2ccccc2o1)c1ccccc1OC(F)(F)F)c1ccc2c(c1)OCCCO2]

A 10 mL round-bottomed flask was charged with 1-bromo-2-(trifluoromethoxy)benzene (0.17 g, 0.70 mmol, Sigma-Aldrich, St. Louis, Mo.) and tetrahydrofuran (2.0 mL). The solution was cooled to −78° C., n-butyllithium (0.28 mL of a 2.5 M solution with hexane, 0.70 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.10 g, 0.28 mmol) and tetrahydrofuran (3.0 mL) was added. After stirring for 1 h, water (0.10 mL) was added, the reaction mixture was warmed to room temperature and concentrated. The residue was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(2-(trifluoromethoxy)phenyl)methyl)-3,4-dihydro-2H-1,5-benzodioxepine-7-sulfonamide (0.11 g) as a yellow oil (racemic mixture).

¹H NMR (300 MHz, methanol-d₄) δ 7.74 (dd, J=3.0, 6.0 Hz, 1H), 7.48-7.14 (m, 9H), 6.85 (d, J=6.0 Hz, 1H), 6.37 (s, 1H), 6.07 (s, 1H), 4.11 (t, J=6.0 Hz, 2H), 4.01 (t, J=6.0 Hz, 2H), 2.12 (quin, J=6.0 Hz, 2H). m/z (ESI, +ve ion) 542.0 (M+Na)⁺. GK-GKRP EC₅₀ (LCMS/MS)=0.34 μM. GK-GKRP IC₅₀ (Binding)=0.97 μM.

### Example 19

### N-(1-benzofuran-2-yl(2-(methylsulfanyl)phenyl)methyl)-3,4-dihydro-2H-1,5-benzodioxepine-7-sulfonamide

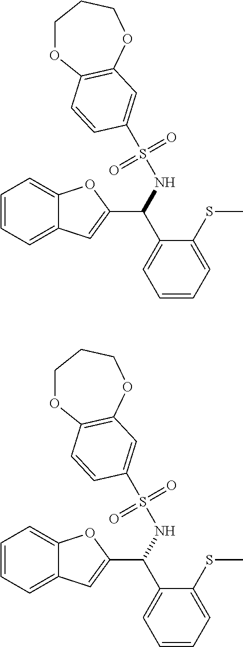 [SMILES: CSc1ccccc1[C@H](NS(=O)(=O)c1ccc2c(c1)OCCCO2)c1cc2ccccc2o1 ; CSc1ccccc1[C@@H](NS(=O)(=O)c1ccc2c(c1)OCCCO2)c1cc2ccccc2o1]

A 10 mL round-bottomed flask was charged with N-(1-benzofuran-2-ylmethylidene)-3,4-dihydro-2H-1,5-benzodioxepine-7-sulfonamide (0.10 g, 0.28 mmol), tetrahydrofuran (2.8 mL), and bromo(2-(methylsulfanyl)phenyl)magnesium (0.62 mL of a 0.50 M solution with tetrahydrofuran, 0.31 mmol, Rieke Metals, Lincoln, Nebr.), and then the reaction mixture was stirred for 15 min. After that time, methanol (1.0 mL) and silica gel (0.60 g) were added sequentially and the volatiles were removed under a vacuum. The residue was subjected to flash chromatography on silica gel (6.0 g of silica gel, 4:1 hexane-ethyl acetate) to give N-(1-benzofuran-2-yl(2-(methylsulfanyl)phenyl)methyl)-3,4-dihydro-2H-1,5-benzodioxepine-7-sulfonamide (0.078 g) as a colorless solid (racemic mixture).

¹H NMR (400 MHz, DMSO-d₆) δ 8.94 (d, J=9.0 Hz, 1H), 7.50 (d, J=7.2 Hz, 1H), 7.48-7.37 (m, 2H), 7.35-7.07 (m, 7H), 6.89 (d, J=8.4 Hz, 1H), 6.30 (s, 1H), 6.16 (d, J=9.0 Hz, 1H), 4.18-4.05 (m, 2H), 4.05-3.90 (m, 2H), 2.44 (s, 3H), 2.07 (quin, J=5.6 Hz, 2H). m/z (ESI, +ve ion) 503.9 (M+Na)⁺. GK-GKRP EC₅₀ (LCMS/MS)=0.038 μM. GK-GKRP IC₅₀ (Binding)=0.16 μM.

This racemic mixture was resolved using preparative SFC (Chiralpak® AS-H column (Daicel, Inc., Fort Lee, N.J.) (250 mm×21 mm, 5 μm) eluting with 58% liquid CO₂ in 42% methanol (with 0.20% diethylamine) at a flow rate of 70 mL/min) to give two products in greater than 99% enantiomeric excess.

### N—((S)-1-benzofuran-2-yl(2-(methylsulfanyl)phenyl)methyl)-3,4-dihydro-2H-1,5-benzodioxepine-7-sulfonamide

First Eluting Peak (Peak #1)

¹H NMR (400 MHz, chloroform-d) δ 7.43 (d, J=7.8 Hz, 1H), 7.37-7.07 (m, 9H), 6.81 (d, J=9.0 Hz, 1H), 6.41 (s, 1H), 6.25 (d, J=8.0 Hz, 1H), 5.66 (d, J=8.0 Hz, 1H), 4.27-3.98 (m, 4H), 2.42 (s, 3H), 2.16 (m, 2H). m/z (ESI, +ve ion) 503.8 (M+Na)⁺. GK-GKRP EC₅₀ (LCMS/MS)=0.29 μM. GK-GKRP IC₅₀ (Binding)=0.97 μM.

### N—((R)-1-benzofuran-2-yl(2-(methylsulfanyl)phenyl)methyl)-3,4-dihydro-2H-1,5-benzodioxepine-7-sulfonamide

Second Eluting Peak (Peak #2)

¹H NMR (400 MHz, chloroform-d) δ 7.43 (d, J=7.2 Hz, 1H), 7.36-7.07 (m, 9H), 6.86-6.73 (m, 1H), 6.41 (s, 1H), 6.25 (s, 1H), 5.67 (br. s., 1H), 4.27-3.99 (m, 4H), 2.42 (s, 3H), 2.16 (quin, J=5.8 Hz, 2H). m/z (ESI, +ve ion) 503.7 (M+Na)⁺. GK-GKRP EC₅₀ (LCMS/MS)=0.023 μM. GK-GKRP IC₅₀ (Binding)=0.046 μM.

### Example 20

### N-(1-benzofuran-2-yl(2-chlorophenyl)methyl)-3,4-dihydro-2H-1,5-benzodioxepine-7-sulfonamide

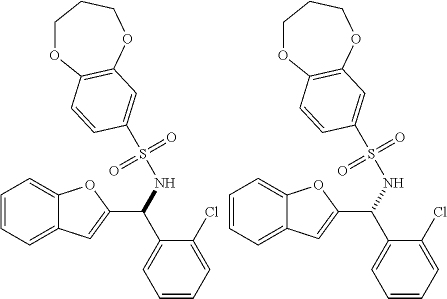

A microwave vial was charged with N-(1-benzofuran-2-ylmethylidene)-3,4-dihydro-2H-1,5-benzodioxepine-7-sulfonamide (0.050 g, 0.14 mmol), magnesium turnings (0.016 g, 0.66 mmol, Strem, Newburyport, Mass.), 1-bromo-2-chlorobenzene (0.073 mL, 0.63 mmol, Fluka, Buchs, Switzerland), and tetrahydrofuran (2.0 mL), and then the reaction mixture was heated in microwave reactor at 80° C. for 10 min. After that time, water (1.0 mL) was added, and then the mixture was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(2-chlorophenyl)methyl)-3,4-dihydro-2H-1,5-benzodioxepine-7-sulfonamide (0.018 g) as a pale yellow oil (racemic mixture).

¹H NMR (300 MHz, methanol-d₄) δ 7.69 (dd, J=3.0, 6.0 Hz, 1H), 7.55-7.45 (m, 3H), 7.38-7.14 (m, 7H), 6.86 (d, J=9.0 Hz, 1H), 6.36 (s, 1H), 6.19 (s, 1H), 4.14 (t, J=6.0 Hz, 2H), 4.05 (m, 2H), 2.14 (quin, J=6.0 Hz, 2H). m/z (ESI, +ve ion) 492.0 (M+Na)⁺. GK-GKRP EC₅₀ (LCMS/MS)=0.070 μM. GK-GKRP IC₅₀ (Binding)=0.22 μM.

This racemic mixture was resolved using preparative SFC (Chiralpak® AS-H column (Daicel, Inc., Fort Lee, N.J.) (250 mm×21 mm, 5 μm) eluting with 60% liquid CO₂ in 40% methanol (with 40 mM NH₃) at a flow rate of 70 mL/min) to give two products in greater than 99% enantiomeric excess.

### N—((S)-1-benzofuran-2-yl(2-chlorophenyl)methyl)-3,4-dihydro-2H-1,5-benzodioxepine-7-sulfonamide

First Eluting Peak (Peak #1)

¹H NMR (400 MHz, chloroform-d) δ 7.44 (d, J=7.2 Hz, 1H), 7.41-7.36 (m, 1H), 7.36-7.29 (m, 4H), 7.28-7.14 (m, 4H), 6.84 (d, J=8.6 Hz, 1H), 6.43 (s, 1H), 6.15 (d, J=7.8 Hz, 1H), 5.40 (d, J=7.8 Hz, 1H), 4.19 (t, J=5.8 Hz, 2H), 4.11 (t, J=5.8 Hz, 2H), 2.17 (quin, J=5.8 Hz, 2H). m/z (ESI, +ve ion) 491.7 (M+Na)⁺. GK-GKRP EC₅₀ (LCMS/MS)>20 μM. GK-GKRP IC₅₀ (Binding)=5.7 μM.

### N—((R)-1-benzofuran-2-yl(2-chlorophenyl)methyl)-3,4-dihydro-2H-1,5-benzodioxepine-7-sulfonamide

Second Eluting Peak (Peak #2)

¹H NMR (400 MHz, chloroform-d) δ 7.44 (d, J=7.4 Hz, 1H), 7.41-7.36 (m, 1H), 7.36-7.29 (m, 4H), 7.29-7.14 (m, 4H), 6.84 (d, J=8.6 Hz, 1H), 6.43 (s, 1H), 6.15 (d, J=7.6 Hz, 1H), 5.40 (d, J=7.8 Hz, 1H), 4.19 (t, J=5.8 Hz, 2H), 4.11 (t, J=5.8 Hz, 2H), 2.17 (quin, J=5.8 Hz, 2H). m/z (ESI, +ve ion) 491.7 (M+Na)⁺. GK-GKRP EC₅₀ (LCMS/MS)=0.030 μM. GK-GKRP IC₅₀ (Binding)=0.078 μM.

### Example 21

### N-(1-benzofuran-2-yl(2-iodophenyl)methyl)-3,4-dihydro-2H-1,5-benzodioxepine-7-sulfonamide

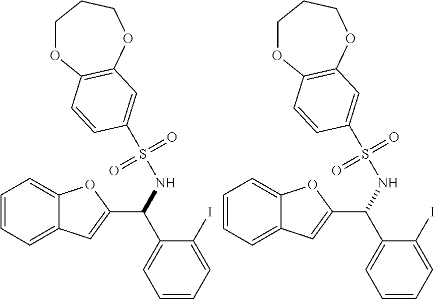

A 10 mL round-bottomed flask was charged with 1,2-diiodobenzene (0.20 g, 0.62 mmol, Oakwood, West Columbia, S.C.) and tetrahydrofuran (1.6 mL). The solution was cooled to −40° C., isopropylmagnesium chloride lithium chloride complex (0.52 mL of a 1.3 M solution with tetrahydrofuran, 0.67 mmol, Sigma-Aldrich, St. Louis, Mo.) was added, and then the reaction mixture was warmed to −20° C. for 20 min. After that time, a solution of N-(1-benzofuran-2-ylmethylidene)-3,4-dihydro-2H-1,5-benzodioxepine-7-sulfonamide (intermediate A) (0.20 g, 0.56 mmol) in tetrahydrofuran (1.6 mL) was added, and then the reaction mixture was allowed to warm to room temperature. After stirring for 90 min, methanol (1.0 mL) and silica gel (1.6 g) were added sequentially and the volatiles were removed under a vacuum. The residue was subjected to flash chromatography on silica gel (16 g of silica gel, 4:1 hexane-ethyl acetate) to give N-(1-benzofuran-2-yl(2-iodophenyl)methyl)-3,4-dihydro-2H-1,5-benzodioxepine-7-sulfonamide (0.17 g) as a colorless solid (racemic mixture).

¹H NMR (400 MHz, DMSO-d₆) δ 9.06 (d, J=8.0 Hz, 1H), 7.79 (d, J=7.2 Hz, 1H), 7.59-7.41 (m, 3H), 7.38-7.17 (m, 4H), 7.14 (d, J=2.3 Hz, 1H), 7.07-6.94 (m, 1H), 6.89 (d, J=8.4 Hz, 1H), 6.30 (s, 1H), 5.93 (d, J=7.8 Hz, 1H), 4.22-3.92 (m, 4H), 2.09 (quin, J=5.6 Hz, 2H). m/z (ESI, +ve ion) 583.7 (M+Na)⁺. GK-GKRP EC₅₀ (LCMS/MS)=0.076 μM. GK-GKRP IC₅₀ (Binding)=0.228 μM.

This racemic mixture was resolved using preparative SFC (Chiralpak® AD-H column (Daicel, Inc., Fort Lee, N.J.) (150 mm×20 mm, 5 μm) eluting with 50% liquid CO₂ in 50% methanol:ethanol:isopropanol (1:1:1 with 0.1% diethylamine) at a flow rate of 65 mL/min) to give two products in greater than 99% enantiomeric excess.

### N—((S)-1-benzofuran-2-yl(2-iodophenyl)methyl)-3,4-dihydro-2H-1,5-benzodioxepine-7-sulfonamide

First Eluting Peak (Peak #1)

¹H NMR (300 MHz, DMSO-d₆) δ 9.06 (br. s., 1H), 7.79 (dd, J=1.0, 7.9 Hz, 1H), 7.57-7.39 (m, 3H), 7.37-7.15 (m, 4H), 7.14 (d, J=2.3 Hz, 1H), 7.01 (dt, J=1.6, 7.6 Hz, 1H), 6.89 (d, J=8.5 Hz, 1H), 6.30 (s, 1H), 5.93 (br. s., 1H), 4.24-3.87 (m, 4H), 2.09 (quin, J=5.6 Hz, 2H). m/z (ESI, +ve ion) 583.7 (M+Na)⁺. GK-GKRP EC₅₀ (LCMS/MS)>20 μM. GK-GKRP IC₅₀ (Binding)>33 μM.

### N—((R)-1-benzofuran-2-yl(2-iodophenyl)methyl)-3,4-dihydro-2H-1,5-benzodioxepine-7-sulfonamide

Second Eluting Peak (Peak #2)

¹H NMR (300 MHz, DMSO-d₆) δ 9.06 (br. s., 1H), 7.79 (dd, J=1.0, 7.9 Hz, 1H), 7.59-7.40 (m, 3H), 7.37-7.16 (m, 4H), 7.14 (d, J=2.3 Hz, 1H), 7.01 (dt, J=1.6, 7.7 Hz, 1H), 6.89 (d, J=8.5 Hz, 1H), 6.30 (s, 1H), 5.93 (br. s., 1H), 4.25-3.91 (m, 4H), 2.20-1.94 (m, 2H). m/z (ESI, +ve ion) 583.7 (M+Na)⁺. GK-GKRP EC₅₀ (LCMS/MS)=0.042 μM. GK-GKRP IC₅₀ (Binding)=0.040 μM.

### Example 22

### N-(1-benzofuran-2-yl(2-bromophenyl)methyl)-3,4-dihydro-2H-1,5-benzodioxepine-7-sulfonamide

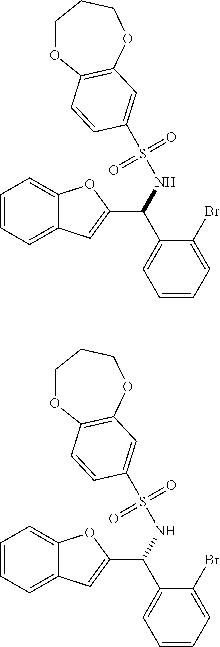 [SMILES: O=S(=O)(N[C@H](c1cc2ccccc2o1)c1ccccc1Br)c1ccc2c(c1)OCCCO2 ; O=S(=O)(N[C@@H](c1cc2ccccc2o1)c1ccccc1Br)c1ccc2c(c1)OCCCO2]

A 10 mL round-bottomed flask was charged with isopropylmagnesium chloride lithium chloride complex (0.52 mL of a 1.3 M solution with tetrahydrofuran, 0.67 mmol, Sigma-Aldrich, St. Louis, Mo.), the solution was cooled to −15° C., 1,2-dibromobenzene (0.15 g, 0.62 mmol, Alfa Aesar, Heysham, United Kingdom) was added, and then the reaction mixture was stirred for 90 min. After that time, a solution of N-(1-benzofuran-2-ylmethylidene)-3,4-dihydro-2H-1,5-benzodioxepine-7-sulfonamide (intermediate A) (0.20 g, 0.56 mmol) and tetrahydrofuran (2.2 mL) was added and then the reaction mixture was allowed to warm to room temperature. After stirring for 90 min, methanol (1.0 mL) and silica gel (1.4 g) were added sequentially and the volatiles were removed under a vacuum. The residue was subjected to flash chromatography on silica gel (15 g of silica gel, 4:1 hexane-ethyl acetate), and then the isolated material was re-subjected to flash chromatography on silica gel (3.5 g of silica gel, dichloromethane) to give N-(1-benzofuran-2-yl(2-bromophenyl)methyl)-3,4-dihydro-2H-1,5-benzodioxepine-7-sulfonamide (0.038 g) as a colorless solid (racemic mixture).

¹H NMR (400 MHz, DMSO-d₆) δ 9.10 (d, J=9.4 Hz, 1H), 7.61-7.50 (m, 3H), 7.46 (d, J=8.2 Hz, 1H), 7.37-7.16 (m, 5H), 7.12 (d, J=2.0 Hz, 1H), 6.89 (d, J=8.4 Hz, 1H), 6.37 (s, 1H), 6.05 (d, J=9.4 Hz, 1H), 4.18-3.92 (m, 4H), 2.15-1.97 (m, 2H). m/z (ESI, +ve ion) 535.7, 537.7 (M+Na)⁺. GK-GKRP EC₅₀ (LCMS/MS)=0.058 μM. GK-GKRP IC₅₀ (Binding)=0.15 μM.

This racemic mixture was resolved using preparative SFC (Chiralpak® AD-H column (Daicel, Inc., Fort Lee, N.J.) (250 mm×21 mm, 5 μm) eluting with 60% liquid CO₂ in 40% methanol (with 2.0 mM NH₃) at a flow rate of 70 mL/min) to give two products in greater than 99% enantiomeric excess.

### N—((S)-1-benzofuran-2-yl(2-bromophenyl)methyl)-3,4-dihydro-2H-1,5-benzodioxepine-7-sulfonamide

First Eluting Peak (Peak #1)

¹H NMR (400 MHz, chloroform-d) δ 7.54-7.47 (m, 1H), 7.44 (d, J=7.8 Hz, 1H), 7.40 (dd, J=1.6, 7.8 Hz, 1H), 7.37-7.29 (m, 3H), 7.28-7.09 (m, 4H), 6.84 (d, J=8.2 Hz, 1H), 6.43 (s, 1H), 6.16 (d, J=7.4 Hz, 1H), 5.46 (d, J=7.4 Hz, 1H), 4.19 (t, J=5.8 Hz, 2H), 4.11 (t, J=5.8 Hz, 2H), 2.17 (quin, J=5.8 Hz, 2H). m/z (ESI, +ve ion) 535.7, 537.7 (M+Na)⁺. GK-GKRP EC₅₀ (LCMS/MS)>20 μM. GK-GKRP IC₅₀ (Binding)=4.5 μM.

### N—((R)-1-benzofuran-2-yl(2-bromophenyl)methyl)-3,4-dihydro-2H-1,5-benzodioxepine-7-sulfonamide

Second Eluting Peak (Peak #2)

¹H NMR (400 MHz, chloroform-d) δ 7.51 (d, J=8.0 Hz, 1H), 7.44 (d, J=7.6 Hz, 1H), 7.42-7.37 (m, 1H), 7.37-7.29 (m, 3H), 7.29-7.09 (m, 4H), 6.84 (d, J=8.2 Hz, 1H), 6.43 (s, 1H), 6.16 (d, J=7.4 Hz, 1H), 5.46 (d, J=7.4 Hz, 1H), 4.20 (t, J=5.8 Hz, 2H), 4.11 (t, J=5.8 Hz, 2H), 2.17 (quin, J=5.8 Hz, 2H). m/z (ESI, +ve ion) 535.7, 537.7 (M+Na)⁺. GK-GKRP EC₅₀ (LCMS/MS)=0.026 μM. GK-GKRP IC₅₀ (Binding)=0.083 μM.

### Example 23

### N-(1-benzofuran-2-yl(2-fluorophenyl)methyl)-3,4-dihydro-2H-1,5-benzodioxepine-7-sulfonamide

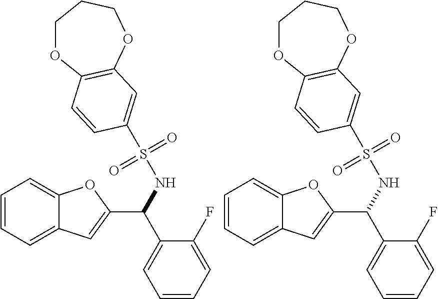

A 10 mL round-bottomed flask was charged with 1-bromo-2-fluorobenzene (0.059 g, 0.34 mmol, Sigma-Aldrich, St. Louis, Mo.) and tetrahydrofuran (1.4 mL). The solution was cooled to −78° C., n-butyllithium (0.12 mL of a 2.5 M solution with toluene, 0.31 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.10 g, 0.28 mmol) and tetrahydrofuran (1.4 mL) was added. After stirring for 15 min, methanol (1.0 mL) and silica gel (0.65 g) were added sequentially and the volatiles were removed under a vacuum. The residue was subjected to flash chromatography on silica gel (6.5 g of silica gel, 4:1 hexane-ethyl acetate) to give N-(1-benzofuran-2-yl(2-fluorophenyl)methyl)-3,4-dihydro-2H-1,5-benzodioxepine-7-sulfonamide (0.090 g) as a colorless solid (racemic mixture).

¹H NMR (400 MHz, DMSO-d₆) δ 9.04 (d, J=6.5 Hz, 1H), 7.53 (d, J=7.4 Hz, 1H), 7.49-7.37 (m, 2H), 7.32 (m, 1H), 7.28-7.03 (m, 6H), 6.89 (d, J=8.4 Hz, 1H), 6.53 (s, 1H), 5.92 (d, J=6.1 Hz, 1H), 4.07 (t, J=5.6 Hz, 2H), 3.99 (t, J=5.6 Hz, 2H), 2.06 (quin., J=5.6 Hz, 2H). m/z (ESI, +ve ion) 475.9 (M+Na)⁺. GK-GKRP EC₅₀ (LCMS/MS)=0.45 μM. GK-GKRP IC₅₀ (Binding)=0.49 μM.

### Example 24

### N-(1-benzofuran-2-yl(3-fluorophenyl)methyl)-3,4-dihydro-2H-1,5-benzodioxepine-7-sulfonamide

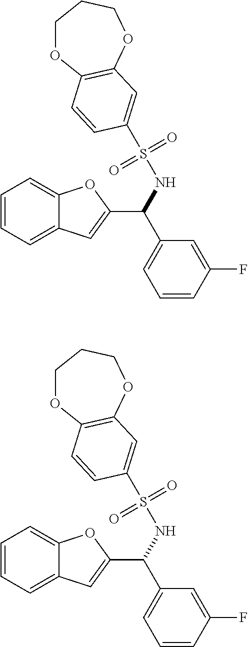 [SMILES: O=S(=O)(N[C@@H](c1cccc(F)c1)c1cc2ccccc2o1)c1ccc2c(c1)OCCCO2 ; O=S(=O)(N[C@H](c1cccc(F)c1)c1cc2ccccc2o1)c1ccc2c(c1)OCCCO2]

A 10 mL round-bottomed flask was charged with N-(1-benzofuran-2-ylmethylidene)-3,4-dihydro-2H-1,5-benzodioxepine-7-sulfonamide (intermediate A) (0.050 g, 0.14 mmol), 3-fluoroboronic acid (0.039 g, 0.28 mmol, Sigma-Aldrich, St. Louis, Mo.), 1,4-dioxane (2.0 mL), and bis(acetonitrile)(1,5-cyclooctadiene)rhodium(I) tetrafluoroborate (0.0053 g, 0.014 mmol, Sigma-Aldrich, St. Louis, Mo.), and then the reaction mixture was heated at 90° C. for 1 h. After that time, the reaction mixture was filtered and the filtrate was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(3-fluorophenyl)methyl)-3,4-dihydro-2H-1,5-benzodioxepine-7-sulfonamide (0.037 g) as an amorphous solid (racemic mixture).

¹H NMR (300 MHz, DMSO-d₆) δ 8.95 (d, J=12.0 Hz, 1H), 7.55-7.53 (m, 1H), 7.43 (m, 1H), 7.36-7.05 (m, 8H), 6.88 (d, J=9.0 Hz, 1H), 6.52 (s, 1H), 5.81 (d, J=9.0 Hz, 1H), 4.06 (t, J=6.0 Hz, 2H), 3.97 (t, J=6.0 Hz, 2H), 2.06 (quin, J=6.0 Hz, 2H). m/z (ESI, +ve ion) 476.0 (M+Na)⁺. GK-GKRP EC₅₀ (LCMS/MS)=1.8 μM.

### Example 25

### N-(1-benzofuran-2-yl(2-pyridinyl)methyl)-3,4-dihydro-2H-1,5-benzo dioxepine-7-sulfonamide

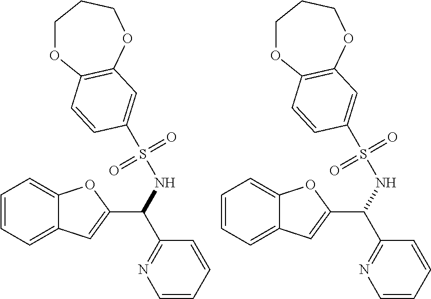

A 10 mL round-bottomed flask was charged with 2-bromopyridine (0.015 mL, 0.15 mmol, Sigma-Aldrich, St. Louis, Mo.) and tetrahydrofuran (2.0 mL). The solution was cooled to −78° C., n-butyllithium (0.12 mL of a 2.5 M solution with hexane, 0.31 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.050 g, 0.14 mmol) and tetrahydrofuran (1.0 mL) was added. After stirring for 10 min, water (0.10 mL) was added, and then the mixture was warmed to room temperature and subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(2-pyridinyl)methyl)-3,4-dihydro-2H-1,5-benzodioxepine-7-sulfonamide (0.014 g) as a colorless solid (racemic mixture).

¹H NMR (300 MHz, chloroform-d) δ 8.76 (dd, J=3.0, 6.0 Hz, 1H), 8.27 (td, J=3.0, 6.0 Hz, 1H), 7.96 (d, J=6.0 Hz, 1H), 7.72 (m, 1H), 7.55 (br s, 1H), 7.48 (d, J=6.0 Hz, 1H), 7.39-7.17 (m, 5H), 6.89 (m, 1H), 6.70 (s, 1H), 6.15 (s, 1H), 4.22-4.18 (m, 2H), 4.14-4.10 (m, 2H), 2.21-2.12 (m, 2H). m/z (ESI, +ve ion) 437.0 (M+H)⁺. GK-GKRP EC₅₀ (LCMS/MS)=0.27 μM. GK-GKRP IC₅₀ (Binding)=1.4 μM.

This racemic mixture was resolved using preparative SFC (Chiralpak® AD-H column (Daicel, Inc., Fort Lee, N.J.) (250 mm×21 mm, 5 μm) eluting with 60% liquid CO₂ in 40% methanol (with 2.0 mM NH₃) at a flow rate of 70 mL/min) to give two products in greater than 99% enantiomeric excess.

### N—((S)-1-benzofuran-2-yl(2-pyridinyl)methyl)-3,4-dihydro-2H-1,5-benzodioxepine-7-sulfonamide

First Eluting Peak (Peak #1)

¹H NMR (400 MHz, chloroform-d) δ 8.53 (br. s., 1H), 7.69 (t, J=7.7 Hz, 1H), 7.45 (d, J=7.4 Hz, 1H), 7.37-7.22 (m, 5H), 7.22-7.10 (m, 2H), 6.93 (d, J=6.1 Hz, 1H), 6.75 (d, J=8.2 Hz, 1H), 6.61 (s, 1H), 5.84 (d, J=6.3 Hz, 1H), 4.15-3.92 (m, 4H), 2.18-2.01 (m, 2H). m/z (ESI, +ve ion) 436.9 (M+H)⁺. GK-GKRP EC₅₀ (LCMS/MS)=6.0 μM. GK-GKRP IC₅₀ (Binding)=3.8 μM.

### N—((R)-1-benzofuran-2-yl(2-pyridinyl)methyl)-3,4-dihydro-2H-1,5-benzodioxepine-7-sulfonamide

Second Eluting Peak (Peak #2)

¹H NMR (400 MHz, chloroform-d) δ 8.54 (br. s., 1H), 7.65 (m, 1H), 7.44 (d, J=7.0 Hz, 1H), 7.37-7.22 (m, 5H), 7.22-7.07 (m, 2H), 6.86 (m, 1H), 6.74 (d, J=8.2 Hz, 1H), 6.59 (s, 1H), 5.80 (m, 1H), 4.15-3.91 (m, 4H), 2.10 (m, 2H). m/z (ESI, +ve ion) 436.9 (M+H)⁺. GK-GKRP EC₅₀ (LCMS/MS)=0.29 μM. GK-GKRP IC₅₀ (Binding)=0.21 μM.

### Example 26

### N-(1-benzofuran-2-yl(3-methyl-2-pyridinyl)methyl)-3,4-dihydro-2H-1,5-benzodioxepine-7-sulfonamide

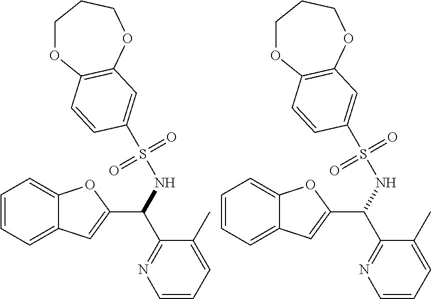

A 10 mL round-bottomed flask was charged with N-(1-benzofuran-2-ylmethylidene)-3,4-dihydro-2H-1,5-benzodioxepine-7-sulfonamide (intermediate A) (0.050 g, 0.14 mmol), tetrahydrofuran (2.0 mL), and bromo(3-methyl-2-pyridinyl)magnesium (0.67 mL of a 0.25 M solution with tetrahydrofuran, 0.17 mmol, Rieke Metals, Lincoln, Nebr.) and then the reaction mixture was stirred for 30 min. After that time, saturated aqueous ammonium chloride (1.0 mL) and dichloromethane (1.0 mL) were added, the mixture was filtered through a Radleys phase separation column (Brinkmann Instruments, Westbury, N.Y.), and then the filtrate was subjected to flash chromatography on silica gel (Isco CombiFlash® system, 12 g RediSep® normal phase column, gradient elution of hexane to 25% ethyl acetate-hexane, Teledyne Isco, Lincoln, Nebr.) to give N-(1-benzofuran-2-yl(3-methyl-2-pyridinyl)methyl)-3,4-dihydro-2H-1,5-benzodioxepine-7-sulfonamide (0.031 g) as a colorless solid (racemic mixture).

¹H NMR (300 MHz, methanol-d₄) δ 8.72 (d, J=9.0 Hz, 1H), 8.30 (m, 1H), 7.57 (m, 1H), 7.50 (m, 1H), 7.38 (m, 1H), 7.26-7.14 (m, 5H), 6.81 (d, J=9.0 Hz, 1H), 6.54 (s, 1H), 5.94 (d, J=9.0 Hz, 1H), 4.07-3.87 (m, 4H), 2.32 (s, 3H), 2.04 (m, 2H). m/z (ESI, +ve ion) 450.8 (M+H)⁺. GK-GKRP EC₅₀ (LCMS/MS)=0.19 μM. GK-GKRP IC₅₀ (Binding)=0.42 μM.

This racemic mixture was resolved using preparative SFC (Chiralpak® AD-H column (Daicel, Inc., Fort Lee, N.J.) (250 mm×21 mm, 5 μm) eluting with 62% liquid CO₂ in 38% methanol (with 2.0 mM NH₃) at a flow rate of 70 mL/min) to give two products in greater than 99% enantiomeric excess.

### N—((S)-1-benzofuran-2-yl(3-methyl-2-pyridinyl)methyl)-3,4-dihydro-2H-1,5-benzodioxepine-7-sulfonamide

First Eluting Peak (Peak #1)

¹H NMR (400 MHz, chloroform-d) δ 8.39 (d, J=4.7 Hz, 1H), 7.41 (d, J=7.4 Hz, 2H), 7.32-7.22 (m, 3H), 7.22-7.08 (m, 3H), 7.05 (d, J=8.0 Hz, 1H), 6.69 (d, J=8.6 Hz, 1H), 6.43 (s, 1H), 5.91 (d, J=8.0 Hz, 1H), 4.15-3.88 (m, 4H), 2.28 (s, 3H), 2.10 (m, 2H). m/z (ESI, +ve ion) 450.9 (M+H)⁺. GK-GKRP IC₅₀ (Binding)>33 μM.

### N—((R)-1-benzofuran-2-yl(3-methyl-2-pyridinyl)methyl)-3,4-dihydro-2H-1,5-benzodioxepine-7-sulfonamide

Second Eluting Peak (Peak #2)

¹H NMR (400 MHz, chloroform-d) δ 8.39 (d, J=4.1 Hz, 1H), 7.41 (d, J=7.4 Hz, 2H), 7.32-7.23 (m, 3H), 7.23-7.08 (m, 3H), 7.05 (d, J=8.0 Hz, 1H), 6.69 (d, J=8.6 Hz, 1H), 6.43 (s, 1H), 5.91 (d, J=8.0 Hz, 1H), 4.17-3.89 (m, 4H), 2.28 (s, 3H), 2.10 (m, 2H). m/z (ESI, +ve ion) 450.9 (M+H)⁺. GK-GKRP EC₅₀ (LCMS/MS)=0.085 μM. GK-GKRP IC₅₀ (Binding)=0.12 μM.

### Example 27

### N-(1-benzofuran-2-yl(2-chloro-3-pyridinyl)methyl)-3,4-dihydro-2H-1,5-benzodioxepine-7-sulfonamide

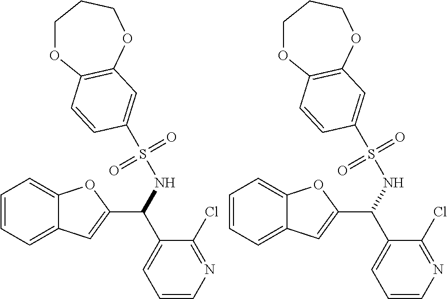

A 10 mL reaction vial was charged with tetrahydrofuran (2.0 mL) and diisopropylamine (0.040 mL, 0.28 mmol, Sigma-Aldrich, St. Louis, Mo.), cooled to −78° C., n-butyllithium (0.19 mL of a 1.6 M solution with hexane, 0.31 mmol, Sigma-Aldrich, St. Louis, Mo.) was added, and then the reaction mixture was stirred for 20 min. After that time, a solution of 2-chloropyridine (0.026 mL, 0.28 mmol, Sigma-Aldrich, St. Louis, Mo.) and tetrahydrofuran (2.0 mL) was added, and the reaction mixture was stirred for 30 min. After that time, a solution of N-(1-benzofuran-2-ylmethylidene)-3,4-dihydro-2H-1,5-benzodioxepine-7-sulfonamide (intermediate A) (0.10 g, 0.28 mmol) in tetrahydrofuran (2.0 mL) was added, and then the reaction mixture was warmed to room temperature for 2 h. After that time, the reaction mixture was cooled to 0° C., saturated aqueous sodium bicarbonate (1.0 mL) and dichloromethane (1.0 mL) were added, the mixture was filtered through a Radleys phase separation column (Brinkmann Instruments, Inc., Westbury, N.Y.), and the filtrate was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(2-chloro-3-pyridinyl)methyl)-3,4-dihydro-2H-1,5-benzodioxepine-7-sulfonamide (0.054 g) as a yellow oil (racemic mixture).

¹H NMR (300 MHz, DMSO-d₆) δ 9.16 (d, J=9.0 Hz, 1H), 8.33 (dd, J=3.0, 6.0 Hz, 1H), 7.95 (dd, J=3.0, 6.0 Hz, 1H), 7.55 (d, J=6.0 Hz, 1H), 7.48 (d, J=9.0 Hz, 1H), 7.44 (dd, J=3.0, 6.0 Hz, 1H), 7.32-7.19 (m, 3H), 7.15 (d, J=3.0 Hz, 1H), 6.92 (d, J=9.0 Hz, 1H), 6.51 (s, 1H), 5.99 (d, J=9.0 Hz, 1H), 4.15-4.00 (m, 4H), 2.14-2.08 (m, 2H). m/z (ESI, +ve ion) 471.0 (M+H)⁺. GK-GKRP EC₅₀ (LCMS/MS)=1.2 μM.

### Example 28

### N-(1-benzofuran-2-yl(3-chloro-4-pyridinyl)methyl)-3,4-dihydro-2H-1,5-benzodioxepine-7-sulfonamide

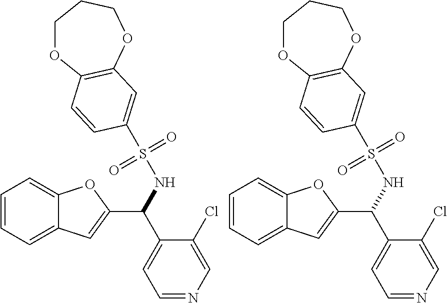

A 10 mL reaction vial was charged with tetrahydrofuran (2.0 mL) and diisopropylamine (0.040 mL, 0.28 mmol, Sigma-Aldrich, St. Louis, Mo.), cooled to −78° C., n-butyllithium (0.19 mL of a 1.6 M solution with hexane, 0.31 mmol, Sigma-Aldrich, St. Louis, Mo.) was added, and then the reaction mixture was stirred for 20 min. After that time, a solution of 3-chloropyridine (0.026 mL, 0.28 mmol, Alfa Aesar, Ward Hill, Mass.) in tetrahydrofuran (2.0 mL) was added, and the reaction mixture was stirred for 30 min. After that time, a solution of N-(1-benzofuran-2-ylmethylidene)-3,4-dihydro-2H-1,5-benzodioxepine-7-sulfonamide (intermediate A) (0.10 g, 0.28 mmol) and tetrahydrofuran (2.0 mL) was added, and then the reaction mixture was warmed to room temperature for 2 h. After that time, the reaction mixture was cooled to 0° C., saturated aqueous sodium bicarbonate (1.0 mL) and dichloromethane (1.0 mL) were added, the mixture was filtered through a Radleys phase separation column (Brinkmann Instruments, Inc., Westbury, N.Y.), and the filtrate was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(3-chloro-4-pyridinyl)methyl)-3,4-dihydro-2H-1,5-benzodioxepine-7-sulfonamide (0.043 g) as a yellow oil (racemic mixture).

¹H NMR (300 MHz, DMSO-d₆) δ 9.25 (d, J=9.0 Hz, 1H), 8.58 (s, 1H), 8.47 (d, J=6.0 Hz, 1H), 7.56-7.53 (m, 2H), 7.48 (d, J=9.0 Hz, 1H), (7.32-7.19 (m, 3H), 7.13 (d, J=1.7 Hz, 1H), 6.92 (d, J=9.0 Hz, 1H), 6.54 (s, 1H), 6.01 (d, J=9.0 Hz, 1H), 4.14-3.98 (m, 4H), 2.13-2.06 (m, 2H). m/z (ESI, +ve ion) 471.0 (M+H)⁺. GK-GKRP EC₅₀ (LCMS/MS)=1.6 μM.

### Example 29

### N-(1-benzofuran-2-yl(2-pyrimidinyl)methyl)-3,4-dihydro-2H-1,5-benzodioxepine-7-sulfonamide

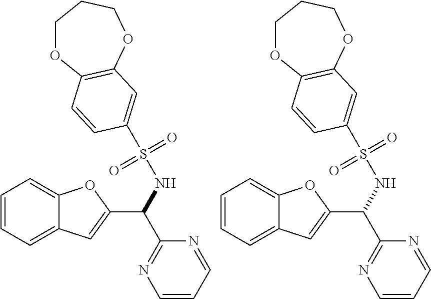

A 10 mL round-bottomed flask was charged with 2-bromopyrimidine (0.053 g, 0.34 mmol, Sigma-Aldrich, St. Louis, Mo.) and dichloromethane (2.8 mL). The solution was cooled to −78° C., n-butyllithium (0.12 mL of a 2.5 M solution with toluene, 0.31 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.10 g, 0.28 mmol) in dichloromethane (1.4 mL) was added and then the reaction mixture was allowed to warm to room temperature. After stirring for 40 min, methanol (1.0 mL) and silica gel (0.61 g) were added sequentially and the volatiles were removed under a vacuum. The residue was subjected to flash chromatography on silica gel (6.5 g of silica gel, gradient elution 2:1 to 1:1 hexane-ethyl acetate), and then the isolated material was re-subjected to flash chromatography on silica gel (2.8 g of silica gel, 99:1 dichloromethane-methanol) to give N-(1-benzofuran-2-yl(2-pyrimidinyl)methyl)-3,4-dihydro-2H-1,5-benzodioxepine-7-sulfonamide (0.012 g) as a colorless solid (racemic mixture).

¹H NMR (400 MHz, chloroform-d) δ 8.69 (d, J=4.9 Hz, 2H), 7.50-7.41 (m, 1H), 7.33-7.23 (m, 3H), 7.23-7.09 (m, 3H), 6.71 (d, J=8.6 Hz, 1H), 6.66 (s, 1H), 6.61 (d, J=7.6 Hz, 1H), 5.97 (d, J=7.8 Hz, 1H), 4.14-3.86 (m, 4H), 2.09 (quin, J=5.6 Hz, 2H). m/z (ESI, +ve ion) 437.9 (M+H)⁺. GK-GKRP EC₅₀ (LCMS/MS)=0.99 μM. GK-GKRP IC₅₀ (Binding)=1.8 μM.

### Example 30

### N-(1-benzofuran-2-yl(3-methoxy-2-pyridinyl)methyl)-3,4-dihydro-2H-1,5-benzodioxepine-7-sulfonamide

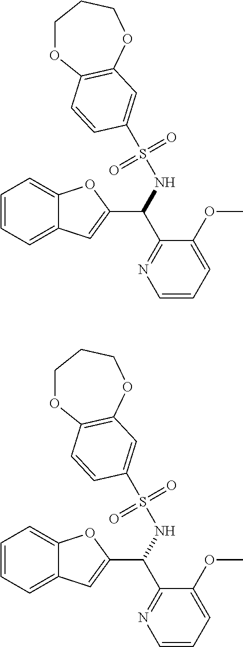 [SMILES: COc1cccnc1[C@H](NS(=O)(=O)c1ccc2c(c1)OCCCO2)c1cc2ccccc2o1 ; COc1cccnc1[C@@H](NS(=O)(=O)c1ccc2c(c1)OCCCO2)c1cc2ccccc2o1]

A 25 mL round-bottomed flask was charged with 2-bromo-1,3,5-trimethylbenzene (0.56 g, 2.8 mmol, Sigma-Aldrich, St. Louis, Mo.) and tetrahydrofuran (5.6 mL). The solution was cooled to −78° C., t-butyllithium (3.5 mL of a 1.7 M solution with pentane, 5.6 mmol, Sigma-Aldrich, St. Louis, Mo.) was added, and then the reaction mixture was stirred for 1 h. After that time, the reaction mixture was warmed to −23° C., 3-methoxypyridine (0.31 g, 2.8 mmol, Sigma-Aldrich, St. Louis, Mo.) was added, and then the reaction mixture was stirred for 3 h. After that time, an aliquot of this reaction mixture (1.0 mL) was added to a solution of N-(1-benzofuran-2-ylmethylidene)-3,4-dihydro-2H-1,5-benzodioxepine-7-sulfonamide (intermediate A) (0.10 g, 0.28 mmol) in tetrahydrofuran (2.8 mL) at 0° C. After stirring for 75 min, methanol (1.0 mL) and silica gel (0.50 g) were added sequentially and the volatiles were removed under a vacuum. The residue was subjected to flash chromatography on silica gel (6.5 g of silica gel, gradient elution 6:1 to 2:1 hexane-ethyl acetate), and then the isolated material was re-subjected to flash chromatography on silica gel (3.0 g of silica gel, 99.5:0.5 dichloromethane-methanol) to give N-(1-benzofuran-2-yl(3-methoxy-2-pyridinyl)methyl)-3,4-dihydro-2H-1,5-benzodioxepine-7-sulfonamide (0.020 g) as a pale yellow tar (racemic mixture).

¹H NMR (400 MHz, chloroform-d) δ 8.16-8.08 (m, 1H), 7.44-7.38 (m, 1H), 7.32-7.22 (m, 3H), 7.20-7.05 (m, 4H), 6.77-6.63 (m, 2H), 6.44 (s, 1H), 6.19 (d, J=8.8 Hz, 1H), 4.14-3.96 (m, 3H), 3.96-3.87 (m, 1H), 3.79 (s, 3H), 2.20-1.94 (m, 2H). m/z (ESI, +ve ion) 466.9 (M+H)⁺. GK-GKRP EC₅₀ (LCMS/MS)=0.15 μM. GK-GKRP IC₅₀ (Binding)=0.31 μM.

### Example 31

### N-(1-benzofuran-2-yl(2-thiophenyl)methyl)-3,4-dihydro-2H-1,5-benzodioxepine-7-sulfonamide

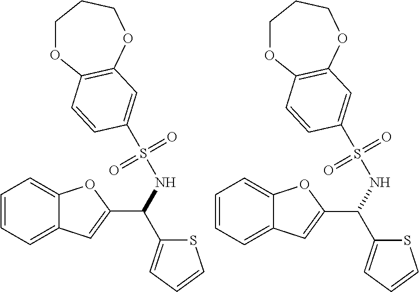

A 10 mL round-bottomed flask was charged with 2-bromothiophene (0.025 g, 0.15 mmol, Sigma-Aldrich, St. Louis, Mo.) and tetrahydrofuran (2.0 mL). The solution was cooled to −78° C., n-butyllithium (0.12 mL of a 2.5 M solution with hexane, 0.31 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.050 g, 0.14 mmol) in tetrahydrofuran (1.0 mL) was added. After stirring for 10 min, water (0.10 mL) was added, the reaction mixture was warmed to room temperature and concentrated. The residue was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(2-thiophenyl)methyl)-3,4-dihydro-2H-1,5-benzodioxepine-7-sulfonamide (0.038 g) as an amorphous solid (racemic mixture).

¹H NMR (300 MHz, chloroform-d) δ 7.47 (dd, J=3.0, 9.0 Hz, 1H), 7.34-7.16 (m, 6H), 6.92-6.89 (m, 2H), 6.78 (d, J=6.0 Hz 1H), 6.54 (s, 1H), 6.00 (d, J=6.0 Hz, 1H), 5.30 (d, J=9.0 Hz, 1H), 4.09 (t, J=6.0 Hz, 2H), 3.99 (t, J=6.0 Hz, 2H), 2.11 (quin, J=6.0 Hz, 2H). m/z (ESI, +ve ion) 464.0 (M+Na)⁺. GK-GKRP EC₅₀ (LCMS/MS)=0.74 μM. GK-GKRP IC₅₀ (Binding)=3.2 μM.

### Example 32

### N-(1-benzofuran-2-yl(3-thiophenyl)methyl)-3,4-dihydro-2H-1,5-benzodioxepine-7-sulfonamide

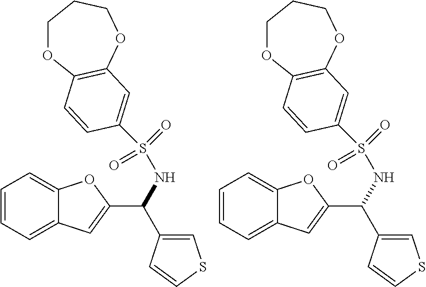

A 10 mL round-bottomed flask was charged with N-(1-benzofuran-2-ylmethylidene)-3,4-dihydro-2H-1,5-benzodioxepine-7-sulfonamide (0.20 g, 0.56 mmol), 3-thiophenylboronic acid (0.14 g, 1.1 mmol, Sigma-Aldrich, St. Louis, Mo.), 1,4-dioxane (2.2 mL), and bis(acetonitrile) (1,5-cyclooctadiene)rhodium(I) tetrafluoroborate (0.021 g, 0.056 mmol, Sigma-Aldrich, St. Louis, Mo.), and then the reaction mixture was heated at 95° C. for 22 h. After that time, the reaction mixture was partitioned between ethyl acetate (20 mL) and saturated aqueous sodium bicarbonate (20 mL), the layers were separated, the organic material was washed sequentially with saturated aqueous sodium bicarbonate (20 mL) and brine (20 mL), dried (sodium sulfate), filtered, and the filtrate was concentrated. The residue was subjected to flash chromatography on silica gel (13 g of silica gel, 4:1 hexane-ethyl acetate) to give N-(1-benzofuran-2-yl(3-thiophenyl)methyl)-3,4-dihydro-2H-1,5-benzodioxepine-7-sulfonamide (0.050 g) as a tan solid (racemic mixture).

¹H NMR (400 MHz, chloroform-d) δ 7.45 (d, J=7.2 Hz, 1H), 7.37-7.16 (m, 6H), 7.12 (br. s., 1H), 6.99 (d, J=4.3 Hz, 1H), 6.77 (d, J=8.2 Hz, 1H), 6.46 (s, 1H), 5.83 (d, J=8.4 Hz, 1H), 5.24 (d, J=8.2 Hz, 1H), 4.09 (t, J=5.1 Hz, 2H), 3.98 (t, J=5.5 Hz, 2H), 2.21-2.00 (m, 2H). m/z (ESI, +ve ion) 463.9 (M+Na)⁺. GK-GKRP EC₅₀ (LCMS/MS)=1.4 μM.

### Example 33

### N-(1-benzofuran-2-yl(3-methyl-2-thiophenyl)methyl)-3,4-dihydro-2H-1,5-benzodioxepine-7-sulfonamide

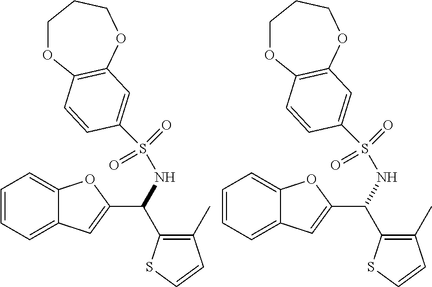

A 10 mL round-bottomed flask was charged with N-(1-benzofuran-2-ylmethylidene)-3,4-dihydro-2H-1,5-benzodioxepine-7-sulfonamide (intermediate A) (0.050 g, 0.14 mmol), tetrahydrofuran (2.0 mL), and bromo(3-methyl-2-thiophenyl)magnesium (0.28 mL of a 0.50 M solution with tetrahydrofuran, 0.14 mmol, Rieke Metals, Lincoln, Nebr.), and then the reaction mixture was stirred for 1 h. After that time, water (1.0 mL) was added, and then the mixture was subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(3-methyl-2-thiophenyl)methyl)-3,4-dihydro-2H-1,5-benzodioxepine-7-sulfonamide (0.055 g) as a colorless solid (racemic mixture).

¹H NMR (300 MHz, methanol-d₄) δ 7.47 (m, 1H), 7.38-7.31 (m, 2H), 7.27-7.15 (m, 3H), 6.83 (d, 2H), 6.83 (d, J=9.0 Hz, 1H), 6.79 (d, J=6.0 Hz, 1H), 6.50 (s, 1H), 6.00 (s, 1H), 4.09-3.93 (m, 4H), 2.22 (s, 3H), 2.10 (quin, J=6.0 Hz, 2H). m/z (ESI, +ve ion) 478.0 (M+Na)⁺. GK-GKRP EC₅₀ (LCMS/MS)=1.2 μM. GK-GKRP IC₅₀ (Binding)=1.4 μM.

### Example 34

### N-(1-benzofuran-2-yl(1,3-thiazol-2-yl)methyl)-3,4-dihydro-2H-1,5-benzodioxepine-7-sulfonamide

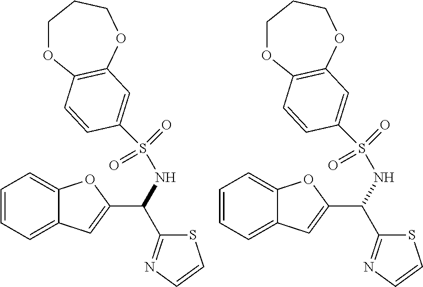

A 5 mL round-bottomed flask was charged with 2-bromo-1,3-thiazole (0.025 g, 0.15 mmol, Sigma-Aldrich, St. Louis, Mo.) and tetrahydrofuran (2.0 mL). The solution was cooled to −78° C., n-butyllithium (0.12 mL of a 2.5 M solution with hexane, 0.31 mmol, Sigma-Aldrich, St. Louis, Mo.) was added, and then the reaction mixture was stirred for 30 min. After that time, a solution of N-(1-benzofuran-2-ylmethylidene)-3,4-dihydro-2H-1,5-benzodioxepine-7-sulfonamide (intermediate A) (0.050 g, 0.14 mmol) in tetrahydrofuran (2.0 mL) was added. After stirring for 30 min, water (0.10 mL) was added, and then the mixture was warmed to room temperature and subjected to reversed-phase preparative HPLC (Phenomenex Gemini C18 column (Phenomenex, Inc., Torrance, Calif.) (150×30 mm, 5 μm) eluting with 0.10% trifluoroacetic acid in acetonitrile-water, gradient of 10% to 90% over 10 min) to give N-(1-benzofuran-2-yl(1,3-thiazol-2-yl)methyl)-3,4-dihydro-2H-1,5-benzodioxepine-7-sulfonamide (0.0047 g) as an amorphous solid (racemic mixture).

¹H NMR (300 MHz, chloroform-d) δ 7.73 (d, J=3.0 Hz, 1H), 7.47 (m, 1H), 7.35-7.31 (m, 4H), 7.27-7.15 (m, 2H), 6.81 (m, 1H), 6.60 (s, 1H), 6.18 (d, J=9.0 Hz, 1H), 6.09 (d, J=9.0 Hz, 1H), 4.14-4.10 (m, 2H), 4.03 (t, J=6.0 Hz 2H), 2.17-2.08 (m, 2H). m/z (ESI, +ve ion) 443.0 (M+H)⁺. GK-GKRP EC₅₀ (LCMS/MS)=2.8 μM. GK-GKRP IC₅₀ (Binding)=6.6 μM.

### Example 35

### N-(1-benzofuran-2-yl(phenyl)methyl)-3-fluoro-3,4-dihydro-2H-1,5-benzodioxepine-7-sulfonamide

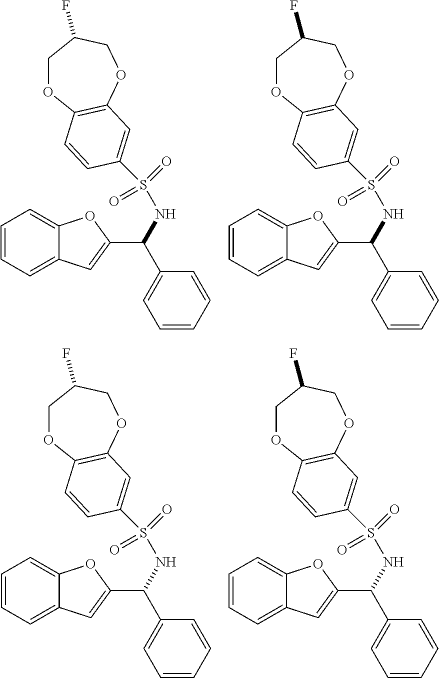

Step 1.

A 250 mL round-bottomed flask was charged with 1,3-dibromo-2-propanol (10 g, 46 mmol, Sigma-Aldrich, St. Louis, Mo.), dichloromethane (92 mL), N-ethyl-N-(1-methylethyl)-2-propanamine (24 mL, 140 mmol, EMD, Gibbstown, N.J.), and tris(1-methylethyl)silyl trifluoromethanesulfonate (14 mL, 51 mmol, Sigma-Aldrich, St. Louis, Mo.), and then the reaction mixture was stirred for 24 h. After that time, silica gel (34 g) was added, and the volatiles were removed under a vacuum. The residue was subjected to flash chromatography on silica gel (340 g of silica gel, hexane) to give (2-bromo-1-(bromomethyl)ethoxy)(tris(1-methylethyl))silane (14 g) as a clear, colorless oil.

Step 2.

A 150 mL round-bottomed flask was charged with (2-bromo-1-(bromomethyl)ethoxy)(tris(1-methylethyl))silane (9.9 g, 27 mmol), N,N-dimethylformamide (53 mL), 4-bromo-1,2-benzenediol (5.0 g, 27 mmol, Best PharmaTech, Schaumburg, Ill.), and potassium carbonate (11 g, 79 mmol, Sigma-Aldrich, St. Louis, Mo.), and then the reaction mixture was heated to 80° C. for 18 h. After that time, the reaction mixture was cooled to room temperature, partitioned between ethyl acetate (250 mL) and saturated aqueous sodium bicarbonate (250 mL), the layers were separated, the organic material was washed sequentially with saturated aqueous sodium bicarbonate (3×250 mL) and brine (2×250 mL), dried (sodium sulfate), filtered, and the filtrate was concentrated. The residue was subjected to flash chromatography on silica gel (500 g of silica gel, gradient elution of 9:1 to 4:1 hexane-ethyl acetate), the isolated material was re-subjected to flash chromatography on silica gel (100 g of silica gel, hexane), and then the isolated material was dissolved with hexane (50 mL) and the solution was filtered through a plug of silica gel (20 g) to afford ((7-bromo-3,4-dihydro-2H-1,5-benzodioxepin-3-yl)oxy)(tris(1-methylethyl))silane (3.4 g) as a clear, colorless oil.

Step 3.

A 250 mL round-bottomed flask was charged with tetrahydrofuran (65 mL), cooled to −78° C., t-butyllithium (10 mL of a 1.7 M solution with pentane, Sigma-Aldrich, St. Louis, Mo.) and a solution of ((7-bromo-3,4-dihydro-2H-1,5-benzodioxepin-3-yl)oxy)(tris(1-methylethyl))silane (3.4 g, 8.5 mmol) and tetrahydrofuran (25 mL) were added sequentially, the reaction mixture was stirred for 15 min, sparged with sulfur dioxide (Sigma-Aldrich, St. Louis, Mo.) for 5 min, and then warmed to room temperature. After stirring for 90 min, the reaction mixture was concentrated, the residue was dissolved with dichloromethane (85 mL), 1-chloro-2,5-pyrrolidinedione (1.3 g, 9.4 mmol, Sigma-Aldrich, St. Louis, Mo.) was added, and then the reaction mixture was stirred for 90 min. After that time, 1-(1-benzofuran-2-yl)-1-phenylmethanamine hydrochloride (2.2 g, 8.6 mmol, Enamine, Kiev, Ukraine) and N-ethyl-N-(1-methylethyl)-2-propanamine (7.5 mL, 43 mmol, EMD, Gibbstown, N.J.) were added, and the reaction mixture was stirred for 18 h. After that time, the reaction mixture was concentrated, the residue was partitioned between ethyl acetate (100 mL) and saturated aqueous sodium bicarbonate (100 mL), the layers were separated, the organic material was washed sequentially with saturated aqueous sodium bicarbonate (100 mL) and brine (100 mL), dried (sodium sulfate), filtered, and the filtrate was concentrated. The residue was subjected to flash chromatography on silica gel (250 g of silica gel, 9:1 hexane-ethyl acetate) to give N-(1-benzofuran-2-yl(phenyl)methyl)-3-((tris(1-methylethyl)silyl)oxy)-3,4-dihydro-2H-1,5-benzodioxepine-7-sulfonamide (2.9 g) as pale, peach-colored solid.

Step 4. A 150 mL round-bottomed flask was charged with N-(1-benzofuran-2-yl(phenyl)methyl)-3-((tris(1-methylethyl)silyl)oxy)-3,4-dihydro-2H-1,5-benzodioxepine-7-sulfonamide (2.9 g, 4.8 mmol), tetrahydrofuran (48 mL), and tetrabutylammonium fluoride (4.8 mL of a 1.0 M solution with tetrahydrofuran, 4.8 mmol, Sigma-Aldrich, St. Louis, Mo.), and then the reaction mixture was stirred for 90 min. After that time, the reaction mixture was partitioned between ethyl acetate (50 mL) and saturated aqueous sodium bicarbonate (50 mL), the layers were separated, the organic material was washed sequentially with saturated aqueous sodium bicarbonate (50 mL) and brine (2×50 mL), dried (sodium sulfate), filtered, and the filtrate was concentrated. The residue was subjected to flash chromatography on silica gel (110 g of silica gel, gradient elution of 1.5:1 to 1:1 hexane-ethyl acetate) to give N-(1-benzofuran-2-yl(phenyl)methyl)-3-hydroxy-3,4-dihydro-2H-1,5-benzodioxepine-7-sulfonamide (2.0 g) as a colorless solid.

Step 5. A 10 mL round-bottomed flask was charged with N-(1-benzofuran-2-yl(phenyl)methyl)-3-hydroxy-3,4-dihydro-2H-1,5-benzodioxepine-7-sulfonamide (0.10 g, 0.22 mmol), tetrabutylammonium dihydrogentrifluoride (1.1 mL of a 50-55 wt % solution with dichloroethane, Acros, Geel, Belgium), and diethylaminosulfur trifluoride (0.088 mL, 0.66 mmol, Sigma-Aldrich, St. Louis, Mo.) and then the reaction mixture was stirred for 30 min. After that time, the reaction mixture was added to saturated aqueous sodium bicarbonate (50 mL), the mixture was stirred vigorously for 15 h, ethyl acetate (50 mL) was added, the layers were separated, the organic material was washed sequentially with saturated aqueous sodium bicarbonate (50 mL) and brine (50 mL), dried (sodium sulfate), filtered, and the filtrate was concentrated. The residue was subjected to flash chromatography on silica gel (6.5 g of silica gel, 9:1 hexane-ethyl acetate), and then the isolated material was subjected to reversed-phase preparative HPLC (Phenomenex Gemini C18 column (Phenomenex, Inc., Torrance, Calif.) (100×30 mm, 5 μm) eluting with 0.10% trifluoroacetic acid in acetonitrile-water, gradient of 10% to 90% over 20 min) to give N-(1-benzofuran-2-yl(phenyl)methyl)-3-fluoro-3,4-dihydro-2H-1,5-benzodioxepine-7-sulfonamide (0.019 g) as a colorless solid (mixture of four stereoisomers).

¹H NMR (400 MHz, chloroform-d) δ 7.45 (d, J=7.4 Hz, 1H), 7.34-7.27 (m, 7H), 7.26-7.15 (m, 3H), 6.78 (d, J=8.6 Hz, 1H), 6.44 (s, 1H), 5.77 (dd, J=3.3, 8.2 Hz, 1H), 5.31 (dd, J=4.4, 8.1 Hz, 1H), 4.95 (m, 1H), 4.34-4.05 (m, 4H). m/z (ESI, +ve ion) 475.9 (M+Na)⁺. GK-GKRP EC₅₀ (LCMS/MS)=3.1 μM. GK-GKRP IC₅₀ (Binding)=2.3 μM.

### Intermediate B

### N-(1-benzothiophen-2-ylmethylidene)-3,4-dihydro-2H-1,5-benzodioxepine-7-sulfonamide

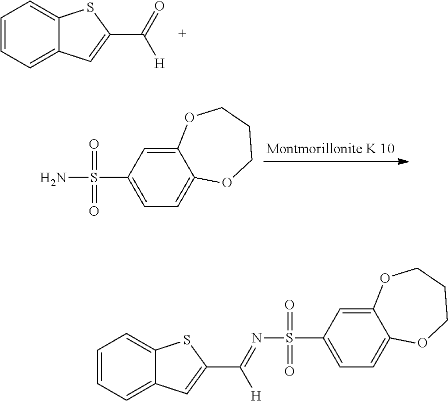 [SMILES: O=Cc1cc2ccccc2s1 ; NS(=O)(=O)c1ccc2c(c1)OCCCO2 ; O=S(=O)(/N=C/c1cc2ccccc2s1)c1ccc2c(c1)OCCCO2]

A 100 mL round-bottomed flask was charged with 1-benzothiophene-2-carbaldehyde (2.0 g, 12 mmol, Maybridge, Tintagel, United Kingdom), 3,4-dihydro-2H-1,5-benzodioxepine-7-sulfonamide (2.8 g, 12 mmol, Enamine, Kiev, Ukraine), Montmorillonite K 10 (2.0 g, Sigma-Aldrich, St. Louis, Mo.), and toluene (62 mL), fitted with a Dean-Stark trap and a reflux condenser, and then the reaction mixture was heated at reflux for 2 h. After that time, the reaction mixture was filtered, the filtrate was allowed to cool to room temperature, filtered, and the filter cake was collected to give N-(1-benzothiophen-2-ylmethylidene)-3,4-dihydro-2H-1,5-benzodioxepine-7-sulfonamide (3.3 g) as a pale yellow solid.

### Example 36

### N-(1-benzothiophen-2-yl(2-methylphenyl)methyl)-3,4-dihydro-2H-1,5-benzodioxepine-7-sulfonamide

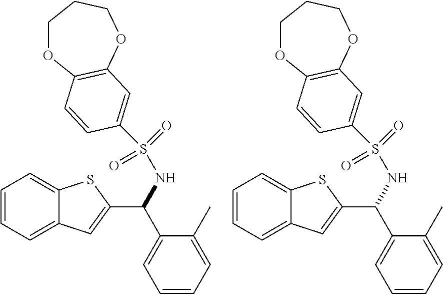

A 10 mL round-bottomed flask was charged with N-(1-benzothiophen-2-ylmethylidene)-3,4-dihydro-2H-1,5-benzodioxepine-7-sulfonamide (intermediate B) (0.10 g, 0.27 mmol), tetrahydrofuran (2.7 mL), and bromo(2-methylphenyl)magnesium (0.15 mL of a 2.0 M solution with diethyl ether, 0.30 mmol, Sigma-Aldrich, St. Louis, Mo.), and then the reaction mixture was stirred for 75 min. After that time, methanol (1.0 mL) and silica gel (0.60 g) were added sequentially and the volatiles were removed under a vacuum. The residue was subjected to flash chromatography on silica gel (6.5 g of silica gel, 4:1 hexane-ethyl acetate) to give N-(1-benzothiophen-2-yl(2-methylphenyl)methyl)-3,4-dihydro-2H-1,5-benzodioxepine-7-sulfonamide (0.094 g) as a colorless solid (racemic mixture).

¹H NMR (400 MHz, DMSO-d₆) δ 8.94 (br. s., 1H), 7.92-7.79 (m, 1H), 7.74-7.58 (m, 1H), 7.37-7.24 (m, 3H), 7.22 (dd, J=2.3, 8.4 Hz, 1H), 7.18-7.00 (m, 4H), 6.91-6.76 (m, 2H), 5.91 (br. s., 1H), 4.13-4.00 (m, 2H), 4.00-3.86 (m, 2H), 2.24 (s, 3H), 2.13-2.00 (m, 2H). m/z (ESI, +ve ion) 487.9 (M+Na)⁺. GK-GKRP EC₅₀ (LCMS/MS)=0.15 μM. GK-GKRP IC₅₀ (Binding)=0.29 μM.

This racemic mixture was resolved using preparative SFC (Chiralpak® AS-H column (Daicel, Inc., Fort Lee, N.J.) (250 mm×21 mm, 5 μm) eluting with 58% liquid CO₂ in 42% methanol (with 0.20% diethylamine) at a flow rate of 70 mL/min) to give two products in greater than 99% enantiomeric excess.

### N—((S)-1-benzothiophen-2-yl(2-methylphenyl)methyl)-3,4-dihydro-2H-1,5-benzodioxepine-7-sulfonamide

First Eluting Peak (Peak #1)

¹H NMR (400 MHz, chloroform-d) δ 7.69 (d, J=7.6 Hz, 1H), 7.64-7.52 (m, 1H), 7.35-7.22 (m, 4H), 7.22-7.14 (m, 2H), 7.14-7.04 (m, 2H), 6.90-6.76 (m, 2H), 6.07 (d, J=7.6 Hz, 1H), 5.27 (d, J=7.6 Hz, 1H), 4.27-3.97 (m, 4H), 2.30 (s, 3H), 2.24-2.07 (m, 2H). m/z (ESI, +ve ion) 487.9 (M+Na)⁺. GK-GKRP EC₅₀ (LCMS/MS)>20 μM. GK-GKRP IC₅₀ (Binding)=4.0 μM.

### N—((R)-1-benzothiophen-2-yl(2-methylphenyl)methyl)-3,4-dihydro-2H-1,5-benzodioxepine-7-sulfonamide

Second Eluting Peak (Peak #2)

¹H NMR (400 MHz, chloroform-d) δ 7.69 (d, J=7.6 Hz, 1H), 7.58 (d, J=7.2 Hz, 1H), 7.35-7.22 (m, 4H), 7.22-7.13 (m, 2H), 7.13-7.05 (m, 2H), 6.89-6.74 (m, 2H), 6.07 (d, J=7.2 Hz, 1H), 5.28 (d, J=7.6 Hz, 1H), 4.27-3.97 (m, 4H), 2.30 (s, 3H), 2.23-2.01 (m, 2H). m/z (ESI, +ve ion) 487.9 (M+Na)⁺. GK-GKRP EC₅₀ (LCMS/MS)=0.062 μM. GK-GKRP IC₅₀ (Binding)=0.24 μM.

### Example 37

### N-(1-benzothiophen-2-yl(2-ethylphenyl)methyl)-3,4-dihydro-2H-1,5-benzodioxepine-7-sulfonamide

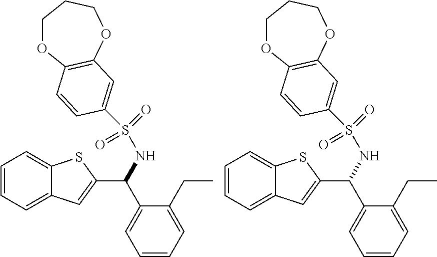

A 10 mL round-bottomed flask was charged with N-(1-benzothiophen-2-ylmethylidene)-3,4-dihydro-2H-1,5-benzodioxepine-7-sulfonamide (intermediate B) (0.10 g, 0.27 mmol), tetrahydrofuran (2.7 mL), and bromo(2-ethylphenyl)magnesium (a total of 1.8 mL of a 0.50 M solution with tetrahydrofuran, added in three equal portions, 0.89 mmol, Rieke Metals, Lincoln, Nebr.), and then the reaction mixture was stirred for 30 min. After that time, methanol (1.0 mL) and silica gel (0.60 g) were added sequentially and the volatiles were removed under a vacuum. The residue was subjected to flash chromatography on silica gel (6.5 g of silica gel, 4:1 hexane-ethyl acetate) to give N-(1-benzothiophen-2-yl(2-ethylphenyl)methyl)-3,4-dihydro-2H-1,5-benzodioxepine-7-sulfonamide (0.084 g) as a colorless solid (racemic mixture).

¹H NMR (400 MHz, DMSO-d₆) δ 8.98 (d, J=8.0 Hz, 1H), 7.92-7.78 (m, 1H), 7.73-7.61 (m, 1H), 7.40-7.24 (m, 3H), 7.24-7.01 (m, 5H), 6.87 (d, J=8.4 Hz, 1H), 6.81 (s, 1H), 5.96 (d, J=7.6 Hz, 1H), 4.06 (t, J=6.8 Hz, 2H), 4.01-3.87 (m, 2H), 2.66-2.53 (m, 2H), 2.14-1.96 (m, 2H), 1.11 (t, J=7.5 Hz, 3H). m/z (ESI, +ve ion) 501.8 (M+Na)⁺. GK-GKRP EC₅₀ (LCMS/MS)=0.16 μM. GK-GKRP IC₅₀ (Binding)=0.30 μM.

This racemic mixture was resolved using preparative SFC (Chiralpak® AS-H column (Daicel, Inc., Fort Lee, N.J.) (250 mm×21 mm, 5 μm) eluting with 58% liquid CO₂ in 42% methanol (with 0.20% diethylamine) at a flow rate of 70 mL/min) to give two products in greater than 99% enantiomeric excess.

### N—((S)-1-benzothiophen-2-yl(2-ethylphenyl)methyl)-3,4-dihydro-2H-1,5-benzodioxepine-7-sulfonamide

First Eluting Peak (Peak #1)

¹H NMR (400 MHz, chloroform-d) δ 7.76-7.63 (m, 1H), 7.63-7.53 (m, 1H), 7.34-7.13 (m, 7H), 7.13-7.02 (m, 1H), 6.92-6.76 (m, 2H), 6.14 (d, J=7.6 Hz, 1H), 5.26 (d, J=7.6 Hz, 1H), 4.28-3.99 (m, 4H), 2.65 (q, J=7.6 Hz, 2H), 2.26-2.04 (m, 2H), 1.20 (t, J=7.6 Hz, 3H). m/z (ESI, +ve ion) 501.8 (M+Na)⁺. GK-GKRP EC₅₀ (LCMS/MS)>20 μM. GK-GKRP IC₅₀ (Binding)=1.3 μM.

### N—((R)-1-benzothiophen-2-yl(2-ethylphenyl)methyl)-3,4-dihydro-2H-1,5-benzodioxepine-7-sulfonamide

Second Eluting Peak (Peak #2)

¹H NMR (400 MHz, chloroform-d) δ 7.75-7.64 (m, 1H), 7.63-7.53 (m, 1H), 7.35-7.13 (m, 7H), 7.13-7.03 (m, 1H), 6.92-6.77 (m, 2H), 6.14 (s, 1H), 5.27 (br. s., 1H), 4.28-3.98 (m, 4H), 2.65 (q, J=7.6 Hz, 2H), 2.16 (m, 2H), 1.19 (t, J=7.5 Hz, 3H). m/z (ESI, +ve ion) 501.8 (M+Na)⁺. GK-GKRP EC₅₀ (LCMS/MS)=0.038 μM. GK-GKRP IC₅₀ (Binding)=0.13 μM.

### Example 38

### N-(1-benzothiophen-2-yl(2-ethenylphenyl)methyl)-3,4-dihydro-2H-1,5-benzodioxepine-7-sulfonamide

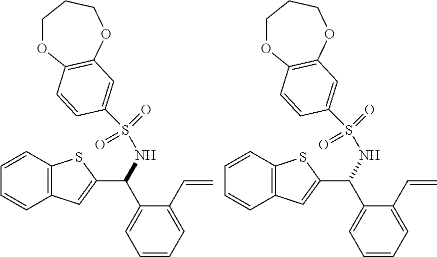

A 15 mL round-bottomed flask was charged with 1-bromo-2-ethenylbenzene (0.074 g, 0.40 mmol, Sigma-Aldrich, St. Louis, Mo.) and tetrahydrofuran (3.0 mL). The solution was cooled to −78° C., n-butyllithium (0.25 mL of a 1.6 M solution with hexane, 0.40 mmol, Sigma-Aldrich, St. Louis, Mo.) was added, and then the reaction mixture was stirred for 1 h. After that time, a solution of N-(1-benzothiophen-2-ylmethylidene)-3,4-dihydro-2H-1,5-benzodioxepine-7-sulfonamide (intermediate B) (0.15 g, 0.40 mmol) in tetrahydrofuran (3.0 mL) was added. After stirring for 5 min, saturated aqueous sodium bicarbonate (5.0 mL) was added, the mixture was warmed to room temperature, partitioned between ethyl acetate (50 mL) and saturated aqueous sodium bicarbonate (50 mL), the layers were separated, the organic material was washed with brine (50 mL), dried (magnesium sulfate), filtered, silica gel (2.0 g) was added to the filtrate, and the volatiles were removed under a vacuum. The residue was subjected to flash chromatography on silica gel (Isco CombiFlash® system, 40 g RediSep® normal phase column, gradient elution of 10% to 30% ethyl acetate-hexane, Teledyne Isco, Lincoln, Nebr.) to give N-(1-benzothiophen-2-yl(2-ethenylphenyl)methyl)-3,4-dihydro-2H-1,5-benzodioxepine-7-sulfonamide (0.0021 g) as an off-white oil (racemic mixture).

¹H NMR (400 MHz, acetonitrile-d₃) δ 7.84-7.74 (m, 1H), 7.68-7.59 (m, 1H), 7.50-7.39 (m, 1H), 7.36-7.17 (m, 6H), 7.13 (d, J=2.4 Hz, 1H), 6.97 (dd, J=17.1, 11.1 Hz, 1H), 6.87-6.79 (m, 2H), 6.70 (d, J=8.6 Hz, 1H), 6.10 (d, J=8.4 Hz, 1H), 5.68-5.54 (m, 1H), 5.32 (dd, J=11.0, 0.98 Hz, 1H), 4.21-3.94 (m, 4H), 2.11 (s, 2H). m/z (ESI, +ve ion) 499.9 (M+Na)⁺. GK-GKRP EC₅₀ (LCMS/MS)=0.031 μM. GK-GKRP IC₅₀ (Binding)=0.025 μM.

### Example 39

### N-(1-benzothiophen-2-yl(2-ethynylphenyl)methyl)-3,4-dihydro-2H-1,5-benzodioxepine-7-sulfonamide

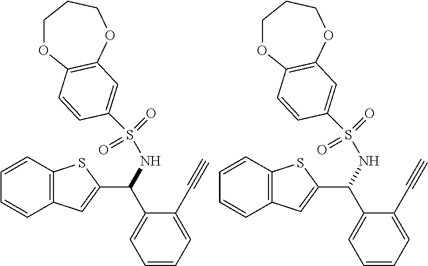

Step 1.

A 15 mL round-bottomed flask was charged with ((2-bromophenyl)ethynyl)(trimethyl)silane (0.20 g, 0.80 mmol, Sigma-Aldrich, St. Louis, Mo.) and tetrahydrofuran (3.0 mL). The solution was cooled to −78° C., n-butyllithium (0.50 mL of a 1.6 M solution with hexane, 0.80 mmol, Sigma-Aldrich, St. Louis, Mo.) was added, and then the reaction mixture was stirred for 1 h. After that time, a solution of N-(1-benzothiophen-2-ylmethylidene)-3,4-dihydro-2H-1,5-benzodioxepine-7-sulfonamide (intermediate B) (0.15 g, 0.40 mmol) and tetrahydrofuran (3.0 mL) was added. After stirring for 1 min, saturated aqueous sodium bicarbonate (2.0 mL) was added, the mixture was warmed to room temperature, partitioned between ethyl acetate (50 mL) and water (50 mL), the layers were separated, the organic material was washed with brine (25 mL), dried (magnesium sulfate), filtered, and the filtrate was concentrated. The isolated material (0.22 g) was used in the next step of the synthesis without purification.

Step 2.

A 15 mL round-bottomed flask was charged with the isolated material (0.22 g) from Step 1, tetrahydrofuran (2.0 mL), and tetrabutylammonium fluoride (0.40 mL of a 1.0 M solution with tetrahydrofuran, 0.40 mmol, Sigma-Aldrich, St. Louis, Mo.), and then the reaction mixture was stirred for 1 h. After that time, the reaction mixture was partitioned between saturated aqueous ammonium chloride (50 mL) and ethyl acetate (50 mL), the layers were separated, the organic material was washed with brine (50 mL), dried (magnesium sulfate), filtered, and the filtrate was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 20 min) to give N-(1-benzothiophen-2-yl(2-ethynylphenyl)methyl)-3,4-dihydro-2H-1,5-benzodioxepine-7-sulfonamide (0.018 g) as a tan solid (racemic mixture).

¹H NMR (400 MHz, DMSO-d₆) δ 9.10 (d, J=10 Hz, 1H), 7.94-7.81 (m, 1H), 7.75-7.63 (m, 1H), 7.49 (d, J=7.8 Hz, 1H), 7.43-7.36 (m, 1H), 7.34-7.21 (m, 4H), 7.17 (dd, J=8.4, 2.4 Hz, 1H), 7.08 (d, J=2.2 Hz, 1H), 6.85 (d, J=8.4 Hz, 1H), 6.79 (s, 1H), 6.26 (d, J=9.6 Hz, 1H), 4.47 (s, 1H), 4.15-3.92 (m, 4H), 2.13-2.01 (m, 2H). m/z (ESI, +ve ion) 497.9 (M+Na)⁺. GK-GKRP EC₅₀ (LCMS/MS)=0.037 μM. GK-GKRP IC₅₀ (Binding)=0.017 μM.

### Example 40

### N-(1-benzothiophen-2-yl(2-methoxyphenyl)methyl)-3,4-dihydro-2H-1,5-benzodioxepine-7-sulfonamide

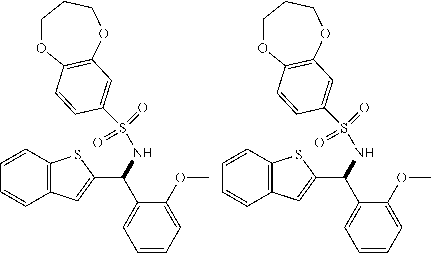

A 25 mL round-bottomed flask was charged with N-(1-benzothiophen-2-ylmethylidene)-3,4-dihydro-2H-1,5-benzodioxepine-7-sulfonamide (intermediate B) (0.20 g, 0.54 mmol), tetrahydrofuran (8.0 mL), and bromo(2-methoxyphenyl)magnesium (0.54 mL of a 1.0 M solution with diethyl ether, 0.54 mmol, Sigma-Aldrich, St. Louis, Mo.), and then the reaction mixture was stirred for 1 h. After that time, the reaction mixture was added to saturated aqueous sodium bicarbonate (50 mL), ethyl acetate (50 mL) was added, the layers were separated, the organic material was washed with brine (30 mL), dried (sodium sulfate), filtered, and the filtrate was concentrated. The residue was subjected to recrystallization (methanol) to give N-(1-benzothiophen-2-yl(2-methoxyphenyl)methyl)-3,4-dihydro-2H-1,5-benzodioxepine-7-sulfonamide (0.086 g) as a pale yellow solid (racemic mixture).

¹H NMR (400 MHz, DMSO-d₆) δ 8.85 (d, J=9.8 Hz, 1H), 7.84 (d, J=7.2 Hz, 1H), 7.67 (d, J=6.9 Hz, 1H), 7.42-7.13 (m, 5H), 7.07 (s, 1H), 6.96-6.72 (m, 4H), 6.13 (d, J=9.6 Hz, 1H), 4.17-3.90 (m, 4H), 3.72 (s, 3H), 2.18-1.93 (m, 2H). m/z (ESI, +ve ion) 503.9 (M+Na)⁺. GK-GKRP EC₅₀ (LCMS/MS)=0.046 μM. GK-GKRP IC₅₀ (Binding)=0.11 μM.

This racemic mixture was resolved using preparative SFC (Chiralpak® AD-H column (Daicel, Inc., Fort Lee, N.J.) (150 mm×20 mm, 5 μm) eluting with 60% liquid CO₂ in 40% methanol (with 0.20% diethylamine) at a flow rate of 75 mL/min) to give two products in greater than 95% enantiomeric excess.

### N—((S)-1-benzothiophen-2-yl(2-methoxyphenyl)methyl)-3,4-dihydro-2H-1,5-benzodioxepine-7-sulfonamide

First Eluting Peak (Peak #1)

¹H NMR (400 MHz, DMSO-d₆) δ 8.83 (br. s., 1H), 7.90-7.79 (m, 1H), 7.72-7.62 (m, 1H), 7.39-7.14 (m, 5H), 7.06 (d, J=2.2 Hz, 1H), 6.93-6.77 (m, 4H), 6.13 (s, 1H), 4.07 (t, J=5.6 Hz, 2H), 3.99 (t, J=5.6 Hz, 2H), 3.72 (s, 3H), 2.06 (quin, J=5.6 Hz, 2H). m/z (ESI, +ve ion) 503.9 (M+Na)⁺. GK-GKRP EC₅₀ (LCMS/MS)=0.13 μM. GK-GKRP IC₅₀ (Binding)=0.36 μM.

### N—((R)-1-benzothiophen-2-yl(2-methoxyphenyl)methyl)-3,4-dihydro-2H-1,5-benzodioxepine-7-sulfonamide

Second Eluting Peak (Peak #2)

¹H NMR (400 MHz, DMSO-d₆) δ 8.83 (br. s., 1H), 7.89-7.79 (m, 1H), 7.71-7.63 (m, 1H), 7.39-7.15 (m, 5H), 7.06 (d, J=2.2 Hz, 1H), 6.94-6.78 (m, 4H), 6.13 (br. s., 1H), 4.07 (t, J=5.5 Hz, 2H), 3.99 (t, J=5.5 Hz, 2H), 3.72 (s, 3H), 2.06 (quin, J=5.5 Hz, 2H). m/z (ESI, +ve ion) 503.9 (M+Na)⁺. GK-GKRP EC₅₀ (LCMS/MS)=0.026 μM. GK-GKRP IC₅₀ (Binding)=0.028 μM.

### Example 41

### N-(1-benzothiophen-2-yl(2-(methylsulfanyl)phenyl)methyl)-3,4-dihydro-2H-1,5-benzodioxepine-7-sulfonamide

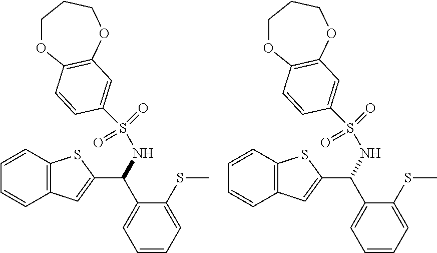

A 15 mL round-bottomed flask was charged with N-(1-benzothiophen-2-ylmethylidene)-3,4-dihydro-2H-1,5-benzodioxepine-7-sulfonamide (intermediate B) (0.15 g, 0.40 mmol), tetrahydrofuran (4.0 mL), and bromo(2-(methylsulfanyl)phenyl)magnesium (0.80 mL of a 0.50 M solution with tetrahydrofuran, 0.40 mmol, Rieke Metals, Lincoln, Nebr.), and then the reaction mixture was stirred for 1 h. After that time, saturated aqueous sodium bicarbonate (10 mL) was added, the mixture was partitioned between water (30 mL) and ethyl acetate (30 mL), the layers were separated, the aqueous material was washed with ethyl acetate (2×30 mL), the combined organic extract was washed with brine (30 mL), dried (sodium sulfate), filtered, silica gel (1.0 g) was added to the filtrate, and the volatiles were removed under a vacuum. The residue was subjected to flash chromatography on silica gel (Isco CombiFlash® system, 40 g RediSep® normal phase column, gradient elution of 10% to 30% ethyl acetate-hexane, Teledyne Isco, Lincoln, Nebr.) to give N-(1-benzothiophen-2-yl(2-(methylsulfanyl)phenyl)methyl)-3,4-dihydro-2H-1,5-benzodioxepine-7-sulfonamide (0.022 g) as a colorless solid (racemic mixture).

¹H NMR (400 MHz, methanol-d₄) δ 7.79-7.67 (m, 1H), 7.63-7.52 (m, 1H), 7.34 (d, J=7.8 Hz, 2H), 7.31-7.21 (m, 4H), 7.19 (d, J=2.4 Hz, 1H), 7.14-7.05 (m, 1H), 6.83 (d, J=8.4 Hz, 1H), 6.71 (s, 1H), 6.42 (s, 1H), 4.14 (t, J=5.7 Hz, 2H), 4.05 (t, J=5.7 Hz, 2H), 2.44 (s, 3H), 2.13 (m, 2H). m/z (ESI, +ve ion) 519.9 (M+Na)⁺. GK-GKRP EC₅₀ (LCMS/MS)=0.027 μM. GK-GKRP IC₅₀ (Binding)=0.057 μM.

This racemic mixture was resolved using preparative SFC (Chiralpak® AS-H column (Daicel, Inc., Fort Lee, N.J.) (250 mm×21 mm, 5 μm) eluting with 55% liquid CO₂ in 45% methanol (with 20 mM NH₃) at a flow rate of 60 mL/min) to give two products in greater than 95% enantiomeric excess.

### N—((S)-1-benzothiophen-2-yl(2-(methylsulfanyl)phenyl)methyl)-3,4-dihydro-2H-1,5-benzodioxepine-7-sulfonamide

First Eluting Peak (Peak #1)

¹H NMR (400 MHz, DMSO-d₆) δ 9.05 (s, 1H), 7.91-7.81 (m, 1H), 7.73-7.62 (m, 1H), 7.43 (d, J=7.8 Hz, 1H), 7.35-7.15 (m, 5H), 7.14-7.02 (m, 2H), 6.88 (d, J=8.4 Hz, 1H), 6.76 (s, 1H), 6.25 (s, 1H), 4.18-3.90 (m, 4H), 2.44 (s, 3H), 2.12-2.01 (m, 2H). m/z (ESI, +ve ion) 519.9 (M+Na)⁺. GK-GKRP EC₅₀ (LCMS/MS)=0.040 μM. GK-GKRP IC₅₀ (Binding)=0.14 μM.

### N—((R)-1-benzothiophen-2-yl(2-(methylsulfanyl)phenyl)methyl)-3,4-dihydro-2H-1,5-benzodioxepine-7-sulfonamide

Second Eluting Peak (Peak #2)

¹H NMR (400 MHz, DMSO-d₆) δ 9.05 (d, J=9.0 Hz, 1H), 7.92-7.80 (m, 1H), 7.73-7.62 (m, 1H), 7.43 (d, J=7.8 Hz, 1H), 7.35-7.16 (m, 5H), 7.14-7.03 (m, 2H), 6.87 (d, J=8.4 Hz, 1H), 6.76 (s, 1H), 6.26 (d, J=9.0 Hz, 1H), 4.16-3.89 (m, 4H), 2.44 (s, 3H), 2.14-2.00 (m, 2H). m/z (ESI, +ve ion) 519.9 (M+Na)⁺. GK-GKRP EC₅₀ (LCMS/MS)=0.013 μM. GK-GKRP IC₅₀ (Binding)=0.008 μM.

### Example 42

### N-(1-benzothiophen-2-yl(2-chlorophenyl)methyl)-3,4-dihydro-2H-1,5-benzodioxepine-7-sulfonamide

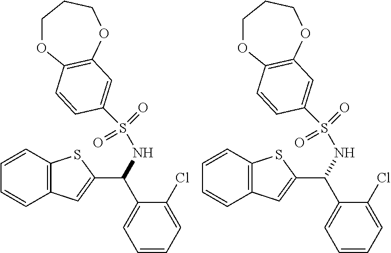

A 15 mL round-bottomed flask was charged with 1-bromo-2-chlorobenzene (0.10 g, 0.54 mmol, Fluka, Buchs, Switzerland) and tetrahydrofuran (5.4 mL). The solution was cooled to −78° C., n-butyllithium (0.33 mL of a 1.6 M solution with hexane, 0.54 mmol, Sigma-Aldrich, St. Louis, Mo.) was added, and then the reaction mixture was stirred for 20 min. After that time, a solution of N-(1-benzothiophen-2-ylmethylidene)-3,4-dihydro-2H-1,5-benzodioxepine-7-sulfonamide (intermediate B) (0.20 g, 0.54 mmol) and tetrahydrofuran (5.0 mL) was added. After stirring for 5 min, saturated aqueous ammonium chloride (5.0 mL) was added, the reaction mixture was allowed to warm to room temperature, partitioned between water (30 mL) and ethyl acetate (30 mL), the layers were separated, the aqueous material was washed with ethyl acetate (30 mL). The combined organic extract was washed with brine (20 mL), dried (sodium sulfate), filtered, and the filtrate was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 24 min) to give N-(1-benzothiophen-2-yl(2-chlorophenyl)methyl)-3,4-dihydro-2H-1,5-benzodioxepine-7-sulfonamide (0.023 g) as a colorless solid (racemic mixture).

¹H NMR (400 MHz, methanol-d₄) δ 7.81-7.71 (m, 1H), 7.65-7.55 (m, 1H), 7.48-7.39 (m, 1H), 7.37-7.31 (m, 1H), 7.31-7.16 (m, 6H), 6.84 (d, J=8.4 Hz, 1H), 6.75 (s, 1H), 6.28 (s, 1H), 4.21-3.99 (m, 4H), 2.20-2.07 (m, 2H). m/z (ESI, +ve ion) 507.8 (M+Na)⁺. GK-GKRP EC₅₀ (LCMS/MS)=0.041 μM. GK-GKRP IC₅₀ (Binding)=0.11 μM.

### Example 42

### Enantioselective Synthesis

### N—((R)-1-Benzothiophen-2-yl(2-chlorophenyl)methyl)-3,4-dihydro-2H-1,5-benzodioxepine-7-sulfonamide

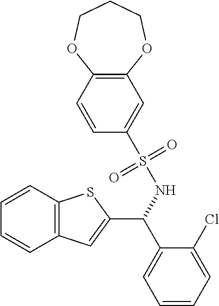

A 15 mL round-bottomed flask was charged with (R)-1-(1-benzothiophen-2-yl)-1-(2-chlorophenyl)methanamine hydrochloride (Intermediate 2) (0.10 g, 0.32 mmol), dichloromethane (3.2 mL), N-ethyl-N-(1-methylethyl)-2-propanamine (0.17 mL, 0.97 mmol, Sigma-Aldrich, St. Louis, Mo.), and 3,4-dihydro-2H-1,5-benzodioxepine-7-sulfonyl chloride (0.080 g, 0.32 mmol, ChemBridge, San Diego, Calif.) and then the reaction mixture was stirred for 2 h. After that time, N,N-dimethyl-4-pyridinamine (0.0039 g, 0.032 mmol, Sigma-Aldrich, St. Louis, Mo.) was added and then the reaction mixture was stirred for 3 h. After that time, more N-ethyl-N-(1-methylethyl)-2-propanamine (0.17 mL, 0.97 mmol) was added and then the reaction mixture was stirred for 30 min. After that time, more 3,4-dihydro-2H-1,5-benzodioxepine-7-sulfonyl chloride (0.080 g, 0.32 mmol) was added and then the reaction mixture was stirred for 23 h. After that time, the reaction mixture was partitioned between ethyl acetate (50 mL) and saturated aqueous sodium bicarbonate (50 mL), the layers were separated, the organic material was washed sequentially with saturated aqueous sodium bicarbonate (50 mL) and brine (50 mL), dried (sodium sulfate), filtered, and the filtrate was concentrated. The residue was dissolved with dichloromethane (5.0 mL), silica gel (1.0 g) was added, and the volatiles were removed under a vacuum. The residue was subjected to flash chromatography on silica gel (10 g of silica gel, 4:1 hexane-ethyl acetate) to give N—((R)-1-benzothiophen-2-yl(2-chlorophenyl)methyl)-3,4-dihydro-2H-1,5-benzodioxepine-7-sulfonamide (0.12 g) as a colorless solid.

¹H NMR (400 MHz, DMSO-d₆) δ 9.16 (br. s., 1H), 7.93-7.84 (m, 1H), 7.75-7.66 (m, 1H), 7.56-7.49 (m, 1H), 7.42-7.35 (m, 1H), 7.35-7.22 (m, 4H), 7.20 (dd, J=2.2, 8.5 Hz, 1H), 7.09 (d, J=2.3 Hz, 1H), 6.88 (d, J=8.4 Hz, 1H), 6.82 (s, 1H), 6.16 (br. s., 1H), 4.17-3.96 (m, 4H), 2.08 (quin, J=5.6 Hz, 2H). m/z (ESI, +ve ion) 507.8 (M+Na)⁺. GK-GKRP IC₅₀ (Binding)=0.0060 μM.

### Example 43

### N-(1-benzothiophen-2-yl(3-methoxy-2-pyridinyl)methyl)-3,4-dihydro-2H-1,5-benzodioxepine-7-sulfonamide

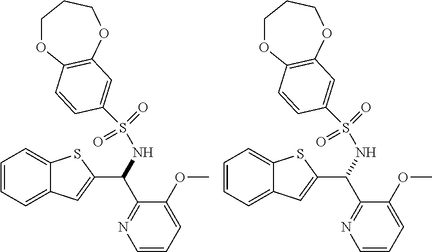

A 25 mL round-bottomed flask was charged with 2-bromo-1,3,5-trimethylbenzene (0.53 g, 2.7 mmol, Sigma-Aldrich, St. Louis, Mo.) and tetrahydrofuran (5.6 mL). The solution was cooled to −78° C., t-butyllithium (3.4 mL of a 1.7 M solution with pentane, 5.4 mmol, Sigma-Aldrich, St. Louis, Mo.) was added, and then the reaction mixture was stirred for 1 h. After that time, the reaction mixture was warmed to −23° C., 3-methoxypyridine (0.29 g, 2.7 mmol, Sigma-Aldrich, St. Louis, Mo.) was added, and then the reaction mixture was stirred for 3 h. After that time, an aliquot of this reaction mixture (1.0 mL) was added to a solution of N-(1-benzothiophen-2-ylmethylidene)-3,4-dihydro-2H-1,5-benzodioxepine-7-sulfonamide (intermediate B) (0.10 g, 0.27 mmol) and tetrahydrofuran (2.8 mL) at 0° C. After stirring for 35 min, methanol (1.0 mL) and silica gel (0.50 g) were added sequentially and the volatiles were removed under a vacuum. The residue was subjected to flash chromatography on silica gel (6.5 g of silica gel, gradient elution with 6:1 to 2:1 hexane-ethyl acetate), and then the isolated material was re-subjected to flash chromatography on silica gel (3.0 g of silica gel, 99.5:0.5 dichloromethane-methanol) to give N-(1-benzothiophen-2-yl(3-methoxy-2-pyridinyl)methyl)-3,4-dihydro-2H-1,5-benzodioxepine-7-sulfonamide (0.024 g) as a pale yellow tar (racemic mixture).

¹H NMR (400 MHz, chloroform-d) δ 8.09 (d, J=4.7 Hz, 1H), 7.65 (d, J=7.8 Hz, 1H), 7.59 (d, J=7.4 Hz, 1H), 7.32-7.17 (m, 4H), 7.15 (dd, J=4.7, 8.2 Hz, 1H), 7.07 (d, J=8.2 Hz, 1H), 7.03 (s, 1H), 6.79-6.67 (m, 2H), 6.30 (d, J=9.0 Hz, 1H), 4.15-3.95 (m, 3H), 3.89 (m, 1H), 3.80 (s, 3H), 2.17-2.00 (m, 2H). m/z (ESI, +ve ion) 482.9 (M+H)⁺. GK-GKRP EC₅₀ (LCMS/MS)=0.047 μM. GK-GKRP IC₅₀ (Binding)=0.041 μM.

### Intermediate C

### N-((3-methyl-1-benzofuran-2-yl)methylidene)-3,4-dihydro-2H-1,5-benzodioxepine-7-sulfonamide

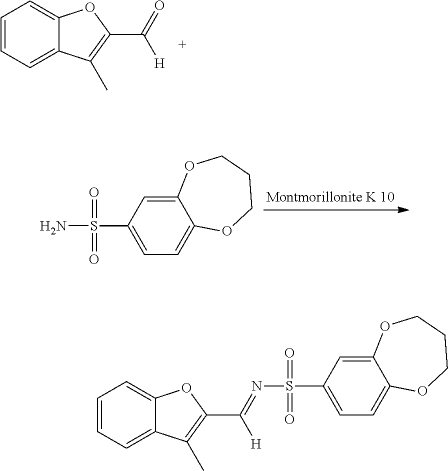 [SMILES: Cc1c(C=O)oc2ccccc12 ; NS(=O)(=O)c1ccc2c(c1)OCCCO2 ; Cc1c(/C=N/S(=O)(=O)c2ccc3c(c2)OCCCO3)oc2ccccc12]

A 50 mL round-bottomed flask was charged with 3-methyl-1-benzofuran-2-carbaldehyde (0.45 g, 2.8 mmol, ChemBridge, San Diego, Calif.), 3,4-dihydro-2H-1,5-benzodioxepine-7-sulfonamide (0.64 g, 2.8 mmol, Enamine, Kiev, Ukraine), Montmorillonite K 10 (0.45 g, Sigma-Aldrich, St. Louis, Mo.), and toluene (14 mL), fitted with a Dean-Stark trap and a reflux condenser, and then the reaction mixture was heated at reflux for 1.5 h. After that time, the reaction mixture was filtered, the filtrate was concentrated under a vacuum to a volume of ˜5 mL, hexane (50 mL) was added, the resulting slurry was filtered, and the filter cake was collected to provide N-((3-methyl-1-benzofuran-2-yl)methylidene)-3,4-dihydro-2H-1,5-benzodioxepine-7-sulfonamide (0.78 g) as an orange solid.

### Example 44

### N-((3-methyl-1-benzofuran-2-yl)(phenyl)methyl)-3,4-dihydro-2H-1,5-benzodioxepine-7-sulfonamide

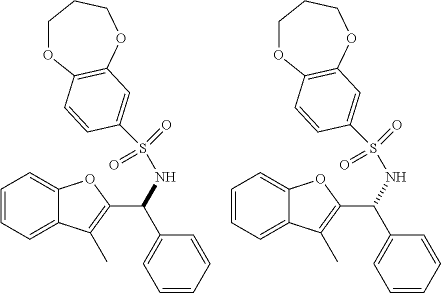

A 10 mL round-bottomed flask was charged with N-((3-methyl-1-benzofuran-2-yl)methylidene)-3,4-dihydro-2H-1,5-benzodioxepine-7-sulfonamide (intermediate C) (0.10 g, 0.27 mmol), tetrahydrofuran (2.7 mL), and bromo(phenyl)magnesium (0.30 mL of a 1.0 M solution with tetrahydrofuran, 0.30 mmol, Sigma-Aldrich, St. Louis, Mo.), and then the reaction mixture was stirred for 15 min. After that time, methanol (1.0 mL) and silica gel (0.50 g) were added sequentially and the volatiles were removed under a vacuum. The residue was subjected to flash chromatography on silica gel (6.0 g of silica gel, 4:1 hexane-ethyl acetate) to give N-((3-methyl-1-benzofuran-2-yl)(phenyl)methyl)-3,4-dihydro-2H-1,5-benzodioxepine-7-sulfonamide (0.080 g) as a colorless solid (racemic mixture).

¹H NMR (400 MHz, DMSO-d₆) δ 8.90 (d, J=9.4 Hz, 1H), 7.43 (d, J=7.4 Hz, 1H), 7.41-7.35 (m, 2H), 7.35-7.29 (m, 3H), 7.29-7.14 (m, 4H), 7.12 (d, J=2.2 Hz, 1H), 6.73 (d, J=8.4 Hz, 1H), 5.75 (d, J=9.2 Hz, 1H), 4.02-3.82 (m, 3H), 3.82-3.69 (m, 1H), 2.06 (s, 3H), 2.03-1.86 (m, 2H). m/z (ESI, +ve ion) 471.9 (M+Na)⁺. GK-GKRP EC₅₀ (LCMS/MS)=0.48 μM. GK-GKRP IC₅₀ (Binding)=1.0 μM.

### Example 45

### N-((3-methyl-1-benzofuran-2-yl)(2-methylphenyl)methyl)-3,4-dihydro-2H-1,5-benzodioxepine-7-sulfonamide

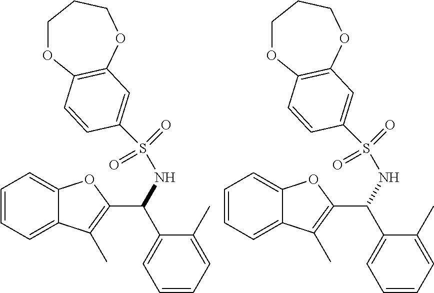

A 10 mL round-bottomed flask was charged with N-((3-methyl-1-benzofuran-2-yl)methylidene)-3,4-dihydro-2H-1,5-benzodioxepine-7-sulfonamide (intermediate C) (0.10 g, 0.27 mmol), tetrahydrofuran (2.7 mL), and bromo(2-methylphenyl)magnesium (0.15 mL of a 2.0 M solution with diethyl ether, 0.30 mmol, Sigma-Aldrich, St. Louis, Mo.), and then the reaction mixture was stirred for 30 min. After that time, methanol (1.0 mL) and silica gel (0.50 g) were added sequentially and the volatiles were removed under a vacuum. The residue was subjected to flash chromatography on silica gel (6.5 g of silica gel, 4:1 hexane-ethyl acetate) to give N-((3-methyl-1-benzofuran-2-yl)(2-methylphenyl)methyl)-3,4-dihydro-2H-1,5-benzodioxepine-7-sulfonamide (0.086 g) as a colorless solid (racemic mixture).

¹H NMR (400 MHz, DMSO-d₆) δ 8.83 (d, J=8.8 Hz, 1H), 7.56-7.47 (m, 1H), 7.44 (d, J=7.0 Hz, 1H), 7.34 (d, J=8.0 Hz, 1H), 7.27-7.08 (m, 7H), 6.77 (d, J=8.4 Hz, 1H), 5.83 (d, J=8.8 Hz, 1H), 4.04-3.75 (m, 4H), 2.28 (s, 3H), 2.01 (m, 5H). m/z (ESI, +ve ion) 485.9 (M+Na)⁺. GK-GKRP EC₅₀ (LCMS/MS)=2.0 μM. GK-GKRP IC₅₀ (Binding)=1.7 μM.

### Intermediate D

### N-(thieno[2,3-b]pyridin-2-ylmethylidene)-3,4-dihydro-2H-1,5-benzodioxepine-7-sulfonamide

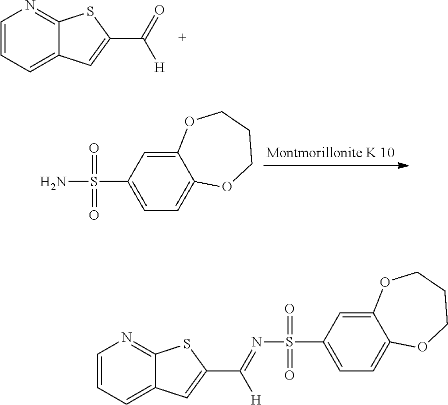 [SMILES: O=Cc1cc2cccnc2s1 ; NS(=O)(=O)c1ccc2c(c1)OCCCO2 ; O=S(=O)(/N=C/c1cc2cccnc2s1)c1ccc2c(c1)OCCCO2]

A 100 mL round-bottomed flask was charged with thieno[2,3-b]pyridine-2-carbaldehyde (1.0 g, 6.1 mmol, Maybridge, Tintagel, United Kingdom), 3,4-dihydro-2H-1,5-benzodioxepine-7-sulfonamide (1.4 g, 6.1 mmol, Enamine, Kiev, Ukraine), Montmorillonite K 10 (1.4 g, Sigma-Aldrich, St. Louis, Mo.), and toluene (31 mL), fitted with a Dean-Stark trap and a reflux condenser, and then the reaction mixture was heated at reflux for 5 h. After that time, the reaction mixture was filtered, the filtrate was allowed to cool to room temperature, filtered, and the filter cake was collected to give N-(thieno[2,3-b]pyridin-2-ylmethylidene)-3,4-dihydro-2H-1,5-benzodioxepine-7-sulfonamide (1.1 g) as pale, peach-colored solid.

### Example 46

### N-(phenyl(thieno[2,3-b]pyridin-2-yl)methyl)-3,4-dihydro-2H-1,5-benzodioxepine-7-sulfonamide

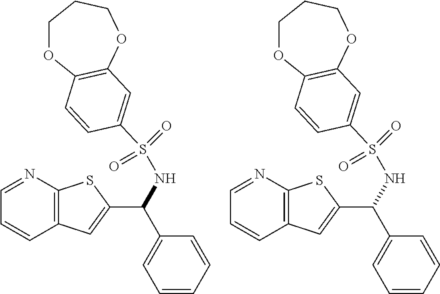

A 15 mL round-bottomed flask was charged with N-(thieno[2,3-b]pyridin-2-ylmethylidene)-3,4-dihydro-2H-1,5-benzodioxepine-7-sulfonamide (intermediate D) (0.074 g, 0.20 mmol), tetrahydrofuran (2.0 mL), and bromo(phenyl)magnesium (a total of 0.40 mL of a 1.0 M solution with tetrahydrofuran, added in two equal portions, 0.40 mmol, Acros, Waltham, Mass.), and then the reaction mixture was stirred for 3 h. After that time, the reaction mixture was partitioned between water (100 mL) and dichloromethane (50 mL), the layers were separated. The aqueous material was washed with dichloromethane (50 mL), the combined organic extract was washed with brine (50 mL), dried (sodium sulfate) and filtered. Silica gel (2.0 g) was added to the filtrate, and the volatiles were removed under a vacuum. The residue was subjected to flash chromatography on silica gel (Isco CombiFlash® system, 40 g RediSep® normal phase column, gradient elution of hexane to 30% ethyl acetate-hexane, Teledyne Isco, Lincoln, Nebr.), and then the isolated material was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 24 min) to give N-(phenyl(thieno[2,3-b]pyridin-2-yl)methyl)-3,4-dihydro-2H-1,5-benzodioxepine-7-sulfonamide (0.0025 g) as a colorless solid (racemic mixture).

¹H NMR (400 MHz, DMSO-d₆) δ 9.02 (d, J=9.8 Hz, 1H), 8.45 (dd, J=4.5, 1.4 Hz, 1H), 8.09 (dd, J=8.0, 1.4 Hz, 1H), 7.40-7.19 (m, 7H), 7.11 (d, J=2.4 Hz, 1H), 6.92 (s, 1H), 6.86 (d, J=8.6 Hz, 1H), 5.87 (d, J=9.6 Hz, 1H), 4.04 (t, J=5.6 Hz, 2H), 3.96 (t, J=5.7 Hz, 2H), 2.05 (m, 2H). m/z (ESI, +ve ion) 452.9 (M+H)⁺. GK-GKRP EC₅₀ (LCMS/MS)=0.22 μM. GK-GKRP IC₅₀ (Binding)=0.36 μM.

### Example 47

### N-((2-methoxyphenyl)(thieno[2,3-b]pyridin-2-yl)methyl)-3,4-dihydro-2H-1,5-benzodioxepine-7-sulfonamide

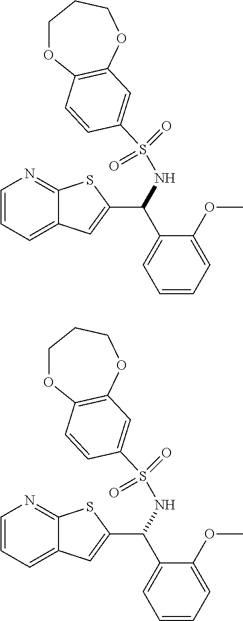 [SMILES: COc1ccccc1[C@H](NS(=O)(=O)c1ccc2c(c1)OCCCO2)c1cc2cccnc2s1 ; COc1ccccc1[C@@H](NS(=O)(=O)c1ccc2c(c1)OCCCO2)c1cc2cccnc2s1]

A 10 mL round-bottomed flask was charged with N-(thieno[2,3-b]pyridin-2-ylmethylidene)-3,4-dihydro-2H-1,5-benzodioxepine-7-sulfonamide (intermediate D) (0.10 g, 0.27 mmol), tetrahydrofuran (2.7 mL), and bromo(2-methoxyphenyl)magnesium (a total of 0.88 mL of a 1.0 M solution with diethyl ether, added in three equal portions, 0.88 mmol, Sigma-Aldrich, St. Louis, Mo.), and then the reaction mixture was stirred for 25 min. After that time, methanol (1.0 mL) and silica gel (0.50 g) were added sequentially and the volatiles were removed under a vacuum. The residue was subjected to flash chromatography on silica gel (6.5 g of silica gel, 99:1 dichloromethane-methanol), and then the isolated material was re-subjected to flash chromatography on silica gel (3.0 g of silica gel, 99:1 dichloromethane-methanol) to give N-((2-methoxyphenyl)(thieno[2,3-b]pyridin-2-yl)methyl)-3,4-dihydro-2H-1,5-benzodioxepine-7-sulfonamide (0.041 g) as a colorless solid (racemic mixture).

¹H NMR (400 MHz, DMSO-d₆) δ 8.89 (d, J=7.6 Hz, 1H), 8.45 (dd, J=1.4, 4.5 Hz, 1H), 8.08 (dd, J=1.4, 8.0 Hz, 1H), 7.38-7.30 (m, 2H), 7.26-7.15 (m, 2H), 7.07 (d, J=2.2 Hz, 1H), 6.91 (d, J=8.2 Hz, 1H), 6.89-6.79 (m, 3H), 6.13 (d, J=7.0 Hz, 1H), 4.08 (t, J=5.6 Hz, 2H), 4.01 (t, J=5.6 Hz, 2H), 3.73 (s, 3H), 2.07 (quin, J=5.6 Hz, 2H). m/z (ESI, +ve ion) 482.9 (M+H)⁺. GK-GKRP EC₅₀ (LCMS/MS)=0.059 μM. GK-GKRP IC₅₀ (Binding)=0.027 μM.

### Example 48

### N-((2-(methylsulfanyl)phenyl)(thieno[2,3-b]pyridin-2-yl)methyl)-3,4-dihydro-2H-1,5-benzodioxepine-7-sulfonamide

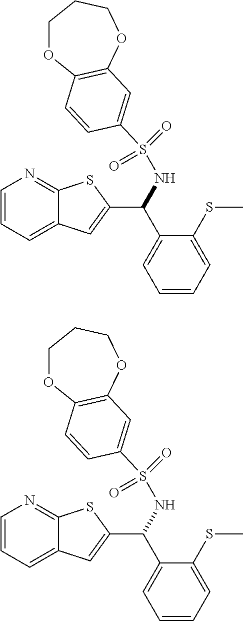 [SMILES: CSc1ccccc1[C@H](NS(=O)(=O)c1ccc2c(c1)OCCCO2)c1cc2cccnc2s1 ; CSc1ccccc1[C@@H](NS(=O)(=O)c1ccc2c(c1)OCCCO2)c1cc2cccnc2s1]

A 10 mL round-bottomed flask was charged with N-(thieno[2,3-b]pyridin-2-ylmethylidene)-3,4-dihydro-2H-1,5-benzodioxepine-7-sulfonamide (intermediate D) (0.10 g, 0.27 mmol), tetrahydrofuran (2.7 mL), and bromo(2-(methylsulfanyl)phenyl)magnesium (a total of 1.2 mL of a 0.50 M solution with tetrahydrofuran, added in two equal portions, 0.59 mmol, Rieke Metals, Lincoln, Nebr.), and then the reaction mixture was stirred for 3 h. After that time, methanol (1.0 mL) and silica gel (0.50 g) were added sequentially and the volatiles were removed under a vacuum. The residue was subjected to flash chromatography on silica gel (6.5 g of silica gel, 99:1 dichloromethane-methanol), and then the isolated material was re-subjected to flash chromatography on silica gel (6.0 g of silica gel, 99:1 dichloromethane-methanol) to give N-((2-(methylsulfanyl)phenyl)(thieno[2,3-b]pyridin-2-yl)methyl)-3,4-dihydro-2H-1,5-benzodioxepine-7-sulfonamide (0.023 g) as a colorless solid (racemic mixture).

¹H NMR (400 MHz, chloroform-d) δ 8.51-8.42 (m, 1H), 7.85 (dd, J=1.4, 8.0 Hz, 1H), 7.34-7.18 (m, 6H), 7.15-7.08 (m, 1H), 6.85-6.78 (m, 2H), 6.29-6.11 (m, 2H), 4.23 (t, J=5.8 Hz, 2H), 4.15 (t, J=5.8 Hz, 2H), 2.37 (s, 3H), 2.19 (quin, J=5.8 Hz, 2H). m/z (ESI, +ve ion) 498.8 (M+H)⁺. GK-GKRP EC₅₀ (LCMS/MS)=0.022 μM. GK-GKRP IC₅₀ (Binding)=0.010 μM.

### Example 49

### N-((3-methoxy-2-pyridinyl)(thieno[2,3-b]pyridin-2-yl)methyl)-3,4-dihydro-2H-1,5-benzodioxepine-7-sulfonamide

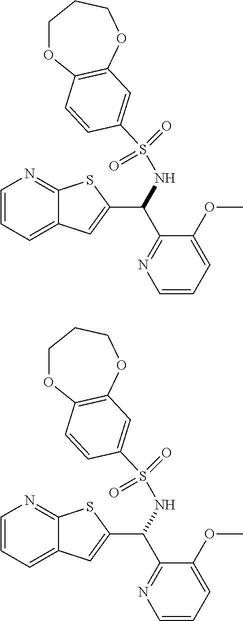 [SMILES: COc1cccnc1[C@H](NS(=O)(=O)c1ccc2c(c1)OCCCO2)c1cc2cccnc2s1 ; COc1cccnc1[C@@H](NS(=O)(=O)c1ccc2c(c1)OCCCO2)c1cc2cccnc2s1]

A 10 mL round-bottomed flask was charged with 2-bromo-3-methoxypyridine (0.25 g, 1.3 mmol, Sigma-Aldrich, St. Louis, Mo.) and toluene (2.7 mL). The solution was cooled to −78° C., n-butyllithium (0.53 mL of a 2.5 M solution with toluene, 1.3 mmol, Sigma-Aldrich, St. Louis, Mo.) was added, and then the reaction mixture was stirred for 2 h. After that time, an aliquot of this reaction mixture (a total of 1.2 mL added in two equal portions) was added to a solution of N-(thieno[2,3-b]pyridin-2-ylmethylidene)-3,4-dihydro-2H-1,5-benzodioxepine-7-sulfonamide (intermediate D) (0.10 g, 0.27 mmol) and tetrahydrofuran (2.7 mL). After stirring for 15 h, methanol (1.0 mL) and silica gel (0.50 g) were added sequentially and the volatiles were removed under a vacuum. The residue was subjected to flash chromatography on silica gel (6.5 g of silica gel, 99:1 dichloromethane-methanol), and then the isolated material was re-subjected to flash chromatography on silica gel (4.0 g of silica gel, gradient elution of 4:1 to 3:1 to 2:1 to 1:1 hexane-ethyl acetate) to give N-((3-methoxy-2-pyridinyl)(thieno[2,3-b]pyridin-2-yl)methyl)-3,4-dihydro-2H-1,5-benzodioxepine-7-sulfonamide (0.016 g) as a pale yellow solid (racemic mixture).

¹H NMR (400 MHz, chloroform-d) δ 8.46-8.38 (m, 1H), 8.09 (d, J=4.5 Hz, 1H), 7.87 (d, J=8.0 Hz, 1H), 7.30-7.23 (m, 2H), 7.23-7.13 (m, 2H), 7.13-7.06 (m, 1H), 7.04 (s, 1H), 6.78 (d, J=8.8 Hz, 1H), 6.73-6.66 (m, 1H), 6.28 (d, J=8.8 Hz, 1H), 4.19-4.02 (m, 3H), 4.02-3.92 (m, 1H), 3.82 (s, 3H), 2.19-2.03 (m, 2H). m/z (ESI, +ve ion) 483.8 (M+H)⁺. GK-GKRP EC₅₀ (LCMS/MS)=0.15 μM. GK-GKRP IC₅₀ (Binding)=0.093 μM.

### Example 50

### N-((3-methoxy-4-pyridinyl)(thieno[2,3-b]pyridin-2-yl)methyl)-3,4-dihydro-2H-1,5-benzodioxepine-7-sulfonamide

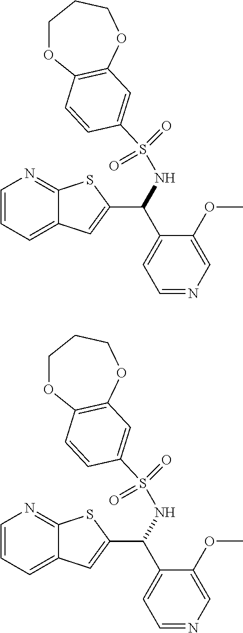 [SMILES: COc1cnccc1[C@H](NS(=O)(=O)c1ccc2c(c1)OCCCO2)c1cc2cccnc2s1 ; COc1cnccc1[C@@H](NS(=O)(=O)c1ccc2c(c1)OCCCO2)c1cc2cccnc2s1]

A 25 mL round-bottomed flask was charged with tetrahydrofuran (12 mL) and t-butyllithium (4.2 mL of a 1.7 M solution with pentane, 7.1 mmol, Sigma-Aldrich, St. Louis, Mo.) at −78° C., 2-bromo-1,3,5-trimethylbenzene (0.70 g, 3.5 mmol, Sigma-Aldrich, St. Louis, Mo.) was added, and then the reaction mixture was stirred for 30 min. After that time, 3-methoxypyridine (0.39 g, 3.5 mmol, Sigma-Aldrich, St. Louis, Mo.) was added, and then the reaction mixture was warmed to −23° C. After stirring for 3 h, an aliquot of this reaction mixture (1.0 mL) was added to a solution of N-(thieno[2,3-b]pyridin-2-ylmethylidene)-3,4-dihydro-2H-1,5-benzodioxepine-7-sulfonamide (intermediate D) (0.44 g, 1.2 mmol) and tetrahydrofuran (12 mL). After stirring for 40 min, methanol (1.0 mL) and silica gel (0.50 g) were added sequentially and the volatiles were removed under a vacuum. The residue was subjected to flash chromatography on silica gel (28 g of silica gel, 49:1 dichloromethane-methanol), and then the isolated material was re-subjected to flash chromatography on silica gel (4.0 g of silica gel, 49:1 dichloromethane-methanol) to give N-((3-methoxy-4-pyridinyl)(thieno[2,3-b]pyridin-2-yl)methyl)-3,4-dihydro-2H-1,5-benzodioxepine-7-sulfonamide (0.043 g) as a pale yellow solid (racemic mixture).

¹H NMR (400 MHz, DMSO-d₆) δ 9.10 (br. s., 1H), 8.48 (dd, J=1.6, 4.5 Hz, 1H), 8.28 (s, 1H), 8.14-8.05 (m, 2H), 7.41-7.29 (m, 2H), 7.19 (dd, J=2.2, 8.3 Hz, 1H), 7.06 (d, J=2.3 Hz, 1H), 6.92-6.83 (m, 2H), 6.08 (br. s., 1H), 4.11 (t, J=5.6 Hz, 2H), 4.04 (t, J=5.8 Hz, 2H), 3.86 (s, 3H), 2.09 (quin, J=5.6 Hz, 2H). m/z (ESI, +ve ion) 483.8 (M+H)⁺. GK-GKRP EC₅₀ (LCMS/MS)=0.48 μM. GK-GKRP IC₅₀ (Binding)=0.41 μM.

### Intermediate E

### N-(1,3-benzothiazol-2-ylmethylidene)-3,4-dihydro-2H-1,5-benzodioxepine-7-sulfonamide

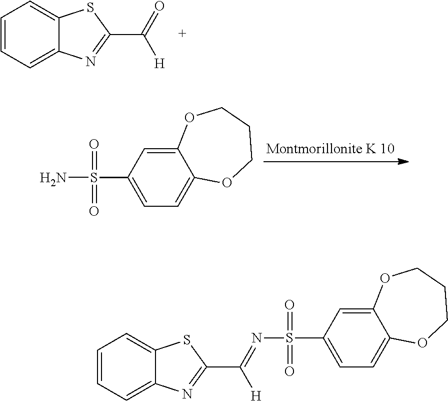 [SMILES: O=Cc1nc2ccccc2s1 ; NS(=O)(=O)c1ccc2c(c1)OCCCO2 ; O=S(=O)(/N=C/c1nc2ccccc2s1)c1ccc2c(c1)OCCCO2]

A 100 mL round-bottomed flask was charged with 1,3-benzothiazole-2-carbaldehyde (1.5 g, 9.2 mmol, ASDI, Newark, Del.), 3,4-dihydro-2H-1,5-benzodioxepine-7-sulfonamide (1.4 g, 6.1 mmol, Enamine, Kiev, Ukraine), Montmorillonite K 10 (1.4 g, Sigma-Aldrich, St. Louis, Mo.), and toluene (31 mL), fitted with a Dean-Stark trap and a reflux condenser, and then the reaction mixture was heated at reflux for 3 h. After that time, the reaction mixture was filtered, the filtrate was allowed to cool to room temperature, concentrated under a stream of dry nitrogen to a volume of ˜10 mL, filtered, and the filter cake was collected to give N-(1,3-benzothiazol-2-ylmethylidene)-3,4-dihydro-2H-1,5-benzodioxepine-7-sulfonamide (0.97 g) as a pale yellow solid.

### Example 51

### N-(1,3-benzothiazol-2-yl(2-methoxyphenyl)methyl)-3,4-dihydro-2H-1,5-benzodioxepine-7-sulfonamide

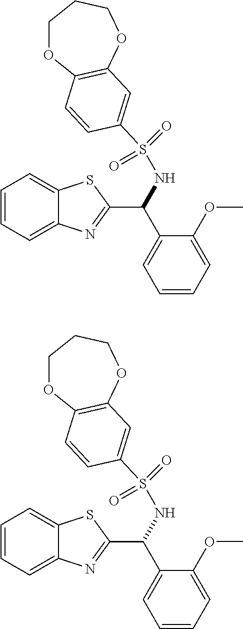 [SMILES: COc1ccccc1[C@H](NS(=O)(=O)c1ccc2c(c1)OCCCO2)c1nc2ccccc2s1 ; COc1ccccc1[C@@H](NS(=O)(=O)c1ccc2c(c1)OCCCO2)c1nc2ccccc2s1]

A 10 mL round-bottomed flask was charged with N-(1,3-benzothiazol-2-ylmethylidene)-3,4-dihydro-2H-1,5-benzodioxepine-7-sulfonamide (intermediate E) (0.10 g, 0.27 mmol), tetrahydrofuran (2.7 mL), and bromo(2-methoxyphenyl)magnesium (a total of 0.44 mL of a 1.0 M solution with diethyl ether, added in two portions, 0.44 mmol, Sigma-Aldrich, St. Louis, Mo.), and then the reaction mixture was stirred for 90 min. After that time, methanol (1.0 mL) and silica gel (0.50 g) were added sequentially and the volatiles were removed under a vacuum. The residue was subjected to flash chromatography on silica gel (6.5 g of silica gel, 99.5:0.5 dichloromethane-methanol), the isolated material was subjected to reversed-phase preparative HPLC (Phenomenex Gemini C18 column (Phenomenex, Inc., Torrance, Calif.) (100×30 mm, 5 μm) eluting with 0.10% trifluoroacetic acid in acetonitrile-water, gradient of 10% to 90% over 20 min), and then the isolated material was re-subjected to flash chromatography on silica gel (3.0 g of silica gel, 4:1 hexane-ethyl acetate) to give N-(1,3-benzothiazol-2-yl(2-methoxyphenyl)methyl)-3,4-dihydro-2H-1,5-benzodioxepine-7-sulfonamide (0.039 g) as a colorless solid (racemic mixture).

¹H NMR (400 MHz, chloroform-d) δ 7.90 (d, J=8.2 Hz, 1H), 7.79 (d, J=8.0 Hz, 1H), 7.46-7.38 (m, 1H), 7.37-7.28 (m, 3H), 7.22 (dd, J=6.1, 7.6 Hz, 2H), 6.86 (t, J=7.4 Hz, 1H), 6.81-6.78 (m, 2H), 6.48 (d, J=8.8 Hz, 1H), 6.05 (d, J=8.6 Hz, 1H), 4.14 (m, 2H), 4.08 (m, 2H), 3.75 (s, 3H), 2.14 (quin, J=5.8 Hz, 2H). m/z (ESI, +ve ion) 482.9 (M+H)⁺. GK-GKRP EC₅₀ (LCMS/MS)=0.068 μM. GK-GKRP IC₅₀ (Binding)=0.042 μM.

### Example 52

### N-(1,3-benzothiazol-2-yl(2-(methylsulfanyl)phenyl)methyl)-3,4-dihydro-2H-1,5-benzodioxepine-7-sulfonamide

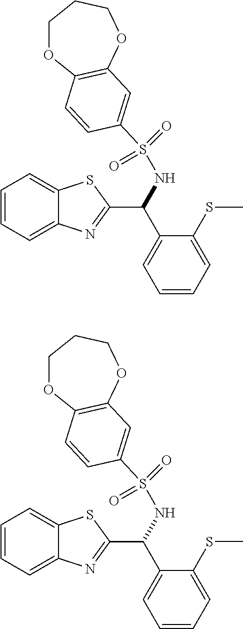 [SMILES: CSc1ccccc1[C@H](NS(=O)(=O)c1ccc2c(c1)OCCCO2)c1nc2ccccc2s1 ; CSc1ccccc1[C@@H](NS(=O)(=O)c1ccc2c(c1)OCCCO2)c1nc2ccccc2s1]

A 10 mL round-bottomed flask was charged with N-(1,3-benzothiazol-2-ylmethylidene)-3,4-dihydro-2H-1,5-benzodioxepine-7-sulfonamide (intermediate E) (0.10 g, 0.27 mmol), tetrahydrofuran (2.7 mL), and bromo(2-(methylsulfanyl)phenyl)magnesium (a total of 0.89 mL of a 0.50 M solution with tetrahydrofuran, added in two portions, 0.44 mmol, Rieke Metals, Lincoln, Nebr.), and then the reaction mixture was stirred for 1 h. After that time, methanol (1.0 mL) and silica gel (0.50 g) were added sequentially and the volatiles were removed under a vacuum. The residue was subjected to flash chromatography on silica gel (6.5 g of silica gel, 99.5:0.5 dichloromethane-methanol), the isolated material was subjected to reversed-phase preparative HPLC (Phenomenex Gemini C18 column (Phenomenex, Inc., Torrance, Calif.) (100×30 mm, 5 μm) eluting with 0.10% trifluoroacetic acid in acetonitrile-water, gradient of 10% to 90% over 20 min), and then the isolated material was re-subjected to flash chromatography on silica gel (3.0 g of silica gel, 4:1 hexane-ethyl acetate) to give N-(1,3-benzothiazol-2-yl(2-(methylsulfanyl)phenyl)methyl)-3,4-dihydro-2H-1,5-benzodioxepine-7-sulfonamide (0.026 g) as a colorless solid (racemic mixture).

¹H NMR (400 MHz, chloroform-d) δ 7.93 (d, J=8.2 Hz, 1H), 7.76 (d, J=8.0 Hz, 1H), 7.44 (t, J=7.5 Hz, 1H), 7.39-7.29 (m, 4H), 7.29-7.18 (m, 2H), 7.14-7.04 (m, 1H), 6.81 (d, J=8.6 Hz, 1H), 6.65 (d, J=6.8 Hz, 1H), 6.41 (d, J=6.8 Hz, 1H), 4.15 (t, J=5.8 Hz, 2H), 4.09 (t, J=5.8 Hz, 2H), 2.47 (s, 3H), 2.15 (quin, J=5.8 Hz, 2H). m/z (ESI, +ve ion) 498.8 (M+H)⁺. GK-GKRP EC₅₀ (LCMS/MS)=0.029 μM. GK-GKRP IC₅₀ (Binding)=0.006 μM.

### Example 53

### N-(1,3-benzothiazol-2-yl(3-methoxy-2-pyridinyl)methyl)-3,4-dihydro-2H-1,5-benzodioxepine-7-sulfonamide

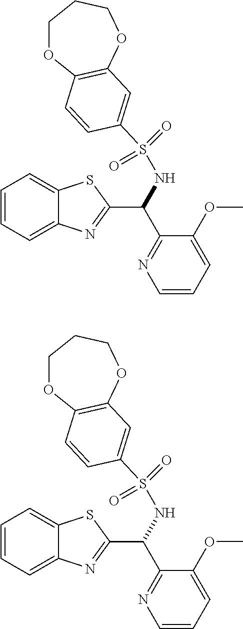 [SMILES: COc1cccnc1[C@H](NS(=O)(=O)c1ccc2c(c1)OCCCO2)c1nc2ccccc2s1 ; COc1cccnc1[C@@H](NS(=O)(=O)c1ccc2c(c1)OCCCO2)c1nc2ccccc2s1]

A 10 mL round-bottomed flask was charged with 2-bromo-3-methoxypyridine (0.25 g, 1.3 mmol, Sigma-Aldrich, St. Louis, Mo.) and toluene (2.7 mL). The solution was cooled to −78° C., n-butyllithium (0.53 mL of a 2.5 M solution with toluene, 1.3 mmol, Sigma-Aldrich, St. Louis, Mo.) was added, and then the reaction mixture was stirred for 3 h. After that time, an aliquot of this reaction mixture (a total of 0.90 mL added in two equal portions) was added to a solution of N-(1,3-benzothiazol-2-ylmethylidene)-3,4-dihydro-2H-1,5-benzodioxepine-7-sulfonamide (intermediate E) (0.10 g, 0.27 mmol) and tetrahydrofuran (2.7 mL) at 0° C. After stirring for 1 h, methanol (1.0 mL) and silica gel (0.50 g) were added sequentially and the volatiles were removed under a vacuum. The residue was subjected to flash chromatography on silica gel (6.5 g of silica gel, 99:1 dichloromethane-methanol), the isolated material was re-subjected to flash chromatography on silica gel (6.5 g of silica gel, 99.5:0.5 dichloromethane-methanol), the isolated material was again re-subjected to flash chromatography on silica gel (3.0 g of silica gel, 99.5:0.5 dichloromethane-methanol), the isolated material was subjected to reversed-phase preparative HPLC (Phenomenex Gemini C18 column (Phenomenex, Inc., Torrance, Calif.) (100×30 mm, 5 μm) eluting with 0.10% trifluoroacetic acid in acetonitrile-water, gradient of 10% to 90% over 20 min), and then the isolated material was partitioned between ethyl acetate (20 mL) and aqueous sodium carbonate (20 mL), the layers were separated. The organic material was washed sequentially with 10% aqueous sodium carbonate (2×) and brine, dried (sodium sulfate), filtered, and the filtrate was concentrated to give N-(1,3-benzothiazol-2-yl(3-methoxy-2-pyridinyl)methyl)-3,4-dihydro-2H-1,5-benzodioxepine-7-sulfonamide (0.0051 g) as a pale yellow solid (racemic mixture).

¹H NMR (400 MHz, chloroform-d) δ 8.11 (dd, J=1.0, 4.7 Hz, 1H), 7.87 (d, J=8.0 Hz, 1H), 7.78 (d, J=7.8 Hz, 1H), 7.42-7.35 (m, 1H), 7.34-7.27 (m, 3H), 7.19 (dd, J=4.6, 8.3 Hz, 1H), 7.12 (d, J=8.2 Hz, 1H), 7.02-6.87 (m, 1H), 6.72 (d, J=8.4 Hz, 1H), 6.42 (d, J=9.2 Hz, 1H), 4.15-3.90 (m, 4H), 3.83 (s, 3H), 2.16-2.04 (m, 2H). m/z (ESI, +ve ion) 483.9 (M+H)⁺. GK-GKRP EC₅₀ (LCMS/MS)=0.36 μM. GK-GKRP IC₅₀ (Binding)=0.27 μM.

### Intermediate F

### N-(1,3-benzoxazol-2-ylmethylidene)-3,4-dihydro-2H-1,5-benzodioxepine-7-sulfonamide

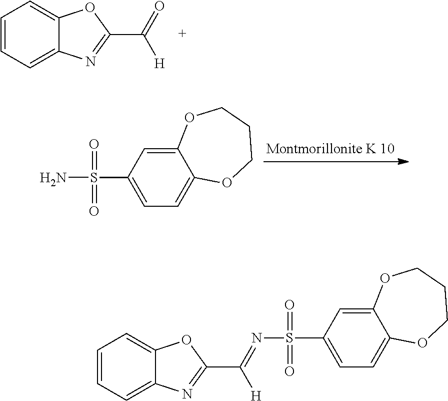 [SMILES: O=Cc1nc2ccccc2o1 ; NS(=O)(=O)c1ccc2c(c1)OCCCO2 ; O=S(=O)(/N=C/c1nc2ccccc2o1)c1ccc2c(c1)OCCCO2]

A 50 mL round-bottomed flask was charged with 1,3-benzoxazole-2-carbaldehyde (0.39 g, 2.6 mmol, Biofine International, Vancouver, BC), 3,4-dihydro-2H-1,5-benzodioxepine-7-sulfonamide (0.30 g, 1.3 mmol, Enamine, Kiev, Ukraine), Montmorillonite K 10 (0.30 g, Sigma-Aldrich, St. Louis, Mo.), and toluene (15 mL), fitted with a Dean-Stark trap and a reflux condenser, and then the reaction mixture was heated at reflux for 4 h. After that time, the reaction mixture was cooled to room temperature, filtered, the filtrate was concentrated under a vacuum until a precipitate was observed, cooled to −20° C., filtered, and the filter cake was collected to give N-(1,3-benzoxazol-2-ylmethylidene)-3,4-dihydro-2H-1,5-benzodioxepine-7-sulfonamide (0.34 g) as a colorless solid.

### Example 54

### N-(1,3-benzoxazol-2-yl(phenyl)methyl)-3,4-dihydro-2H-1,5-benzo dioxepine-7-sulfonamide

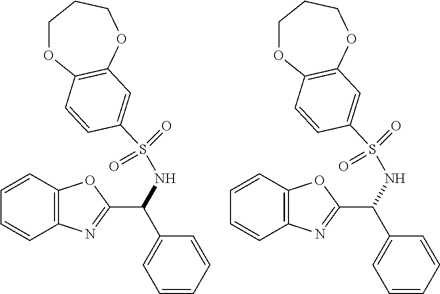

A 15 mL round-bottomed flask was charged with N-(1,3-benzoxazol-2-ylmethylidene)-3,4-dihydro-2H-1,5-benzodioxepine-7-sulfonamide (intermediate F) (0.10 g, 0.28 mmol), tetrahydrofuran (3.0 mL), and bromo(phenyl)magnesium (a total of 0.17 mL of a 1.0 M solution with tetrahydrofuran, added in three equal portions, 0.17 mmol, Sigma-Aldrich, St. Louis, Mo.), and then the reaction mixture was stirred for 15 h. After that time, saturated aqueous ammonium chloride (5.0 mL) was added, the mixture was partitioned between saturated aqueous sodium bicarbonate (50 mL) and ethyl acetate (50 mL), the layers were separated, and the organic material was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 24 min) to give N-(1,3-benzoxazol-2-yl(phenyl)methyl)-3,4-dihydro-2H-1,5-benzodioxepine-7-sulfonamide (0.020 g) as a pale pink solid (racemic mixture).

¹H NMR (400 MHz, chloroform-d) δ 7.68-7.60 (m, 1H), 7.46-7.39 (m, 1H), 7.37-7.27 (m, 9H), 6.77 (d, J=8.4 Hz, 1H), 5.93-5.82 (m, 2H), 4.15-3.94 (m, 4H), 2.16-2.05 (m, 2H). m/z (ESI, +ve ion) 437.0 (M+H)⁺. GK-GKRP EC₅₀ (LCMS/MS)=0.75 μM. GK-GKRP IC₅₀ (Binding)=1.1 μM.

### Intermediate G

### :N-(2-naphthalenylmethylidene)-3,4-dihydro-2H-1,5-benzodioxepine-7-sulfonamide

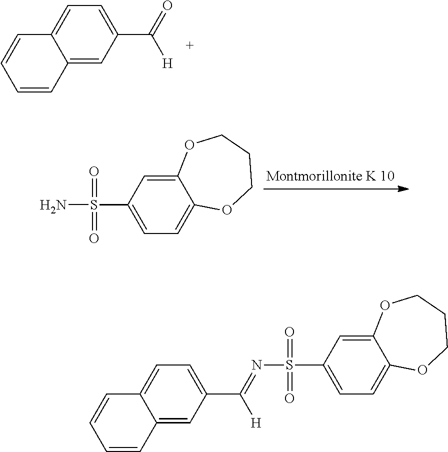 [SMILES: O=Cc1ccc2ccccc2c1 ; NS(=O)(=O)c1ccc2c(c1)OCCCO2 ; O=S(=O)(/N=C/c1ccc2ccccc2c1)c1ccc2c(c1)OCCCO2]

A 100 mL round-bottomed flask was charged with 2-naphthalenecarbaldehyde (1.0 g, 6.5 mmol, Sigma-Aldrich, St. Louis, Mo.), 3,4-dihydro-2H-1,5-benzodioxepine-7-sulfonamide (1.0 g, 4.4 mmol, Enamine, Kiev, Ukraine), Montmorillonite K 10 (1.0 g, Sigma-Aldrich, St. Louis, Mo.), and toluene (22 mL), fitted with a Dean-Stark trap and a reflux condenser, and then the reaction mixture was heated at reflux for 2 h. After that time, the reaction mixture was allowed to cool to room temperature, filtered, and the filtrate was concentrated to give N-(2-naphthalenylmethylidene)-3,4-dihydro-2H-1,5-benzodioxepine-7-sulfonamide (2.0 g) as a pale yellow oil.

### Example 55

### N-(2-naphthalenyl(phenyl)methyl)-3,4-dihydro-2H-1,5-benzo dioxepine-7-sulfonamide

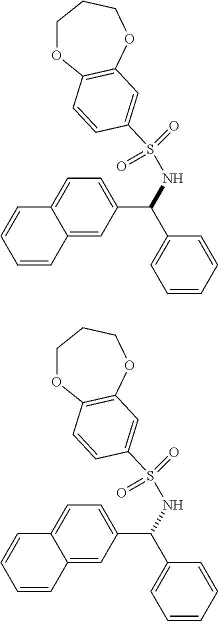 [SMILES: O=S(=O)(N[C@@H](c1ccccc1)c1ccc2ccccc2c1)c1ccc2c(c1)OCCCO2 ; O=S(=O)(N[C@H](c1ccccc1)c1ccc2ccccc2c1)c1ccc2c(c1)OCCCO2]

A 10 mL round-bottomed flask was charged with N-(2-naphthalenylmethylidene)-3,4-dihydro-2H-1,5-benzodioxepine-7-sulfonamide (intermediate G) (0.20 g, 0.54 mmol), tetrahydrofuran (6.0 mL), and bromo(phenyl)magnesium (0.54 mL of a 1.0 M solution with tetrahydrofuran, 0.54 mmol, Sigma-Aldrich, St. Louis, Mo.), and then the reaction mixture was stirred for 1 h. After that time, the reaction mixture was partitioned between saturated aqueous sodium bicarbonate (10 mL) and dichloromethane (10 mL), the layers were separated, the aqueous material was washed with dichloromethane (2×10 mL), the combined organic extract was washed with brine (10 mL), dried (sodium sulfate), filtered, and the filtrate was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 24 min) to give N-(2-naphthalenyl(phenyl)methyl)-3,4-dihydro-2H-1,5-benzodioxepine-7-sulfonamide (0.035 g) as a colorless solid (racemic mixture).

¹H NMR (400 MHz, methanol-d₄) δ 7.81-7.73 (m, 1H), 7.73-7.64 (m, 2H), 7.52 (s, 1H), 7.48-7.39 (m, 2H), 7.30-7.16 (m, 7H), 7.08-7.01 (m, 1H), 6.74 (d, J=8.4 Hz, 1H), 5.71 (s, 1H), 3.98-3.69 (m, 4H), 2.06-1.90 (m, 2H). m/z (ESI, +ve ion) 467.9 (M+Na)⁺. GK-GKRP EC₅₀ (LCMS/MS)=0.24 μM. GK-GKRP IC₅₀ (Binding)=0.86 μM.

### Intermediate H

### N-(2-quinolinylmethylidene)-3,4-dihydro-2H-1,5-benzo dioxepine-7-sulfonamide

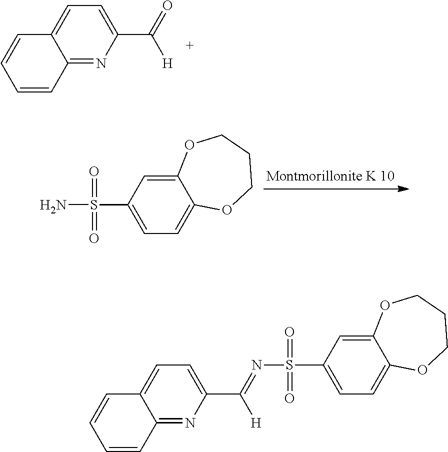 [SMILES: O=Cc1ccc2ccccc2n1 ; NS(=O)(=O)c1ccc2c(c1)OCCCO2 ; O=S(=O)(/N=C/c1ccc2ccccc2n1)c1ccc2c(c1)OCCCO2]

A 100 mL round-bottomed flask was charged with 2-quinolinecarbaldehyde (1.0 g, 6.5 mmol, Alfa Aesar, Ward Hill, Mass.), 3,4-dihydro-2H-1,5-benzodioxepine-7-sulfonamide (1.0 g, 4.4 mmol, Enamine, Kiev, Ukraine), Montmorillonite K 10 (1.0 g, Sigma-Aldrich, St. Louis, Mo.), and toluene (22 mL), fitted with a Dean-Stark trap and a reflux condenser, and then the reaction mixture was heated at reflux for 1.5 h. After that time, the reaction mixture was cooled to room temperature, filtered, and the filtrate was concentrated to give N-(2-quinolinylmethylidene)-3,4-dihydro-2H-1,5-benzodioxepine-7-sulfonamide (1.6 g) as a brown oil.

### Example 56

### N-(phenyl(2-quinolinyl)methyl)-3,4-dihydro-2H-1,5-benzodioxepine-7-sulfonamide

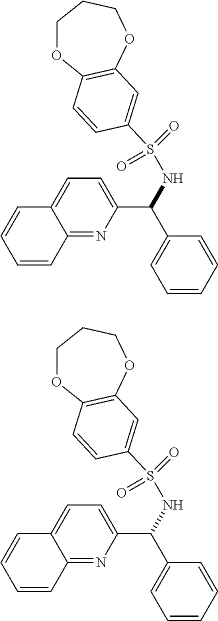 [SMILES: O=S(=O)(N[C@@H](c1ccccc1)c1ccc2ccccc2n1)c1ccc2c(c1)OCCCO2 ; O=S(=O)(N[C@H](c1ccccc1)c1ccc2ccccc2n1)c1ccc2c(c1)OCCCO2]

A 15 mL round-bottomed flask was charged with N-(2-quinolinylmethylidene)-3,4-dihydro-2H-1,5-benzodioxepine-7-sulfonamide (intermediate H) (0.30 g, 0.81 mmol), tetrahydrofuran (5.0 mL), and bromo(phenyl)magnesium (0.81 mL of a 1.0 M solution with tetrahydrofuran, 0.81 mmol, Sigma-Aldrich, St. Louis, Mo.), and then the reaction mixture was stirred for 15 h. After that time, saturated aqueous sodium bicarbonate (5.0 mL) was added, the aqueous material was washed with ethyl acetate (3×10 mL), the combined organic extracts were washed with brine (10 mL), dried (sodium sulfate), filtered, and the filtrate was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 24 min) to give N-(phenyl(2-quinolinyl)methyl)-3,4-dihydro-2H-1,5-benzodioxepine-7-sulfonamide, trifluoroacetic acid salt (0.0042 g) as a pink solid (racemic mixture).

¹H NMR (400 MHz, methanol-d₄) δ 8.33 (d, J=8.6 Hz, 1H), 8.05 (d, J=8.4 Hz, 1H), 7.95 (d, J=8.2 Hz, 1H), 7.85 (t, J=7.7 Hz, 1H), 7.66 (t, J=7.5 Hz, 1H), 7.51 (d, J=8.6 Hz, 1H), 7.35-7.19 (m, 6H), 7.16 (s, 1H), 6.73 (d, J=8.4 Hz, 1H), 5.85 (s, 1H), 4.08-3.76 (m, 4H), 2.11-1.92 (m, 2H). m/z (ESI, +ve ion) 447.0 (M+H)⁺. GK-GKRP EC₅₀ (LCMS/MS)=0.72 μM. GK-GKRP IC₅₀ (Binding)=1.4 μM.

### Intermediate I

### N-(phenylmethylidene)-3,4-dihydro-2H-1,5-benzodioxepine-7-sulfonamide

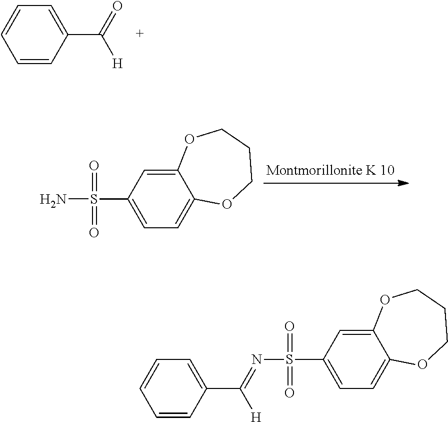 [SMILES: O=Cc1ccccc1 ; NS(=O)(=O)c1ccc2c(c1)OCCCO2 ; O=S(=O)(/N=C/c1ccccc1)c1ccc2c(c1)OCCCO2]

A 100 mL round-bottomed flask was charged with benzaldehyde (2.8 g, 26 mmol, Sigma-Aldrich, St. Louis, Mo.), 3,4-dihydro-2H-1,5-benzodioxepine-7-sulfonamide (5.0 g, 22 mmol, Enamine, Kiev, Ukraine), Montmorillonite K 10 (5.0 g, Sigma-Aldrich, St. Louis, Mo.), and toluene (110 mL), fitted with a Dean-Stark trap and a reflux condenser, and then the reaction mixture was heated at reflux for 1.5 h. After that time, the reaction mixture was filtered, the filtrate was allowed to cool to room temperature, concentrated under a vacuum to a volume of ˜20 mL, filtered, and the filter cake was collected to give N-(phenylmethylidene)-3,4-dihydro-2H-1,5-benzodioxepine-7-sulfonamide (5.2 g) as a colorless solid.

### Example 57

### N-(1-benzothiophen-2-yl(phenyl)methyl)-3,4-dihydro-2H-1,5-benzodioxepine-7-sulfonamide

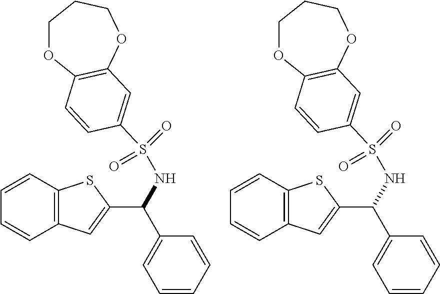

A 25 mL round-bottomed flask was charged with 1-benzothiophene (0.042 g, 0.32 mmol, Sigma-Aldrich, St. Louis, Mo.) and tetrahydrofuran (3.2 mL). The solution was cooled to −78° C., n-butyllithium (0.20 mL of a 1.6 M solution with hexane, 0.32 mmol, Sigma-Aldrich, St. Louis, Mo.) was added, and then the reaction mixture was stirred for 5 min. After that time, a solution of N-(phenylmethylidene)-3,4-dihydro-2H-1,5-benzodioxepine-7-sulfonamide (intermediate I) (0.10 g, 0.32 mmol) and tetrahydrofuran (2.0 mL) was added. After stirring for 5 min, water (2.0 mL) was added, the mixture was warmed to room temperature, partitioned between ethyl acetate (10 mL) and water (10 mL), the layers were separated, the aqueous material was washed with ethyl acetate (2×10 mL). The combined organic extract was washed with brine (10 mL), dried (sodium sulfate), filtered, silica gel (2.0 g) was added to the filtrate, and the volatiles were removed under a vacuum. The residue was subjected to flash chromatography on silica gel (Isco CombiFlash® system, 40 g RediSep® normal phase column, gradient elution of hexane to 30% ethyl acetate-hexane, Teledyne Isco, Lincoln, Nebr.) to give N-(1-benzothiophen-2-yl(phenyl)methyl)-3,4-dihydro-2H-1,5-benzodioxepine-7-sulfonamide (0.020 g) as a colorless solid (racemic mixture).

¹H NMR (400 MHz, DMSO-d₆) δ 8.98 (d, J=9.6 Hz, 1H), 7.84 (d, J=8.4 Hz, 1H), 7.68 (d, J=8.6 Hz, 1H), 7.35-7.20 (m, 8H), 7.10 (d, J=2.2 Hz, 1H), 6.91 (s, 1H), 6.86 (d, J=8.4 Hz, 1H), 5.83 (d, J=9.2 Hz, 1H), 4.02 (t, J=5.6 Hz, 2H), 3.93 (t, J=5.7 Hz, 2H), 2.07-1.99 (m, 2H). m/z (ESI, +ve ion) 473.9 (M+Na)⁺. GK-GKRP EC₅₀ (LCMS/MS)=0.11 μM. GK-GKRP IC₅₀ (Binding)=0.31 μM.

This racemic mixture was resolved using preparative SFC (Chiralpak® AS-H column (Daicel, Inc., Fort Lee, N.J.) (250 mm×21 mm, 5 μm) eluting with 60% liquid CO₂ in 40% methanol:ethanol:isopropanol (1:1:1 with 0.10% diethylamine) at a flow rate of 70 mL/min) to give two products in greater than 95% enantiomeric excess.

### N—((S)-1-benzothiophen-2-yl(phenyl)methyl)-3,4-dihydro-2H-1,5-benzodioxepine-7-sulfonamide

First Eluting Peak (Peak #1)

¹H NMR (400 MHz, methanol-d₄) δ 7.73 (d, J=7.4 Hz, 1H), 7.60 (d, J=6.9 Hz, 1H), 7.33-7.19 (m, 8H), 7.15 (d, J=2.4 Hz, 1H), 6.87 (s, 1H), 6.81 (d, J=8.6 Hz, 1H), 5.83 (s, 1H), 4.55 (s, 1H), 4.09-3.85 (m, 4H), 2.13-2.00 (m, 2H). m/z (ESI, +ve ion) 473.9 (M+Na)⁺. GK-GKRP EC₅₀ (LCMS/MS)=0.70 μM. GK-GKRP IC₅₀ (Binding)=3.0 μM.

### N—((R)-1-benzothiophen-2-yl(phenyl)methyl)-3,4-dihydro-2H-1,5-benzodioxepine-7-sulfonamide

Second Eluting Peak (Peak #2)

¹H NMR (400 MHz, methanol-d₄) δ 7.73 (d, J=7.2 Hz, 1H), 7.60 (d, J=7.0 Hz, 1H), 7.31-7.20 (m, 8H), 7.15 (d, J=2.4 Hz, 1H), 6.87 (s, 1H), 6.81 (d, J=8.4 Hz, 1H), 5.83 (s, 1H), 4.56 (s, 1H), 4.09-3.83 (m, 4H), 2.12-2.00 (m, 2H). m/z (ESI, +ve ion) 473.9 (M+Na)⁺. GK-GKRP EC₅₀ (LCMS/MS)=0.087 μM. GK-GKRP IC₅₀ (Binding)=1.9 μM.

### Example 58

### N-((5-methoxy-1-benzothiophen-2-yl)(phenyl)methyl)-3,4-dihydro-2H-1,5-benzodioxepine-7-sulfonamide

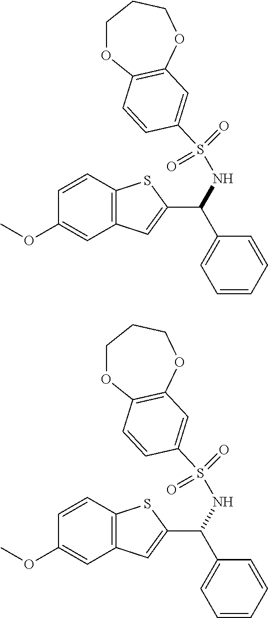 [SMILES: COc1ccc2sc([C@@H](NS(=O)(=O)c3ccc4c(c3)OCCCO4)c3ccccc3)cc2c1 ; COc1ccc2sc([C@H](NS(=O)(=O)c3ccc4c(c3)OCCCO4)c3ccccc3)cc2c1]

A 15 mL round-bottomed flask was charged with 5-methoxy-1-benzothiophene (0.16 g, 0.95 mmol, Small Molecules, Hoboken, N.J.) and tetrahydrofuran (5.0 mL). The solution was cooled to −78° C., n-butyllithium (0.59 mL of a 1.6 M solution with hexane, 0.95 mmol, Sigma-Aldrich, St. Louis, Mo.) was added, and then the reaction mixture was stirred for 20 min. After that time, a solution of N-(phenylmethylidene)-3,4-dihydro-2H-1,5-benzodioxepine-7-sulfonamide (intermediate I) (0.30 g, 0.95 mmol) and tetrahydrofuran (5.0 mL) was added. After stirring for 1 min, saturated aqueous sodium bicarbonate (5.0 mL) was added, the mixture was warmed to room temperature, partitioned between ethyl acetate (100 mL) and water (100 mL), the layers were separated, the organic material was washed with brine (50 mL), dried (sodium sulfate), filtered, silica gel (1.0 g) was added to the filtrate, and the volatiles were removed under a vacuum. The residue was subjected to flash chromatography on silica gel (Isco CombiFlash® system, 40 g RediSep® normal phase column, gradient elution of hexane to 50% ethyl acetate-hexane, Teledyne Isco, Lincoln, Nebr.) to give N-((5-methoxy-1-benzothiophen-2-yl)(phenyl)methyl)-3,4-dihydro-2H-1,5-benzodioxepine-7-sulfonamide (0.24 g) as a colorless solid (racemic mixture).

¹H NMR (400 MHz, DMSO-d₆) δ 8.96 (d, J=9.0 Hz, 1H), 7.71 (d, J=8.8 Hz, 1H), 7.35-7.17 (m, 7H), 7.10 (d, J=2.2 Hz, 1H), 6.91 (dd, J=8.8, 2.4 Hz, 1H), 6.86 (d, J=8.4 Hz, 1H), 6.82 (s, 1H), 5.78 (t, J=8.9 Hz, 1H), 4.04 (t, J=5.5 Hz, 2H), 3.96 (t, J=5.6 Hz, 2H), 3.75 (s, 3H), 2.09-2.00 (m, 2H). m/z (ESI, +ve ion) 503.9 (M+Na)⁺. GK-GKRP EC₅₀ (LCMS/MS)=0.20 μM. GK-GKRP IC₅₀ (Binding)=0.79 μM.

### Example 59

### N-(1,3-benzothiazol-2-yl(phenyl)methyl)-3,4-dihydro-2H-1,5-benzodioxepine-7-sulfonamide

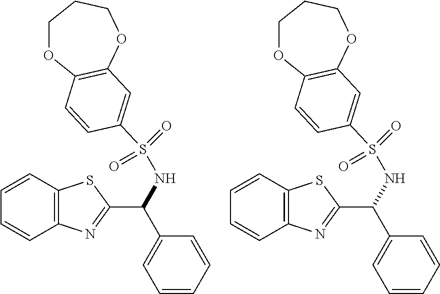

A 15 mL round-bottomed flask was charged with 1,3-benzothiazole (0.32 g, 0.24 mmol, Sigma-Aldrich, St. Louis, Mo.) and diethyl ether (2.4 mL). The solution was cooled to −78° C., n-butyllithium (0.15 mL of a 1.6 M solution with hexane, 0.24 mmol, Sigma-Aldrich, St. Louis, Mo.) was added, and then the reaction mixture was stirred for 5 min. After that time, a solution of N-(phenylmethylidene)-3,4-dihydro-2H-1,5-benzodioxepine-7-sulfonamide (intermediate I) (0.075 g, 0.24 mmol) and tetrahydrofuran (1.0 mL) was added. After stirring for 1 min, water (1.0 mL) was added, the mixture was warmed to room temperature and partitioned between ethyl acetate (10 mL) and water (10 mL). After the layers were separated, the aqueous material was washed with dichloromethane (10 mL), and the combined organic extracts were washed with brine (10 mL), dried (sodium sulfate), filtered, and the filtrate was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 20 min) to give N-(1,3-benzothiazol-2-yl(phenyl)methyl)-3,4-dihydro-2H-1,5-benzodioxepine-7-sulfonamide (0.0010 g) as a colorless solid (racemic mixture).

¹H NMR (400 MHz, chloroform-d) δ 7.95 (d, J=8.2 Hz, 1H), 7.78 (d, J=8.0 Hz, 1H), 7.47 (t, J=8.2 Hz, 1H), 7.36 (t, J=7.7 Hz, 1H), 7.32-7.27 (m, 7H), 6.81 (d, J=8.2 Hz, 1H), 6.24 (d, J=6.3 Hz, 1H), 5.86 (d, J=6.5 Hz, 1H), 4.19-4.01 (m, 4H), 2.18-2.09 (m, 2H). m/z (ESI, +ve ion) 452.9 (M+H)⁺. GK-GKRP EC₅₀ (LCMS/MS)=0.30 μM. GK-GKRP IC₅₀ (Binding)=0.63 μM.

### Example 60

### N-(phenyl(thieno[2,3-c]pyridin-2-yl)methyl)-3,4-dihydro-2H-1,5-benzodioxepine-7-sulfonamide

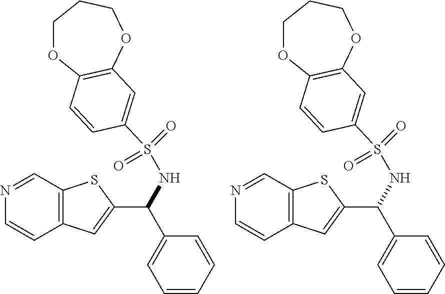

A 10 mL round-bottomed flask was charged with thieno[2,3-c]pyridine (0.075 g, 0.56 mmol, Frontier Scientific, Logan, Utah) and tetrahydrofuran (3.0 mL). The solution was cooled to −78° C., n-butyllithium (0.35 mL of a 1.6 M solution with hexane, 0.56 mmol, Sigma-Aldrich, St. Louis, Mo.) was added, and then the reaction mixture was stirred for 5 min. After that time, a solution of N-(phenylmethylidene)-3,4-dihydro-2H-1,5-benzodioxepine-7-sulfonamide (intermediate I) (0.18 g, 0.56 mmol) and tetrahydrofuran (3.0 mL) was added. After stirring for 1 min, the reaction mixture was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 24 min) to give N-(phenyl(thieno[2,3-c]pyridin-2-yl)methyl)-3,4-dihydro-2H-1,5-benzodioxepine-7-sulfonamide (0.060 g) as a colorless solid (racemic mixture).

¹H NMR (400 MHz, chloroform-d) δ 9.40 (s, 1H), 8.54 (d, J=6.3 Hz, 1H), 7.91 (d, J=6.1 Hz, 1H), 7.36-7.28 (m, 6H), 7.21-7.11 (m, 2H), 6.89 (d, J=8.4 Hz, 1H), 6.40 (d, J=8.0 Hz, 1H), 5.92 (d, J=7.6 Hz, 1H), 4.33-4.09 (m, 4H), 2.29-2.13 (m, 2H). m/z (ESI, +ve ion) 452.9 (M+H)⁺. GK-GKRP EC₅₀ (LCMS/MS)=0.57 μM. GK-GKRP IC₅₀ (Binding)=0.36 μM.

### Example 61

### N-(phenyl(thieno[3,2-b]pyridin-2-yl)methyl)-3,4-dihydro-2H-1,5-benzodioxepine-7-sulfonamide

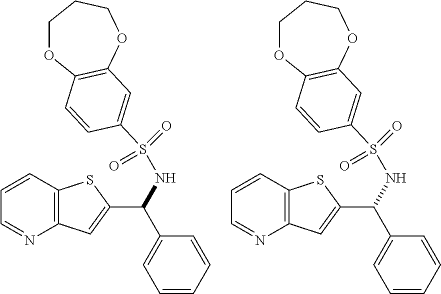

A 15 mL round-bottomed flask was charged with thieno[3,2-b]pyridine (0.20 g, 1.5 mmol, Aces Pharma, Branford, Conn.) and tetrahydrofuran (5.0 mL). The solution was cooled to −78° C., n-butyllithium (1.0 mL of a 1.6 M solution with hexane, 1.6 mmol, Sigma-Aldrich, St. Louis, Mo.) was added, and then the reaction mixture was stirred for 10 min. After that time, a solution of N-(phenylmethylidene)-3,4-dihydro-2H-1,5-benzodioxepine-7-sulfonamide (intermediate I) (0.47 g, 1.5 mmol) and tetrahydrofuran (5.0 mL) was added. After stirring for 1 min, saturated aqueous sodium bicarbonate (5.0 mL) was added, the mixture was warmed to room temperature, partitioned between ethyl acetate (50 mL) and saturated aqueous sodium bicarbonate (50 mL). The layers were separated, the organic material was washed with brine (50 mL), dried (sodium sulfate), filtered, and the filtrate was concentrated. The residue was subjected to flash chromatography on silica gel (Isco CombiFlash® system, 40 g RediSep® normal phase column, gradient elution of 20% to 50% ethyl acetate-hexane, Teledyne Isco, Lincoln, Nebr.) to give N-(phenyl(thieno[3,2-b]pyridin-2-yl)methyl)-3,4-dihydro-2H-1,5-benzodioxepine-7-sulfonamide (0.31 g) as an off-white solid (racemic mixture).

¹H NMR (400 MHz, DMSO-d₆) δ 9.06 (d, J=9.6 Hz, 1H), 8.56 (d, J=4.5 Hz, 1H), 8.33 (d, J=8.0 Hz, 1H), 7.40-7.20 (m, 7H), 7.14 (d, J=2.2 Hz, 1H), 7.05 (s, 1H), 6.85 (d, J=8.4 Hz, 1H), 5.91 (d, J=9.6 Hz, 1H), 4.09-3.87 (m, 4H), 2.12-1.99 (m, 2H). m/z (ESI, +ve ion) 452.9 (M+H)⁺. GK-GKRP EC₅₀ (LCMS/MS)=2.5 μM. GK-GKRP IC₅₀ (Binding)=2.3 μM.

### Example 62

### N-(phenyl(thieno[3,2-c]pyridin-2-yl)methyl)-3,4-dihydro-2H-1,5-benzodioxepine-7-sulfonamide

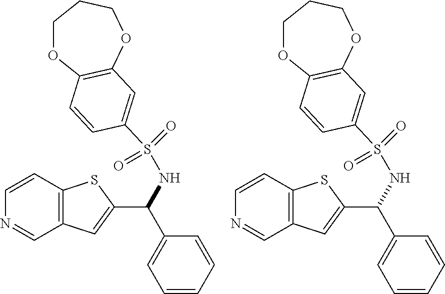

A 15 mL round-bottomed flask was charged with thieno[3,2-c]pyridine (0.085 g, 0.63 mmol, Aces Pharma, Branford, Conn.) and tetrahydrofuran (10 mL). the solution was cooled to −78° C., n-butyllithium (0.39 mL of a 1.6 M solution with hexane, 0.63 mmol, Sigma-Aldrich, St. Louis, Mo.) was added, and then the reaction mixture was stirred for 5 min. After that time, a solution of N-(phenylmethylidene)-3,4-dihydro-2H-1,5-benzodioxepine-7-sulfonamide (intermediate I) (0.20 g, 0.63 mmol) and tetrahydrofuran (3.0 mL) was added, and then the reaction mixture was warmed to room temperature. After stirring for 30 min, the reaction mixture was concentrated. The residue was subjected to reversed-phase preparative HPLC (Phenomenex Gemini C18 column (Phenomenex, Inc., Torrance, Calif.) (150×30 mm, 10 μm) eluting with 0.10% trifluoroacetic acid in acetonitrile-water, gradient of 0% to 100% over 24 min) to give N-(phenyl(thieno[3,2-c]pyridin-2-yl)methyl)-3,4-dihydro-2H-1,5-benzodioxepine-7-sulfonamide (0.080 g) as a colorless solid (racemic mixture).

¹H NMR (400 MHz, chloroform-d) δ 9.21 (s, 1H), 8.53 (d, J=6.3 Hz, 1H), 8.11 (d, J=6.3 Hz, 1H), 7.38-7.28 (m, 6H), 7.21-7.11 (m, 2H), 6.89 (d, J=8.2 Hz, 1H), 6.07-5.97 (m, 1H), 5.87 (d, J=7.4 Hz, 1H), 4.33-4.10 (m, 4H), 2.29-2.14 (m, 2H). m/z (ESI, +ve ion) 452.9 (M+H)⁺. GK-GKRP EC₅₀ (LCMS/MS)=4.6 μM. GK-GKRP IC₅₀ (Binding)=11 μM.

### Example 63

### N-((5-hydroxy-1-benzothiophen-2-yl)(phenyl)methyl)-3,4-dihydro-2H-1,5-benzodioxepine-7-sulfonamide

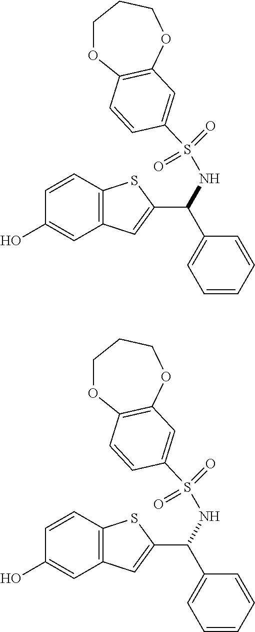 [SMILES: O=S(=O)(N[C@@H](c1ccccc1)c1cc2cc(O)ccc2s1)c1ccc2c(c1)OCCCO2 ; O=S(=O)(N[C@H](c1ccccc1)c1cc2cc(O)ccc2s1)c1ccc2c(c1)OCCCO2]

Step 1.

A 5.0 mL reaction vial was charged with 5-methoxy-1-benzothiophene (0.64 g, 3.9 mmol, Small Molecules, Hoboken, N.J.) and pyridine hydrochloride (1.3 g, 12 mmol, Acros, Waltham, Mass.), and then the reaction mixture was heated at 190° C. for 3.5 h. After that time, the reaction mixture was cooled to room temperature, partitioned between ethyl acetate (100 mL) water (50 mL), the layers were separated, the organic material was washed with brine (25 mL), dried (magnesium sulfate), filtered, and the filtrate was concentrated. The isolated material (0.64 g) was used in the next step of the synthesis without purification.

Step 2.

A 15 mL round-bottomed flask was charged with the isolated material (0.64 g) from Step 1 (5-hydroxybenzothiophene), cesium carbonate (2.8 g, 8.5 mmol, Alfa Aesar, Ward Hill, Mass.), N,N-dimethylformamide (10 mL), and (bromomethyl)benzene (0.51 mL, 4.3 mmol, Sigma-Aldrich, St. Louis, Mo.), and then the reaction mixture was stirred for 2 h. After that time, the reaction mixture was partitioned between diethyl ether (100 mL) and water (100 mL), the layers were separated, the organic material was washed with brine (50 mL), dried (magnesium sulfate), filtered, and the filtrate was concentrated. The isolated material (0.82 g) was used in the next step of the synthesis without purification.

Step 3.

A 15 mL round-bottomed flask was charged with a portion of the isolated material (0.30 g) from Step 2 and tetrahydrofuran (6.0 mL), cooled to −78° C., n-butyllithium (0.79 mL of a 1.6 M solution with hexane, 1.3 mmol, Sigma-Aldrich, St. Louis, Mo.) was added, and then the reaction mixture was stirred for 1 h. After that time, a solution of N-(phenylmethylidene)-3,4-dihydro-2H-1,5-benzodioxepine-7-sulfonamide (intermediate I) (0.40 g, 1.3 mmol) and tetrahydrofuran (5.0 mL) was added and the reaction mixture was stirred for 5 min. After that time, saturated aqueous sodium bicarbonate (10 mL) was added, the mixture was warmed to room temperature, partitioned between water (50 mL) and ethyl acetate (50 mL). The layers were separated, and the aqueous material was washed with ethyl acetate (50 mL). The combined organic extracts were washed with brine (25 mL), dried (magnesium sulfate), filtered, silica gel (2.0 g) was added to the filtrate and the volatiles were removed under a vacuum. The residue was subjected to flash chromatography on silica gel (Isco CombiFlash® system, 40 g RediSep® normal phase column, gradient elution of 0% to 35% ethyl acetate-hexane, Teledyne Isco, Lincoln, Nebr.) to give N-((5-(benzyloxy)-1-benzothiophen-2-yl)(phenyl)methyl)-3,4-dihydro-2H-1,5-benzodioxepine-7-sulfonamide (0.17 g) as a colorless solid.

Step 4.

A 150 mL round-bottomed flask was charged with N-((5-(benzyloxy)-1-benzothiophen-2-yl)(phenyl)methyl)-3,4-dihydro-2H-1,5-benzodioxepine-7-sulfonamide (0.17 g, 0.31 mmol), dichloromethane (10 mL), ethanol (10 mL), and palladium 10 wt % (dry basis) on activated carbon (wet) Degussa type E101 NE/W (0.50 g, Sigma-Aldrich, St. Louis, Mo.), under a hydrogen atmosphere (1 atm; 101 kpascal), and then the reaction mixture was stirred for 15 h. After that time, the reaction mixture was filtered, silica gel (2.0 g) was added to the filtrate, and the volatiles were removed under a vacuum. The residue was subjected to flash chromatography on silica gel (Isco CombiFlash® system, 40 g RediSep® normal phase column, gradient elution of 0% to 50% ethyl acetate-hexane, Teledyne Isco, Lincoln, Nebr.) to give N-((5-hydroxy-1-benzothiophen-2-yl)(phenyl)methyl)-3,4-dihydro-2H-1,5-benzodioxepine-7-sulfonamide (0.030 g) as a pale yellow solid (racemic mixture).

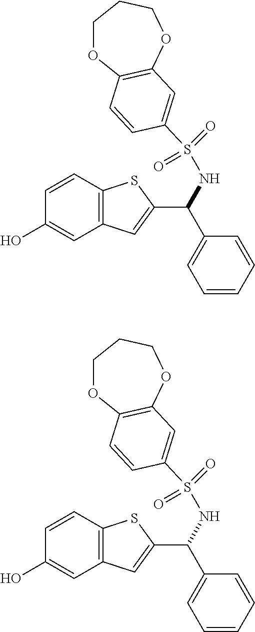 [SMILES: O=S(=O)(N[C@@H](c1ccccc1)c1cc2cc(O)ccc2s1)c1ccc2c(c1)OCCCO2 ; O=S(=O)(N[C@H](c1ccccc1)c1cc2cc(O)ccc2s1)c1ccc2c(c1)OCCCO2]

¹H NMR (400 MHz, acetonitrile-d₃) δ 7.57 (d, J=8.8 Hz, 1H), 7.34-7.20 (m, 6H), 7.16 (d, J=2.2 Hz, 1H), 7.04 (d, J=2.4 Hz, 1H), 6.91 (s, 1H), 6.87-6.80 (m, 2H), 6.77 (s, 1H), 6.63 (d, J=9.4 Hz, 1H), 5.80 (d, J=9.2 Hz, 1H), 4.15-4.08 (m, 2H), 4.01 (t, J=5.7 Hz, 2H), 2.13-2.05 (m, 2H). m/z (ESI, +ve ion) 490.0 (M+Na)⁺. GK-GKRP EC₅₀ (LCMS/MS)=3.2 μM. GK-GKRP IC₅₀ (Binding)=4.2 μM.

### Intermediate J

### N-((2-(methylsulfanyl)phenyl)methylidene)-3,4-dihydro-2H-1,5-benzodioxepine-7-sulfonamide

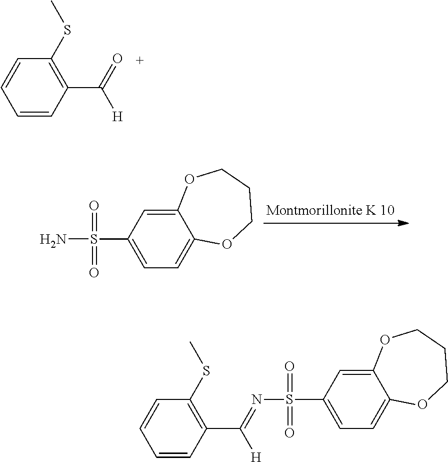

A 150 mL round-bottomed flask was charged with 2-(methylsulfanyl)benzaldehyde (2.0 g, 13 mmol, Sigma-Aldrich, St. Louis, Mo.), 3,4-dihydro-2H-1,5-benzodioxepine-7-sulfonamide (3.0 g, 13 mmol, Enamine, Kiev, Ukraine), Montmorillonite K 10 (3.0 g, Sigma-Aldrich, St. Louis, Mo.), and toluene (65 mL), fitted with a Dean-Stark trap and a reflux condenser, and then the reaction mixture was heated at reflux for 20 h. After that time, the reaction mixture was filtered, the filtrate was allowed to cool to room temperature, filtered, and the filtrate was concentrated to give N-((2-(methylsulfanyl)phenyl)methylidene)-3,4-dihydro-2H-1,5-benzodioxepine-7-sulfonamide (3.3 g) as a yellow oil.

### Example 64

### N-((2-(methylsulfanyl)phenyl)(thieno[2,3-c]pyridin-2-yl)methyl)-3,4-dihydro-2H-1,5-benzodioxepine-7-sulfonamide

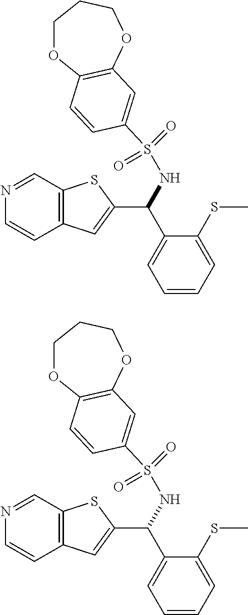 [SMILES: CSc1ccccc1[C@H](NS(=O)(=O)c1ccc2c(c1)OCCCO2)c1cc2ccncc2s1 ; CSc1ccccc1[C@@H](NS(=O)(=O)c1ccc2c(c1)OCCCO2)c1cc2ccncc2s1]

A 15 mL round-bottomed flask was charged with thieno[2,3-c]pyridine (0.10 g, 0.74 mmol, Frontier Scientific, Logan, Utah) and tetrahydrofuran (3.7 mL), the solution was cooled to −78° C., n-butyllithium (0.33 mL of a 2.5 M solution with toluene, 0.81 mmol, Sigma-Aldrich, St. Louis, Mo.) was added, and then the reaction mixture was stirred for 15 min. After that time, a solution of N-((2-(methylsulfanyl)phenyl)methylidene)-3,4-dihydro-2H-1,5-benzodioxepine-7-sulfonamide (intermediate J) (0.27 g, 0.74 mmol) and tetrahydrofuran (3.7 mL) was added. After stirring for 5 min, methanol (1.0 mL) and silica gel (0.50 g) were added sequentially, and the volatiles were removed under a vacuum. The residue was subjected to flash chromatography on silica gel (19 g of silica gel; 1:1 hexane-ethyl acetate) to give N-((2-(methylsulfanyl)phenyl)(thieno[2,3-c]pyridin-2-yl)methyl)-3,4-dihydro-2H-1,5-benzodioxepine-7-sulfonamide (0.12 g) as a colorless solid (racemic mixture).

¹H NMR (400 MHz, DMSO-d₆) δ 9.17 (br. s., 1H), 9.12 (s, 1H), 8.39 (d, J=5.3 Hz, 1H), 7.68 (dd, J=0.9, 5.4 Hz, 1H), 7.46-7.40 (m, 1H), 7.35-7.29 (m, 1H), 7.29-7.22 (m, 1H), 7.20 (dd, J=2.2, 8.4 Hz, 1H), 7.14-7.05 (m, 2H), 6.91 (s, 1H), 6.87 (d, J=8.4 Hz, 1H), 6.31 (br. s., 1H), 4.09 (t, J=5.7 Hz, 2H), 4.01 (qd, J=6.2, 12.5 Hz, 2H), 2.45 (s, 3H), 2.07 (quin, J=5.7 Hz, 2H). m/z (ESI, +ve ion) 498.8 (M+H)⁺. GK-GKRP EC₅₀ (LCMS/MS)=0.020 μM. GK-GKRP IC₅₀ (Binding)=0.0040 μM.

The racemic mixture was resolved using preparative SFC (Chiralpak® AS-H column (Daicel, Inc., Fort Lee, N.J.) (250 mm×21 mm, 5 μm) eluting with 60% liquid CO₂ in 40% methanol (with 40 mM NH₃) at a flow rate of 75 mL/min) to give two products in greater than 99% enantiomeric excess. Product 1 (first eluting peak, peak #1) was dissolved with methanol (3.0 mL) and then the solution was filtered through a StratoSpheres SPE PL-HCO₃ MP-Resin column (Agilent Technologies, Santa Clara, Calif.), the isolated material was dissolved with methanol (3.0 mL) and then the solution was filtered through a SPE-R66030B-06S Si-Carbonate (6 mL, 1 g) column (SiliCycle, Quebec City, Canada) and the filtrate was concentrated. Product 2 (second eluting peak, peak #2) was dissolved with methanol (3.0 mL) and then the solution was filtered through a SPE-R66030B-06S Si-Carbonate (6 mL, 1 g) column (SiliCycle, Quebec City, Canada) and the filtrate was concentrated.

### N—((S)-(2-(methylsulfanyl)phenyl)(thieno[2,3-c]pyridin-2-yl)methyl)-3,4-dihydro-2H-1,5-benzodioxepine-7-sulfonamide

First Eluting Peak (Peak #1)

¹H NMR (400 MHz, chloroform-d) δ 8.99 (s, 1H), 8.43 (d, J=5.3 Hz, 1H), 7.48 (d, J=5.3 Hz, 1H), 7.33-7.22 (m, 4H), 7.20 (d, J=7.6 Hz, 1H), 7.13 (t, J=7.0 Hz, 1H), 6.94 (s, 1H), 6.83 (d, J=8.2 Hz, 1H), 6.25 (d, J=9.2 Hz, 1H), 6.19 (d, J=8.8 Hz, 1H), 4.23 (t, J=5.6 Hz, 2H), 4.15 (t, J=5.6 Hz, 2H), 2.37 (s, 3H), 2.19 (quin, J=5.6 Hz, 2H). m/z (ESI, +ve ion) 498.8 (M+H)⁺. GK-GKRP EC₅₀ (LCMS/MS)=0.18 μM. GK-GKRP IC₅₀ (Binding)=0.43 μM.

### N—((R)-(2-(methylsulfanyl)phenyl)(thieno[2,3-c]pyridin-2-yl)methyl)-3,4-dihydro-2H-1,5-benzodioxepine-7-sulfonamide

Second Eluting Peak (Peak #2)

¹H NMR (400 MHz, chloroform-d) δ 8.98 (s, 1H), 8.42 (d, J=5.3 Hz, 1H), 7.47 (d, J=5.3 Hz, 1H), 7.34-7.22 (m, 4H), 7.22-7.16 (m, 1H), 7.16-7.08 (m, 1H), 6.93 (s, 1H), 6.82 (d, J=8.8 Hz, 1H), 6.29 (br. s., 1H), 6.20 (br. s., 1H), 4.22 (t, J=5.7 Hz, 2H), 4.14 (t, J=5.7 Hz, 2H), 2.36 (s, 3H), 2.19 (quin, J=5.7 Hz, 2H). m/z (ESI, +ve ion) 498.8 (M+H)⁺. GK-GKRP EC₅₀ (LCMS/MS)=0.010 μM. GK-GKRP IC₅₀ (Binding)=0.0010 μM.

### Example 65

### N-((2-(methylsulfanyl)phenyl)([1,3]thiazolo[5,4-b]pyridin-2-yl)methyl)-3,4-dihydro-2H-1,5-benzodioxepine-7-sulfonamide

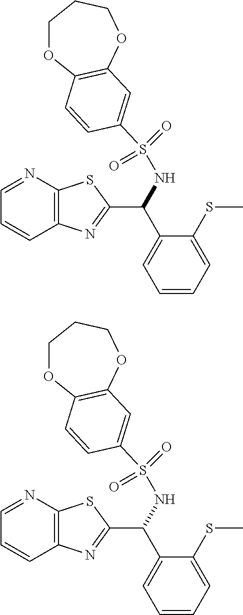 [SMILES: CSc1ccccc1[C@H](NS(=O)(=O)c1ccc2c(c1)OCCCO2)c1nc2cccnc2s1 ; CSc1ccccc1[C@@H](NS(=O)(=O)c1ccc2c(c1)OCCCO2)c1nc2cccnc2s1]

A 15 mL round-bottomed flask was charged with [1,3]thiazolo[5,4-b]pyridine (0.10 g, 0.73 mmol, Atlantic, Stratton, UK) and tetrahydrofuran (3.7 mL), the solution was cooled to −78° C., n-butyllithium (0.32 mL of a 2.5 M solution with toluene, 0.81 mmol, Sigma-Aldrich, St. Louis, Mo.) was added, and then the reaction mixture was stirred for 15 min. After that time, a solution of N-((2-(methylsulfanyl)phenyl)methylidene)-3,4-dihydro-2H-1,5-benzodioxepine-7-sulfonamide (intermediate J) (0.27 g, 0.73 mmol) and tetrahydrofuran (3.7 mL) was added. After stirring for 10 min, the reaction mixture was warmed to room temperature, saturated aqueous sodium bicarbonate (2.0 mL) was added, the mixture was partitioned between ethyl acetate (50 mL) and saturated aqueous sodium bicarbonate (50 mL), the layers were separated, the organic material was washed sequentially with saturated aqueous sodium bicarbonate (50 mL) and brine (50 mL), dried (sodium sulfate), filtered, the filtrate was concentrated, the residue was dissolved with dichloromethane (5.0 mL), silica gel (0.50 g) was added, and the volatiles were removed under vacuum. The residue was subjected to flash chromatography on silica gel (19 g of silica gel; 1:1 hexane-ethyl acetate), and then the isolated material was re-subjected to flash chromatography on silica gel (10 g of silica gel; 99:1 dichloromethane-methanol) to give N-((2-(methylsulfanyl)phenyl)([1,3]thiazolo[5,4-b]pyridin-2-yl)methyl)-3,4-dihydro-2H-1,5-benzodioxepine-7-sulfonamide (0.090 g) as a colorless solid (racemic mixture).

¹H NMR (400 MHz, DMSO-d₆) δ 9.33 (br. s., 1H), 8.58 (dd, J=1.4, 4.7 Hz, 1H), 8.27 (dd, J=1.6, 8.2 Hz, 1H), 7.54 (dd, J=4.6, 8.3 Hz, 1H), 7.45-7.37 (m, 1H), 7.37-7.30 (m, 1H), 7.30-7.20 (m, 2H), 7.18-7.07 (m, 2H), 6.90 (d, J=8.4 Hz, 1H), 6.42 (br. s., 1H), 4.10 (t, J=5.6 Hz, 2H), 4.03 (t, J=5.6 Hz, 2H), 2.44 (s, 3H), 2.09 (quin, J=5.6 Hz, 2H). m/z (ESI, +ve ion) 499.9 (M+H)⁺. GK-GKRP EC₅₀ (LCMS/MS)=0.037 μM. GK-GKRP IC₅₀ (Binding)=0.0090 μM.

The racemic mixture was resolved using preparative SFC (Chiralpak® AS-H column (Daicel, Inc., Fort Lee, N.J.) (150 mm×20 mm, 5 μm) eluting with 65% liquid CO₂ in 35% methanol:ethanol:isopropanol (1:1:1 with 0.10% diethylamine) at a flow rate of 70 mL/min) to give two products in greater than 99% enantiomeric excess.

### N—((S)-(2-(methylsulfanyl)phenyl)([1,3]thiazolo[5,4-b]pyridin-2-yl)methyl)-3,4-dihydro-2H-1,5-benzodioxepine-7-sulfonamide

First Eluting Peak (Peak #1)

¹H NMR (400 MHz, DMSO-d₆) δ 9.30 (br. s., 1H), 8.62-8.54 (m, 1H), 8.30-8.21 (m, 1H), 7.54 (dd, J=4.7, 8.2 Hz, 1H), 7.41 (d, J=7.6 Hz, 1H), 7.38-7.31 (m, 1H), 7.31-7.19 (m, 2H), 7.18-7.06 (m, 2H), 6.89 (d, J=8.4 Hz, 1H), 6.42 (s, 1H), 4.10 (t, J=5.5 Hz, 2H), 4.03 (t, J=5.5 Hz, 2H), 2.44 (s, 3H), 2.09 (quin, J=5.5 Hz, 2H). m/z (ESI, +ve ion) 499.9 (M+H)⁺. GK-GKRP IC₅₀ (Binding)=0.90 μM.

### N—((R)-(2-(methylsulfanyl)phenyl)([1,3]thiazolo[5,4-b]pyridin-2-yl)methyl)-3,4-dihydro-2H-1,5-benzodioxepine-7-sulfonamide

Second Eluting Peak (Peak #2)

Component 2: ¹H NMR (400 MHz, DMSO-d₆) δ 9.32 (br. s., 1H), 8.58 (d, J=3.7 Hz, 1H), 8.27 (d, J=8.2 Hz, 1H), 7.54 (dd, J=4.7, 8.2 Hz, 1H), 7.41 (d, J=7.6 Hz, 1H), 7.37-7.31 (m, 1H), 7.31-7.20 (m, 2H), 7.19-7.06 (m, 2H), 6.89 (d, J=8.4 Hz, 1H), 6.42 (s, 1H), 4.10 (t, J=5.5 Hz, 2H), 4.03 (t, J=5.5 Hz, 2H), 2.44 (s, 3H), 2.09 (quin, J=5.5 Hz, 2H). m/z (ESI, +ve ion) 499.9 (M+H)⁺. GK-GKRP EC₅₀ (LCMS/MS)=0.015 μM. GK-GKRP IC₅₀ (Binding)=0.0070 μM.

### Intermediate K

### N-((2-methoxyphenyl)methylidene)-3,4-dihydro-2H-1,5-benzodioxepine-7-sulfonamide

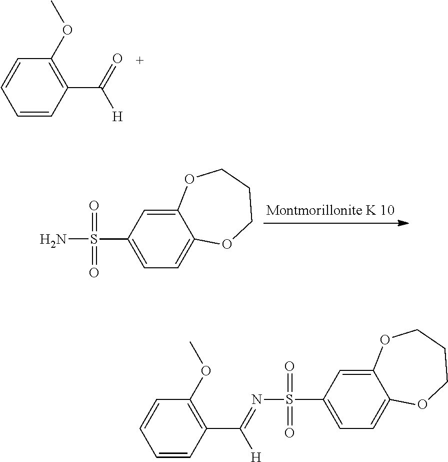

A 150 mL round-bottomed flask was charged with 2-methoxybenzaldehyde (1.8 g, 13 mmol, Sigma-Aldrich, St. Louis, Mo.), 3,4-dihydro-2H-1,5-benzodioxepine-7-sulfonamide (3.0 g, 13 mmol, Enamine, Kiev, Ukraine), Montmorillonite K 10 (3.0 g, Sigma-Aldrich, St. Louis, Mo.), and toluene (65 mL), fitted with a Dean-Stark trap and a reflux condenser, and then the reaction mixture was heated at reflux for 3 h. After that time, the reaction mixture was filtered and the filtrate was concentrated under a stream of dry nitrogen to give N-((2-methoxyphenyl)methylidene)-3,4-dihydro-2H-1,5-benzodioxepine-7-sulfonamide (3.6 g) as an off-white solid.

### Example 66

### N-((2-methoxyphenyl)(thieno[2,3-c]pyridin-2-yl)methyl)-3,4-dihydro-2H-1,5-benzodioxepine-7-sulfonamide

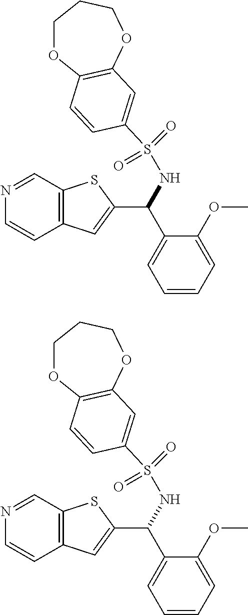 [SMILES: COc1ccccc1[C@H](NS(=O)(=O)c1ccc2c(c1)OCCCO2)c1cc2ccncc2s1 ; COc1ccccc1[C@@H](NS(=O)(=O)c1ccc2c(c1)OCCCO2)c1cc2ccncc2s1]

A 15 mL round-bottomed flask was charged with thieno[2,3-c]pyridine (0.10 g, 0.74 mmol, Frontier Scientific, Logan, Utah) and tetrahydrofuran (3.7 mL), the solution was cooled to −78° C., n-butyllithium (0.33 mL of a 2.5 M solution with toluene, 0.81 mmol, Sigma-Aldrich, St. Louis, Mo.) was added, and then the reaction mixture was stirred for 15 min. After that time, a solution of N-((2-methoxyphenyl)methylidene)-3,4-dihydro-2H-1,5-benzodioxepine-7-sulfonamide (intermediate K) (0.26 g, 0.74 mmol) and tetrahydrofuran (3.7 mL) was added. After stirring for 5 min, methanol (1.0 mL) was added, the mixture was warmed to room temperature, silica gel (0.50 g) was added, and then the volatiles were removed under vacuum. The residue was subjected to flash chromatography on silica gel (19 g of silica gel; 1:1 hexane-ethyl acetate) to give N-((2-methoxyphenyl)(thieno[2,3-c]pyridin-2-yl)methyl)-3,4-dihydro-2H-1,5-benzodioxepine-7-sulfonamide (0.21 g) as a colorless solid (racemic mixture).

¹H NMR (400 MHz, DMSO-d₆) δ 9.10 (s, 1H), 8.95 (d, J=5.7 Hz, 1H), 8.38 (d, J=5.5 Hz, 1H), 7.67 (d, J=5.3 Hz, 1H), 7.36-7.29 (m, 1H), 7.27-7.15 (m, 2H), 7.07 (d, J=2.2 Hz, 1H), 6.98 (s, 1H), 6.91 (d, J=8.2 Hz, 1H), 6.89-6.79 (m, 2H), 6.25-6.09 (m, 1H), 4.13-4.04 (m, 2H), 4.01 (t, J=5.7 Hz, 2H), 3.73 (s, 3H), 2.07 (quin, J=5.7 Hz, 2H). m/z (ESI, +ve ion) 482.9 (M+H)⁺. GK-GKRP EC₅₀ (LCMS/MS)=0.082 μM. GK-GKRP IC₅₀ (Binding)=0.072 μM.

### Example 67

### N-((2-methoxyphenyl)([1,3]thiazolo[5,4-b]pyridin-2-yl)methyl)-3,4-dihydro-2H-1,5-benzodioxepine-7-sulfonamide

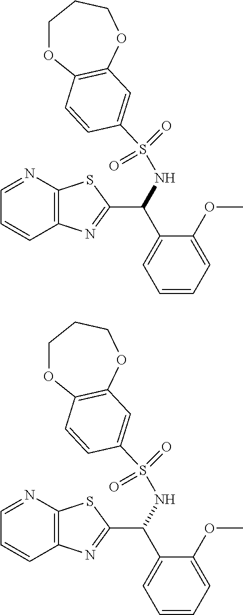 [SMILES: COc1ccccc1[C@H](NS(=O)(=O)c1ccc2c(c1)OCCCO2)c1nc2cccnc2s1 ; COc1ccccc1[C@@H](NS(=O)(=O)c1ccc2c(c1)OCCCO2)c1nc2cccnc2s1]

A 15 mL round-bottomed flask was charged with [1,3]thiazolo[5,4-b]pyridine (0.10 g, 0.73 mmol, Atlantic, Stratton, UK) and tetrahydrofuran (3.7 mL), the solution was cooled to −78° C., n-butyllithium (0.32 mL of a 2.5 M solution with toluene, 0.81 mmol, Sigma-Aldrich, St. Louis, Mo.) was added, and then the reaction mixture was stirred for 15 min. After that time, a solution of N-((2-methoxyphenyl)methylidene)-3,4-dihydro-2H-1,5-benzodioxepine-7-sulfonamide (intermediate K) (0.26 g, 0.73 mmol) and tetrahydrofuran (3.7 mL) was added. After stirring for 10 min, the reaction mixture was warmed to room temperature, saturated aqueous sodium bicarbonate (2.0 mL) was added, the mixture was partitioned between ethyl acetate (50 mL) and saturated aqueous sodium bicarbonate (50 mL), the layers were separated, the organic material was washed sequentially with saturated aqueous sodium bicarbonate (50 mL) and brine (50 mL), dried (sodium sulfate), filtered, the filtrate was concentrated, the residue was dissolved with dichloromethane (5.0 mL), silica gel (0.50 g) was added, and the volatiles were removed under vacuum. The residue was subjected to flash chromatography on silica gel (19 g of silica gel; 1:1 hexane-ethyl acetate) to give N-((2-methoxyphenyl)([1,3]thiazolo[5,4-b]pyridin-2-yl)methyl)-3,4-dihydro-2H-1,5-benzodioxepine-7-sulfonamide (0.081 g) as a pale yellow solid (racemic mixture).

¹H NMR (400 MHz, DMSO-d₆) δ 9.06 (d, J=7.4 Hz, 1H), 8.58 (dd, J=1.2, 4.7 Hz, 1H), 8.25 (d, J=8.2 Hz, 1H), 7.53 (dd, J=4.7, 8.2 Hz, 1H), 7.31-7.20 (m, 3H), 7.16 (d, J=2.2 Hz, 1H), 6.91 (d, J=8.4 Hz, 2H), 6.84 (t, J=7.4 Hz, 1H), 6.17 (d, J=6.8 Hz, 1H), 4.17-4.07 (m, 2H), 4.07-3.98 (m, 2H), 3.67 (s, 3H), 2.09 (quin, J=5.6 Hz, 2H). m/z (ESI, +ve ion) 483.8 (M+H)⁺. GK-GKRP EC₅₀ (LCMS/MS)=0.095 μM. GK-GKRP IC₅₀ (Binding)=0.058 μM.

The racemic mixture obtained was resolved using preparative SFC (Chiralcel® OD-H column (Sepax, Inc., Newark, Del.) (250 mm×20 mm, 5 μm) eluting with 70% liquid CO₂ in 30% methanol (with 20 mM NH₃) at a flow rate of 70 mL/min) to give two products in greater than 99% enantiomeric excess.

### N—((S)-(2-methoxyphenyl)([1,3]thiazolo[5,4-b]pyridin-2-yl)methyl)-3,4-dihydro-2H-1,5-benzodioxepine-7-sulfonamide

First Eluting Peak (Peak #1)

¹H NMR (400 MHz, chloroform-d) δ 8.51 (d, J=4.5 Hz, 1H), 8.12 (d, J=8.2 Hz, 1H), 7.36 (dd, J=4.7, 8.2 Hz, 1H), 7.33-7.28 (m, 2H), 7.28-7.21 (m, 1H), 7.18 (d, J=7.6 Hz, 1H), 6.87 (t, J=7.5 Hz, 1H), 6.80 (d, J=8.2 Hz, 2H), 6.42 (d, J=8.8 Hz, 1H), 6.01 (d, J=8.8 Hz, 1H), 4.17 (t, J=5.7 Hz, 2H), 4.11 (t, J=5.7 Hz, 2H), 3.74 (s, 3H), 2.16 (quin, J=5.7 Hz, 2H). m/z (ESI, +ve ion) 483.8 (M+H)⁺. GK-GKRP IC₅₀ (Binding)=2.8 μM.

### N—((R)-(2-methoxyphenyl)([1,3]thiazolo[5,4-b]pyridin-2-yl)methyl)-3,4-dihydro-2H-1,5-benzodioxepine-7-sulfonamide

Second Eluting Peak (Peak #2)

¹H NMR (400 MHz, chloroform-d) δ 8.51 (d, J=4.7 Hz, 1H), 8.12 (d, J=8.2 Hz, 1H), 7.36 (dd, J=4.7, 8.0 Hz, 1H), 7.33-7.28 (m, 2H), 7.28-7.21 (m, 1H), 7.18 (d, J=7.4 Hz, 1H), 6.87 (t, J=7.5 Hz, 1H), 6.80 (d, J=8.6 Hz, 2H), 6.42 (d, J=8.8 Hz, 1H), 6.01 (d, J=8.8 Hz, 1H), 4.17 (t, J=5.8 Hz, 2H), 4.11 (t, J=5.8 Hz, 2H), 3.74 (s, 3H), 2.16 (quin, J=5.8 Hz, 2H). m/z (ESI, +ve ion) 483.8 (M+H)⁺. GK-GKRP EC₅₀ (LCMS/MS)=0.024 μM. GK-GKRP IC₅₀ (Binding)=0.048 μM.

### Intermediate L

### N-((2-chlorophenyl)methylidene)-3,4-dihydro-2H-1,5-benzodioxepine-7-sulfonamide

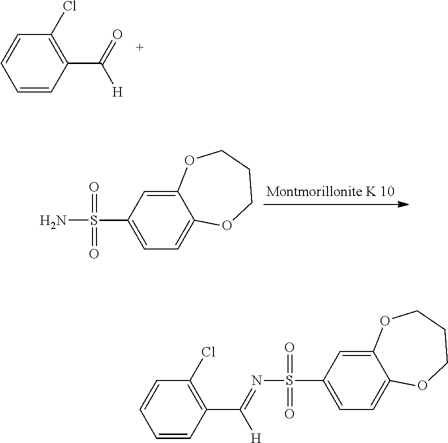 [SMILES: O=Cc1ccccc1Cl ; NS(=O)(=O)c1ccc2c(c1)OCCCO2 ; O=S(=O)(/N=C/c1ccccc1Cl)c1ccc2c(c1)OCCCO2]

A 250 mL round-bottomed flask was charged with 2-chlorobenzaldehyde (2.5 g, 17 mmol, Sigma-Aldrich, St. Louis, Mo.), 3,4-dihydro-2H-1,5-benzodioxepine-7-sulfonamide (4.0 g, 17 mmol, Enamine, Kiev, Ukraine), Montmorillonite K 10 (4.0 g, Sigma-Aldrich, St. Louis, Mo.), and toluene (87 mL), fitted with a Dean-Stark trap and a reflux condenser, and then the reaction mixture was heated at reflux for 19 h. After that time, the reaction mixture was filtered, the filtrate was allowed to cool to room temperature, filtered, and the filtrate was concentrated to give N-((2-chlorophenyl)methylidene)-3,4-dihydro-2H-1,5-benzodioxepine-7-sulfonamide (3.9 g) as an off-white solid.

### Example 68

### N-((2-chlorophenyl)([1,3]thiazolo[5,4-b]pyridin-2-yl)methyl)-3,4-dihydro-2H-1,5-benzodioxepine-7-sulfonamide

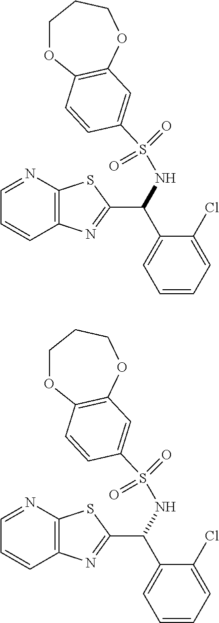 [SMILES: O=S(=O)(N[C@H](c1nc2cccnc2s1)c1ccccc1Cl)c1ccc2c(c1)OCCCO2 ; O=S(=O)(N[C@@H](c1nc2cccnc2s1)c1ccccc1Cl)c1ccc2c(c1)OCCCO2]

A 15 mL round-bottomed flask was charged with [1,3]thiazolo[5,4-b]pyridine (0.10 g, 0.73 mmol, Atlantic, Stratton, UK) and tetrahydrofuran (3.7 mL), the solution was cooled to −78° C., n-butyllithium (0.32 mL of a 2.5 M solution with toluene, 0.81 mmol, Sigma-Aldrich, St. Louis, Mo.) was added, and then the reaction mixture was stirred for 10 min. After that time, a solution of N-((2-chlorophenyl)methylidene)-3,4-dihydro-2H-1,5-benzodioxepine-7-sulfonamide (intermediate L) (0.26 g, 0.73 mmol) and tetrahydrofuran (3.7 mL) was added. After stirring for 10 min, the reaction mixture was warmed to room temperature, methanol (1.0 mL) and silica gel (0.50 g) were added sequentially, and the volatiles were removed under vacuum. The residue was subjected to flash chromatography on silica gel (19 g of silica gel; gradient elution of 3:1 to 2:1 to 1:1 hexane-ethyl acetate), and then the isolated material was re-subjected to flash chromatography on silica gel (10 g of silica gel; 99:1 dichloromethane-methanol) to give N-((2-chlorophenyl)([1,3]thiazolo[5,4-b]pyridin-2-yl)methyl)-3,4-dihydro-2H-1,5-benzodioxepine-7-sulfonamide (0.054 g) as an off-white solid (racemic mixture).

¹H NMR (400 MHz, DMSO-d₆) δ 9.46 (br. s., 1H), 8.60 (dd, J=1.6, 4.7 Hz, 1H), 8.29 (dd, J=1.6, 8.3 Hz, 1H), 7.56 (dd, J=4.7, 8.3 Hz, 1H), 7.47 (dd, J=2.0, 7.4 Hz, 1H), 7.39 (dd, J=1.6, 7.4 Hz, 1H), 7.35-7.20 (m, 3H), 7.16 (d, J=2.0 Hz, 1H), 6.91 (d, J=8.3 Hz, 1H), 6.28 (s, 1H), 4.17-4.09 (m, 2H), 4.05 (t, J=5.6 Hz, 2H), 2.10 (quin, J=5.6 Hz, 2H). m/z (ESI, +ve ion) 487.9 (M+H)⁺. GK-GKRP EC₅₀ (LCMS/MS)=0.063 μM. GK-GKRP IC₅₀ (Binding)=0.054 μM.

The racemic mixture was resolved using preparative SFC (Chiralpak® AS-H column (Daicel, Inc., Fort Lee, N.J.) (250 mm×21 mm, 5 μm) eluting with 60% liquid CO₂ in 40% isopropanol at a flow rate of 65 mL/min) to give two products in greater than 99% enantiomeric excess.

### N—((S)-(2-chlorophenyl)([1,3]thiazolo[5,4-b]pyridin-2-yl)methyl)-3,4-dihydro-2H-1,5-benzodioxepine-7-sulfonamide

First Eluting Peak (Peak #1)

¹H NMR (400 MHz, DMSO-d₆) δ 9.46 (d, J=9.2 Hz, 1H), 8.61 (d, J=3.7 Hz, 1H), 8.30 (d, J=8.2 Hz, 1H), 7.56 (dd, J=4.7, 8.0 Hz, 1H), 7.47 (d, J=6.8 Hz, 1H), 7.40 (d, J=7.4 Hz, 1H), 7.34-7.21 (m, 3H), 7.16 (br. s., 1H), 6.91 (d, J=8.2 Hz, 1H), 6.29 (d, J=9.2 Hz, 1H), 4.12 (m, 2H), 4.06 (m, 2H), 2.10 (m, 2H). m/z (ESI, +ve ion) 487.9 (M+H)⁺. GK-GKRP IC₅₀ (Binding)=2.0 μM.

### N—((R)-(2-chlorophenyl)([1,3]thiazolo[5,4-b]pyridin-2-yl)methyl)-3,4-dihydro-2H-1,5-benzodioxepine-7-sulfonamide

Second Eluting Peak (Peak #2)

Component 2: ¹H NMR (400 MHz, DMSO-d₆) δ 9.46 (d, J=9.2 Hz, 1H), 8.61 (d, J=3.3 Hz, 1H), 8.30 (d, J=8.2 Hz, 1H), 7.56 (dd, J=4.6, 7.7 Hz, 1H), 7.47 (d, J=6.8 Hz, 1H), 7.40 (d, J=7.4 Hz, 1H), 7.35-7.20 (m, 3H), 7.16 (br. s., 1H), 6.91 (d, J=8.2 Hz, 1H), 6.29 (d, J=9.0 Hz, 1H), 4.12 (m, 2H), 4.06 (m, 2H), 2.10 (m, 2H). m/z (ESI, +ve ion) 487.9 (M+H)⁺. GK-GKRP EC₅₀ (LCMS/MS)=0.029 μM. GK-GKRP IC₅₀ (Binding)=0.020 μM.

### Intermediate M

### N-((3-methoxy-2-pyridinyl)methylidene)-3,4-dihydro-2H-1,5-benzodioxepine-7-sulfonamide

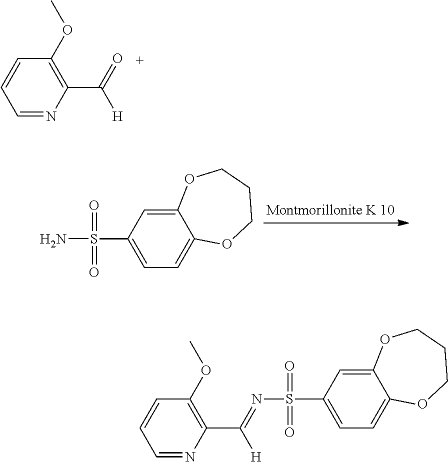

A 150 mL round-bottomed flask was charged with 3-methoxy-2-pyridinecarbaldehyde (1.8 g, 13 mmol, Chem-Impex, Wood Dale, Ill.), 3,4-dihydro-2H-1,5-benzodioxepine-7-sulfonamide (3.0 g, 13 mmol, Enamine, Kiev, Ukraine), Montmorillonite K 10 (3.0 g, Sigma-Aldrich, St. Louis, Mo.), and toluene (65 mL), fitted with a Dean-Stark trap and a reflux condenser, and then the reaction mixture was heated at reflux for 4 h. After that time, the reaction mixture was filtered, the filtrate was allowed to cool to room temperature, the mother liquor was decanted, and the remaining residue was collected to give N-((3-methoxy-2-pyridinyl)methylidene)-3,4-dihydro-2H-1,5-benzodioxepine-7-sulfonamide (0.73 g) as a yellow solid.

### Example 69

### N-((3-methoxy-2-pyridinyl)([1,3]thiazolo[5,4-b]pyridin-2-yl)methyl)-3,4-dihydro-2H-1,5-benzodioxepine-7-sulfonamide

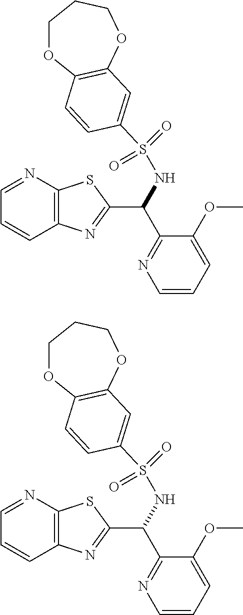 [SMILES: COc1cccnc1[C@H](NS(=O)(=O)c1ccc2c(c1)OCCCO2)c1nc2cccnc2s1 ; COc1cccnc1[C@@H](NS(=O)(=O)c1ccc2c(c1)OCCCO2)c1nc2cccnc2s1]

A 15 mL round-bottomed flask was charged with [1,3]thiazolo[5,4-b]pyridine (0.10 g, 0.73 mmol, Atlantic, Stratton, UK) and tetrahydrofuran (3.7 mL), the solution was cooled to −78° C., n-butyllithium (0.32 mL of a 2.5 M solution with toluene, 0.81 mmol, Sigma-Aldrich, St. Louis, Mo.) was added, and then the reaction mixture was stirred for 15 min. After that time, a solution of N-((3-methoxy-2-pyridinyl)methylidene)-3,4-dihydro-2H-1,5-benzodioxepine-7-sulfonamide (intermediate M) (0.26 g, 0.73 mmol) and tetrahydrofuran (3.7 mL) was added. After stirring for 6 h, methanol (1.0 mL) was added, the reaction mixture was warmed to room temperature, silica gel (0.50 g) was added, and the volatiles were removed under vacuum. The residue was subjected to flash chromatography on silica gel (19 g of silica gel; 99:1 dichloromethane-methanol), the isolated material was re-subjected to flash chromatography on silica gel (10 g of silica gel; 1:1.5 hexane-ethyl acetate), and then the isolated material was re-subjected to flash chromatography on silica gel (2.5 g of silica gel; 99:1 dichloromethane-methanol) to give N-((3-methoxy-2-pyridinyl)([1,3]thiazolo[5,4-b]pyridin-2-yl)methyl)-3,4-dihydro-2H-1,5-benzodioxepine-7-sulfonamide (0.00080 g) as an off-white solid (racemic mixture).

¹H NMR (400 MHz, chloroform-d) δ 8.51 (d, J=4.3 Hz, 1H), 8.16-8.07 (m, 2H), 7.39-7.31 (m, 3H), 7.31-7.12 (m, 3H), 6.75 (d, J=8.8 Hz, 1H), 6.46 (d, J=9.0 Hz, 1H), 4.22-3.98 (m, 4H), 3.87 (s, 3H), 2.14 (br. s., 2H). m/z (ESI, +ve ion) 484.9 (M+H)⁺. GK-GKRP IC₅₀ (Binding)=1.5 μM.

### Example 70

### N-(phenyl(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide

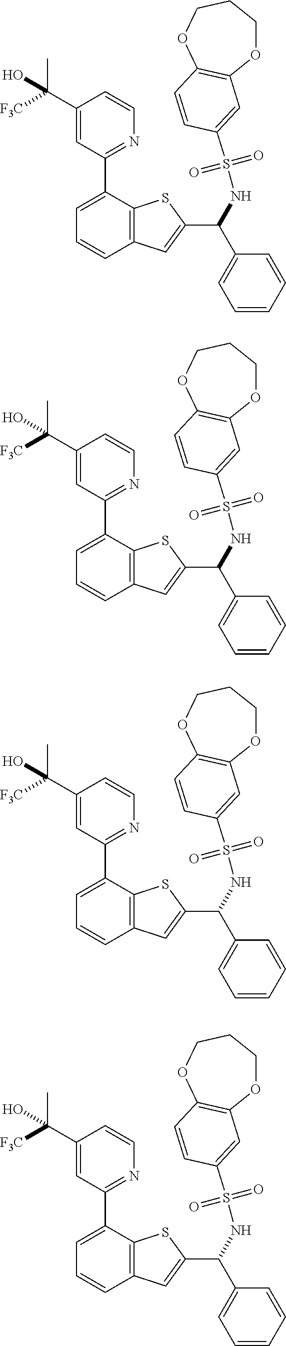

Step 1.

A 50 mL round-bottomed flask was charged with 4-bromo-2-chloropyridine (0.58 mL, 5.2 mmol, Sigma-Aldrich, St. Louis, Mo.) and tetrahydrofuran (26 mL). The solution was cooled to −78° C., n-butyllithium (3.3 mL of a 1.6 M solution with hexane, 5.2 mmol, Sigma-Aldrich, St. Louis, Mo.) was added, and then the reaction mixture was stirred for 10 min. After that time, 1,1,1-trifluoroacetone (0.93 mL, 9.9 mmol, Sigma-Aldrich, St. Louis, Mo.) was added. After stirring for 20 min, saturated aqueous sodium bicarbonate (5.0 mL) was added, the mixture was warmed to room temperature and concentrated under a vacuum. The residue was partitioned between ethyl acetate (100 mL) and saturated aqueous sodium bicarbonate (50 mL), the layers were separated. The organic material was washed with brine (50 mL), dried (magnesium sulfate), filtered, and the filtrate was concentrated under vacuum. The isolated material (0.75 g) was used in the next step of the synthesis without purification.

Step 2.

A microwave vial was charged with a portion of the isolated material (0.20 g) from Step 1, 1-benzothiophen-7-ylboronic acid (0.16 g, 0.89 mmol, Focus Synthesis, San Diego, Calif.), [1,1-bis(diphenylphosphino)ferrocene]palladium(II) chloride 1:1 complex with dichloromethane (0.072 g, 0.089 mmol, Sigma-Aldrich, St. Louis, Mo.), cesium carbonate (0.87 g, 2.7 mmol, Alfa Aesar, Ward Hill, Mass.), tetrahydrofuran (3.0 mL), and water (1.0 mL). Then the reaction mixture was stirred and heated 100° C. (100 W) in a microwave reactor (Personal Chemistry Initiator model, Biotage AB, Upssala, Sweden) for 30 min. After that time, silica gel (2.0 g) was added, the volatiles were removed under vacuum, and the residue was subjected to flash chromatography on silica gel (Isco CombiFlash® system, 40 g RediSep® normal phase column, gradient elution of 0% to 20% ethyl acetate-hexane, Teledyne Isco, Lincoln, Nebr.) to give 2-(2-(benzo[b]thiophen-7-yl)pyridin-4-yl)-1,1,1-trifluoropropan-2-ol (0.12 g) as a colorless oil.

Step 3.

A 25 mL round-bottomed flask was charged with 2-(2-(1-benzothiophen-7-yl)-4-pyridinyl)-1,1,1-trifluoro-2-propanol (from step 2) (0.12 g, 0.37 mmol) and tetrahydrofuran (4.0 mL), the solution was cooled to −78° C., n-butyllithium (0.46 mL of a 1.6 M solution with hexane, 0.74 mmol, Sigma-Aldrich, St. Louis, Mo.) was added, and then the reaction mixture was stirred for 15 min. After that time, a solution of N-(phenylmethylidene)-3,4-dihydro-2H-1,5-benzodioxepine-7-sulfonamide (intermediate I) (0.12 g, 0.37 mmol) and tetrahydrofuran (2.0 mL) was added. After stirring for an additional for 5 min, saturated aqueous ammonium chloride (1.0 mL) was added, the mixture was warmed to room temperature. Silica gel (1.0 g) was added, and the volatiles were removed under vacuum. The residue was subjected to flash chromatography on silica gel (Isco CombiFlash® system, 40 g RediSep® normal phase column, gradient elution of 0% to 40% ethyl acetate-hexane, Teledyne Isco, Lincoln, Nebr.) to afford N-(phenyl(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide (0.13 g) as a white solid (mixture of four stereoisomers).

¹H NMR (400 MHz, methanol-d₄) δ 8.73 (d, J=5.3 Hz, 1H), 8.23 (s, 1H), 7.86 (d, J=7.4 Hz, 1H), 7.70 (d, J=7.6 Hz, 1H), 7.56 (d, J=5.3 Hz, 1H), 7.49-7.40 (m, 1H), 7.35-7.19 (m, 6H), 7.17 (d, J=2.3 Hz, 1H), 6.94 (s, 1H), 6.76 (d, J=8.4 Hz, 1H), 5.86 (s, 1H), 3.92 (t, J=5.5 Hz, 2H), 3.79 (t, J=5.7 Hz, 2H), 2.00-1.92 (m, 2H), 1.80 (s, 3H). m/z (ESI, +ve ion) 640.8 (M+H)⁺. GK-GKRP EC₅₀ (LCMS/MS)=0.026 μM. GK-GKRP IC₅₀ (Binding)=0.021 μM.

The resulting mixture of four stereoisomers was resolved using preparative SFC (Chiralpak® AD-H column (Daicel, Inc., Fort Lee, N.J.) (250 mm×21 mm, 5 μm) eluting with 50% liquid CO₂ in 50% isopropanol (with 20 mM NH₃) at a flow rate of 50 mL/min) to give four products in greater than 99% enantiomeric excess.

### N—((R)-Phenyl(7-(4-((R)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide

First Eluting Peak (Peak #1)

¹H NMR (300 MHz, acetonitrile-d₃) δ 8.76 (d, J=5.3 Hz, 1H), 8.21 (s, 1H), 7.98 (d, J=7.6 Hz, 1H), 7.76 (d, J=7.9 Hz, 1H), 7.59-7.42 (m, 2H), 7.35-7.23 (m, 6H), 7.18 (d, J=2.3 Hz, 1H), 7.03-6.93 (m, 1H), 6.84 (d, J=8.5 Hz, 1H), 6.66 (d, J=9.1 Hz, 1H), 5.89 (d, J=9.1 Hz, 1H), 4.67 (s, 1H), 4.01 (t, J=5.6 Hz, 2H), 3.90 (t, J=5.7 Hz, 2H), 2.04 (quin, J=5.7 Hz, 2H), 1.81 (s, 3H). m/z (ESI, +ve ion) 640.8 (M+H)⁺. GK-GKRP EC₅₀ (LCMS/MS)=0.017 μM. GK-GKRP IC₅₀ (Binding)=0.0070 μM.

### N—((R)-Phenyl(7-(4-((S)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide

Second Eluting Peak (Peak #2)

¹H NMR (300 MHz, acetonitrile-d₃) δ 8.76 (d, J=5.3 Hz, 1H), 8.20 (s, 1H), 7.98 (d, J=7.6 Hz, 1H), 7.76 (d, J=7.7 Hz, 1H), 7.59-7.42 (m, 2H), 7.37-7.23 (m, 6H), 7.18 (d, J=2.2 Hz, 1H), 6.99 (s, 1H), 6.84 (d, J=8.3 Hz, 1H), 6.66 (d, J=9.2 Hz, 1H), 5.89 (d, J=9.1 Hz, 1H), 4.67 (s, 1H), 4.01 (t, J=5.6 Hz, 2H), 3.90 (t, J=5.7 Hz, 2H), 2.03 (quin, J=5.7 Hz, 2H), 1.81 (s, 3H). m/z (ESI, +ve ion) 640.8 (M+H)⁺. GK-GKRP EC₅₀ (LCMS/MS)=0.022 μM. GK-GKRP IC₅₀ (Binding)=0.0060 μM.

### N—((S)-Phenyl(7-(4-((R)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide

Third Eluting Peak (Peak #3)

¹H NMR (300 MHz, acetonitrile-d₃) δ 8.76 (d, J=5.1 Hz, 1H), 8.21 (s, 1H), 7.98 (d, J=6.9 Hz, 1H), 7.76 (d, J=7.3 Hz, 1H), 7.54 (d, J=4.8 Hz, 1H), 7.52-7.43 (m, 1H), 7.37-7.23 (m, 6H), 7.18 (d, J=2.2 Hz, 1H), 7.01-6.96 (m, 1H), 6.84 (d, J=8.5 Hz, 1H), 6.66 (d, J=9.1 Hz, 1H), 5.89 (d, J=8.9 Hz, 1H), 4.67 (s, 1H), 4.01 (t, J=5.6 Hz, 2H), 3.90 (t, J=5.7 Hz, 2H), 2.04 (quin, J=5.7 Hz, 2H), 1.81 (s, 3H). m/z (ESI, +ve ion) 640.8 (M+H)⁺. GK-GKRP EC₅₀ (LCMS/MS)=0.17 μM. GK-GKRP IC₅₀ (Binding)=0.33 μM.

### N—((S)-Phenyl(7-(4-((S)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide

Fourth Eluting Peak (Peak #4)

¹H NMR (300 MHz, acetonitrile-d₃) δ 8.76 (d, J=5.3 Hz, 1H), 8.21 (s, 1H), 7.98 (d, J=7.5 Hz, 1H), 7.76 (d, J=7.7 Hz, 1H), 7.58-7.41 (m, 2H), 7.36-7.23 (m, 6H), 7.18 (d, J=2.3 Hz, 1H), 6.99 (s, 1H), 6.84 (d, J=8.5 Hz, 1H), 6.66 (d, J=8.9 Hz, 1H), 5.89 (d, J=8.8 Hz, 1H), 4.68 (s, 1H), 4.01 (t, J=5.6 Hz, 2H), 3.91 (t, J=5.7 Hz, 2H), 2.04 (quin, J=5.6 Hz, 2H), 1.81 (s, 3H). m/z (ESI, +ve ion) 640.8 (M+H)⁺. GK-GKRP EC₅₀ (LCMS/MS)=0.074 μM. GK-GKRP IC₅₀ (Binding)=0.11 μM.

### Example 71

### N-(phenyl(7-(2-pyridinyl)-1-benzothiophen-2-yl)methyl)-3,4-dihydro-2H-1,5-benzodioxepine-7-sulfonamide

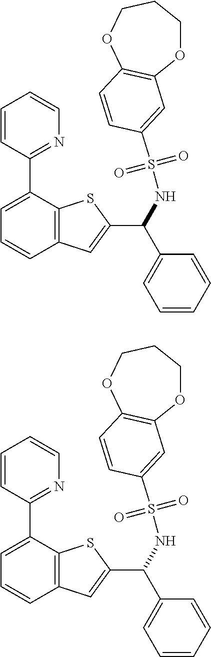

Step 1.

A microwave vial was charged with 2-(1-benzothiophen-7-yl)-4,4,5,5-tetramethyl-1,3,2-dioxaborolane (0.30 g, 1.2 mmol, See Wang, B.; Akay, S.; Yang, W.; Wang, J. Chem. Biol. Drug Des. 2007, 70, 279-289), 2-bromopyridine (0.11 mL, 1.2 mmol, Alfa Aesar, Ward Hill, Mass.), [1,1-bis(diphenylphosphino)ferrocene]palladium(II) chloride 1:1 complex with dichloromethane (0.047 g, 0.058 mmol, Sigma-Aldrich, St. Louis, Mo.), cesium carbonate (1.1 g, 3.5 mmol, Alfa Aesar, Ward Hill, Mass.), tetrahydrofuran (3.0 mL), and water (1.0 mL). Then the reaction mixture was stirred and heated 100° C. (100 W) in a microwave reactor (Personal Chemistry Initiator model, Biotage AB, Upssala, Sweden) for 30 min. After that time, the reaction mixture was partitioned between dichloromethane (100 mL) and water (100 mL), the layers were separated. The organic material was washed with brine (50 mL), dried (magnesium sulfate) and filtered. Silica gel (1.0 g) was added to the filtrate, the volatiles were removed under a vacuum. The residue was subjected to flash chromatography on silica gel (Isco CombiFlash® system, 40 g RediSep® normal phase column, gradient elution of 0% to 20% ethyl acetate-hexane, Teledyne Isco, Lincoln, Nebr.) to afford 2-(1-benzothiophen-7-yl)pyridine (0.18 g) as a colorless oil.

Step 2.

A 25 mL round-bottomed flask was charged with 2-(1-benzothiophen-7-yl)pyridine (from step 1) (0.12 g, 0.57 mmol) and tetrahydrofuran (3.0 mL), the solution was cooled to −78° C., n-butyllithium (0.31 mL of a 1.6 M solution with hexane, 0.49 mmol, Sigma-Aldrich, St. Louis, Mo.) was added, and then the reaction mixture was stirred for 30 min. After that time, a solution of N-(phenylmethylidene)-3,4-dihydro-2H-1,5-benzodioxepine-7-sulfonamide (intermediate I) (0.13 g, 0.41 mmol) and tetrahydrofuran (3.0 mL) was added. After stirring for an additional 5 min, saturated aqueous ammonium chloride (5.0 mL) was added, the mixture was warmed to room temperature and partitioned between ethyl acetate (50 mL) and saturated aqueous sodium bicarbonate (50 mL). The layers were separated, the organic material was washed with brine (50 mL), dried (magnesium sulfate) and filtered. Silica gel (1.0 g) was added to the filtrate, the volatiles were removed under vacuum, and the residue was subjected to flash chromatography on silica gel (Isco CombiFlash® system, 40 g RediSep® normal phase column, gradient elution of 20% to 40% ethyl acetate-hexane, Teledyne Isco, Lincoln, Nebr.) to afford N-(phenyl(7-(2-pyridinyl)-1-benzothiophen-2-yl)methyl)-3,4-dihydro-2H-1,5-benzodioxepine-7-sulfonamide (0.13 g) as white solid (racemic mixture).

¹H NMR (400 MHz, DMSO-d₆) δ 8.97 (d, J=9.6 Hz, 1H), 8.77 (d, J=4.3 Hz, 1H), 8.18 (d, J=8.2 Hz, 1H), 8.03 (d, J=7.6 Hz, 1H), 7.99-7.91 (m, 1H), 7.78 (d, J=7.6 Hz, 1H), 7.53-7.37 (m, 2H), 7.36-7.17 (m, 6H), 7.11 (d, J=2.4 Hz, 1H), 6.98 (s, 1H), 6.84 (d, J=8.4 Hz, 1H), 5.84 (d, J=9.6 Hz, 1H), 3.98 (t, J=5.5 Hz, 2H), 3.87 (t, J=5.6 Hz, 2H), 1.99 (quin, J=5.5 Hz, 2H). m/z (ESI, +ve ion) 529.0 (M+H)⁺. GK-GKRP EC₅₀ (LCMS/MS)=0.29 μM. GK-GKRP IC₅₀ (Binding)=0.54 μM.

The racemic mixture was resolved using preparative SFC (Chiralpak® AS-H column (Daicel, Inc., Fort Lee, N.J.) (250 mm×21 mm, 5 μm) eluting with 60% liquid CO₂ in 40% methanol (with 40 mM ammonia) at a flow rate of 70 mL/min) to give two products in greater than 98% enantiomeric excess.

### N—((S)-phenyl(7-(2-pyridinyl)-1-benzothiophen-2-yl)methyl)-3,4-dihydro-2H-1,5-benzodioxepine-7-sulfonamide

First Eluting Peak (Peak #1)

¹H NMR (400 MHz, DMSO-d₆) δ 8.97 (d, J=9.8 Hz, 1H), 8.77 (d, J=4.5 Hz, 1H), 8.18 (d, J=8.2 Hz, 1H), 8.03 (d, J=7.6 Hz, 1H), 7.95 (t, J=7.7 Hz, 1H), 7.78 (d, J=7.8 Hz, 1H), 7.52-7.37 (m, 2H), 7.35-7.17 (m, 6H), 7.11 (d, J=2.2 Hz, 1H), 6.98 (s, 1H), 6.84 (d, J=8.4 Hz, 1H), 5.84 (d, J=9.6 Hz, 1H), 3.98 (t, J=5.5 Hz, 2H), 3.87 (t, J=5.6 Hz, 2H), 1.99 (quin, J=5.4 Hz, 2H). m/z (ESI, +ve ion) 529.0 (M+H)⁺. GK-GKRP IC₅₀ (Binding)=0.79 μM.

### N—((R)-phenyl(7-(2-pyridinyl)-1-benzothiophen-2-yl)methyl)-3,4-dihydro-2H-1,5-benzodioxepine-7-sulfonamide

Second Eluting Peak (Peak #2)

¹H NMR (400 MHz, DMSO-d₆) δ 8.96 (d, J=8.0 Hz, 1H), 8.77 (d, J=4.3 Hz, 1H), 8.18 (d, J=8.2 Hz, 1H), 8.03 (d, J=7.4 Hz, 1H), 7.99-7.91 (m, 1H), 7.78 (d, J=7.8 Hz, 1H), 7.52-7.37 (m, 2H), 7.36-7.17 (m, 6H), 7.11 (d, J=2.0 Hz, 1H), 6.98 (s, 1H), 6.84 (d, J=8.4 Hz, 1H), 5.84 (d, J=7.0 Hz, 1H), 3.98 (t, J=5.4 Hz, 2H), 3.87 (t, J=5.6 Hz, 2H), 1.99 (quin, J=5.4 Hz, 2H). m/z (ESI, +ve ion) 529.0 (M+H)⁺. GK-GKRP EC₅₀ (LCMS/MS)=0.12 μM. GK-GKRP IC₅₀ (Binding)=0.35 μM.

### Intermediate N

### N-((7-chloro-1-benzothiophen-2-yl)methylidene)-3,4-dihydro-2H-1,5-benzodioxepine-7-sulfonamide

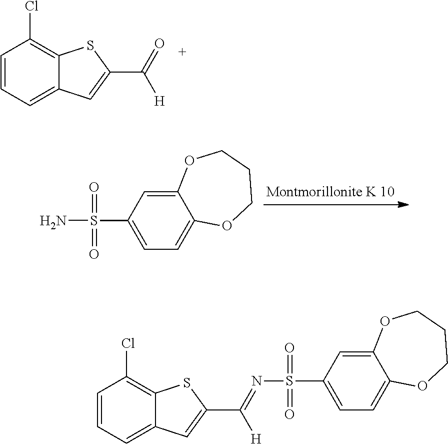 [SMILES: O=Cc1cc2cccc(Cl)c2s1 ; NS(=O)(=O)c1ccc2c(c1)OCCCO2 ; O=S(=O)(/N=C/c1cc2cccc(Cl)c2s1)c1ccc2c(c1)OCCCO2]

A 100 mL round-bottomed flask was charged with 3,4-dihydro-2H-1,5-benzodioxepine-7-sulfonamide (1.2 g, 5.1 mmol, Enamine, Kiev, Ukraine), 7-chloro-1-benzothiophene-2-carbaldehyde (1.0 g, 5.1 mmol, Aurigene, Bangalore, India), toluene (25 mL), and Montmorillonite K 10 (1.0 g, Sigma-Aldrich, St. Louis, Mo.). The reaction was fitted with a Dean-Stark trap and a reflux condenser, and then the reaction mixture was heated at reflux for 4 h. After that time, the reaction mixture was cooled to room temperature, filtered, the filtrate cooled to −20° C. for 15 h, the mixture was filtered, and the filter cake was collected to afford N-((7-chloro-1-benzothiophen-2-yl)methylidene)-3,4-dihydro-2H-1,5-benzodioxepine-7-sulfonamide (1.4 g) as a light yellow solid.

### Example 72

### N-((7-chloro-1-benzothiophen-2-yl)(phenyl)methyl)-3,4-dihydro-2H-1,5-benzodioxepine-7-sulfonamide

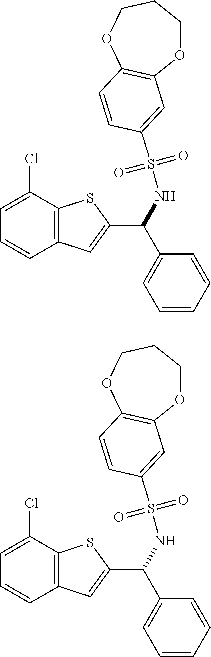

A 25 mL round-bottomed flask was charged with N-((7-chloro-1-benzothiophen-2-yl)methylidene)-3,4-dihydro-2H-1,5-benzodioxepine-7-sulfonamide (intermediate N) (0.50 g, 1.2 mmol), tetrahydrofuran (6.0 mL), bromo(phenyl)magnesium (1.4 mL of a 1.0 M solution with tetrahydrofuran, 1.3 mmol, Sigma-Aldrich, St. Louis, Mo.). The reaction mixture was stirred at room temperature for 1 h. After that time, the reaction mixture was added to saturated aqueous sodium bicarbonate (50 mL). After stirring vigorously for 15 h, ethyl acetate (30 mL) was added, the layers were separated, the aqueous material was washed with ethyl acetate (2×30 mL). The combined organic extract was washed with brine (50 mL), dried (magnesium sulfate) and filtered. Silica gel (1.0 g) was added to the filtrate, and the volatiles were removed under vacuum. The residue was subjected to flash chromatography on silica gel (Isco CombiFlash® system, 40 g RediSep® normal phase column, gradient elution of 0% to 30% ethyl acetate-hexane, Teledyne Isco, Lincoln, Nebr.) to afford N-((7-chloro-1-benzothiophen-2-yl)(phenyl)methyl)-3,4-dihydro-2H-1,5-benzodioxepine-7-sulfonamide (0.46 g) as a colorless solid.

¹H NMR (400 MHz, methanol-d₄) δ 7.58 (m, 1H), 7.31-7.25 (m, 8H), 7.17 (m, 1H), 6.96 (br. s., 1H), 6.81 (d, J=7.8 Hz, 1H), 5.86 (br. s., 1H), 4.06 (m, 2H), 3.96 (m, 2H), 2.09 (m, 2H). m/z (ESI, +ve ion) 507.9 (M+Na)⁺. GK-GKRP EC₅₀ (LCMS/MS)=0.48 μM. GK-GKRP IC₅₀ (Binding)=1.0 μM.

### Example 73

### N-(phenyl(7-phenyl-1-benzothiophen-2-yl)methyl)-3,4-dihydro-2H-1,5-benzodioxepine-7-sulfonamide

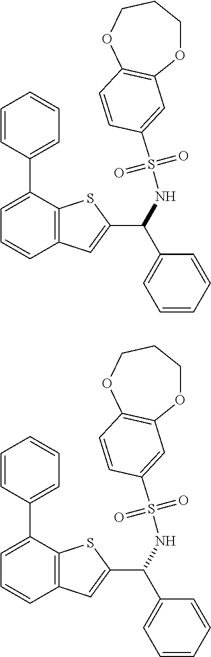

A 10 mL round-bottomed flask was charged with N-((7-chloro-1-benzothiophen-2-yl)(phenyl)methyl)-3,4-dihydro-2H-1,5-benzodioxepine-7-sulfonamide (Example 72) (0.93 g, 1.9 mmol), phenylboronic acid (0.44 g, 3.6 mmol, Sigma-Aldrich, St. Louis, Mo.), 1,4-dioxane (9.6 mL), 1,3-bis(2,4,6-trimethylphenyl)imidazolium chloride (0.13 g, 0.38 mmol), tris(dibenzylideneacetone)dipalladium(0) (0.18 g, 0.19 mmol, Strem, Newburyport, Mass.), cesium carbonate (1.5 g, 4.6 mmol, Alfa Aesar, Ward Hill, Mass.). The reaction was fitted with a reflux condenser, and then heated at reflux for 15 h. After that time, the reaction mixture was partitioned between ethyl acetate (100 mL) and water (100 mL) and the layers were separated. The organic material was washed with brine (50 mL), dried (magnesium sulfate) and filtered. Silica gel (1.0 g) was added to the filtrate, and the volatiles were removed under vacuum. The residue was subjected to flash chromatography on silica gel (Isco CombiFlash® system, 120 g RediSep® normal phase column, gradient elution of 0% to 40% ethyl acetate-hexane, Teledyne Isco, Lincoln, Nebr.) to afford N-(phenyl(7-phenyl-1-benzothiophen-2-yl)methyl)-3,4-dihydro-2H-1,5-benzodioxepine-7-sulfonamide (0.0053 g) as a colorless solid (racemic mixture).

¹H NMR (400 MHz, methanol-d₄) δ 7.61 (t, J=7.2 Hz, 3H), 7.47 (t, J=7.5 Hz, 2H), 7.43-7.35 (m, 2H), 7.33-7.21 (m, 7H), 7.16 (d, J=2.2 Hz, 1H), 6.95 (s, 1H), 6.81 (d, J=8.4 Hz, 1H), 5.84 (s, 1H), 4.05-3.92 (m, 2H), 3.92-3.78 (m, 2H), 2.08-1.98 (m, 2H), m/z (ESI, +ve ion) 550.0 (M+Na)⁺. GK-GKRP EC₅₀ (LCMS/MS)=1.0 μM. GK-GKRP IC₅₀ (Binding)=1.2 μM.

### Intermediate O

### 1-(1-benzothiophen-2-yl)-1-(2-(methylsulfanyl)phenyl)methanamine

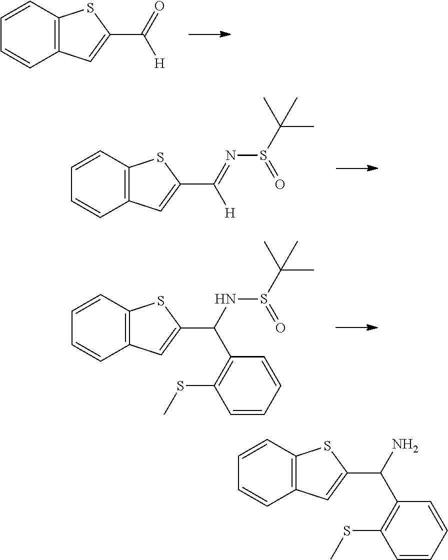

Step 1.

A 250 mL round-bottomed flask was charged with 1-benzothiophene-2-carbaldehyde (4.1 g, 25 mmol, Maybridge, Tintagel, United Kingdom), 2-methylpropane-2-sulfinamide (3.7 g, 31 mmol, AK Scientific, Mountain View, Calif.), copper(II) sulfate (3.8 mL, 76 mmol, Sigma-Aldrich, St. Louis, Mo.), and dichloromethane (51 mL). The reaction mixture was stirred for 15 h at room temperature. After that time, volatiles were removed under vacuum. Toluene (100 mL) was added to the resulting residue, the reaction vessel was fitted with a Dean-Stark trap and a reflux condenser, and then the reaction mixture was heated at reflux for 2 h. After that time, the mixture was cooled to room temperature, and the suspension was filtered. Silica gel (3.0 g) was added to the filtrate, and the volatiles were removed under vacuum. The residue was subjected to flash chromatography on silica gel (Isco CombiFlash® system, 80 g RediSep® normal phase column, gradient elution of 0% to 10% ethyl acetate-hexane, Teledyne Isco, Lincoln, Nebr.) to afford N-(1-benzothiophen-2-ylmethylidene)-2-methyl-2-propanesulfinamide (3.3 g) as light-yellow solid.

Step 2.

A 150 mL round-bottomed flask was charged with N-(1-benzothiophen-2-ylmethylidene)-2-methyl-2-propanesulfinamide (from step 1) (2.5 g, 9.4 mmol), tetrahydrofuran (19 mL), (2-(methylthio)phenyl)magnesium bromide (38 ml of a 0.50 M solution with tetrahydrofuran, 19 mmol, Rieke Metals, Lincoln, Nebr.). The reaction mixture was stirred for 96 h at room temperature. After that time, the reaction mixture was added to saturated aqueous sodium bicarbonate (100 mL), and then the volatiles were removed under a vacuum. The aqueous material was extracted with ethyl acetate (3×100 mL), the combined organic extract was washed with brine (50 mL), dried (magnesium sulfate) and filtered. Silica gel (5.0 g) was added to the filtrate, and the volatiles were removed under vacuum. The residue was subjected to flash chromatography on silica gel (Isco CombiFlash® system, 120 g RediSep® normal phase column, gradient elution of 0% to 70% ethyl acetate-hexane, Teledyne Isco, Lincoln, Nebr.) to afford N-(1-benzothiophen-2-yl(2-(methylsulfanyl)phenyl)methyl)-2-methyl-2-propanesulfinamide (2.1 g) as a yellow oil.

Step 3.

A 250 mL round-bottomed flask was charged with N-(1-benzothiophen-2-yl(2-(methylsulfanyl)phenyl)methyl)-2-methyl-2-propanesulfinamide (from step 2) (2.1 g, 6.4 mmol), diethyl ether (32 mL), hydrogen chloride, (13 mL of a 1.0 M with diethyl ether, 13 mmol, Sigma-Aldrich, St. Louis, Mo.), and then the reaction mixture was stirred for 3 h at room temperature. After that time, the volatiles were removed under vacuum, and the residue was partitioned between ethyl acetate (200 mL) and saturated aqueous sodium bicarbonate (100 mL). The layers were separated, the organic material was washed with brine (100 mL), dried (magnesium sulfate), filtered, and the filtrate was concentrated under vacuum to afford 1-(1-benzothiophen-2-yl)-1-(2-(methylsulfanyl)phenyl)methanamine (1.3 g) as a dark orange oil.

### Example 74

### N-(1-benzothiophen-2-yl(2-(methylsulfanyl)phenyl)methyl)-3,4-dihydro-2H-1,5-benzodioxepine-7-sulfonamide

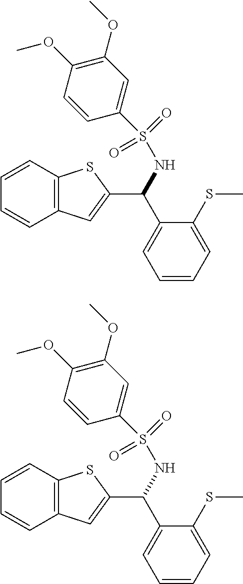

A 10 mL round-bottomed flask was charged 1-(1-benzothiophen-2-yl)-1-(2-(methylsulfanyl)phenyl)methanamine (intermediate O) (0.050 g, 0.18 mmol), N-ethyl-N-(1-methylethyl)-2-propanamine (0.091 mL, 0.53 mmol, EMD, Gibbstown, N.J.), 3,4-dimethoxybenzenesulfonyl chloride (0.037 g, 0.16 mmol, Sigma-Aldrich, St. Louis, Mo.), dichloromethane (5.0 mL). The reaction mixture was stirred and heated at reflux for 48 h. After that time, the reaction mixture was cooled to room temperature, silica gel (1.0 g) was added, and the volatiles were removed under vacuum. The residue was subjected to flash chromatography on silica gel (Isco CombiFlash® system, 40 g RediSep® normal phase column, gradient elution of 20% to 40% ethyl acetate-hexane, Teledyne Isco, Lincoln, Nebr.) to afford N-(1-benzothiophen-2-yl(2-(methylsulfanyl)phenyl)methyl)-3,4-dihydro-2H-1,5-benzodioxepine-7-sulfonamide (0.025 g) as an off-white solid (racemic mixture).

¹H NMR (400 MHz, acetonitrile-d₃) δ 7.82-7.74 (m, 1H), 7.65-7.58 (m, 1H), 7.41-7.22 (m, 6H), 7.18-7.09 (m, 2H), 6.87 (d, J=8.6 Hz, 1H), 6.78 (s, 1H), 6.72 (d, J=8.6 Hz, 1H), 6.24 (d, J=8.4 Hz, 1H), 3.81 (s, 3H), 3.71 (s, 3H), 2.39 (s, 3H). m/z (ESI, +ve ion) 507.9 (M+Na)⁺. GK-GKRP IC₅₀ (Binding)=2.7 μM.

### Example 75

### N-(1-benzothiophen-2-yl(2-(methylsulfanyl)phenyl)methyl)-2,3-dihydro-1,4-benzodioxine-6-sulfonamide

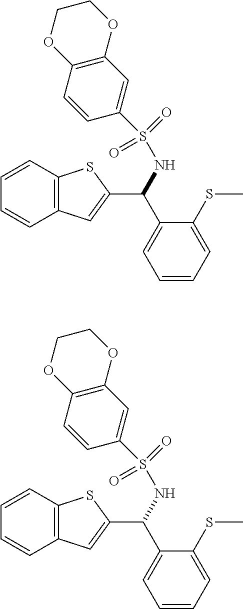

A 10 mL round-bottomed flask was charged with 1-(1-benzothiophen-2-yl)-1-(2-(methylsulfanyl)phenyl)methanamine (intermediate O) (0.10 g, 0.35 mmol), N-ethyl-N-(1-methylethyl)-2-propanamine (0.12 mL, 0.70 mmol, EMD, Gibbstown, N.J.), 2,3-dihydro-1,4-benzodioxine-6-sulfonyl chloride (0.074 g, 0.32 mmol, Maybridge, Tintagel, United Kingdom), 1,2-dichloroethane (10 mL). The reaction flask was equipped with a reflux condenser, and then the reaction mixture was stirred and heated at reflux for 48 h. After that time, the reaction mixture was cooled to room temperature, silica gel (1.0 g) was added, and the volatiles were removed under vacuum. The residue was subjected to flash chromatography on silica gel (Isco CombiFlash® system, 40 g RediSep® normal phase column, gradient elution of 20% to 40% ethyl acetate-hexane, Teledyne Isco, Lincoln, Nebr.) to afford N-(1-benzothiophen-2-yl(2-(methylsulfanyl)phenyl)methyl)-2,3-dihydro-1,4-benzodioxine-6-sulfonamide (0.027 g) as a yellow solid (racemic mixture).

¹H NMR (400 MHz, acetonitrile-d₃) δ 7.83-7.75 (m, 1H), 7.67-7.60 (m, 1H), 7.37-7.24 (m, 5H), 7.20 (dd, J=2.2, 8.5 Hz, 1H), 7.17-7.09 (m, 2H), 6.83-6.77 (m, 2H), 6.73 (d, J=7.0 Hz, 1H), 6.26 (d, J=7.4 Hz, 1H), 4.26-4.20 (m, 2H), 4.20-4.13 (m, 2H), 2.42 (s, 3H). m/z (ESI, +ve ion) 505.9 (M+Na)⁺. GK-GKRP EC₅₀ (LCMS/MS)=0.34 μM. GK-GKRP IC₅₀ (Binding)=0.95 μM.

### Example 76

### N—((R)-1-Benzothiophen-2-yl(2-chlorophenyl)methyl)-3,3-difluoro-3,4-dihydro-2H-1,5-benzodioxepine-7-sulfonamide

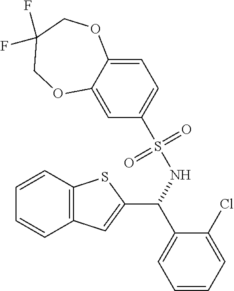

Step 1.

A 250 mL round-bottomed flask was charged with ((7-bromo-3,4-dihydro-2H-1,5-benzodioxepin-3-yl)oxy)(tris(1-methylethyl))silane (Example 35, Step 2) (6.6 g, 16 mmol), tetrahydrofuran (160 mL), and tetrabutylammonium fluoride (18 mL of a 1.0 M solution with tetrahydrofuran, 18 mmol, Sigma-Aldrich, St. Louis, Mo.), and then the reaction mixture was stirred for 2 h. After that time, the reaction mixture was concentrated under a vacuum, the residue was partitioned between ethyl acetate (200 mL) and saturated aqueous sodium bicarbonate (200 mL), the layers were separated, the organic material was washed sequentially with saturated aqueous sodium bicarbonate (200 mL) and brine (2×200 mL), dried (sodium sulfate), filtered, the filtrate was concentrated under a vacuum, the residue was dissolved with dichloromethane (100 mL), silica gel (12 g) was added, and the volatiles were removed under a vacuum. The residue was subjected to flash chromatography on silica gel (200 g of silica gel, 4:1 hexane-ethyl acetate). The isolated material was partitioned between ethyl acetate (100 mL) and saturated aqueous sodium bicarbonate (100 mL), the layers were separated, the organic material was washed sequentially with saturated aqueous sodium bicarbonate (2×100 mL) and brine (2×100 mL), dried (sodium sulfate), filtered, silica gel (20 g) was added to the filtrate, and the volatiles were removed under a vacuum. The residue was subjected to flash chromatography on silica gel (200 g of silica gel, 4:1 hexane-ethyl acetate) to give 7-bromo-3,4-dihydro-2H-1,5-benzodioxepin-3-ol (2.2 g) as a colorless solid.

Step 2.

A 150 mL round-bottomed flask was charged with 7-bromo-3,4-dihydro-2H-1,5-benzodioxepin-3-ol (from Step 1) (1.0 g, 4.1 mmol), dichloromethane (41 mL), and Dess-Martin periodinane (1.9 g, 4.5 mmol, Sigma-Aldrich, St. Louis, Mo.), and then the reaction mixture was stirred for 2 h. After that time, silica gel (5.0 g) was added to the reaction mixture and the volatiles were removed under a vacuum. The residue was subjected to flash chromatography on silica gel (50 g of silica gel, 2:1 hexane-ethyl acetate) to give 7-bromo-2H-1,5-benzodioxepin-3(4H)-one (0.86 g) as a colorless solid.

Step 3.

A 15 mL round-bottomed flask was charged with 7-bromo-2H-1,5-benzodioxepin-3(4H)-one (from Step 2) (0.20 g, 0.82 mmol), dichloromethane (4.1 mL), ethanol (9.6 μL, 0.17 mmol, Pharmaco-Aaper, Brookfield, Conn.), and (diethylamino)sulfur trifluoride (0.54 mL, 4.1 mmol, Sigma-Aldrich, St. Louis, Mo.), and then the reaction mixture was stirred for 22 h. After that time, the reaction mixture was added to saturated aqueous sodium bicarbonate (50 mL), the mixture was stirred vigorously for 1 h, ethyl acetate (50 mL) was added, the layers were separated, the organic material was washed sequentially with saturated aqueous sodium bicarbonate (50 mL) and brine (50 mL), dried (sodium sulfate), filtered, the filtrate was concentrated under a vacuum, the residue was dissolved with dichloromethane (5.0 mL), silica gel (1.1 g) was added, and the volatiles were removed under a vacuum. The residue was subjected to flash chromatography on silica gel (11 g of silica gel, hexane) to give 7-bromo-3,3-difluoro-3,4-dihydro-2H-1,5-benzodioxepine (0.15 g) as a colorless solid.

Step 4.

A 15 mL round-bottomed flask was charged with 7-bromo-3,3-difluoro-3,4-dihydro-2H-1,5-benzodioxepine (from Step 3) (0.15 g, 0.57 mmol) and tetrahydrofuran (5.7 mL), the solution was cooled to −78° C., n-butyllithium (0.25 mL of a 2.5 M solution with toluene, 0.62 mmol, Sigma-Aldrich, St. Louis, Mo.) was added, and then the reaction mixture was stirred for 10 min. After that time, the reaction mixture was sparged with sulfur dioxide (Sigma-Aldrich, St. Louis, Mo.) for 5 min. After stirring for an additional 10 min, the reaction mixture was warmed to room temperature and then concentrated under a vacuum. The residue was dissolved with dichloromethane (5.7 mL), 1-chloro-2,5-pyrrolidinedione (0.083 g, 0.62 mmol, Sigma-Aldrich, St. Louis, Mo.) was added, and then the reaction mixture was stirred for 2 h. After that time, N-ethyl-N-(1-methylethyl)-2-propanamine (0.59 mL, 3.4 mmol, Sigma-Aldrich, St. Louis, Mo.) and (R)-1-(1-benzothiophen-2-yl)-1-(2-chlorophenyl)methanamine hydrochloride (Intermediate 2) (0.18 g, 0.57 mmol) were added, and the reaction mixture was stirred for 1 h. After that time, N,N-dimethyl-4-pyridinamine (0.0069 g, 0.057 mmol, Sigma-Aldrich, St. Louis, Mo.) was added and then the reaction mixture was stirred for 90 min. After that time, the reaction mixture was partitioned between ethyl acetate (50 mL) and saturated aqueous sodium bicarbonate (50 mL), the layers were separated, the organic material was washed sequentially with saturated aqueous sodium bicarbonate (50 mL) and brine (50 mL), dried (sodium sulfate), filtered, the filtrate was concentrated under a vacuum, the residue was dissolved with dichloromethane (5.0 mL), silica gel (1.0 g) was added, and the volatiles were removed under a vacuum. The residue was subjected to flash chromatography on silica gel (15 g of silica gel, 4:1 hexane-ethyl acetate). The isolated material was dissolved with dichloromethane (5.0 mL), silica gel (1.0 g) was added, and the volatiles were removed under a vacuum. The residue was subjected to flash chromatography on silica gel (15 g of silica gel, 6:1 hexane-ethyl acetate). The isolated material was subjected to reversed-phase preparative HPLC (Phenomenex Gemini C₁₈ column (Phenomenex, Inc., Torrance, Calif.) (150×30 mm, 10 μm) eluting with 0.10% trifluoroacetic acid in acetonitrile-water, gradient of 10% to 90% over 20 min) to give N—((R)-1-benzothiophen-2-yl(2-chlorophenyl)methyl)-3,3-difluoro-3,4-dihydro-2H-1,5-benzodioxepine-7-sulfonamide (0.048 g) as an off-white solid.

¹H NMR (400 MHz, DMSO-d₆) δ 9.25 (d, J=9.7 Hz, 1H), 7.94-7.83 (m, 1H), 7.76-7.67 (m, 1H), 7.56-7.47 (m, 1H), 7.42-7.38 (m, 1H), 7.36-7.22 (m, 5H), 7.13 (d, J=2.2 Hz, 1H), 7.00 (d, J=8.6 Hz, 1H), 6.85 (s, 1H), 6.18 (d, J=9.7 Hz, 1H), 4.51-4.31 (m, 4H). m/z (ESI, +ve ion) 543.7 (M+Na)⁺. GK-GKRP IC₅₀ (Binding)=0.62 μM.

### Example 77

### N—((R)-1-Benzothiophen-2-yl(2-chlorophenyl)methyl)-3-hydroxy-3,4-dihydro-2H-1,5-benzodioxepine-7-sulfonamide

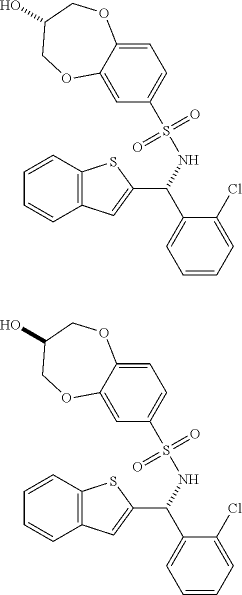

Step 1.

A 50 mL round-bottomed flask was charged with ((7-bromo-3,4-dihydro-2H-1,5-benzodioxepin-3-yl)oxy)(tris(1-methylethyl))silane (Example 35, Step 2) (0.61 g, 1.5 mmol) and tetrahydrofuran (15 mL), the solution was cooled to −78° C., n-butyllithium (0.67 mL of a 2.5 M solution with toluene, 1.7 mmol, Sigma-Aldrich, St. Louis, Mo.) was added, and then the reaction mixture was stirred for 10 min. After that time, the reaction mixture was sparged with sulfur dioxide (Sigma-Aldrich, St. Louis, Mo.) for 5 min. After stirring for an additional 10 min, the reaction mixture was warmed to room temperature and then concentrated under a vacuum. The residue was dissolved with dichloromethane (15 mL), 1-chloro-2,5-pyrrolidinedione (0.22 g, 1.7 mmol, Sigma-Aldrich, St. Louis, Mo.) was added, and then the reaction mixture was stirred for 2 h. After that time, N-ethyl-N-(1-methylethyl)-2-propanamine (1.6 mL, 9.1 mmol, Sigma-Aldrich, St. Louis, Mo.), (R)-1-(1-benzothiophen-2-yl)-1-(2-chlorophenyl)methanamine hydrochloride (Intermediate 2) (0.47 g, 1.5 mmol), and N,N-dimethyl-4-pyridinamine (0.019 g, 0.15 mmol, Sigma-Aldrich, St. Louis, Mo.) were added and then the reaction mixture was stirred for 18 h. After that time, the reaction mixture was partitioned between ethyl acetate (200 mL) and saturated aqueous sodium bicarbonate (200 mL), the layers were separated, the organic material was washed sequentially with saturated aqueous sodium bicarbonate (200 mL) and brine (200 mL), dried (sodium sulfate), filtered, and the filtrate was concentrated under a vacuum. The isolated material (0.68 g) was used in the next step of the synthesis without purification.

Step 2.

A 50 mL round-bottomed flask was charged with the isolated material from Step 1 (0.68 g), tetrahydrofuran (10 mL), and tetrabutylammonium fluoride (2.3 mL of a 1.0 M solution with tetrahydrofuran, 2.3 mmol, Sigma-Aldrich, St. Louis, Mo.), and then the reaction mixture was stirred for 18 h. After that time, more tetrabutylammonium fluoride (1.1 mL of a 1.0 M solution with tetrahydrofuran, 1.1 mmol, Sigma-Aldrich, St. Louis, Mo.), and then the reaction mixture was stirred for 16 h. After that time, the reaction mixture was partitioned between ethyl acetate (50 mL) and saturated aqueous sodium bicarbonate (50 mL), the layers were separated, the organic material was washed sequentially with saturated aqueous sodium bicarbonate (2×50 mL) and brine (3×50 mL), dried (sodium sulfate), filtered, the filtrate was concentrated under a vacuum, the residue was dissolved with dichloromethane (20 mL), silica gel (2.5 g) was added, and the volatiles were removed under a vacuum. The residue was subjected to flash chromatography on silica gel (25 g of silica gel, gradient elution of 1.5:1 to 1:1 hexane-ethyl acetate). The isolated material was subjected to reversed-phase preparative HPLC (Phenomenex Gemini C₁₈ column (Phenomenex, Inc., Torrance, Calif.) (150×30 mm, 10 μm) eluting with 0.10% trifluoroacetic acid in acetonitrile-water, gradient of 10% to 90% over 20 min) to give N-(1-benzofuran-2-yl(phenyl)methyl)-3-hydroxy-3,4-dihydro-2H-1,5-benzodioxepine-7-sulfonamide (0.087 g) as an off-white solid.

¹H NMR (400 MHz, DMSO-d₆) δ 9.15 (br. s., 1H), 7.93-7.82 (m, 1H), 7.75-7.66 (m, 1H), 7.55-7.46 (m, 1H), 7.42-7.35 (m, 1H), 7.35-7.20 (m, 4H), 7.17 (td, J=2.3, 8.4 Hz, 1H), 7.05 (d, J=1.6 Hz, 1H), 6.85 (dd, J=2.3, 8.4 Hz, 1H), 6.82 (s, 1H), 6.15 (br. s., 1H), 5.39 (d, J=5.1 Hz, 1H), 4.25-4.05 (m, 3H), 4.00-3.79 (m, 2H). m/z (ESI, +ve ion) 523.7 (M+Na)⁺. GK-GKRP IC₅₀ (Binding)=0.097 μM.

### Example 78

### N—((R)-1-Benzothiophen-2-yl(2-chlorophenyl)methyl)-3-fluoro-3,4-dihydro-2H-1,5-benzodioxepine-7-sulfonamide

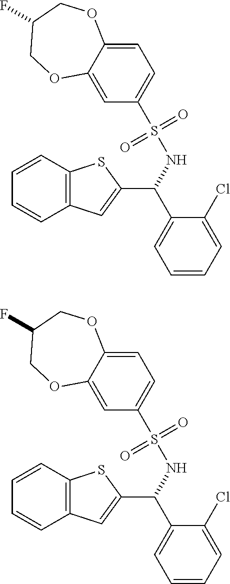

A 10 mL round-bottomed flask was charged with N-(1-benzofuran-2-yl(phenyl)methyl)-3-hydroxy-3,4-dihydro-2H-1,5-benzodioxepine-7-sulfonamide (Example 77) (0.069 g, 0.14 mmol), tetrabutylammonium dihydrogentrifluoride (1.1 mL of a 50-55 wt % solution with dichloroethane, Acros, Geel, Belgium), and (diethylamino)sulfur trifluoride (0.054 mL, 0.41 mmol, Sigma-Aldrich, St. Louis, Mo.), and then the reaction mixture was stirred for 30 min. After that time, the reaction mixture was added to saturated aqueous sodium bicarbonate (25 mL), the resulting mixture was stirred for 1 h, ethyl acetate (25 mL) was added, the layers were separated, the organic material was washed with saturated aqueous sodium bicarbonate (25 mL) and brine (25 mL), dried (sodium sulfate), filtered, the filtrate was concentrated, the residue was dissolved with dichloromethane (10 mL), silica gel (0.50 g) was added, and the volatiles were removed under a vacuum. The residue was subjected to flash chromatography on silica gel (5.0 g of silica gel, 2:1 dichloromethane-pentane). The isolated material was subjected to reversed-phase preparative HPLC (Phenomenex Gemini C₁₈ column (Phenomenex, Inc., Torrance, Calif.) (150×30 mm, 5 μm) eluting with 0.10% trifluoroacetic acid in acetonitrile-water, gradient of 10% to 90% over 20 min). The isolated material was dissolved with methanol (5.0 mL), the solution was filtered through a SPE-R66030B-06S Si-Carbonate (6 mL, 1 g) column (SiliCycle, Quebec City, Canada), and the filtrate was concentrated. The isolated material was subjected to reversed-phase preparative HPLC (Phenomenex Gemini C₁₈ column (Phenomenex, Inc., Torrance, Calif.) (150×50 mm, 10 μm) eluting with 0.10% trifluoroacetic acid in acetonitrile-water, gradient of 10% to 95% over 16 min) to give N—((R)-1-benzothiophen-2-yl(2-chlorophenyl)methyl)-3-fluoro-3,4-dihydro-2H-1,5-benzodioxepine-7-sulfonamide (0.011 g) as a pale yellow viscous liquid.

¹H NMR (400 MHz, chloroform-d) δ 7.71 (d, J=7.6 Hz, 1H), 7.60 (d, J=7.0 Hz, 1H), 7.42-7.17 (m, 8H), 6.92-6.81 (m, 2H), 6.20 (d, J=8.2 Hz, 1H), 5.59 (d, J=6.8 Hz, 1H), 5.03 (m, 1H), 4.48-4.18 (m, 4H). m/z (ESI, +ve ion) 525.8 (M+Na)⁺. GK-GKRP IC₅₀ (Binding)=0.047 μM.

### Example 79

### N-((6-amino-2-pyridinyl)(1-benzofuran-2-yl)methyl)-3,4-dihydro-2H-1,5-benzodioxepine-7-sulfonamide

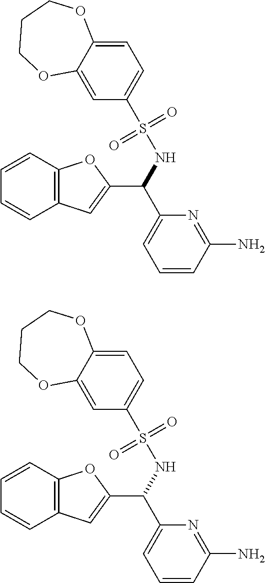

Step 1.

A 15 mL round-bottomed flask was charged with N-(1-benzofuran-2-ylmethylidene)-3,4-dihydro-2H-1,5-benzodioxepine-7-sulfonamide (Intermediate A) (0.20 g, 0.56 mmol), di-tert-butyl (6-bromo-2-pyridinyl)imidodicarbonate (0.23 g, 0.62 mmol, Sigma-Aldrich, St. Louis, Mo.), and tetrahydrofuran (5.6 mL). The solution was cooled to −78° C., t-butyllithium (0.72 mL of a 1.7 M solution with pentane, 1.2 mmol, Sigma-Aldrich, St. Louis, Mo.) was added, and then the reaction mixture was stirred for 10 min. After that time, methanol (2.0 mL) was added, the reaction mixture was warmed to room temperature, and then the volatiles were removed under a vacuum. The isolated material (0.37 g) was used in the next step of the synthesis without purification.

Step 2.

A 15 mL round-bottomed flask was charged with the isolated material from Step 1 (0.37 g), dichloromethane (5.7 mL), and hydrogen chloride (1.4 mL of a 4.0 M solution with 1,4-dioxane, 5.7 mmol, Sigma-Aldrich, St. Louis, Mo.), and the reaction mixture was stirred for 20 h. After that time, the reaction mixture was concentrated, the residue was partitioned between ethyl acetate (25 mL) and saturated aqueous sodium bicarbonate (25 mL), the layers were separated, the organic material was washed sequentially with saturated aqueous sodium bicarbonate (25 mL) and brine (25 mL), dried (sodium sulfate), filtered, the filtrate was concentrated under a vacuum, silica gel (1.3 g) was added, and the volatiles were removed under a vacuum. The residue was subjected to flash chromatography on silica gel (13 g of silica gel, 1:1 hexane-ethyl acetate). The isolated material was dissolved with dichloromethane (5.0 mL), silica gel (0.50 g) was added, and the volatiles were removed under a vacuum. The residue was subjected to flash chromatography on silica gel (6.5 g of silica gel, 49:1 dichloromethane-methanol). The isolated material was dissolved with dichloromethane (5.0 mL), silica gel (0.50 g) was added, and the volatiles were removed under a vacuum. The residue was subjected to flash chromatography on silica gel (6.5 g of silica gel, 99:1 dichloromethane-methanol). The isolated material was subjected to reversed-phase preparative HPLC (Phenomenex Gemini C₁₈ column (Phenomenex, Inc., Torrance, Calif.) (150×30 mm, 5 μm) eluting with 0.10% trifluoroacetic acid in acetonitrile-water, gradient of 10% to 90% over 10 min) to give N-((6-amino-2-pyridinyl)(1-benzofuran-2-yl)methyl)-3,4-dihydro-2H-1,5-benzodioxepine-7-sulfonamide (0.032 g) as an off-white solid.

¹H NMR (400 MHz, DMSO-d₆) δ 8.61 (d, J=7.5 Hz, 1H), 7.50 (dd, J=0.9, 7.5 Hz, 1H), 7.42-7.35 (m, 1H), 7.33-7.12 (m, 5H), 6.88 (d, J=8.4 Hz, 1H), 6.54 (d, J=7.2 Hz, 1H), 6.50 (s, 1H), 6.30 (d, J=8.0 Hz, 1H), 5.91 (s, 2H), 5.43 (d, J=7.5 Hz, 1H), 4.04 (t, J=5.6 Hz, 2H), 3.95 (t, J=5.6 Hz, 2H), 2.04 (quin, J=5.6 Hz, 2H). m/z (ESI, +ve ion) 451.9 (M+H)⁺. GK-GKRP IC₅₀ (Binding)=0.098 μM.

### Example 80

### N-((7-Chlorothieno[2,3-c]pyridin-2-yl)(phenyl)methyl)-3,4-dihydro-2H-1,5-benzodioxepine-7-sulfonamide

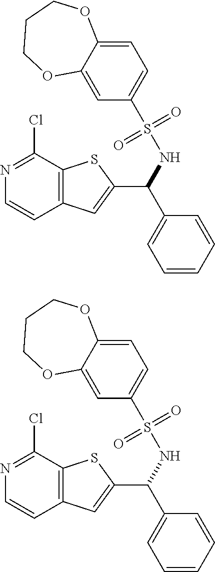

A 25 mL round-bottomed flask was charged with 7-chlorothieno[2,3-c]pyridine (0.25 g, 1.5 mmol, Ellanova, Hamden, Conn.) and tetrahydrofuran (7.4 mL). The solution was cooled to −78° C., n-butyllithium (0.65 mL of a 2.5 M solution with toluene, 1.6 mmol, Sigma-Aldrich, St. Louis, Mo.) was added, and then the reaction mixture was stirred for 15 min. After that time, a solution of N-(phenylmethylidene)-3,4-dihydro-2H-1,5-benzodioxepine-7-sulfonamide (Intermediate I) (0.51 g, 1.6 mmol) and tetrahydrofuran (7.4 mL) was added. After stirring for 10 min, methanol (2.0 mL) was added, the reaction mixture was removed from the cooling bath and allowed to warm to room temperature, silica gel (3.5 g) was added, and the volatiles were removed under a vacuum. The residue was subjected to flash chromatography on silica gel (35 g of silica gel, gradient elution, 3:1 to 2:1 hexane-ethyl acetate). The isolated material was dissolved with dichloromethane (20 mL), silica gel (3.5 g) was added, and the volatiles were removed under a vacuum. The residue was subjected to flash chromatography on silica gel (35 g of silica gel, 49:1 dichloromethane-diethyl ether) to give N-((7-chlorothieno[2,3-c]pyridin-2-yl)(phenyl)methyl)-3,4-dihydro-2H-1,5-benzodioxepine-7-sulfonamide (0.42 g) as a colorless solid.

¹H NMR (400 MHz, DMSO-d₆) δ 9.11 (br. s., 1H), 8.26 (d, J=5.3 Hz, 1H), 7.75 (d, J=5.5 Hz, 1H), 7.36-7.21 (m, 6H), 7.19 (d, J=1.0 Hz, 1H), 7.13 (d, J=2.3 Hz, 1H), 6.87 (d, J=8.4 Hz, 1H), 5.98 (br. s., 1H), 4.05 (t, J=5.7 Hz, 2H), 4.01-3.90 (m, 2H), 2.11-1.98 (m, 2H). m/z (ESI, +ve ion) 486.8 (M+H)⁺. GK-GKRP IC₅₀ (Binding)=1.1 μM.

### Example 81

### N—((R)-(2-Chlorophenyl)(7-(4-(2,2,2-trifluoro-1-hydroxy-1-(trifluoromethyl)ethyl)-2-pyridinyl)-1-benzothiophen-2-yl)methyl)-3,4-dihydro-2H-1,5-benzodioxepine-7-sulfonamide

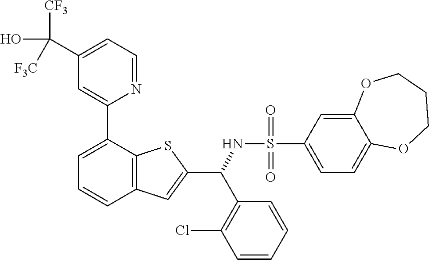

N,N-Dimethyl-4-pyridinamine (1.06 mg, 8.67 μmol, Sigma-Aldrich, St. Louis, Mo.) was added to a mixture of 2-(2-(2-((R)-amino(2-chlorophenyl)methyl)-1-benzothiophen-7-yl)-4-pyridinyl)-1,1,1,3,3,3-hexafluoro-2-propanol hydrochloride (Intermediate 3) (48 mg, 0.087 mmol), 3,4-dihydro-2H-1,5-benzodioxepine-7-sulfonyl chloride (32.4 mg, 0.130 mmol, ChemBridge, San Diego, Calif.), and N-ethyl-N-(1-methylethyl)-2-propanamine (45.3 μL, 0.260 mmol, Sigma-Aldrich, St. Louis, Mo.) in DMF (0.7 mL) and the mixture was stirred at room temperature for 1.5 h. The mixture was diluted with MeOH (1 mL), filtered, and purified by reversed phase HPLC (Phenomenex Gemini-NX 10μ C₁₈ 110 Å, 100×50 mm, 10% to 95% H₂O/MeCN, 0.1% TFA). The product containing fractions were combined, basified with solid NaHCO₃, and extracted with DCM (2×20 mL). The combined organic layers were dried (MgSO₄), filtered, and concentrated under reduced pressure to deliver N—((R)-(2-chlorophenyl)(7-(4-(2,2,2-trifluoro-1-hydroxy-1-(trifluoromethyl)ethyl)-2-pyridinyl)-1-benzothiophen-2-yl)methyl)-3,4-dihydro-2H-1,5-benzodioxepine-7-sulfonamide (33 mg) as a white solid.

¹H NMR (400 MHz, chloroform-d) δ 8.90 (d, J=5.3 Hz, 1H), 8.26 (s, 1H), 7.87 (d, J=7.4 Hz, 1H), 7.71 (d, J=7.6 Hz, 1H), 7.58 (d, J=5.1 Hz, 1H), 7.49-7.40 (m, 2H), 7.40-7.33 (m, 2H), 7.33-7.28 (m, 1H), 7.26-7.19 (m, 2H), 6.92 (s, 1H), 6.88 (d, J=8.2 Hz, 1H), 6.24 (d, J=8.0 Hz, 1H), 5.55 (d, J=7.8 Hz, 1H) 4.22 (t, J=5.7 Hz, 2H), 4.14 (t, J=5.7 Hz, 2H), 2.18 (quin, J=5.7 Hz, 2H). m/z (ESI, +ve ion) 728.7 (M+H)⁺. GK-GKRP IC₅₀ (Binding)=0.087 μM.

### Example 82

### N—((R)-(2-Chlorophenyl)(7-(4-(2,2,2-trifluoro-1-hydroxy-1-(trifluoromethyl)ethyl)-2-pyridinyl)-1-benzothiophen-2-yl)methyl)-3,4-dimethoxybenzenesulfonamide

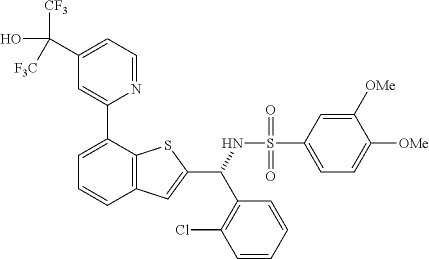

N,N-Dimethyl-4-pyridinamine (1.83 mg, 0.015 mmol, Sigma-Aldrich, St. Louis, Mo.) was added to a mixture of 2-(2-(2-((R)-amino(2-chlorophenyl)methyl)-1-benzothiophen-7-yl)-4-pyridinyl)-1,1,1,3,3,3-hexafluoro-2-propanol hydrochloride (Intermediate 3) (83 mg, 0.150 mmol), 3,4-dimethoxybenzenesulfonyl chloride (46.1 mg, 0.195 mmol, Acros, Geel, Belgium), and N-ethyl-N-(1-methylethyl)-2-propanamine (78 μL, 0.450 mmol, Sigma-Aldrich, St. Louis, Mo.) in DMF (0.7 mL) and the mixture was stirred at room temperature for 4 h. 3,4-Dimethoxybenzenesulfonyl chloride (46.1 mg, 0.195 mmol) was added and the mixture was stirred at room temperature for 3 h. The mixture was diluted with MeOH (1 mL), filtered, and purified by reversed phase HPLC (Phenomenex Gemini-NX 10μ C₁₈ 110 Å, 100×50 mm, 10% to 95% H₂O/MeCN, 0.1% TFA). The product containing fractions were combined, basified with solid NaHCO₃, and extracted with DCM (2×20 mL). The combined organic layers were dried (MgSO₄), filtered, and concentrated under reduced pressure to deliver N—((R)-(2-chlorophenyl)(7-(4-(2,2,2-trifluoro-1-hydroxy-1-(trifluoromethyl)ethyl)-2-pyridinyl)-1-benzothiophen-2-yl)methyl)-3,4-dimethoxybenzenesulfonamide (42 mg) as a white foam.

¹H NMR (400 MHz, chloroform-d) δ 8.89 (d, J=5.3 Hz, 1H), 8.25 (s, 1H), 7.87 (d, J=7.2 Hz, 1H), 7.70 (d, J=7.8 Hz, 1H), 7.59 (d, J=5.1 Hz, 1H), 7.50-7.42 (m, 3H), 7.33-7.28 (m, 1H), 7.26-7.20 (m, 3H), 6.89 (d, J=0.8 Hz, 1H), 6.80 (d, J=8.4 Hz, 1H), 6.23 (d, J=7.6 Hz, 1H), 5.53 (d, J=7.6 Hz, 1H), 3.89 (s, 3H), 3.81 (s, 3H). m/z (ESI, +ve ion) 717.7 (M+H)⁺. GK-GKRP IC₅₀ (Binding)=0.907 μM.

### Example 83

### N—((R)-(2-Chlorophenyl)(7-(4-((1R)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide

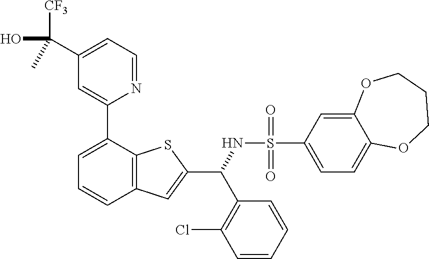

N,N-Dimethyl-4-pyridinamine (1.90 mg, 0.016 mmol, Sigma-Aldrich, St. Louis, Mo.) was added to a mixture of (2R)-2-(2-(2-((R)-amino(2-chlorophenyl)methyl)-1-benzothiophen-7-yl)-4-pyridinyl)-1,1,1-trifluoro-2-propanol (Intermediate 6) (72 mg, 0.156 mmol), 3,4-dihydro-2H-1,5-benzodioxepine-7-sulfonyl chloride (46.4 mg, 0.187 mmol, ChemBridge, San Diego, Calif.), and N-ethyl-N-(1-methylethyl)-2-propanamine (81.0 μL, 0.467 mmol, Sigma-Aldrich, St. Louis, Mo.) in DMF (0.7 mL) and the mixture was stirred at room temperature for 1.5 h. The mixture was diluted with MeOH (1 mL), filtered, and purified by reversed phase HPLC (Phenomenex Gemini-NX 10μ C₁₈ 110 Å, 100×50 mm, 10% to 95% H₂O/MeCN, 0.1% TFA). The product containing fractions were combined, basified with solid NaHCO₃, and extracted with DCM (2×20 mL). The combined organic layers were dried (MgSO₄), filtered, and concentrated under reduced pressure to deliver N—((R)-(2-chlorophenyl)(7-(4-((1R)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide (98 mg) as a white foam.

¹H NMR (400 MHz, chloroform-d) δ 8.82 (d, J=5.3 Hz, 1H), 8.13 (s, 1H), 7.86 (d, J=7.6 Hz, 1H), 7.69 (d, J=7.8 Hz, 1H), 7.48-7.39 (m, 3H), 7.39-7.32 (m, 2H), 7.32-7.28 (m, 1H), 7.25-7.20 (m, 2H), 6.91 (s, 1H), 6.88 (d, J=8.2 Hz, 1H), 6.24 (d, J=7.8 Hz, 1H), 5.53 (d, J=7.8 Hz, 1H), 4.22 (t, J=5.6 Hz, 2H), 4.14 (t, J=5.9 Hz, 2H), 2.18 (quin, J=5.7 Hz, 2H), 1.84 (s, 3H). m/z (ESI, +ve ion) 674.7 (M+H)⁺. GK-GKRP IC₅₀ (Binding)=0.038 μM.

### Example 84

### N—((R)-(2-Chlorophenyl)(7-(4-((1R)-2,2,2-trifluoro-1-hydroxy-1-methylethyl)-2-pyridinyl)-1-benzothiophen-2-yl)methyl)-3,4-dimethoxybenzenesulfonamide

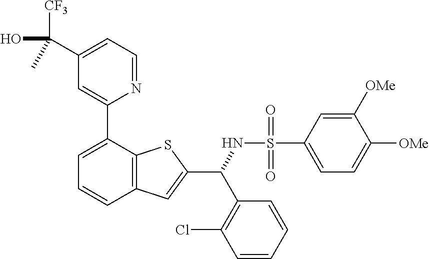

N,N-Dimethyl-4-pyridinamine (1.40 mg, 0.011 mmol, Sigma-Aldrich, St. Louis, Mo.) was added to a mixture of (2R)-2-(2-(2-((R)-amino(2-chlorophenyl)methyl)-1-benzothiophen-7-yl)-4-pyridinyl)-1,1,1-trifluoro-2-propanol (Intermediate 6) (53 mg, 0.114 mmol), 3,4-dimethoxybenzenesulfonyl chloride (35.2 mg, 0.149 mmol, Acros, Geel, Belgium), and N-ethyl-N-(1-methylethyl)-2-propanamine (59.7 μL, 0.343 mmol, Sigma-Aldrich, St. Louis, Mo.) in DMF (0.7 mL) and the mixture was stirred at room temperature for 1.5 h. 3,4-Dimethoxybenzenesulfonyl chloride (35.2 mg, 0.149 mmol) was added and the mixture was stirred at room temperature for 2.5 h. The mixture was diluted with MeOH (1 mL), filtered, and purified by reversed phase HPLC (Phenomenex Gemini-NX 10μ C₁₈ 110 Å, 100×50 mm, 10% to 95% H₂O/MeCN, 0.1% TFA). The product containing fractions were combined, basified with solid NaHCO₃, and extracted with DCM (2×20 mL). The combined organic layers were dried (MgSO₄), filtered, and concentrated under reduced pressure to deliver N—((R)-(2-chlorophenyl)(7-(4-((1R)-2,2,2-trifluoro-1-hydroxy-1-methylethyl)-2-pyridinyl)-1-benzothiophen-2-yl)methyl)-3,4-dimethoxybenzenesulfonamide (66 mg) as a white foam.

¹H NMR (400 MHz, chloroform-d) δ 8.80 (d, J=5.1 Hz, 1H), 8.12 (s, 1H), 7.86 (d, J=7.4 Hz, 1H), 7.67 (d, J=7.8 Hz, 1H), 7.50-7.39 (m, 4H), 7.33-7.28 (m, 1H), 7.25-7.18 (m, 3H), 6.87 (s, 1H), 6.80 (d, J=8.6 Hz, 1H), 6.22 (d, J=7.6 Hz, 1H), 5.55 (d, J=7.6 Hz, 1H), 3.89 (s, 3H), 3.81 (s, 3H), 2.28 (br. s, 1H), 1.84 (s, 3H). m/z (ESI, +ve ion) 662.8 (M+H)⁺. GK-GKRP IC₅₀ (Binding)=0.276 μM.

### Example 85

### N—((R)-(2-Chlorophenyl)(7-(4-((1S)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide

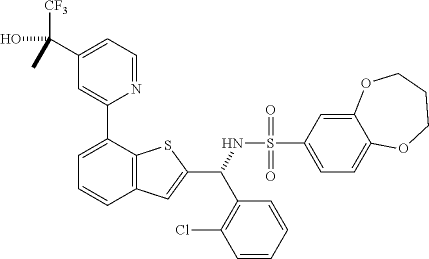

N,N-Dimethyl-4-pyridinamine (1.21 mg, 0.01 mmol, Sigma-Aldrich, St. Louis, Mo.) was added to a mixture of (2S)-2-(2-(2-((R)-amino(2-chlorophenyl)methyl)-1-benzothiophen-7-yl)-4-pyridinyl)-1,1,1-trifluoro-2-propanol (Intermediate 7) (46 mg, 0.099 mmol), 3,4-dihydro-2H-1,5-benzodioxepine-7-sulfonyl chloride (29.7 mg, 0.119 mmol, ChemBridge, San Diego, Calif.), and N-ethyl-N-(1-methylethyl)-2-propanamine (51.9 μL, 0.298 mmol, Sigma-Aldrich, St. Louis, Mo.) in DMF (0.7 mL) and the mixture was stirred at room temperature for 2 h. The mixture was diluted with MeOH (1 mL), filtered, and purified by reversed phase HPLC (Phenomenex Gemini-NX 10μ C₁₈ 110 Å, 100×50 mm, 10% to 95% H₂O/MeCN, 0.1% TFA). The product containing fractions were combined, basified with solid NaHCO₃, and extracted with DCM (2×20 mL). The combined organic layers were dried (MgSO₄), filtered, and concentrated under reduced pressure to deliver N—((R)-(2-Chlorophenyl)(7-(4-((1S)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide (49 mg) as a white solid.

¹H NMR (400 MHz, chloroform-d) δ 8.84 (d, J=5.3 Hz, 1H), 8.15 (s, 1H), 7.87 (d, J=7.6 Hz, 1H), 7.70 (d, J=7.8 Hz, 1H), 7.50-7.40 (m, 3H), 7.40-7.32 (m, 2H), 7.32-7.28 (m, 1H), 7.25-7.19 (m, 2H), 6.91 (s, 1H), 6.89 (d, J=8.2 Hz, 1H), 6.24 (d, J=7.8 Hz, 1H), 5.50 (d, J=7.6 Hz, 1H), 4.22 (t, J=5.7 Hz, 2H), 4.14 (t, J=5.7 Hz, 2H), 2.19 (quin, J=5.7 Hz, 2H), 1.85 (s, 3H). m/z (ESI, +ve ion) 674.7 (M+H)⁺. GK-GKRP IC₅₀ (Binding)=0.023 μM.

### Example 86

### N—((R)-(2-Chlorophenyl)(7-(4-((1S)-2,2,2-trifluoro-1-hydroxy-1-methylethyl)-2-pyridinyl)-1-benzothiophen-2-yl)methyl)-3,4-dimethoxybenzenesulfonamide

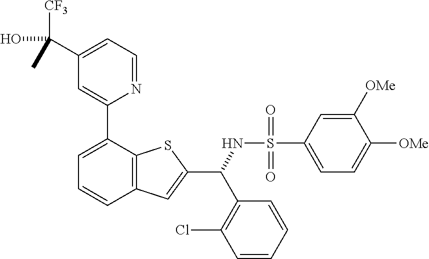

N,N-Dimethyl-4-pyridinamine (1.03 mg, 8.42 μmol, Sigma-Aldrich, St. Louis, Mo.) was added to a mixture of (2S)-2-(2-(2-((R)-amino(2-chlorophenyl)methyl)-1-benzothiophen-7-yl)-4-pyridinyl)-1,1,1-trifluoro-2-propanol (Intermediate 7) (39 mg, 0.084 mmol), 3,4-dimethoxybenzenesulfonyl chloride (23.9 mg, 0.101 mmol, Acros, Geel, Belgium), and N-ethyl-N-(1-methylethyl)-2-propanamine (44.0 μL, 0.253 mmol, Sigma-Aldrich, St. Louis, Mo.) in DMF (0.7 mL) and the mixture was stirred at room temperature for 2 h. 3,4-Dimethoxybenzenesulfonyl chloride (23.9 mg, 0.101 mmol, Acros, Geel, Belgium) and N,N-dimethyl-4-pyridinamine (1.03 mg, 8.42 μmol, Sigma-Aldrich, St. Louis, Mo.) was added and the mixture was stirred at room temperature for 2 h. The mixture was diluted with MeOH (1 mL), filtered, and purified by reversed phase HPLC (Phenomenex Gemini-NX 10μ C₁₈ 110 Å, 100×50 mm, 10% to 95% H₂O/MeCN, 0.1% TFA). The product containing fractions were combined, basified with solid NaHCO₃, and extracted with DCM (2×20 mL). The combined organic layers were dried (MgSO₄), filtered, and concentrated under reduced pressure to deliver N—((R)-(2-chlorophenyl)(7-(4-((1S)-2,2,2-trifluoro-1-hydroxy-1-methylethyl)-2-pyridinyl)-1-benzothiophen-2-yl)methyl)-3,4-dimethoxybenzenesulfonamide (45 mg) as a white solid.

¹H NMR (400 MHz, chloroform-d) δ 8.80 (d, J=5.3 Hz, 1H), 8.14 (s, 1H), 7.86 (d, J=7.4 Hz, 1H), 7.67 (d, J=7.8 Hz, 1H), 7.50-7.41 (m, 4H), 7.33-7.28 (m, 1H), 7.25-7.19 (m, 3H), 6.87 (s, 1H), 6.79 (d, J=8.4 Hz, 1H), 6.22 (d, J=7.6 Hz, 1H), 5.57 (d, J=7.6 Hz, 1H), 3.89 (s, 3H), 3.81 (s, 3H), 2.46 (br. s, 1H), 1.85 (s, 3H). m/z (ESI, +ve ion) 662.8 (M+H)⁺. GK-GKRP IC₅₀ (Binding)=0.072 μM.

### Example 87

### N-((2-Amino-4-pyridinyl)(1-benzofuran-2-yl)methyl)-3,4-dihydro-2H-1,5-benzodioxepine-7-sulfonamide

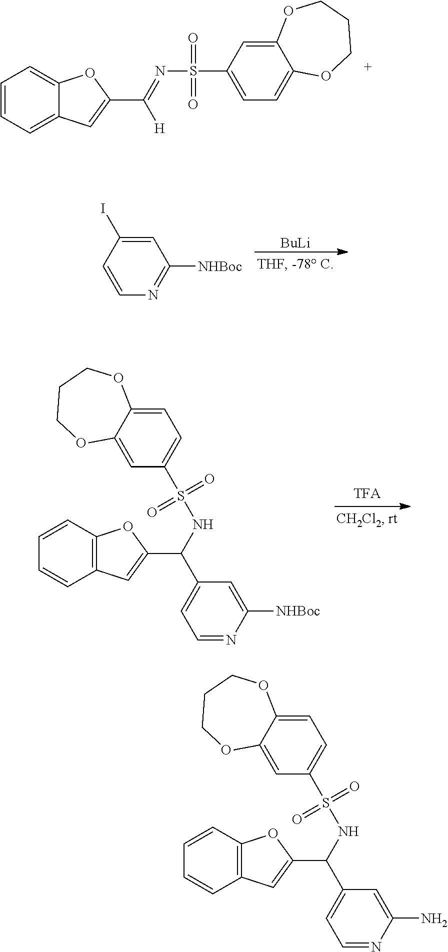

Step 1.

Butyllithium (0.804 mL of a 1.6 M solution in hexane, 1.29 mmol, Sigma-Aldrich, St. Louis, Mo.) was added to a solution of tert-butyl (4-iodo-2-pyridinyl)carbamate (0.188 g, 0.587 mmol, Adesis, New Castle, Del.) in THF (5 mL) at −78° C. and the mixture was stirred at −78° C. for 5 min. A solution of N-((1E)-1-benzofuran-2-ylmethylidene)-3,4-dihydro-2H-1,5-benzodioxepine-7-sulfonamide (Intermediate A) (0.20 g, 0.560 mmol) in THF (10 mL) was added dropwise while the internal reaction mixture was maintained below −65° C. The cold bath was removed and the reaction mixture was warmed to room temperature. The mixture was cooled in an ice bath, saturated aqueous NaHCO₃ (100 mL) was added, and the mixture was warmed to room temperature and extracted with EtOAc (2×100 mL). The combined organic layers were washed with brine (200 mL), dried (Na₂SO₄), filtered, and concentrated under reduced pressure. The residue was taken up in DMSO (9 mL), filtered, and purified by reversed phase HPLC (Phenomenex Gemini-NX 10μ C₁₈ 110 Å, 100×50 mm, 10% to 95% H₂O/MeCN, 0.1% TFA). The product containing fractions were combined, basified with saturated aqueous NaHCO₃ (100 mL), and extracted with DCM (2×80 mL). The combined organic layers were dried (Na₂SO₄), filtered, and concentrated under reduced pressure to deliver tert-butyl (4-(1-benzofuran-2-yl((3,4-dihydro-2H-1,5-benzodioxepin-7-ylsulfonyl)amino)methyl)-2-pyridinyl)carbamate (28 mg) as an off-white solid.

Step 2.

Trifluoroacetic acid (5.66 mL, 76 mmol, Sigma-Aldrich, St. Louis, Mo.) was added to a solution of tert-butyl (4-(1-benzofuran-2-yl((3,4-dihydro-2H-1,5-benzodioxepin-7-ylsulfonyl)amino)methyl)-2-pyridinyl)carbamate (28 mg, 0.051 mmol) in DCM (6 mL) and the mixture was stirred at room temperature for 30 min. The mixture was concentrated under reduced pressure, the residue was taken up in saturated aqueous NaHCO₃ (100 mL), and the mixture was extracted with DCM (2×75 mL). The combined organic layers were washed with brine (100 mL), dried (Na₂SO₄), filtered, and concentrated under reduced pressure to deliver N-((2-amino-4-pyridinyl)(1-benzofuran-2-yl)methyl)-3,4-dihydro-2H-1,5-benzodioxepine-7-sulfonamide (20 mg) as an off-white solid.

¹H NMR (400 MHz, methanol-d₄) δ 7.78 (d, J=5.5 Hz, 1H), 7.46 (d, J=7.6 Hz, 1H), 7.37-7.28 (m, 2H), 7.26-7.13 (m, 3H), 6.81 (d, J=8.4 Hz, 1H), 6.55 (dd, J=5.7, 1.0 Hz, 1H), 6.49 (s, 2H), 5.60 (s, 1H), 4.04 (t, J=5.6 Hz, 2H), 3.98-3.87 (m, 2H), 2.07 (quin, J=5.6 Hz, 2H). m/z (ESI, +ve ion) 451.9 (M+H)⁺. GK-GKRP IC₅₀ (Binding)=4.88 μM.

### GKRP LC MS/MS Biochemical Assay (LCMS/MS)

This assay is used to directly measure the formation of ¹³C-glucose-6-phosphate from ¹³C-glucose by LC MS/MS. Begin by preparing the following solutions: Compound Buffer (CB): 50 mM Tris, pH 7.5/4 mM MgCl₂/6% DMSO/fresh 10 mM DTT from 1M frozen stock. Enzyme Buffer (EB): 50 mM Tris, pH 7.5/4 mM MgCl₂/6% DMSO/fresh 0.1% BSA/fresh 0.01% Brij-35 (10% BSA and 1% Brij-35 stock). GK (Glucokinase) Working Stock (5×): Dilute human His-hepatic GK to 30 nM in EB buffer. Substrate Working Stock (1.47×): Dilute ¹³C-D-glucose (Sigma-Aldrich, St. Louis, Mo.) to 7.35 mM from 1M stock and dilute ATP (EMD Chemical, Gibbstown, N.J.) to 0.3528 mM from frozen 100 mM stock in CB buffer (1M ¹³C-D-glucose=186.11 mg/ml in water). Dilute 20 mM fructose-6-phosphate (F6P) (Sigma-Aldrich, St. Louis, Mo.) to 441 μM in the substrate working stock. GKRP (Glucokinase Regulatory Protein) (10×): Dilute GKRP to 280 nM from 33.366 mM stock in EB buffer. Combine the following reagents in a 96-well polypropylene plate: 34 μL of Substrate Working Stock (1.47×), 5 μl of 280 nM GKRP (10×), and 1 μl of compound or DMSO. Seal the plate and incubate for 30 minutes at room temperature while mixing. After 30 minutes add 10 μl of GK Working Stock (5×). Re-seal the plate and incubate for another 30 minutes at room temperature while mixing. After the second 30 minutes, stop the reaction by the addition of 50 μl of 100% acetonitrile, seal, and mix for 5-10 minutes. Run 10 μl of this sample through the LC MS/MS (API 3200, Applied Biosystems, Carlsbad, Calif.). Detection settings are for 265.2/78.8 atomic mass units.

### GK-GKRP Binding Assay Protocol (Binding)

This assay is used to directly measure the interaction between glucokinase (GK) and glucokinase regulatory protein (GKRP). Begin by preparing the following solutions. Assay Buffer: 20 mM Tris, pH 7.5/0.05% BSA/1 mM DTT/1 μM sorbitol-6-phosphate. Assay Procedure: Dilute avi-tagged GKRP to 10.7 nM in assay buffer. Combine the following reagents in a white 96-well half area plate. Pipette 14 μl of the diluted avi-tagged GKRP into each well. Add 1 μl of compound to be tested and incubate at room temperature for 20 minutes. After 20 minutes, add 5 μl of assay buffer containing 6 nM GK-fluorescein. Add 10 μl of AlphaScreen® beads (Perkin Elmer, Waltham Mass.) that have been diluted 1:333 in assay buffer. Incubate in a dark room for 2 hours at room temperature. After 2 hours read the plate using an Envision plate reader (Perkin Elmer, Waltham Mass.).

### Assay Materials

Recombinant human glucokinase (hepatic) was purified and formulated in 50 mM Tris (pH 7.5), 150 mM NaCl and 20 mM glucose previous to labeling with fluorescein. A one-to-one ratio of 5-bromomethylfluorescein (Invitrogen, Carlsbad, Calif.) was prepared and the reaction mixture was incubated overnight at 4° C. in light protected tubes. Unreacted dye was removed by extensive dialysis into the final formulation buffer: 25 mM Tris (pH 7.2), 150 mM NaCl, 10% glycerol and 2 mM DTT. The human glucokinase regulatory protein construct was designed with a C-terminal avitag and was expressed in a baculovirus system. Purified GKRP was incubated overnight with recombinant biotin ligase in the presence of D-biotin, ATP and MgCl₂. GKRP was isolated from reaction components by chromatography on a Superdex 200 size exclusion column into: 50 mM HEPES, 150 mM NaCl and 5 mM DTT. LCMS analysis demonstrated that GKRP was mono-biotinylated to completion. Proteins were quantitated according to their calculated molar extinction coefficients at A₂₈₀ and were stored at −80° C.

Data obtained using the assays above can be found in the specific Examples. In the Examples, the assays are referred to as “LCMS/MS” or “Binding.”

What is claimed is:

1. A compound of formula I or II, or a pharmaceutically acceptable salt thereof, wherein: 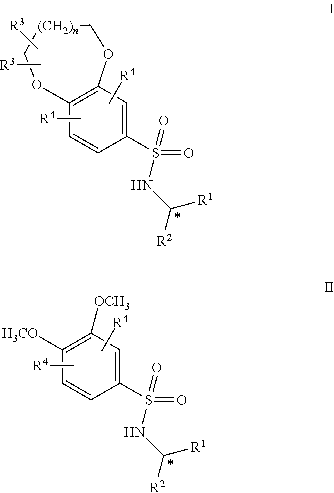
the symbol * represents a chiral center that has the R configuration or is a mixture of the R and S configurations;
n is 0 or 1;
R¹ is a five or six membered monocyclic aryl group or monocyclic heteroaryl group, wherein the heteroatoms are independently selected from N, O or S, which groups can be optionally substituted at the (1) positions with respect to its point of attachment to the rest of the compound, when possible, with a substituent selected from —C₁₋₈alkyl, —C₁₋₈alkoxy, halo, —SC₁₋₈alkyl, —C₂₋₈alkenyl, —C₂₋₈alkynyl, —C₁₋₈hydroxyalkyl, haloalkyl, perfluoroalkyl or —OCF₃; or at the (2) positions, when possible, with a substituent selected from fluorine,
—OH or —NH₂; or at the (3) position, when possible, with fluorine;
R² is a nine or ten membered bicyclic aryl group or bicyclic heteroaryl group, wherein the heteroatoms are independently selected from N, O or S, which groups can be optionally substituted with from 1 to 7 substituents independently selected from halo, C₁₋₆ alkyl, C₁₋₆alkoxy, —OH or a five or six membered aryl or heteroaryl group, which aryl or heteroaryl group that can be optionally substituted with from 1 to 3 substituents independently selected from C₁₋₆ alkyl, C₁₋₆alkoxy, —OH, —NH₂, —C(CF₃)(OH)(CF₃) or —C(CF₃)(OH)(CH₃);
each R³ is independently hydrogen, —OH or halo when n is 1, or hydrogen, —OH, halo or —CH₃ when n is 0; and
each R⁴ is independently hydrogen or halo;
provided that the compound is not N-(1-benzofuran-2-yl(phenyl)methyl)-3,4-dihydro-2H-1,5-benzodioxepine-7-sulfonamide.

2. A compound in accordance with claim 1, or a pharmaceutically acceptable salt thereof, wherein:
the compound has the formula III or IV 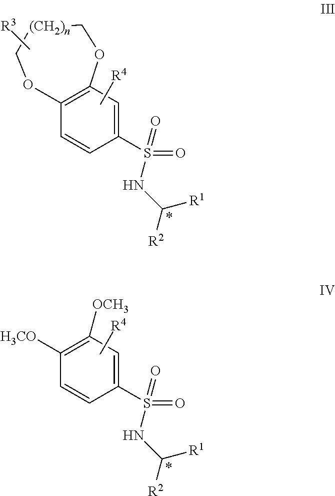
R² is a nine or ten membered bicyclic aryl group or bicyclic heteroaryl group, wherein the heteroatoms are independently selected from N, O or S, which groups can be optionally substituted with from 1 to 7 substituents independently selected from C₁₋₆alkyl, C₁₋₆alkoxy, —OH or a five or six membered aryl or heteroaryl group, which aryl or heteroaryl group that can be optionally substituted with from 1 to 3 substituents independently selected from C₁₋₆ alkyl, C₁₋₆alkoxy, —OH, —NH₂, or —C(CF₃)(OH)(CH₃);
R³ is hydrogen or halo when n is 1, or hydrogen, halo, or —CH₃ when n is 0; and
R⁴ is hydrogen or halo.

3. A compound of claim 1 or 2, or a pharmaceutically acceptable salt thereof, wherein R¹ is phenyl, substituted phenyl, pyridyl, substituted pyridyl, thienyl, substituted thienyl, pyrimidinyl or substituted pyrimidinyl.

4. A compound of claim 1 or 2, or a pharmaceutically acceptable salt thereof, wherein R² is benzofuranyl, substituted benzofuranyl, benzothiophenyl, substituted benzothiophenyl, benzothiazolyl, substituted benzothiazolyl, thieno[2,3-c]pyridinyl, substituted thieno[2,3-c]pyridinyl, benzoxazolyl, substituted benzoxazolyl, thieno[3,2-c]pyridinyl, substituted, thieno[3,2-c]pyridinyl, quinolinyl, substituted quinolinyl, naphthalenyl, substituted naphthalenyl, thieno[3,2-b]pyridinyl, substituted thieno[3,2-b]pyridinyl, thieno[2,3-b]pyridinyl, substituted thieno[2,3-b]pyridinyl, [1,3]thiazolo[5,4-b]pyridinyl, or substituted [1,3]thiazolo[5,4-b]pyridinyl.

5. A compound of claim 1 or 2, or a pharmaceutically acceptable salt thereof, wherein R³ is hydrogen or halogen.

6. A compound of claim 1 or 2, or a pharmaceutically acceptable salt thereof, wherein R⁴ is hydrogen.

7. A compound of claim 1 or 2, or a pharmaceutically acceptable salt thereof, that has the R configuration at the stereocenter designated with a *.

8. A compound of claim 1 or 2, or a pharmaceutically acceptable salt thereof, that is a mixture of the R and S configurations at the stereocenter designated with a *.

9. A compound of claim 1 or 2, or a pharmaceutically acceptable salt thereof, that is a racemic mixture of the R and S configurations at the stereocenter designated with a *.

10. A compound of claim 1 or 2, or a pharmaceutically acceptable salt thereof, wherein:
R¹ is phenyl, substituted phenyl, pyridyl, substituted pyridyl, thienyl, substituted thienyl, pyrimidinyl or substituted pyrimidinyl; and
R² is benzofuranyl, substituted benzofuranyl, benzothiophenyl, substituted benzothiophenyl, benzothiazolyl, substituted benzothiazolyl, thieno[2,3-c]pyridinyl, substituted thieno[2,3-c]pyridinyl, benzoxazolyl, substituted benzoxazolyl, thieno[3,2-c]pyridinyl, substituted, thieno[3,2-c]pyridinyl, quinolinyl, substituted quinolinyl, naphthalenyl, substituted naphthalenyl, thieno[3,2-b]pyridinyl, substituted thieno[3,2-b]pyridinyl, thieno[2,3-b]pyridinyl, substituted thieno[2,3-b]pyridinyl, [1,3]thiazolo[5,4-b]pyridinyl, or substituted [1,3]thiazolo[5,4-b]pyridinyl.

11. A compound of claim 1 or 2, or a pharmaceutically acceptable salt thereof, wherein:
R¹ is phenyl, substituted phenyl, pyridyl, substituted pyridyl, thienyl, substituted thienyl, pyrimidinyl or substituted pyrimidinyl;
R² is benzofuranyl, substituted benzofuranyl, benzothiophenyl, substituted benzothiophenyl, benzothiazolyl, substituted benzothiazolyl, thieno[2,3-c]pyridinyl, substituted thieno[2,3-c]pyridinyl, benzoxazolyl, substituted benzoxazolyl, thieno[3,2-c]pyridinyl, substituted, thieno[3,2-c]pyridinyl, quinolinyl, substituted quinolinyl, naphthalenyl, substituted naphthalenyl, thieno[3,2-b]pyridinyl, substituted thieno[3,2-b]pyridinyl, thieno[2,3-b]pyridinyl, substituted thieno[2,3-b]pyridinyl, [1,3]thiazolo[5,4-b]pyridinyl, or substituted [1,3]thiazolo[5,4-b]pyridinyl.
R³ is hydrogen or halogen;
R⁴ is hydrogen; and the compound has the R configuration at the stereocenter designated with a *.

12. A compound, or a pharmaceutically acceptable salt, selected from:
N-(1-benzofuran-2-yl(2-methylphenyl)methyl)-3,4-dihydro-2H-1,5-benzodioxepine-7-sulfonamide;
N-(1-benzofuran-2-yl(4-fluoro-2-methylphenyl)methyl)-3,4-dihydro-2H-1,5-benzodioxepine-7-sulfonamide;
N-(1-benzofuran-2-yl(5-fluoro-2-methylphenyl)methyl)-3,4-dihydro-2H-1,5-benzodioxepine-7-sulfonamide;
N-(1-benzofuran-2-yl(2-fluoro-6-methylphenyl)methyl)-3,4-dihydro-2H-1,5-benzodioxepine-7-sulfonamide;
N-(1-benzofuran-2-yl(2-ethylphenyl)methyl)-3,4-dihydro-2H-1,5-benzodioxepine-7-sulfonamide;
N-(1-benzofuran-2-yl(2-ethenylphenyl)methyl)-3,4-dihydro-2H-1,5-benzodioxepine-7-sulfonamide;
N-(1-benzofuran-2-yl(2-ethynylphenyl)methyl)-3,4-dihydro-2H-1,5-benzodioxepine-7-sulfonamide;
N-(1-benzofuran-2-yl(2-(1-methylethyl)phenyl)methyl)-3,4-dihydro-2H-1,5-benzodioxepine-7-sulfonamide;
N-(1-benzofuran-2-yl (2,6-dimethylphenyl)methyl)-3,4-dihydro-2H-1,5-benzodioxepine-7-sulfonamide;
N-(1-benzofuran-2-yl(2-propylphenyl)methyl)-3,4-dihydro-2H-1,5-benzodioxepine-7-sulfonamide;
N-(1-benzofuran-2-yl(2-(trifluoromethyl)phenyl)methyl)-3,4-dihydro-2H-1,5-benzodioxepine-7-sulfonamide;
N-(1-benzofuran-2-yl(3-hydroxyphenyl)methyl)-3,4-dihydro-2H-1,5-benzodioxepine-7-sulfonamide;
N-((3-aminophenyl)(1-benzofuran-2-yl)methyl)-3,4-dihydro-2H-1,5-benzodioxepine-7-sulfonamide;
N-(1-benzofuran-2-yl(2-(hydroxymethyl)phenyl)methyl)-3,4-dihydro-2H-1,5-benzodioxepine-7-sulfonamide;
N-(1-benzofuran-2-yl(2-(2-hydroxyethyl)phenyl)methyl)-3,4-dihydro-2H-1,5-benzodioxepine-7-sulfonamide;
N-(1-benzofuran-2-yl(2-methoxyphenyl)methyl)-3,4-dihydro-2H-1,5-benzodioxepine-7-sulfonamide;
N-(1-benzofuran-2-yl(2-(trifluoromethoxy)phenyl)methyl)-3,4-dihydro-2H-1,5-benzodioxepine-7-sulfonamide;
N-(1-benzofuran-2-yl(2-(methylsulfanyl)phenyl)methyl)-3,4-dihydro-2H-1,5-benzodioxepine-7-sulfonamide;
N-(1-benzofuran-2-yl(2-chlorophenyl)methyl)-3,4-dihydro-2H-1,5-benzodioxepine-7-sulfonamide;
N-(1-benzofuran-2-yl(2-iodophenyl)methyl)-3,4-dihydro-2H-1,5-benzodioxepine-7-sulfonamide;
N-(1-benzofuran-2-yl(2-bromophenyl)methyl)-3,4-dihydro-2H-1,5-benzodioxepine-7-sulfonamide;
N-(1-benzofuran-2-yl(2-fluorophenyl)methyl)-3,4-dihydro-2H-1,5-benzodioxepine-7-sulfonamide;
N-(1-benzofuran-2-yl(3-fluorophenyl)methyl)-3,4-dihydro-2H-1,5-benzodioxepine-7-sulfonamide;
N-(1-benzofuran-2-yl(2-pyridinyl)methyl)-3,4-dihydro-2H-1,5-benzodioxepine-7-sulfonamide;
N-(1-benzofuran-2-yl(3-methyl-2-pyridinyl)methyl)-3,4-dihydro-2H-1,5-benzodioxepine-7-sulfonamide;
N-(1-benzofuran-2-yl(2-chloro-3-pyridinyl)methyl)-3,4-dihydro-2H-1,5-benzodioxepine-7-sulfonamide;
N-(1-benzofuran-2-yl(3-chloro-4-pyridinyl)methyl)-3,4-dihydro-2H-1,5-benzodioxepine-7-sulfonamide;
N-(1-benzofuran-2-yl(2-pyrimidinyl)methyl)-3,4-dihydro-2H-1,5-benzodioxepine-7-sulfonamide;
N-(1-benzofuran-2-yl(3-methoxy-2-pyridinyl)methyl)-3,4-dihydro-2H-1,5-benzodioxepine-7-sulfonamide;
N-(1-benzofuran-2-yl(2-thiophenyl)methyl)-3,4-dihydro-2H-1,5-benzodioxepine-7-sulfonamide;
N-(1-benzofuran-2-yl(3-thiophenyl)methyl)-3,4-dihydro-2H-1,5-benzodioxepine-7-sulfonamide;
N-(1-benzofuran-2-yl(3-methyl-2-thiophenyl)methyl)-3,4-dihydro-2H-1,5-benzodioxepine-7-sulfonamide;
N-(1-benzofuran-2-yl(1,3-thiazol-2-yl)methyl)-3,4-dihydro-2H-1,5-benzodioxepine-7-sulfonamide;
N-(1-benzofuran-2-yl(phenyl)methyl)-3-fluoro-3,4-dihydro-2H-1,5-benzodioxepine-7-sulfonamide;
N-(1-benzothiophen-2-yl(2-methylphenyl)methyl)-3,4-dihydro-2H-1,5-benzodioxepine-7-sulfonamide;
N-(1-benzothiophen-2-yl(2-ethylphenyl)methyl)-3,4-dihydro-2H-1,5-benzodioxepine-7-sulfonamide;
N-(1-benzothiophen-2-yl(2-ethenylphenyl)methyl)-3,4-dihydro-2H-1,5-benzodioxepine-7-sulfonamide;
N-(1-benzothiophen-2-yl(2-ethynylphenyl)methyl)-3,4-dihydro-2H-1,5-benzodioxepine-7-sulfonamide;
N-(1-benzothiophen-2-yl(2-methoxyphenyl)methyl)-3,4-dihydro-2H-1,5-benzodioxepine-7-sulfonamide;
N-(1-benzothiophen-2-yl(2-(methylsulfanyl)phenyl)methyl)-3,4-dihydro-2H-1,5-benzodioxepine-7-sulfonamide;
N-(1-benzothiophen-2-yl(2-chlorophenyl)methyl)-3,4-dihydro-2H-1,5-benzodioxepine-7-sulfonamide;
N-(1-benzothiophen-2-yl(3-methoxy-2-pyridinyl)methyl)-3,4-dihydro-2H-1,5-benzodioxepine-7-sulfonamide;
N-((3-methyl-1-benzofuran-2-yl)(phenyl)methyl)-3,4-dihydro-2H-1,5-benzodioxepine-7-sulfonamide;
N-((3-methyl-1-benzofuran-2-yl)(2-methylphenyl)methyl)-3,4-dihydro-2H-1,5-benzodioxepine-7-sulfonamide;
N-(phenyl(thieno[2,3-b]pyridin-2-yl)methyl)-3,4-dihydro-2H-1,5-benzodioxepine-7-sulfonamide;
N-((2-methoxyphenyl)(thieno[2,3-b]pyridin-2-yl)methyl)-3,4-dihydro-2H-1,5-benzodioxepine-7-sulfonamide;
N-((2-(methylsulfanyl)phenyl)(thieno[2,3-b]pyridin-2-yl)methyl)-3,4-dihydro-2H-1,5-benzodioxepine-7-sulfonamide;
N-((3-methoxy-2-pyridinyl)(thieno[2,3-b]pyridin-2-yl)methyl)-3,4-dihydro-2H-1,5-benzodioxepine-7-sulfonamide;
N-((3-methoxy-4-pyridinyl)(thieno[2,3-b]pyridin-2-yl)methyl)-3,4-dihydro-2H-1,5-benzodioxepine-7-sulfonamide;
N-(1,3-benzothiazol-2-yl(2-methoxyphenyl)methyl)-3,4-dihydro-2H-1,5-benzodioxepine-7-sulfonamide;
N-(1,3-benzothiazol-2-yl(2-(methylsulfanyl)phenyl)methyl)-3,4-dihydro-2H-1,5-benzodioxepine-7-sulfonamide;
N-(1,3-benzothiazol-2-yl(3-methoxy-2-pyridinyl)methyl)-3,4-dihydro-2H-1,5-benzodioxepine-7-sulfonamide;
N-(1,3-benzoxazol-2-yl(phenyl)methyl)-3,4-dihydro-2H-1,5-benzodioxepine-7-sulfonamide;
N-(2-naphthalenyl(phenyl)methyl)-3,4-dihydro-2H-1,5-benzodioxepine-7-sulfonamide;
N-(phenyl(2-quinolinyl)methyl)-3,4-dihydro-2H-1,5-benzodioxepine-7-sulfonamide;
N-(1-benzothiophen-2-yl(phenyl)methyl)-3,4-dihydro-2H-1,5-benzodioxepine-7-sulfonamide;
N-((5-methoxy-1-benzothiophen-2-yl)(phenyl)methyl)-3,4-dihydro-2H-1,5-benzodioxepine-7-sulfonamide;
N-(1,3-benzothiazol-2-yl(phenyl)methyl)-3,4-dihydro-2H-1,5-benzodioxepine-7-sulfonamide;
N-(phenyl(thieno[2,3-c]pyridin-2-yl)methyl)-3,4-dihydro-2H-1,5-benzodioxepine-7-sulfonamide;
N-(phenyl(thieno[3,2-b]pyridin-2-yl)methyl)-3,4-dihydro-2H-1,5-benzodioxepine-7-sulfonamide;
N-(phenyl(thieno[3,2-c]pyridin-2-yl)methyl)-3,4-dihydro-2H-1,5-benzodioxepine-7-sulfonamide;
N-((5-hydroxy-1-benzothiophen-2-yl)(phenyl)methyl)-3,4-dihydro-2H-1,5-benzodioxepine-7-sulfonamide;
N-((2-(methylsulfanyl)phenyl)(thieno[2,3-c]pyridin-2-yl)methyl)-3,4-dihydro-2H-1,5-benzodioxepine-7-sulfonamide;
N-((2-(methylsulfanyl)phenyl)([1,3]thiazolo[5,4-b]pyridin-2-yl)methyl)-3,4-dihydro-2H-1,5-benzodioxepine-7-sulfonamide;
N-((2-methoxyphenyl)(thieno[2,3-c]pyridin-2-yl)methyl)-3,4-dihydro-2H-1,5-benzodioxepine-7-sulfonamide;
N-((2-methoxyphenyl)([1,3]thiazolo[5,4-b]pyridin-2-yl)methyl)-3,4-dihydro-2H-1,5-benzodioxepine-7-sulfonamide;
N-((2-chlorophenyl)([1,3]thiazolo[5,4-b]pyridin-2-yl)methyl)-3,4-dihydro-2H-1,5-benzodioxepine-7-sulfonamide;
N-((3-methoxy-2-pyridinyl)([1,3]thiazolo[5,4-b]pyridin-2-yl)methyl)-3,4-dihydro-2H-1,5-benzodioxepine-7-sulfonamide;
N-(phenyl(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N-(phenyl(7-(2-pyridinyl)-1-benzothiophen-2-yl)methyl)-3,4-dihydro-2H-1,5-benzodioxepine-7-sulfonamide;
N-((7-chloro-1-benzothiophen-2-yl)(phenyl)methyl)-3,4-dihydro-2H-1,5-benzodioxepine-7-sulfonamide;
N-(phenyl(7-phenyl-1-benzothiophen-2-yl)methyl)-3,4-dihydro-2H-1,5-benzodioxepine-7-sulfonamide;
N-(1-benzothiophen-2-yl(2-(methylsulfanyl)phenyl)methyl)-3,4-dihydro-2H-1,5-benzodioxepine-7-sulfonamide; or
N-(1-benzothiophen-2-yl(2-(methylsulfanyl)phenyl)methyl)-2,3-dihydro-1,4-benzodioxine-6-sulfonamide.

13. A compound, or a pharmaceutically acceptable salt, selected from:
N-(1-benzothiophen-2-yl(2-chlorophenyl)methyl)-3,3-difluoro-3,4-dihydro-2H-1,5-benzodioxepine-7-sulfonamide;
N-(1-benzothiophen-2-yl(2-chlorophenyl)methyl)-3-hydroxy-3,4-dihydro-2H-1,5-benzodioxepine-7-sulfonamide;
N-(1-benzothiophen-2-yl(2-chlorophenyl)methyl)-3-fluoro-3,4-dihydro-2H-1,5-benzodioxepine-7-sulfonamide;
N-((6-amino-2-pyridinyl)(1-benzofuran-2-yl)methyl)-3,4-dihydro-2H-1,5-benzodioxepine-7-sulfonamide;
N-((7-chlorothieno[2,3-c]pyridin-2-yl)(phenyl)methyl)-3,4-dihydro-2H-1,5-benzodioxepine-7-sulfonamide;
N-((2-chlorophenyl)(7-(4-(2,2,2-trifluoro-1-hydroxy-1-(trifluoromethyl)ethyl)-2-pyridinyl)-1-benzothiophen-2-yl)methyl)-3,4-dihydro-2H-1,5-benzodioxepine-7-sulfonamide;
N-((2-chlorophenyl)(7-(4-(2,2,2-trifluoro-1-hydroxy-1-(trifluoromethyl)ethyl)-2-pyridinyl)-1-benzothiophen-2-yl)methyl)-3,4-dimethoxybenzenesulfonamide;
N-((2-chlorophenyl)(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N-((2-chlorophenyl)(7-(4-(2,2,2-trifluoro-1-hydroxy-1-methylethyl)-2-pyridinyl)-1-benzothiophen-2-yl)methyl)-3,4-dimethoxybenzenesulfonamide;
N-((2-chlorophenyl)(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N-((2-chlorophenyl)(7-(4-(2,2,2-trifluoro-1-hydroxy-1-methylethyl)-2-pyridinyl)-1-benzothiophen-2-yl)methyl)-3,4-dimethoxybenzenesulfonamide; or
N-((2-amino-4-pyridinyl)(1-benzofuran-2-yl)methyl)-3,4-dihydro-2H-1,5-benzodioxepine-7-sulfonamide.

14. A compound, or a pharmaceutically acceptable salt, selected from:
N—((R)-1-benzofuran-2-yl(phenyl)methyl)-3,4-dihydro-2H-1,5-benzodioxepine-7-sulfonamide;
N—((S)-1-benzofuran-2-yl(phenyl)methyl)-3,4-dihydro-2H-1,5-benzodioxepine-7-sulfonamide;
N—((R)-1-benzofuran-2-yl(2-methylphenyl)methyl)-3,4-dihydro-2H-1,5-benzodioxepine-7-sulfonamide;
N—((R)-1-benzofuran-2-yl(4-fluoro-2-methylphenyl)methyl)-3,4-dihydro-2H-1,5-benzodioxepine-7-sulfonamide;
N—((R)-1-benzofuran-2-yl(5-fluoro-2-methylphenyl)methyl)-3,4-dihydro-2H-1,5-benzodioxepine-7-sulfonamide;
N—((R)-1-benzofuran-2-yl(2-fluoro-6-methylphenyl)methyl)-3,4-dihydro-2H-1,5-benzodioxepine-7-sulfonamide;
N—((R)-1-benzofuran-2-yl(2-ethylphenyl)methyl)-3,4-dihydro-2H-1,5-benzodioxepine-7-sulfonamide;
N—((R)-1-benzofuran-2-yl(2-ethenylphenyl)methyl)-3,4-dihydro-2H-1,5-benzodioxepine-7-sulfonamide;
N—((R)-1-benzofuran-2-yl(2-ethynylphenyl)methyl)-3,4-dihydro-2H-1,5-benzodioxepine-7-sulfonamide;
N—((R)-1-benzofuran-2-yl(2-(1-methylethyl)phenyl)methyl)-3,4-dihydro-2H-1,5-benzodioxepine-7-sulfonamide;
N—((R)-1-benzofuran-2-yl (2,6-dimethylphenyl)methyl)-3,4-dihydro-2H-1,5-benzodioxepine-7-sulfonamide;
N—((R)-1-benzofuran-2-yl(2-propylphenyl)methyl)-3,4-dihydro-2H-1,5-benzodioxepine-7-sulfonamide;
N—((R)-1-benzofuran-2-yl(2-(trifluoromethyl)phenyl)methyl)-3,4-dihydro-2H-1,5-benzodioxepine-7-sulfonamide;
N—((R)-1-benzofuran-2-yl(3-hydroxyphenyl)methyl)-3,4-dihydro-2H-1,5-benzodioxepine-7-sulfonamide;
N—((R)-(3-aminophenyl)(1-benzofuran-2-yl)methyl)-3,4-dihydro-2H-1,5-benzodioxepine-7-sulfonamide;
N—((R)-1-benzofuran-2-yl(2-(hydroxymethyl)phenyl)methyl)-3,4-dihydro-2H-1,5-benzodioxepine-7-sulfonamide;
N—((R)-1-benzofuran-2-yl(2-(2-hydroxyethyl)phenyl)methyl)-3,4-dihydro-2H-1,5-benzodioxepine-7-sulfonamide;
N—((R)-1-benzofuran-2-yl(2-methoxyphenyl)methyl)-3,4-dihydro-2H-1,5-benzodioxepine-7-sulfonamide;
N—((R)-1-benzofuran-2-yl(2-(trifluoromethoxy)phenyl)methyl)-3,4-dihydro-2H-1,5-benzodioxepine-7-sulfonamide;
N—((R)-1-benzofuran-2-yl(2-(methylsulfanyl)phenyl)methyl)-3,4-dihydro-2H-1,5-benzodioxepine-7-sulfonamide;
N—((R)-1-benzofuran-2-yl(2-chlorophenyl)methyl)-3,4-dihydro-2H-1,5-benzodioxepine-7-sulfonamide;
N—((R)-1-benzofuran-2-yl(2-iodophenyl)methyl)-3,4-dihydro-2H-1,5-benzodioxepine-7-sulfonamide;
N—((R)-1-benzofuran-2-yl(2-bromophenyl)methyl)-3,4-dihydro-2H-1,5-benzodioxepine-7-sulfonamide;
N—((R)-1-benzofuran-2-yl(2-fluorophenyl)methyl)-3,4-dihydro-2H-1,5-benzodioxepine-7-sulfonamide;
N—((R)-1-benzofuran-2-yl(3-fluorophenyl)methyl)-3,4-dihydro-2H-1,5-benzodioxepine-7-sulfonamide;
N—((R)-1-benzofuran-2-yl(2-pyridinyl)methyl)-3,4-dihydro-2H-1,5-benzodioxepine-7-sulfonamide;
N—((R)-1-benzofuran-2-yl(3-methyl-2-pyridinyl)methyl)-3,4-dihydro-2H-1,5-benzodioxepine-7-sulfonamide;
N—((R)-1-benzofuran-2-yl(2-chloro-3-pyridinyl)methyl)-3,4-dihydro-2H-1,5-benzodioxepine-7-sulfonamide;
N—((R)-1-benzofuran-2-yl(3-chloro-4-pyridinyl)methyl)-3,4-dihydro-2H-1,5-benzodioxepine-7-sulfonamide;
N—((R)-1-benzofuran-2-yl(2-pyrimidinyl)methyl)-3,4-dihydro-2H-1,5-benzodioxepine-7-sulfonamide;
N—((R)-1-benzofuran-2-yl(3-methoxy-2-pyridinyl)methyl)-3,4-dihydro-2H-1,5-benzodioxepine-7-sulfonamide;
N—((R)-1-benzofuran-2-yl(2-thiophenyl)methyl)-3,4-dihydro-2H-1,5-benzodioxepine-7-sulfonamide;
N—((R)-1-benzofuran-2-yl(3-thiophenyl)methyl)-3,4-dihydro-2H-1,5-benzodioxepine-7-sulfonamide;
N—((R)-1-benzofuran-2-yl(3-methyl-2-thiophenyl)methyl)-3,4-dihydro-2H-1,5-benzodioxepine-7-sulfonamide;
N—((R)-1-benzofuran-2-yl(1,3-thiazol-2-yl)methyl)-3,4-dihydro-2H-1,5-benzodioxepine-7-sulfonamide;
N—((R)-1-benzofuran-2-yl(phenyl)methyl)-3-fluoro-3,4-dihydro-2H-1,5-benzodioxepine-7-sulfonamide;
N—((R)-1-benzothiophen-2-yl(2-methylphenyl)methyl)-3,4-dihydro-2H-1,5-benzodioxepine-7-sulfonamide;
N—((R)-1-benzothiophen-2-yl(2-ethylphenyl)methyl)-3,4-dihydro-2H-1,5-benzodioxepine-7-sulfonamide;
N—((R)-1-benzothiophen-2-yl(2-ethenylphenyl)methyl)-3,4-dihydro-2H-1,5-benzodioxepine-7-sulfonamide;
N—((R)-1-benzothiophen-2-yl(2-ethynylphenyl)methyl)-3,4-dihydro-2H-1,5-benzodioxepine-7-sulfonamide;
N—((R)-1-benzothiophen-2-yl(2-methoxyphenyl)methyl)-3,4-dihydro-2H-1,5-benzodioxepine-7-sulfonamide;
N—((R)-1-benzothiophen-2-yl(2-(methylsulfanyl)phenyl)methyl)-3,4-dihydro-2H-1,5-benzodioxepine-7-sulfonamide;
N—((R)-1-benzothiophen-2-yl(2-chlorophenyl)methyl)-3,4-dihydro-2H-1,5-benzodioxepine-7-sulfonamide;
N—((R)-1-benzothiophen-2-yl(3-methoxy-2-pyridinyl)methyl)-3,4-dihydro-2H-1,5-benzodioxepine-7-sulfonamide;
N—((R)-(3-methyl-1-benzofuran-2-yl)(phenyl)methyl)-3,4-dihydro-2H-1,5-benzodioxepine-7-sulfonamide;
N—((R)-(3-methyl-1-benzofuran-2-yl)(2-methylphenyl)methyl)-3,4-dihydro-2H-1,5-benzodioxepine-7-sulfonamide;
N—((R)-phenyl(thieno[2,3-b]pyridin-2-yl)methyl)-3,4-dihydro-2H-1,5-benzodioxepine-7-sulfonamide;
N—((R)-(2-methoxyphenyl)(thieno[2,3-b]pyridin-2-yl)methyl)-3,4-dihydro-2H-1,5-benzodioxepine-7-sulfonamide;
N—((R)-(2-(methylsulfanyl)phenyl)(thieno[2,3-b]pyridin-2-yl)methyl)-3,4-dihydro-2H-1,5-benzodioxepine-7-sulfonamide;
N—((R)-(3-methoxy-2-pyridinyl)(thieno[2,3-b]pyridin-2-yl)methyl)-3,4-dihydro-2H-1,5-benzodioxepine-7-sulfonamide;
N—((R)-(3-methoxy-4-pyridinyl)(thieno[2,3-b]pyridin-2-yl)methyl)-3,4-dihydro-2H-1,5-benzodioxepine-7-sulfonamide;
N—((R)-1,3-benzothiazol-2-yl(2-methoxyphenyl)methyl)-3,4-dihydro-2H-1,5-benzodioxepine-7-sulfonamide;
N—((R)-1,3-benzothiazol-2-yl(2-(methylsulfanyl)phenyl)methyl)-3,4-dihydro-2H-1,5-benzodioxepine-7-sulfonamide;
N—((R)-1,3-benzothiazol-2-yl(3-methoxy-2-pyridinyl)methyl)-3,4-dihydro-2H-1,5-benzodioxepine-7-sulfonamide;
N—((R)-1,3-benzoxazol-2-yl(phenyl)methyl)-3,4-dihydro-2H-1,5-benzodioxepine-7-sulfonamide;
N—((R)-2-naphthalenyl(phenyl)methyl)-3,4-dihydro-2H-1,5-benzodioxepine-7-sulfonamide;
N—((R)-phenyl(2-quinolinyl)methyl)-3,4-dihydro-2H-1,5-benzodioxepine-7-sulfonamide;
N—((R)-1-benzothiophen-2-yl(phenyl)methyl)-3,4-dihydro-2H-1,5-benzodioxepine-7-sulfonamide;
N—((R)-(5-methoxy-1-benzothiophen-2-yl)(phenyl)methyl)-3,4-dihydro-2H-1,5-benzodioxepine-7-sulfonamide;
N—((R)-1,3-benzothiazol-2-yl(phenyl)methyl)-3,4-dihydro-2H-1,5-benzodioxepine-7-sulfonamide;
N—((R)-phenyl(thieno[2,3-c]pyridin-2-yl)methyl)-3,4-dihydro-2H-1,5-benzodioxepine-7-sulfonamide;
N—((R)-phenyl(thieno[3,2-b]pyridin-2-yl)methyl)-3,4-dihydro-2H-1,5-benzodioxepine-7-sulfonamide;
N—((R)-phenyl(thieno[3,2-c]pyridin-2-yl)methyl)-3,4-dihydro-2H-1,5-benzodioxepine-7-sulfonamide;
N—((R)-(5-hydroxy-1-benzothiophen-2-yl)(phenyl)methyl)-3,4-dihydro-2H-1,5-benzodioxepine-7-sulfonamide;
N—((R)-2-(methylsulfanyl)phenyl)(thieno[2,3-c]pyridin-2-yl)methyl)-3,4-dihydro-2H-1,5-benzodioxepine-7-sulfonamide;
N—((R)-2-(methylsulfanyl)phenyl)([1,3]thiazolo[5,4-b]pyridin-2-yl)methyl)-3,4-dihydro-2H-1,5-benzodioxepine-7-sulfonamide;
N—((R)-2-methoxyphenyl)(thieno[2,3-c]pyridin-2-yl)methyl)-3,4-dihydro-2H-1,5-benzodioxepine-7-sulfonamide;
N—((R)-2-methoxyphenyl)([1,3]thiazolo[5,4-b]pyridin-2-yl)methyl)-3,4-dihydro-2H-1,5-benzodioxepine-7-sulfonamide;
N—((R)-2-chlorophenyl)([1,3]thiazolo[5,4-b]pyridin-2-yl)methyl)-3,4-dihydro-2H-1,5-benzodioxepine-7-sulfonamide;
N—((R)-3-methoxy-2-pyridinyl)([1,3]thiazolo[5,4-b]pyridin-2-yl)methyl)-3,4-dihydro-2H-1,5-benzodioxepine-7-sulfonamide;
N—((R)-phenyl(7-(4-(2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N—((R)-phenyl(7-(2-pyridinyl)-1-benzothiophen-2-yl)methyl)-3,4-dihydro-2H-1,5-benzodioxepine-7-sulfonamide;
N—((R)-7-chloro-1-benzothiophen-2-yl)(phenyl)methyl)-3,4-dihydro-2H-1,5-benzodioxepine-7-sulfonamide;
N—((R)-phenyl(7-phenyl-1-benzothiophen-2-yl)methyl)-3,4-dihydro-2H-1,5-benzodioxepine-7-sulfonamide;
N—((R)-1-benzothiophen-2-yl(phenyl)methyl)-3,4-dimethoxybenzenesulfonamide; or
N—((R)-1-benzothiophen-2-yl(phenyl)methyl)-2,3-dihydro-1,4-benzodioxine-6-sulfonamide.

15. A compound, or a pharmaceutically acceptable salt, selected from:
N—((R)-1-benzothiophen-2-yl(2-chlorophenyl)methyl)-3,3-difluoro-3,4-dihydro-2H-1,5-benzodioxepine-7-sulfonamide;
N—((R)-1-benzothiophen-2-yl(2-chlorophenyl)methyl)-3-hydroxy-3,4-dihydro-2H-1,5-benzodioxepine-7-sulfonamide;
N—((R)-1-benzothiophen-2-yl(2-chlorophenyl)methyl)-3-fluoro-3,4-dihydro-2H-1,5-benzodioxepine-7-sulfonamide;
N—((R)-(2-chlorophenyl)(7-(4-(2,2,2-trifluoro-1-hydroxy-1-(trifluoromethyl)ethyl)-2-pyridinyl)-1-benzothiophen-2-yl)methyl)-3,4-dihydro-2H-1,5-benzodioxepine-7-sulfonamide;
N—((R)-(2-chlorophenyl)(7-(4-(2,2,2-trifluoro-1-hydroxy-1-(trifluoromethyl)ethyl)-2-pyridinyl)-1-benzothiophen-2-yl)methyl)-3,4-dimethoxybenzenesulfonamide;
N—((R)-(2-chlorophenyl)(7-(4-((1R)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N—((R)-(2-chlorophenyl)(7-(4-((1R)-2,2,2-trifluoro-1-hydroxy-1-methylethyl)-2-pyridinyl)-1-benzothiophen-2-yl)methyl)-3,4-dimethoxybenzenesulfonamide;
N—((R)-(2-chlorophenyl)(7-(4-((1S)-2,2,2-trifluoro-1-hydroxy-1-methylethyl)-2-pyridinyl)-1-benzothiophen-2-yl)methyl)-3,4-dihydro-2H-1,5-benzodioxepine-7-sulfonamide;
N—((R)-(2-Chlorophenyl)(7-(4-((1S)-2,2,2-trifluoro-1-hydroxy-1-methylethyl)-2-pyridinyl)-1-benzothiophen-2-yl)methyl)-3,4-dimethoxybenzenesulfonamide; or
N-((2-amino-4-pyridinyl)(1-benzofuran-2-yl)methyl)-3,4-dihydro-2H-1,5-benzodioxepine-7-sulfonamide.

16. A method of treating type 2 diabetes, hyperglycemia, impaired glucose tolerance, insulin resistance, retinopathy, nephropathy, neuropathy, cataracts, glaucoma, Syndrome X, or polycystic ovarian syndrome, the method comprising administering to a patient in need thereof a therapeutically effective amount of a compound in accordance with any one of claims 1, 2, and 12 to 15, or a pharmaceutically acceptable salt thereof.

17. The methods of claim 16 wherein the method is the treatment of type 2 diabetes.

18. The methods of claim 16 wherein the methods further comprise administering metformin.

19. The methods of claim 16 wherein the methods further comprise administering a DPP-IV inhibitor, or a pharmaceutically acceptable salt thereof.

20. The methods of claim 16 wherein the methods further comprise administering a sulfonylurea, or a pharmaceutically acceptable salt thereof.

21. The methods of claim 16 wherein the methods further comprise administering a PPARγ agonist, or a pharmaceutically acceptable salt thereof.

22. A pharmaceutical composition comprising a compound in accordance with any one of claims 1, 2, and 12 to 15, or a pharmaceutically acceptable salt thereof, and a pharmaceutically acceptable excipient.
